SEQUENCE LISTING (271 sequences)

SEQ ID NO: 1 - protein, 260 aa, Homo sapiens
MSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 2 - DNA, 1492 bp, Homo sapiens
  source 1..1492: mol_type = other DNA
acacagtgcaggcgcccaagccgccgccgccagatcggtgccgattcctgccctgccccgaccgccagcgcgaccatgtcccatcactgggggtacggcaaacacaacggacctgagcactggcataaggacttccccatcccctgtctgtttcctatgatcaagcaacttccctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccttggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaataagatggtcccatagtctgtatccaaataatgaatcttcgggtgtttccctttagctaagcacagatctaccttggtgatttggaccctggttgctttgtgtctagttttctagacccttcatctcttacttgatagacttactaataaaatgtgaagactagaccaattgtcatgcttgacacaactgctgtggctggttggtgctttgtttatggtagtagtttttctgtaacacagaatataggataagaaataagaataaagtaccttgactttgttcacagcatgtagggtgatgagcactcacaattgttgactaaaatgctgcttttaaaacataggaaagtagaatggttgagtgcaaatccatagcacaagataaattgagctagttaaggcaaatcaggtaaaatagtcatgattctatgtaatgtaaaccagaaaaaataaatgttcatgatttcaagatgttatattaaagaaaaactttaaaaattattatatatttatagcaaagttatcttaaatatgaattctgttgtaatttaatgacttttgaattacagagatataaatgaagtattatctgtaaaaattgttataattagagttgtgatacagagtatatttccattcagacaatatatcataacttaataaatattgtattttagatatattctctaataaaattcagaattcta

SEQ ID NO: 3 - protein, 259 aa, Homo sapiens
SHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 4 - DNA, 777 bp, Homo sapiens
  source 1..777: mol_type = other DNA
tcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaa

SEQ ID NO: 5 - protein, 259 aa, synthetic construct
  REGION 1..259: note = Description of Artificial Sequence: Synthetic polypeptide
SHHWGYGKHNGPEHWHKDFPIAKGERQSPVDIDTHTAKYDPSLKPLSVSYDQATSLRILNNGHAFNVEFDDSQDKAVHKGGPLDGTYRLIQFHFHWGSLDGQGSEHTVDKKKYAAELHLVHWNTKYGDFGKAVQQPDGLAVLGIFLKVGSAKPGHQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 6 - DNA, 616 bp, synthetic construct
  misc_feature 1..616: note = Description of Artificial Sequence: Synthetic polynucleotide
  source 1..616: mol_type = other DNA
tcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgagaatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccatcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtga

SEQ ID NO: 7 - protein, 260 aa, synthetic construct
  REGION 1..260: note = Description of Artificial Sequence: Synthetic polypeptide
MSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 8 - DNA, 780 bp, synthetic construct
  misc_feature 1..780: note = Description of Artificial Sequence: Synthetic polynucleotide
  source 1..780: mol_type = other DNA
atgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaa

SEQ ID NO: 9 - protein, 259 aa, synthetic construct
  REGION 1..259: note = Description of Artificial Sequence: Synthetic polypeptide
SHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 10 - DNA, 777 bp, synthetic construct
  misc_feature 1..777: note = Description of Artificial Sequence: Synthetic polynucleotide
  source 1..777: mol_type = other DNA
tcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaa

SEQ ID NO: 11 - protein, 40 aa, synthetic construct
  REGION 1..40: note = Description of Artificial Sequence: Synthetic polypeptide
LDFACDLLPSWAITLISVNGIFVICCLTYCFAPRCRERRR

SEQ ID NO: 12 - DNA, 120 bp, synthetic construct
  misc_feature 1..120: note = Description of Artificial Sequence: Synthetic polynucleotide
  source 1..120: mol_type = other DNA
ctggactttgcctgcgatctgttgccgagctgggcgattacgcttatcagtgtaaacggcatctttgtaatatgctgtctgacctactgcttcgcaccaaggtgccgggagagaaggagg

SEQ ID NO: 13 - protein, 79 aa, Homo sapiens
TTTPAPRPPTPAPTIASQPLSLRPEACRPAAGGAVHTRGLDFACDIYIWAPLAGTCGVLLLSLVITLYCNHRNRRRVCK

SEQ ID NO: 14 - DNA, 237 bp, Homo sapiens
  source 1..237: mol_type = other DNA
accacaaccccagcacctcgcccaccaacaccagcaccaaccatcgcatcccagccactgtctctgaggcccgaggcatgtaggcctgcagcaggaggcgccgtgcacaccaggggcctggactttgcctgcgatatctatatctgggcaccactggcaggaacatgtggcgtgctgctgctgtccctggtcatcaccctgtattgcaatcatcgcaaccgccgaagggtctgcaag

SEQ ID NO: 15 - protein, 146 aa, Homo sapiens
GDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 16 - DNA, 438 bp, Homo sapiens
  source 1..438: mol_type = other DNA
ggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactc

SEQ ID NO: 17 - protein, 41 aa, Homo sapiens
VHLHTEARARHAWQLTQGATVLGLFRVTPEIPAGLPSPRSE

SEQ ID NO: 18 - protein, 159 aa, Escherichia coli [sequence omitted]

SEQ ID NO: 19 - protein, 61 aa, Homo sapiens
GASSGVRLWATRQAMLGQVHEVPEGWLIFVAEQEELYVRVQNGFRKVQLEARTPLPRGTDN

SEQ ID NO: 20 - DNA, 183 bp, Homo sapiens
  source 1..183: mol_type = other DNA
ggtgcttcttctggtgttagactgtgggccaccagacaggccatgctgggacaagtgcacgaagtgcctgaaggctggctgatcttcgtggccgagcaagaggaactgtacgtgcgggtgcagaacggcttcagaaaggtgcagctggaagccagaacacccctgcctagaggcaccgacaat

SEQ ID NO: 21 - protein, 149 aa, synthetic construct
  REGION 1..149: note = Description of Artificial Sequence: Synthetic polypeptide
SLRQQVEALQGQVQHLQAAFSQYKKVELFPNGQSVGEKIFKTAGFVKPFTEAQLLCTQAGGQLASPRSAENAALQQLVVAKNEAAFLSMTDSKTEGKFTYPTGESLVYSNWAPGEPNDDGGSEDCVEIFTNGKWNDRACGEKRLVVCEF

SEQ ID NO: 22 - DNA, 450 bp, synthetic construct
  misc_feature 1..450: note = Description of Artificial Sequence: Synthetic polynucleotide
  source 1..450: mol_type = other DNA
agcctgagacagcaggttgaagccctgcaaggccaggtgcagcatctgcaggctgccttcagccagtacaagaaggtggaactgttccccaacggccagagcgtgggcgagaagatctttaagaccgccggcttcgtgaagcccttcacagaggctcagctgctgtgtactcaggctggtggacagctggctagccctagatctgccgccgaaaatgctgctctgcagcagctggtggtggccaagaatgaagccgccttcctgagcatgaccgacagcaagaccgagggcaagttcacataccctaccggcgagagcctggtgtacagcaattgggctcctggcgagcccaatgatgatggcggctctgaggactgcgtggaaatcttcaccaacggcaagtggaacgacagagcctgcggagagaagagactggtcgtgtgcgaattt

SEQ ID NO: 23 - protein, 41 aa, Homo sapiens
SGLPDVASLRQQVEALQGQVQHLQAAFSQYKKVELFPNGQS

SEQ ID NO: 24 - DNA, 123 bp, Homo sapiens
  source 1..123: mol_type = other DNA
tctggactgcctgatgtggccagcctgagacagcaggttgaagccctgcaaggccaggtgcagcatctgcaggctgccttcagccagtacaagaaggtggaactgttccccaacggccagagc

SEQ ID NO: 25 - protein, 152 aa, Homo sapiens
SLENMSKLLKRQNDILQVVSQGWKYFKGNFYYFSLIPKTWYSAEQFCVSRNSHLTSVTSESEQEFLYKTAGGLIYWIGLTKAGMEGDWSWVDDTPFNKVQSVRFWIPGEPNNAGNNEHCGNIKAPSLQAWNDAPCDKTFLFICKRPYVPSEP

SEQ ID NO: 26 - DNA, 456 bp, Homo sapiens
  source 1..456: mol_type = other DNA
agcctggaaaacatgagcaagctgctgaagcggcagaacgacatcctgcaggtcgtgtcccaaggctggaagtactttaagggcaacttctactacttcagtctgatccccaagacctggtacagcgccgagcagttctgcgtgtccagaaacagccacctgaccagcgtgacaagcgagagcgagcaagagttcctgtacaagacagccggcggactgatctactggatcggcctgacaaaggccggcatggaaggcgattggagctgggtcgacgacacccctttcaacaaggtgcagagcgtgcggttctggatccctggcgaacctaacaacgccggcaacaatgagcactgcggcaacatcaaggccccatctctgcaggcctggaacgatgccccttgcgacaagaccttcctgttcatctgcaagcggccctacgtgccatctgagcct

SEQ ID NO: 27 - protein, 149 aa, Homo sapiens
MQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 28 - DNA, 447 bp, Homo sapiens
  source 1..447: mol_type = other DNA
atgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactc

SEQ ID NO: 29 - protein, 261 aa, synthetic construct
  REGION 1..261: note = Description of Artificial Sequence: Synthetic polypeptide
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENGGSGGSGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 30 - DNA, 783 bp, synthetic construct
  misc_feature 1..783: note = Description of Artificial Sequence: Synthetic polynucleotide
  source 1..783: mol_type = other DNA
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacggtggaagtggtggaagtggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactc

SEQ ID NO: 31 - protein, 21 aa, Homo sapiens
FINFCLILICLLLICIIVMLL

SEQ ID NO: 32 - DNA, 63 bp, Homo sapiens
  source 1..63: mol_type = other DNA
ttcatcaatttctgcctgatcctgatctgcctgctgctcatctgcattatcgtgatgctgctg

SEQ ID NO: 33 - protein, 46 aa, synthetic construct
  REGION 1..46: note = Description of Artificial Sequence: Synthetic polypeptide
GGGGSSGLPDVASLRQQVEALQGQVQHLQAAFSQYKKVELFPNGQS

SEQ ID NO: 34 - DNA, 138 bp, synthetic construct
  misc_feature 1..138: note = Description of Artificial Sequence: Synthetic polynucleotide
  source 1..138: mol_type = other DNA
ggcggaggcggatcttctggactgcctgatgtggccagcctgagacagcaggttgaagccctgcaaggccaggtgcagcatctgcaggctgccttcagccagtacaagaaggtggaactgttccccaacggccagagc

SEQ ID NO: 35 - protein, 40 aa, Homo sapiens [sequence omitted]

SEQ ID NO: 36 - DNA, 126 bp, Homo sapiens
  source 1..126: mol_type = other DNA
tctagcgactatagcgatttgcaacgggtgaaacaagaactgttggaagaagttaaaaaggagttgcagaaggtcaaggaggaaattattgaagccttcgtccaggaacttaggaaaagggggagc

SEQ ID NO: 37 - protein, 187 aa, Homo sapiens
MVGSLNCIVAVSQNMGIGKNGDLPWPPLRNEFRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

SEQ ID NO: 38 - DNA, 561 bp, Homo sapiens
  source 1..561: mol_type = other DNA
atggttggttcgctaaactgcatcgtcgctgtgtcccagaacatgggcatcggcaagaacggggacctgccctggccaccgctcaggaatgaattcagatatttccagagaatgaccacaacctcttcagtagaaggtaaacagaatctggtgattatgggtaagaagacctggttctccattcctgagaagaatcgacctttaaagggtagaattaatttagttctcagcagagaactcaaggaacctccacaaggagctcattttctttccagaagtctagatgatgccttaaaacttactgaacaaccagaattagcaaataaagtagacatggtctggatagttggtggcagttctgtttataaggaagccatgaatcacccaggccatcttaaactatttgtgacaaggatcatgcaagactttgaaagtgacacgttttttccagaaattgatttggagaaatataaacttctgccagaatacccaggtgttctctctgatgtccaggaggagaaaggcattaagtacaaatttgaagtatatgagaagaatgat

SEQ ID NO: 39 - protein, 29 aa, Homo sapiens
ARQKLQNLFINFCLILICLLLICIIVMLL

SEQ ID NO: 40 - DNA, 87 bp, Homo sapiens
  source 1..87: mol_type = other DNA
gccagacagaagctgcagaacctgttcatcaatttctgcctgatcctgatctgcctgctgctcatctgcattatcgtgatgctgctg

SEQ ID NO: 41 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 42 - DNA, 762 bp, Homo sapiens
  source 1..762: mol_type = other DNA
atggagtacgctagtgatgcatccttagatcctgaagcgccatggccacctgctccacgagctcgagcctgtcgagtgctgccatgggctctggtcgctgggttgctcctccttctgcttttggccgcggcttgtgcagtgtttcttgcttgcccgtgggcagttagcggtgctcgcgcatctcccggaagcgcggcgagtcctcgactcagggaaggtccggagctgagcccagatgaccccgccggtttgctggacctccgccaaggaatgttcgctcaactcgttgcgcaaaacgtacttcttatagacggccctcttagttggtacagtgacccaggattggctggcgttagtttgacaggcggactcagttacaaggaggatactaaggaactggtagtcgctaaggctggggtatactacgtgttctttcaactcgaactgagaagggtggttgcgggagaaggatctggaagtgtatctctcgccctgcacctccaacccctcagaagtgccgccggagcggccgcccttgcccttactgtcgacctgcccccggcttcttcagaagcgcgaaatagtgcattcggcttccaggggcgccttttgcacttgagcgctggacagcgcctcggggtccacctccacacggaagcgcgggcgaggcacgcttggcaactcacacaaggtgcgacggttctcggcttgtttagggttacgcctgagataccggctggcctcccatctccaagatccgag

SEQ ID NO: 43 - protein, 155 aa, Homo sapiens
ERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 44 - DNA, 465 bp, Homo sapiens
  source 1..465: mol_type = other DNA
gagcggagaagccacgaagtgaaccctgccgcacatctgacaggcgccaattctagcctgacaggctctggtggacccctgctgtgggaaactcaactgggactcgccttcctgagaggcctgagctatcatgatggcgccctggtggttaccaaggccggctactactacatctacagcaaggtgcagctcggcggcgtgggatgtcctcttggactggcctctacaatcacccacggcctgtacaagcggacccctagataccccgaggaactggaactgctggtgtcccagcagagcccttgtggcagagccacaagcagctccagagtttggtgggacagcagctttctcggcggagtggtgcatctggaagccggcgaaaaggtggtcgtcagagtgctggatgagagactcgtgcggctgagagatggcacccggtcttacttcggcgccttcatggtg

SEQ ID NO: 45 - protein, 109 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKEN

SEQ ID NO: 46 - DNA, 327 bp, Homo sapiens
  source 1..327: mol_type = other DNA
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaac

SEQ ID NO: 47 - protein, 115 aa, synthetic construct
  REGION 1..115: note = Description of Artificial Sequence: Synthetic polypeptide
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENGGSGGS

SEQ ID NO: 48 - DNA, 345 bp, synthetic construct
  misc_feature 1..345: note = Description of Artificial Sequence: Synthetic polynucleotide
  source 1..345: mol_type = other DNA
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacggtggaagtggtggaagt

SEQ ID NO: 49 - protein, 115 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQK

SEQ ID NO: 50 - DNA, 345 bp, Homo sapiens
  source 1..345: mol_type = other DNA
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaa

SEQ ID NO: 51 - protein, 54 aa, Homo sapiens
KQEHFPDNLLPSWAITLISVNGIFVICCLTYCFAPRCRERRRNERLRRESVRPV

SEQ ID NO: 52 - DNA, 162 bp, Homo sapiens
  source 1..162: mol_type = other DNA
aaacaagagcactttcctgataacctgttgccgagctgggcgattacgcttatcagtgtaaacggcatctttgtaatatgctgtctgacctactgcttcgcaccaaggtgccgggagagaaggagaaatgaaagactgagaagggagagcgtgagacctgtg

SEQ ID NO: 53 - protein, 41 aa, Homo sapiens
KQEHFPDNLLPSWAITLISVNGIFVICCLTYCFAPRCRERR

SEQ ID NO: 54 - DNA, 123 bp, Homo sapiens
  source 1..123: mol_type = other DNA
aaacaagagcactttcctgataacctgttgccgagctgggcgattacgcttatcagtgtaaacggcatctttgtaatatgctgtctgacctactgcttcgcaccaaggtgccgggagagaagg

SEQ ID NO: 55 - protein, 79 aa, Homo sapiens; identical to SEQ ID NO: 13

SEQ ID NO: 56 - DNA, 237 bp, Homo sapiens; identical to SEQ ID NO: 14
  source 1..237: mol_type = other DNA

SEQ ID NO: 57 - protein, 54 aa, Homo sapiens
PREPPPKSGPSLVPGKTPTVRAALICLTLVLVASVLLQAVLYPRFMGTISDVKT

SEQ ID NO: 58 - DNA, 162 bp, Homo sapiens
  source 1..162: mol_type = other DNA
cctagagagcctcctccaaagtctggcccttctctggtgcctggcaagacccctacagttagagccgctctgatctgtctgaccctggtgctggttgctagcgtgctgctgcaggctgtgctgtaccctagattcatgggcaccatcagcgacgtgaaaacc

SEQ ID NO: 59 - protein, 88 aa, synthetic construct
  REGION 1..88: note = Description of Artificial Sequence: Synthetic polypeptide
GSGSGSGSGSGSGSSFEMQKGGSLRPVQAQAQSDCSCSTVSPGVLAGIVMGDLVLTVLIALAVYFLGRLVPRGRGAAEAATRKQRITE

SEQ ID NO: 60 - DNA, 255 bp, synthetic construct
  misc_feature 1..255: note = Description of Artificial Sequence: Synthetic polynucleotide
  source 1..255: mol_type = other DNA
ggatctggttctggttccggaagtggatctggttcagggtccagctttgaaatgcaaaaactcagacctgttcaggcccaggctcagagcgattgcagctgtagcacagtgtctccaggcgtgctggccggaatcgtgatgggagatctggtgctgacagtgctgattgccctggccgtgtacttcctgggtagactggtgcctagaggaagaggcgctgccgaagccgccacaagaaagcagagaatcacagaa

SEQ ID NO: 61 - protein, 65 aa, Homo sapiens
LRPVQAQAQSDCSCSTVSPGVLAGIVMGDLVLTVLIALAVYFLGRLVPRGRGAAEAATRKQRITE

SEQ ID NO: 62 - DNA, 195 bp, Homo sapiens
  source 1..195: mol_type = other DNA
ctcagacctgttcaggcccaggctcagagcgattgcagctgtagcacagtgtctccaggcgtgctggccggaatcgtgatgggagatctggtgctgacagtgctgattgccctggccgtgtacttcctgggtagactggtgcctagaggaagaggcgctgccgaagccgccacaagaaagcagagaatcacagaa

SEQ ID NO: 63 - protein, 86 aa, synthetic construct
  REGION 1..86: note = Description of Artificial Sequence: Synthetic polypeptide
GSGSGSGSGSGSGSSFEMQKGGSLTLRWELSSQPTIPIVGIIAGLVLLGAVITGAVVAAVMWRRKSSDRKGGSYTQAASSDSAQGS

SEQ ID NO: 64 - DNA, 258 bp, synthetic construct
  misc_feature 1..258: note = Description of Artificial Sequence: Synthetic polynucleotide
  source 1..258: mol_type = other DNA
ggatctggttctggttccggaagtggatctggttcagggtccagctttgaaatgcaaaaaggcggtagtctgactcttaggtgggaactgagcagtcagcccactatccccattgtcggtatcattgccggactcgttttgcttggtgctgtgatcactggagcggtggtcgcggcagtgatgtggcggcgaaagtcaagcgacagaaagggcggctcctatactcaagccgcctcaagtgactctgcacagggtagc

SEQ ID NO: 65 - protein, 63 aa, Homo sapiens
LTLRWELSSQPTIPIVGIIAGLVLLGAVITGAVVAAVMWRRKSSDRKGGSYTQAASSDSAQGS

SEQ ID NO: 66 - DNA, 189 bp, Homo sapiens
  source 1..189: mol_type = other DNA
ctgactcttaggtgggaactgagcagtcagcccactatccccattgtcggtatcattgccggactcgttttgcttggtgctgtgatcactggagcggtggtcgcggcagtgatgtggcggcgaaagtcaagcgacagaaagggcggctcctatactcaagccgcctcaagtgactctgcacagggtagc

SEQ ID NO: 67 - protein, 86 aa, synthetic construct
  REGION 1..86: note = Description of Artificial Sequence: Synthetic polypeptide
DGQTDIPFTRLGRSHRRQSCSVARVGLGLLLLLMGAGLAVQGWFLLQLHWRLGEMVTRLPDGPAGSWEQLIQGSGSGSGSGSGSGS

SEQ ID NO: 68 - DNA, 258 bp, synthetic construct
  misc_feature 1..258: note = Description of Artificial Sequence: Synthetic polynucleotide
  source 1..258: mol_type = other DNA
gacggccagacagacatccccttcacaagactgggcagaagccacagacggcagagctgttctgtggccagagttggactgggactgctgctgcttctgatgggagctggactggctgtgcaaggctggtttctgctgcagctccattggagactgggcgagatggtcaccagactgcctgatggacctgccggatcttgggagcagctgatccaaggttccggatctggaagtggatctggtagtggttccggtagt

SEQ ID NO: 69 - protein, 84 aa, synthetic construct
  REGION 1..84: note = Description of Artificial Sequence: Synthetic polypeptide
DGQTDIPFTRLGRSHRRQSCSVARVGLGLLLLLMGAGLAVQGWFLLQLHWRLGEMVTRLPDGPAGSWEQLIQGSGGSGGSGGSG

SEQ ID NO: 70 - DNA, 252 bp, synthetic construct
  misc_feature 1..252: note = Description of Artificial Sequence: Synthetic polynucleotide
  source 1..252: mol_type = other DNA
gacggccagacagacatccccttcacaagactgggcagaagccacagacggcagagctgttctgtggccagagttggactgggactgctgctgcttctgatgggagctggactggctgtgcaaggctggtttctgctgcagctccattggagactgggcgagatggtcaccagactgcctgatggacctgccggatcttgggagcagctgatccaaggaagcggaggttctggtggttctggcggtagcggc

SEQ ID NO: 71 - protein, 21 aa, Homo sapiens
MALPVTALLLPLALLLHAARP

SEQ ID NO: 72 - DNA, 63 bp, Homo sapiens
  source 1..63: mol_type = other DNA
atggctctgcctgtgacagctctgctgctgcctctggctctgcttctgcatgccgccagacct

SEQ ID NO: 73 - protein, 22 aa, Homo sapiens
MCHQQLVISWFSLVFLASPLVA

SEQ ID NO: 74 - DNA, 66 bp, Homo sapiens
  source 1..66: mol_type = other DNA
atgtgccaccaacagctcgtgatcagctggttctccctggtgttcctggctagccccctggtggcc

SEQ ID NO: 75 - protein, 24 aa, Homo sapiens
VGIIAGLVLLGAVITGAVVAAVMW

SEQ ID NO: 76 - DNA, 72 bp, Homo sapiens
  source 1..72: mol_type = other DNA
gtcggtatcattgccggactcgttttgcttggtgctgtgatcactggagcggtggtcgcggcagtgatgtgg

SEQ ID NO: 77 - protein, 21 aa, Homo sapiens
FINFCLILICLLLICIIVMLL

SEQ ID NO: 78 - DNA, 63 bp, Homo sapiens; identical to SEQ ID NO: 32
  source 1..63: mol_type = other DNA

SEQ ID NO: 79 - protein, 21 aa, Homo sapiens
GVLAGIVMGDLVLTVLIALAV

SEQ ID NO: 80 - DNA, 63 bp, Homo sapiens
  source 1..63: mol_type = other DNA
ggcgtgctggccggaatcgtgatgggagatctggtgctgacagtgctgattgccctggccgtg

SEQ ID NO: 81 - protein, 21 aa, Homo sapiens
LLPSWAITLISVNGIFVICCL

SEQ ID NO: 82 - DNA, 63 bp, Homo sapiens
  source 1..63: mol_type = other DNA
ctgttgccgagctgggcgattacgcttatcagtgtaaacggcatctttgtaatatgctgtctg

SEQ ID NO: 83 - protein, 15 aa, Homo sapiens
LTLRWELSSQPTIPI

SEQ ID NO: 84 - DNA, 45 bp, Homo sapiens
  source 1..45: mol_type = other DNA
ctgactcttaggtgggaactgagcagtcagcccactatccccatt

SEQ ID NO: 85 - protein, 8 aa, Homo sapiens
KQEHFPDN

SEQ ID NO: 86 - DNA, 24 bp, Homo sapiens
  source 1..24: mol_type = other DNA
aaacaagagcactttcctgataac

SEQ ID NO: 87 - protein, 17 aa, Homo sapiens
IPFLEQNNSSPNTRTQK

SEQ ID NO: 88 - DNA, 51 bp, Homo sapiens
  source 1..51: mol_type = other DNA
atcccattcctcgagcagaacaacagcagccccaacacacggacccagaag

SEQ ID NO: 89 - protein, 69 aa, Homo sapiens
HRRLDKIEDERNLHEDFVFMKTIQRCNTGERSLSLLNCEEIKSQFEGFVKDIMLNKEETKKENSFEMQK

SEQ ID NO: 90 - DNA, 207 bp, Homo sapiens
  source 1..207: mol_type = other DNA
catagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaa

SEQ ID NO: 91 - protein, 41 aa, synthetic construct
  REGION 1..41: note = Description of Artificial Sequence: Synthetic polypeptide
LQLHWRLGEMVTRLPDGPAGSWEQLIQGSGSGSGSGSGSGS

SEQ ID NO: 92 - DNA, 123 bp, synthetic construct
  misc_feature 1..123: note = Description of Artificial Sequence: Synthetic polynucleotide
  source 1..123: mol_type = other DNA
ctgcagctccattggagactgggcgagatggtcaccagactgcctgatggacctgccggatcttgggagcagctgatccaaggttccggaagcggaagtggatctggtagtggttccggtagt

SEQ ID NO: 93 - protein, 107 aa, Homo sapiens
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 94 - protein, 327 aa, Homo sapiens
MEETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQ

SEQ ID NO: 95 - protein, 875 aa, Homo sapiens
MERAGPSFGQQRQQQQPQQQKQQQRDQDSVEAWLDDHWDFTFSYFVRKATREMVNAWFAERVHTIPVCKEGIRGHTESCSCPLQQSPRADNSAPGTPTRKISASEFDRPLRPIVVKDSEGTVSFLSDSEKKEQMPLTPPRFDHDEGDQCSRLLELVKDISSHLDVTALCHKIFLHIHGLISADRYSLFLVCEDSSNDKFLISRLFDVAEGSTLEEVSNNCIRLEWNKGIVGHVAALGEPLNIKDAYEDPRFNAEVDQITGYKTQSILCMPIKNHREEVVGVAQAINKKSGNGGTFTEKDEKDFAAYLAFCGIVLHNAQLYETSLLENKRNQVLLDLASLIFEEQQSLEVILKKIAATIISFMQVQKCTIFIVDEDCSDSFSSVFHMECEELEKSSDTLTREHDANKINYMYAQYVKNTMEPLNIPDVSKDKRFPWTTENTGNVNQQCIRSLLCTPIKNGKKNKVIGVCQLVNKMEENTGKVKPFNRNDEQFLEAFVIFCGLGIQNTQMYEAVERAMAKQMVTLEVLSYHASAAEEETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQEKMLINGESGQAKRN

SEQ ID NO: 96 - protein, 595 aa, Homo sapiens [sequence omitted]

SEQ ID NO: 97 - protein, 23 aa, synthetic construct
  REGION 1..23: note = Description of Artificial Sequence: Synthetic peptide
GSGSGSGSGSGSGSSFEMQKGGS

SEQ ID NO: 98 - DNA, 59 bp, synthetic construct
  misc_feature 1..59: note = Description of Artificial Sequence: Synthetic oligonucleotide
  source 1..59: mol_type = other DNA
agctttgagatgcagaaaggttccggatctggaagtggatctggtagtggttccggtag

SEQ ID NO: 99 - protein, 22 aa, synthetic construct
  REGION 1..22: note = Description of Artificial Sequence: Synthetic peptide
GSGATNFSLLKQAGDVEENPGP

SEQ ID NO: 100 - DNA, 66 bp, synthetic construct
  misc_feature 1..66: note = Description of Artificial Sequence: Synthetic oligonucleotide
  source 1..66: mol_type = other DNA
ggatctggtgctactaacttcagcctgctgaagcaggctggagacgtggaggagaaccctggacct

SEQ ID NO: 101 - protein, 6 aa, Homo sapiens
SFEMQK

SEQ ID NO: 102 - DNA, 18 bp, Homo sapiens
  source 1..18: mol_type = other DNA
agctttgagatgcagaaa

SEQ ID NO: 103 - protein, 6 aa, synthetic construct
  REGION 1..6: note = Description of Artificial Sequence: Synthetic peptide
GGSGGS

SEQ ID NO: 104 - DNA, 19 bp, synthetic construct
  misc_feature 1..19: note = Description of Artificial Sequence: Synthetic oligonucleotide
  source 1..19: mol_type = other DNA
ggtggtagtggaggctccg

SEQ ID NO: 105 - protein, 24 aa, Homo sapiens
RRKSSDRKGGSYTQAASSDSAQGS

SEQ ID NO: 106 - DNA, 72 bp, Homo sapiens
  source 1..72: mol_type = other DNA
cggcgaaagtcaagcgacagaaagggcggctcctatactcaagccgcctcaagtgactctgcacagggtagc

SEQ ID NO: 107 - protein, 8 aa, Homo sapiens
ARQKLQNL

SEQ ID NO: 108 - DNA, 24 bp, Homo sapiens
  source 1..24: mol_type = other DNA
gcaagacaaaaacttcagaacctg

SEQ ID NO: 109 - protein, 22 aa, Homo sapiens
MIETYNQTSPRSAATGLPISMK

SEQ ID NO: 110 - DNA, 66 bp, Homo sapiens
  source 1..66: mol_type = other DNA
atgatagagacttacaaccaaacctccccaagatccgctgcaaccggcttgccaatatccatgaag

SEQ ID NO: 111 - protein, 25 aa, Homo sapiens
TYCFAPRCRERRRNERLRRESVRPV

SEQ ID NO: 112 - DNA, 75 bp, Homo sapiens
  source 1..75: mol_type = other DNA
acctactgcttcgcaccaaggtgccgggagagaaggagaaatgaaagactgagaagggagagcgtgagacctgtg

SEQ ID NO: 113 - protein, 24 aa, Homo sapiens
DGQTDIPFTRLGRSHRRQSCSVAR

SEQ ID NO: 114 - DNA, 72 bp, Homo sapiens
  source 1..72: mol_type = other DNA
gacggccagacagacatcccattcacaagactgggcagaagccacagacggcagagctgttctgtggccaga

SEQ ID NO: 115 - protein, 15 aa, Homo sapiens
SKQRGTFSEVSLAQD

SEQ ID NO: 116 - DNA, 45 bp, Homo sapiens
  source 1..45: mol_type = other DNA
agcaagcagagaggcaccttcagcgaagtgtctctggcccaggat

SEQ ID NO: 117 - protein, 518 aa, synthetic construct
  REGION 1..518: note = Description of Artificial Sequence: Synthetic polypeptide
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 118 - DNA, 1554 bp, synthetic construct
  misc_feature 1..1554: note = Description of Artificial Sequence: Synthetic polynucleotide
  source 1..1554: mol_type = other DNA
atctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagc

SEQ ID NO: 119 - protein, 486 aa, synthetic construct
  REGION 1..486: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 120 - DNA, 1458 bp, synthetic construct
  misc_feature 1..1458: note = Description of Artificial Sequence: Synthetic polynucleotide
  source 1..1458: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 121 - protein, 486 aa, synthetic construct; identical to SEQ ID NO: 119
  REGION 1..486: note = Description of Artificial Sequence: Synthetic polypeptide

SEQ ID NO: 122 - DNA, 1458 bp, synthetic construct
  misc_feature 1..1458: note = Description of Artificial Sequence: Synthetic polynucleotide
  source 1..1458: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 123 - protein, 1379 aa, synthetic construct
  REGION 1..1379: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSATNFSLLKQAGDVEENPGPMCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGSGSGSGSGSGSGSGSGSGSGSGSGSGSKQEHFPDNLLPSWAITLISVNGIFVICCLTYCFAPRCRERRGSSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGHQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 124 - DNA, 4137 bp, synthetic construct
  misc_feature 1..4137: note = Description of Artificial Sequence: Synthetic polynucleotide
  source 1..4137: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggatcagctactaacttcagcctgctgaagcaggctggagacgtggaggagaaccctggacctatgtgccaccaacagctcgtgatcagctggttctccctggtgttcctggctagccccctggtggccatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggatctggttctggttccggaagtggatctggttcagggtccggtagtggatctgggtcaggaagtggaagcggtagtgggtctggatctaaacaagagcactttcctgataacctgttgccgagctgggcgattacgcttatcagtgtaaacggcatctttgtaatatgctgtctgacctactgcttcgcaccaaggtgccgggagagaaggggatcctcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgagaatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccatcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaa

SEQ ID NO: 125 - protein, 1130 aa, synthetic construct
  REGION 1..1130: note = Description of Artificial Sequence: Synthetic polypeptide
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGSGSGSGSGSGSGSGSGSGSGSGSGSGSLDFACDLLPSWAITLISVNGIFVICCLTYCFAPRCRERRRGSSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGHQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKSRGSGATNFSLLKQAGDVEENPGPLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 126 - DNA, 3390 bp, synthetic construct
  misc_feature 1..3390: note = Description of Artificial Sequence: Synthetic polynucleotide
  source 1..3390: mol_type = other DNA
atgtgccaccaacagctcgtgatcagctggttctccctggtgttcctggctagccccctggtggccatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggatctggttctggttccggaagtggatctggttcagggtccggtagtggatctgggtcaggaagtggaagcggtagtgggtctggatctctggactttgcctgcgatctgttgccgagctgggcgattacgcttatcagtgtaaacggcatctttgtaatatgctgtctgacctactgcttcgcaccaaggtgccgggagagaaggaggggatcctcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgagaatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccatcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaatctagaggtagtggagctactaacttcagcctgctgaagcaggctggagacgtggaggagaaccctggacctttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaag

SEQ ID NO: 127 - protein, 1169 aa, synthetic construct
  REGION 1..1169: note = Description of Artificial Sequence: Synthetic polypeptide
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGSGSGSGSGSGSGSGSGSGSGSGSGSGSTTTPAPRPPTPAPTIASQPLSLRPEACRPAAGGAVHTRGLDFACDIYIWAPLAGTCGVLLLSLVITLYCNHRNRRRVCKGSSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGHQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKSRGSGATNFSLLKQAGDVEENPGPLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 128 - DNA, 3507 bp, synthetic construct
  misc_feature 1..3507: note = Description of Artificial Sequence: Synthetic polynucleotide
  source 1..3507: mol_type = other DNA
atgtgccaccaacagctcgtgatcagctggttctccctggtgttcctggctagccccctggtggccatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggatctggttctggttccggaagtggatctggttcagggtccggtagtggatctgggtcaggaagtggaagcggtagtgggtctggatctaccacaaccccagcacctcgcccaccaacaccagcaccaaccatcgcatcccagccactgtctctgaggcccgaggcatgtaggcctgcagcaggaggcgccgtgcacaccaggggcctggactttgcctgcgatatctatatctgggcaccactggcaggaacatgtggcgtgctgctgctgtccctggtcatcaccctgtattgcaatcatcgcaaccgccgaagggtctgcaagggatcctcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgagaatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccatcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaatctagaggtagtggagctactaacttcagcctgctgaagcaggctggagacgtggaggagaaccctggacctttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaag

SEQ ID NO: 129 - protein, 1322 aa, synthetic construct
  REGION 1..1322: note = Description of Artificial Sequence: Synthetic polypeptide
MSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGMIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENGGSGGSGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGATNFSLLKQAGDVEENPGPLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 130 - DNA, 3966 bp, synthetic construct
  misc_feature 1..3966: note = Description of Artificial Sequence: Synthetic polynucleotide
  source 1..3966: mol_type = other DNA
atgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaaggaggatctggaggtggaagtggaggtggttccggaatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacggtggaagtggtggaagtggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactcggatctggttctggaagtggatctggtagtggttccggttccatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggttctggagctactaacttcagcctgctgaagcaggctggagacgtggaggagaaccctggacctttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaag

SEQ ID NO: 131 - protein, 1322 aa, synthetic construct
  REGION 1..1322: note = Description of Artificial Sequence: Synthetic polypeptide
MSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGMIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENGGSGGSGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGATNFSLLKQAGDVEENPGPLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 132 - DNA, 3969 bp, synthetic construct
  misc_feature 1..3969: note = Description of Artificial Sequence: Synthetic polynucleotide
  source 1..3969: mol_type = other DNA
atgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaaggaggatctggaggtggaagtggaggtggttccggaatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacggtggaagtggtggaagtggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactcggatctggttctggaagtggatctggtagtggttccggttccatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggttctggagctactaacttcagcctgctgaagcaggctggagacgtggaggagaaccctggacctttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaa

SEQ ID NO: 133 - protein, 1311 aa, synthetic construct
  REGION 1..1311: note = Description of Artificial Sequence: Synthetic polypeptide
MSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGMEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGATNFSLLKQAGDVEENPGPLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 134 - DNA, 3933 bp, synthetic construct
  misc_feature 1..3933: note = Description of Artificial Sequence: Synthetic polynucleotide
  source 1..3933: mol_type = other DNA
atgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaaggaggatctggaggtggatctggaatggagtacgctagtgatgcatccttagatcctgaagcgccatggccacctgctccacgagctcgagcctgtcgagtgctgccatgggctctggtcgctgggttgctcctccttctgcttttggccgcggcttgtgcagtgtttcttgcttgcccgtgggcagttagcggtgctcgcgcatctcccggaagcgcggcgagtcctcgactcagggaaggtccggagctgagcccagatgaccccgccggtttgctggacctccgccaaggaatgttcgctcaactcgttgcgcaaaacgtacttcttatagacggccctcttagttggtacagtgacccaggattggctggcgttagtttgacaggcggactcagttacaaggaggatactaaggaactggtagtcgctaaggctggggtatactacgtgttctttcaactcgaactgagaagggtggttgcgggagaaggatctggaagtgtatctctcgccctgcacctccaacccctcagaagtgccgccggagcggccgcccttgcccttactgtcgacctgcccccggcttcttcagaagcgcgaaatagtgcattcggcttccaggggcgccttttgcacttgagcgctggacagcgcctcggggtccacctccacacggaagcgcgggcgaggcacgcttggcaactcacacaaggtgcgacggttctcggcttgtttagggttacgcctgagataccggctggcctcccatctccaagatccgagggatctggttctggaagtggatctggtagtggttccggttccatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggttctggagctactaacttcagcctgctgaagcaggctggagacgtggaggagaaccctggacctttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaag

SEQ ID NO: 135 - protein, 1311 aa, synthetic construct
  REGION 1..1311: note = Description of Artificial Sequence: Synthetic polypeptide
MSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGMEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGATNFSLLKQAGDVEENPGPLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 136 - DNA, 3933 bp, synthetic construct
  misc_feature 1..3933: note = Description of Artificial Sequence: Synthetic polynucleotide
  source 1..3933: mol_type = other DNA
atgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaaggaggatctggaggtggatctggaatggagtacgctagtgatgcatccttagatcctgaagcgccatggccacctgctccacgagctcgagcctgtcgagtgctgccatgggctctggtcgctgggttgctcctccttctgcttttggccgcggcttgtgcagtgtttcttgcttgcccgtgggcagttagcggtgctcgcgcatctcccggaagcgcggcgagtcctcgactcagggaaggtccggagctgagcccagatgaccccgccggtttgctggacctccgccaaggaatgttcgctcaactcgttgcgcaaaacgtacttcttatagacggccctcttagttggtacagtgacccaggattggctggcgttagtttgacaggcggactcagttacaaggaggatactaaggaactggtagtcgctaaggctggggtatactacgtgttctttcaactcgaactgagaagggtggttgcgggagaaggatctggaagtgtatctctcgccctgcacctccaacccctcagaagtgccgccggagcggccgcccttgcccttactgtcgacctgcccccggcttcttcagaagcgcgaaatagtgcattcggcttccaggggcgccttttgcacttgagcgctggacagcgcctcggggtccacctccacacggaagcgcgggcgaggcacgcttggcaactcacacaaggtgcgacggttctcggcttgtttagggttacgcctgagataccggctggcctcccatctccaagatccgagggatctggttctggaagtggatctggtagtggttccggttccatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggttctggagctactaacttcagcctgctgaagcaggctggagacgtggaggagaaccctggacctttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaag

SEQ ID NO: 137 - protein, 1247 aa, synthetic construct
  REGION 1..1247: note = Description of Artificial Sequence: Synthetic polypeptide
MSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGMIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENGGSGGSGGGGSGGGGSGASSGVRLWATRQAMLGQVHEVPEGWLIFVAEQEELYVRVQNGFRKVQLEARTPLPRGTDNGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGATNFSLLKQAGDVEENPGPLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 138 - DNA, 3741 bp, synthetic construct
  misc_feature 1..3741: note = Description of Artificial Sequence: Synthetic polynucleotide
  source 1..3741: mol_type = other DNA
atgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaaggaggatctggaggtggaagtggaggtggttccggaatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacggaggaagcggaggatctggcggaggtggtagcggcggaggcggaagtggtgcttcttctggtgttagactgtgggccaccagacaggccatgctgggacaagtgcacgaagtgcctgaaggctggctgatcttcgtggccgagcaagaggaactgtacgtgcgggtgcagaacggcttcagaaaggtgcagctggaagccagaacacccctgcctagaggcaccgacaatggatctggttctggaagtggatctggtagtggttccggttccatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggttctggagctactaacttcagcctgctgaagcaggctggagacgtggaggagaaccctggacctttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaag

SEQ ID NO: 139 - protein, 1247 aa, synthetic construct
  REGION 1..1247: note = Description of Artificial Sequence: Synthetic polypeptide
MSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGMIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENGGSGGSGGGGSGGGGSGASSGVRLWATRQAMLGQVHEVPEGWLIFVAEQEELYVRVQNGFRKVQLEARTPLPRGTDNGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGATNFSLLKQAGDVEENPGPLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 140 - DNA, 3741 bp, synthetic construct
  misc_feature 1..3741: note = Description of Artificial Sequence: Synthetic polynucleotide
  source 1..3741: mol_type = other DNA
atgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaaggaggatctggaggtggaagtggaggtggttccggaatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacggaggaagcggaggatctggcggaggtggtagcggcggaggcggaagtggtgcttcttctggtgttagactgtgggccaccagacaggccatgctgggacaagtgcacgaagtgcctgaaggctggctgatcttcgtggccgagcaagaggaactgtacgtgcgggtgcagaacggcttcagaaaggtgcagctggaagccagaacacccctgcctagaggcaccgacaatggatctggttctggaagtggatctggtagtggttccggttccatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggttctggagctactaacttcagcctgctgaagcaggctggagacgtggaggagaaccctggacctttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaag

SEQ ID NO: 141 - protein, 1146 aa, synthetic construct
  REGION 1..1146: note = Description of Artificial Sequence: Synthetic polypeptide
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGSGSGSGSGSGSLRPVQAQAQSDCSCSTVSPGVLAGIVMGDLVLTVLIALAVYFLGRLVPRGRGAAEAATRKQRITEGGSGGGSGSSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGHQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKSRGSGATNFSLLKQAGDVEENPGPLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 142 - DNA, 3438 bp, synthetic construct
  misc_feature 1..3438: note = Description of Artificial Sequence: Synthetic polynucleotide
  source 1..3438: mol_type = other DNA
atgtgccaccaacagctcgtgatcagctggttctccctggtgttcctggctagccccctggtggccatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggatctggttctggttccggaagtggatctggttcagggtccctcagacctgttcaggcccaggctcagagcgattgcagctgtagcacagtgtctccaggcgtgctggccggaatcgtgatgggagatctggtgctgacagtgctgattgccctggccgtgtacttcctgggtagactggtgcctagaggaagaggcgctgccgaagccgccacaagaaagcagagaatcacagaaggcggcagcggcggaggatctggatcctcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgagaatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccatcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaatctagaggtagtggagctactaacttcagcctgctgaagcaggctggagacgtggaggagaaccctggacctttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaag

SEQ ID NO: 143 - protein, 1147 aa, synthetic construct
  REGION 1..1147: note = Description of Artificial Sequence: Synthetic polypeptide
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGSGSGSGSGSGSLRPVQAQAQSDCSCSTVSPGVLAGIVMGDLVLTVLIALAVYFLGRLVPRGRGAAEAATRKQRITEGGSGGGSGSMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKSRGSGATNFSLLKQAGDVEENPGPLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 144 - DNA, 3438 bp, synthetic construct
  misc_feature 1..3438: note = Description of Artificial Sequence: Synthetic polynucleotide
  source 1..3438: mol_type = other DNA
atgtgccaccaacagctcgtgatcagctggttctccctggtgttcctggctagccccctggtggccatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggatctggttctggttccggaagtggatctggttcagggtccctcagacctgttcaggcccaggctcagagcgattgcagctgtagcacagtgtctccaggcgtgctggccggaatcgtgatgggagatctggtgctgacagtgctgattgccctggccgtgtacttcctgggtagactggtgcctagaggaagaggcgctgccgaagccgccacaagaaagcagagaatcacagaaggcggcagcggcggaggatctggatcctcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaatctagaggtagtggagctactaacttcagcctgctgaagcaggctggagacgtggaggagaaccctggacctttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaag

SEQ ID NO: 145 - protein, 1146 aa, synthetic construct
  REGION 1..1146: note = Description of Artificial Sequence: Synthetic polypeptide
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGSGSGSGSGSGSLRPVQAQAQSDCSCSTVSPGVLAGIVMGDLVLTVLIALAVYFLGRLVPRGRGAAEAATRKQRITEGGSGGGSGSSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKSRGSGATNFSLLKQAGDVEENPGPLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 146 - DNA, 3438 bp, synthetic construct
  misc_feature 1..3438: note = Description of Artificial Sequence: Synthetic polynucleotide
  source 1..3438: mol_type = other DNA
atgtgccaccaacagctcgtgatcagctggttctccctggtgttcctggctagccccctggtggccatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggatctggttctggttccggaagtggatctggttcagggtccctcagacctgttcaggcccaggctcagagcgattgcagctgtagcacagtgtctccaggcgtgctggccggaatcgtgatgggagatctggtgctgacagtgctgattgccctggccgtgtacttcctgggtagactggtgcctagaggaagaggcgctgccgaagccgccacaagaaagcagagaatcacagaaggcggcagcggcggaggatctggatcctcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaatctagaggtagtggagctactaacttcagcctgctgaagcaggctggagacgtggaggagaaccctggacctttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaag

SEQ ID NO: 147 - protein, 1135 aa, synthetic construct
  REGION 1..1135: note = Description of Artificial Sequence: Synthetic polypeptide
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGSGSGSGSGSGSKQEHFPDNGVLAGIVMGDLVLTVLIALAVTYCFAPRCRERRRNERLRRESVRPVGGSGGGSGSSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGHQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKSRGSGATNFSLLKQAGDVEENPGPLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 148 - DNA, 3405 bp, synthetic construct
  misc_feature 1..3405: note = Description of Artificial Sequence: Synthetic polynucleotide
  source 1..3405: mol_type = other DNA
atgtgccaccaacagctcgtgatcagctggttctccctggtgttcctggctagccccctggtggccatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggatctggttctggttccggaagtggatctggttcagggtccaagcaagagcacttccccgacaatggcgtgctggccggaatcgtgatgggagatctggtgctgacagtgctgattgccctggccgtgacctactgcttcgcccctagatgcagagagcggcggagaaacgaacggctgagaagagaatctgtgcggcccgttggcggcagcggcggaggatctggatcctcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgagaatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccatcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaatctagaggtagtggagctactaacttcagcctgctgaagcaggctggagacgtggaggagaaccctggacctttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaag

SEQ ID NO: 149 - protein, 1135 aa, synthetic construct
  REGION 1..1135: note = Description of Artificial Sequence: Synthetic polypeptide
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGSGSGSGSGSGSKQEHFPDNGVLAGIVMGDLVLTVLIALAVTYCFAPRCRERRRNERLRRESVRPVGGSGGGSGSSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKSRGSGATNFSLLKQAGDVEENPGPLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 150 - DNA, 3405 bp, synthetic construct
  misc_feature 1..3405: note = Description of Artificial Sequence: Synthetic polynucleotide
  source 1..3405: mol_type = other DNA
atgtgccaccaacagctcgtgatcagctggttctccctggtgttcctggctagccccctggtggccatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggatctggttctggttccggaagtggatctggttcagggtccaagcaagagcacttccccgacaatggcgtgctggccggaatcgtgatgggagatctggtgctgacagtgctgattgccctggccgtgacctactgcttcgcccctagatgcagagagcggcggagaaacgaacggctgagaagagaatctgtgcggcccgttggcggcagcggcggaggatctggatcctcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaatctagaggtagtggagctactaacttcagcctgctgaagcaggctggagacgtggaggagaaccctggacctttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaag

SEQ ID NO: 151 - protein, 1135 aa, synthetic construct
  REGION 1..1135: note = Description of Artificial Sequence: Synthetic polypeptide
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGSGSGSGSGSGSKQEHFPDNGVLAGIVMGDLVLTVLIALAVTYCFAPRCRERRRNERLRRESVRPVGGSGGGSGSSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKSRGSGATNFSLLKQAGDVEENPGPLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 152 - DNA, 3405 bp, synthetic construct
  misc_feature 1..3405: note = Description of Artificial Sequence: Synthetic polynucleotide
  source 1..3405: mol_type = other DNA
atgtgccaccaacagctcgtgatcagctggttctccctggtgttcctggctagccccctggtggccatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggatctggttctggttccggaagtggatctggttcagggtccaagcaagagcacttccccgacaatggcgtgctggccggaatcgtgatgggagatctggtgctgacagtgctgattgccctggccgtgacctactgcttcgcccctagatgcagagagcggcggagaaacgaacggctgagaagagaatctgtgcggcccgttggcggcagcggcggaggatctggatcctcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaatctagaggtagtggagctactaacttcagcctgctgaagcaggctggagacgtggaggagaaccctggacctttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaag

SEQ ID NO: 153 - protein, 1285 aa, synthetic construct
  REGION 1..1285: note = Description of Artificial Sequence: Synthetic polypeptide
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGSGSGSGSGSGSSLRQQVEALQGQVQHLQAAFSQYKKVELFPNGQSVGEKIFKTAGFVKPFTEAQLLCTQAGGQLASPRSAENAALQQLVVAKNEAAFLSMTDSKTEGKFTYPTGESLVYSNWAPGEPNDDGGSEDCVEIFTNGKWNDRACGEKRLVVCEFGGGSGGGSKQEHFPDNLLPSWAITLISVNGIFVICCLTYCFAPRCRERRRNERLRRESVRPVGSSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKSRGSGATNFSLLKQAGDVEENPGPLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 154 - DNA, 3858 bp, synthetic construct
  misc_feature 1..3858: note = Description of Artificial Sequence: Synthetic polynucleotide
  source 1..3858: mol_type = other DNA
atgtgccaccaacagctcgtgatcagctggttctccctggtgttcctggctagccccctggtggccatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggatctggttctggttccggaagtggatctggttcagggtccagcctgagacagcaggttgaagccctgcaaggccaggtgcagcatctgcaggctgccttcagccagtacaagaaggtggaactgttccccaacggccagagcgtgggcgagaagatctttaagaccgccggcttcgtgaagcccttcacagaggctcagctgctgtgtactcaggctggtggacagctggctagccctagatctgccgccgaaaatgctgctctgcagcagctggtggtggccaagaatgaagccgccttcctgagcatgaccgacagcaagaccgagggcaagttcacataccctaccggcgagagcctggtgtacagcaattgggctcctggcgagcccaatgatgatggcggctctgaggactgcgtggaaatcttcaccaacggcaagtggaacgacagagcctgcggagagaagagactggtcgtgtgcgaatttggcggcggatctggcggaggatctaaacaagagcactttcctgataacctgttgccgagctgggcgattacgcttatcagtgtaaacggcatctttgtaatatgctgtctgacctactgcttcgcaccaaggtgccgggagagaaggagaaatgaaagactgagaagggagagcgtgagacctgtgggatcctcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggattctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaatctagaggtagtggagctactaacttcagcctgctgaagcaggctggagacgtggaggagaaccctggacctttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaag

SEQ ID NO: 155 - protein, 1286 aa, synthetic construct
  REGION 1..1286: note = Description of Artificial Sequence: Synthetic polypeptide
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGSGSGSGSGSGSSLRQQVEALQGQVQHLQAAFSQYKKVELFPNGQSVGEKIFKTAGFVKPFTEAQLLCTQAGGQLASPRSAAENAALQQLVVAKNEAAFLSMTDSKTEGKFTYPTGESLVYSNWAPGEPNDDGGSEDCVEIFTNGKWNDRACGEKRLVVCEFGGGSGGGSKQEHFPDNLLPSWAITLISVNGIFVICCLTYCFAPRCRERRRNERLRRESVRPVGSSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKSRGSGATNFSLLKQAGDVEENPGPLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 156 - DNA, 3858 bp, synthetic construct
  misc_feature 1..3858: note = Description of Artificial Sequence: Synthetic polynucleotide
  source 1..3858: mol_type = other DNA
atgtgccaccaacagctcgtgatcagctggttctccctggtgttcctggctagccccctggtggccatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggatctggttctggttccggaagtggatctggttcagggtccagcctgagacagcaggttgaagccctgcaaggccaggtgcagcatctgcaggctgccttcagccagtacaagaaggtggaactgttccccaacggccagagcgtgggcgagaagatctttaagaccgccggcttcgtgaagcccttcacagaggctcagctgctgtgtactcaggctggtggacagctggctagccctagatctgccgccgaaaatgctgctctgcagcagctggtggtggccaagaatgaagccgccttcctgagcatgaccgacagcaagaccgagggcaagttcacataccctaccggcgagagcctggtgtacagcaattgggctcctggcgagcccaatgatgatggcggctctgaggactgcgtggaaatcttcaccaacggcaagtggaacgacagagcctgcggagagaagagactggtcgtgtgcgaatttggcggcggatctggcggaggatctaaacaagagcactttcctgataacctgttgccgagctgggcgattacgcttatcagtgtaaacggcatctttgtaatatgctgtctgacctactgcttcgcaccaaggtgccgggagagaaggagaaatgaaagactgagaagggagagcgtgagacctgtgggatcctcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgaggattctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaatctagaggtagtggagctactaacttcagcctgctgaagcaggctggagacgtggaggagaaccctggacctttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaag

SEQ ID NO: 157 - protein, 1221 aa, synthetic construct
  REGION 1..1221: note = Description of Artificial Sequence: Synthetic polypeptide
MSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGMIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENGGGGSGGGGSSGLPDVASLRQQVEALQGQVQHLQAAFSQYKKVELFPNGQSGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGATNFSLLKQAGDVEENPGPLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 158 - DNA, 3663 bp, synthetic construct
  misc_feature 1..3663: note = Description of Artificial Sequence: Synthetic polynucleotide
  source 1..3663: mol_type = other DNA
atgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaaggaggatctggaggtggaagtggaggtggttccggaatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacggaggcggaggatctggcggaggcggatcttctggactgcctgatgtggccagcctgagacagcaggttgaagccctgcaaggccaggtgcagcatctgcaggctgccttcagccagtacaagaaggtggaactgttccccaacggccagagcggatctggttctggaagtggatctggtagtggttccggtagtatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggttctggagctactaacttcagcctgctgaagcaggctggagacgtggaggagaaccctggacctttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaag

SEQ ID NO: 159 - protein, 1221 aa, synthetic construct
  REGION 1..1221: note = Description of Artificial Sequence: Synthetic polypeptide
MSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGMIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENGGGGSGGGGSSGLPDVASLRQQVEALQGQVQHLQAAFSQYKKVELFPNGQSGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGATNFSLLKQAGDVEENPGPLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 160 - DNA, 3663 bp, synthetic construct
  misc_feature 1..3663: note = Description of Artificial Sequence: Synthetic polynucleotide
  source 1..3663: mol_type = other DNA
atgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaaggaggatctggaggtggaagtggaggtggttccggaatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacggaggcggaggatctggcggaggcggatcttctggactgcctgatgtggccagcctgagacagcaggttgaagccctgcaaggccaggtgcagcatctgcaggctgccttcagccagtacaagaaggtggaactgttccccaacggccagagcggatctggttctggaagtggatctggtagtggttccggtagtatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggttctggagctactaacttcagcctgctgaagcaggctggagacgtggaggagaaccctggacctttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaag

SEQ ID NO: 161 - protein, 1270 aa, synthetic construct
  REGION 1..1270: note = Description of Artificial Sequence: Synthetic polypeptide
MSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGPREPPPKSGPSLVPGKTPTVRAALICLTLVLVASVLLQAVLYPRFMGTISDVKTGGSSLENMSKLLKRQNDILQVVSQGWKYFKGNFYYFSLIPKTWYSAEQFCVSRNSHLTSVTSESEQEFLYKTAGGLIYWIGLTKAGMEGDWSWVDDTPFNKVQSVRFWIPGEPNNAGNNEHCGNIKAPSLQAWNDAPCDKTFLFICKRPYVPSEPGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGATNFSLLKQAGDVEENPGPLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 162 - DNA, 3810 bp, synthetic construct
  misc_feature 1..3810: note = Description of Artificial Sequence: Synthetic polynucleotide
  source 1..3810: mol_type = other DNA
atgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaaggaggatctggaggtggaagtggaggtggttccggacctagagagcctcctccaaagtctggcccttctctggtgcctggcaagacccctacagttagagccgctctgatctgtctgaccctggtgctggttgctagcgtgctgctgcaggctgtgctgtaccctagattcatgggcaccatcagcgacgtgaaaaccggcggcagcagcctggaaaacatgagcaagctgctgaagcggcagaacgacatcctgcaggtcgtgtcccaaggctggaagtactttaagggcaacttctactacttcagtctgatccccaagacctggtacagcgccgagcagttctgcgtgtccagaaacagccacctgaccagcgtgacaagcgagagcgagcaagagttcctgtacaagacagccggcggactgatctactggatcggcctgacaaaggccggcatggaaggcgattggagctgggtcgacgacacccctttcaacaaggtgcagagcgtgcggttctggatccctggcgaacctaacaacgccggcaacaatgagcactgcggcaacatcaaggccccatctctgcaggcctggaacgatgccccttgcgacaagaccttcctgttcatctgcaagcggccctacgtgccatctgagcctggatctggttctggaagtggatctggtagtggttccggtagtatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggttctggagctactaacttcagcctgctgaagcaggctggagacgtggaggagaaccctggacctttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaag

SEQ ID NO: 163 - protein, 1270 aa, synthetic construct
  REGION 1..1270: note = Description of Artificial Sequence: Synthetic polypeptide
MSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGPREPPPKSGPSLVPGKTPTVRAALICLTLVLVASVLLQAVLYPRFMGTISDVKTGGSSLENMSKLLKRQNDILQVVSQGWKYFKGNFYYFSLIPKTWYSAEQFCVSRNSHLTSVTSESEQEFLYKTAGGLIYWIGLTKAGMEGDWSWVDDTPFNKVQSVRFWIPGEPNNAGNNEHCGNIKAPSLQAWNDAPCDKTFLFICKRPYVPSEPGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGATNFSLLKQAGDVEENPGPLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 164 - DNA, 3810 bp, synthetic construct
  misc_feature 1..3810: note = Description of Artificial Sequence: Synthetic polynucleotide
  source 1..3810: mol_type = other DNA
atgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaaggaggatctggaggtggaagtggaggtggttccggacctagagagcctcctccaaagtctggcccttctctggtgcctggcaagacccctacagttagagccgctctgatctgtctgaccctggtgctggttgctagcgtgctgctgcaggctgtgctgtaccctagattcatgggcaccatcagcgacgtgaaaaccggcggcagcagcctggaaaacatgagcaagctgctgaagcggcagaacgacatcctgcaggtcgtgtcccaaggctggaagtactttaagggcaacttctactacttcagtctgatccccaagacctggtacagcgccgagcagttctgcgtgtccagaaacagccacctgaccagcgtgacaagcgagagcgagcaagagttcctgtacaagacagccggcggactgatctactggatcggcctgacaaaggccggcatggaaggcgattggagctgggtcgacgacacccctttcaacaaggtgcagagcgtgcggttctggatccctggcgaacctaacaacgccggcaacaatgagcactgcggcaacatcaaggccccatctctgcaggcctggaacgatgccccttgcgacaagaccttcctgttcatctgcaagcggccctacgtgccatctgagcctggatctggttctggaagtggatctggtagtggttccggtagtatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggttctggagctactaacttcagcctgctgaagcaggctggagacgtggaggagaaccctggacctttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaag

SEQ ID NO: 165 - protein, 1322 aa, synthetic construct
  REGION 1..1322: note = Description of Artificial Sequence: Synthetic polypeptide
MSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGMIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGATNFSLLKQAGDVEENPGPLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 166 - DNA, 3966 bp, synthetic construct
  misc_feature 1..3966: note = Description of Artificial Sequence: Synthetic polynucleotide
  source 1..3966: mol_type = other DNA
atgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaaggaggatctggaggtggaagtggaggtggttccggaatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactcggatctggttctggaagtggatctggtagtggttccggttccatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggttctggagctactaacttcagcctgctgaagcaggctggagacgtggaggagaaccctggacctttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaag

SEQ ID NO: 167 - protein, 1197 aa, synthetic construct
  REGION 1..1197: note = Description of Artificial Sequence: Synthetic polypeptide
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGSGSGSGSGSGSGASSGVRLWATRQAMLGQVHEVPEGWLIFVAEQEELYVRVQNGFRKVQLEARTPLPRGTDNGGGSGGGSKQEHFPDNLLPSWAITLISVNGIFVICCLTYCFAPRCRERRRNERLRRESVRPVGSSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKSRGSGATNFSLLKQAGDVEENPGPLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 168 - DNA, 3591 bp, synthetic construct
  misc_feature 1..3591: note = Description of Artificial Sequence: Synthetic polynucleotide
  source 1..3591: mol_type = other DNA
atgtgccaccaacagctcgtgatcagctggttctccctggtgttcctggctagccccctggtggccatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggatctggttctggttccggaagtggatctggttcagggtccggtgcttcttctggtgttagactgtgggccaccagacaggccatgctgggacaagtgcacgaagtgcctgaaggctggctgatcttcgtggccgagcaagaggaactgtacgtgcgggtgcagaacggcttcagaaaggtgcagctggaagccagaacacccctgcctagaggcaccgacaatggcggcggatctggcggaggatctaaacaagagcactttcctgataacctgttgccgagctgggcgattacgcttatcagtgtaaacggcatctttgtaatatgctgtctgacctactgcttcgcaccaaggtgccgggagagaaggagaaatgaaagactgagaagggagagcgtgagacctgtgggatcctcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggattctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaatctagaggtagtggagctactaacttcagcctgctgaagcaggctggagacgtggaggagaaccctggacctttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaag

SEQ ID NO: 169 - protein, 1197 aa, synthetic construct
  REGION 1..1197: note = Description of Artificial Sequence: Synthetic polypeptide
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGSGSGSGSGSGSGASSGVRLWATRQAMLGQVHEVPEGWLIFVAEQEELYVRVQNGFRKVQLEARTPLPRGTDNGGGSGGGSKQEHFPDNLLPSWAITLISVNGIFVICCLTYCFAPRCRERRRNERLRRESVRPVGSSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKSRGSGATNFSLLKQAGDVEENPGPLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 170 - DNA, 3591 bp, synthetic construct
  misc_feature 1..3591: note = Description of Artificial Sequence: Synthetic polynucleotide
  source 1..3591: mol_type = other DNA
atgtgccaccaacagctcgtgatcagctggttctccctggtgttcctggctagccccctggtggccatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggatctggttctggttccggaagtggatctggttcagggtccggtgcttcttctggtgttagactgtgggccaccagacaggccatgctgggacaagtgcacgaagtgcctgaaggctggctgatcttcgtggccgagcaagaggaactgtacgtgcgggtgcagaacggcttcagaaaggtgcagctggaagccagaacacccctgcctagaggcaccgacaatggcggcggatctggcggaggatctaaacaagagcactttcctgataacctgttgccgagctgggcgattacgcttatcagtgtaaacggcatctttgtaatatgctgtctgacctactgcttcgcaccaaggtgccgggagagaaggagaaatgaaagactgagaagggagagcgtgagacctgtgggatcctcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgaggattctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaatctagaggtagtggagctactaacttcagcctgctgaagcaggctggagacgtggaggagaaccctggacctttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaag

SEQ ID NO: 171 - protein, 1382 aa, synthetic construct
  REGION 1..1382: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGSKQRGTFSEVSLAQDARQKLQNLFINFCLILICLLLICIIVMLLIPFLEQNNSSPNTRTQKGGSGGSGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 172 - DNA, 4146 bp, synthetic construct
  misc_feature 1..4146: note = Description of Artificial Sequence: Synthetic polynucleotide
  source 1..4146: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatctggtgctactaacttcagcctgctgaagcaggctggagacgtggaggagaaccctggacctggaggatccatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaagggaggttccggaggtggaagtggaggtggatcaggtagcaagcagagaggcaccttcagcgaagtgtctctggcccaggatgccagacagaagctgcagaacctgttcatcaatttctgcctgatcctgatctgcctgctgctcatctgcattatcgtgatgctgctgatcccattcctcgagcagaacaacagcagccccaacacacggacccagaagggtggtagtggaggctccggttccggatctggaagtggatctggtagtggttccggtagtatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagc

SEQ ID NO: 173 - protein, 1382 aa, synthetic construct
  REGION 1..1382: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGSKQRGTFSEVSLAQDARQKLQNLFINFCLILICLLLICIIVMLLIPFLEQNNSSPNTRTQKSFEMQKGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 174 - DNA, 4146 bp, synthetic construct
  misc_feature 1..4146: note = Description of Artificial Sequence: Synthetic polynucleotide
  source 1..4146: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatctggtgctactaacttcagcctgctgaagcaggctggagacgtggaggagaaccctggacctggaggatccatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaagggaggttccggaggtggaagtggaggtggatcaggtagcaagcagagaggcaccttcagcgaagtgtctctggcccaggatgccagacagaagctgcagaacctgttcatcaatttctgcctgatcctgatctgcctgctgctcatctgcattatcgtgatgctgctgatcccattcctcgagcagaacaacagcagccccaacacacggacccagaagagctttgagatgcagaaaggttccggatctggaagtggatctggtagtggttccggtagtatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagc

SEQ ID NO: 175 - protein, 1509 aa, synthetic construct
  REGION 1..1509: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGSGGSGGSGGSGGSGGSGGASSGVRLWATRQAMLGQVHEVPEGWLIFVAEQEELYVRVQNGFRKVQLEARTPLPRGTDNGGSGGGSGGGSGMIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 176 - DNA, 4527 bp, synthetic construct
  misc_feature 1..4527: note = Description of Artificial Sequence: Synthetic polynucleotide
  source 1..4527: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatctggtgctactaacttcagcctgctgaagcaggctggagacgtggaggagaaccctggacctggaggatccatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaagggaagcggaggttctggtggttctggcggtagcggcggcagcggaggaagtggcggtgcttcttctggtgttagactgtgggccaccagacaggccatgctgggacaagtgcacgaagtgcctgaaggctggctgatcttcgtggccgagcaagaggaactgtacgtgcgggtgcagaacggcttcagaaaggtgcagctggaagccagaacacccctgcctagaggcaccgacaatggaggttccggaggtggaagtggaggtggatcaggtatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggttccggatctggaagtggatctggtagtggttccggtagtatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagc

SEQ ID NO: 177 - protein, 1508 aa, synthetic construct
  REGION 1..1508: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMGASSGVRLWATRQAMLGQVHEVPEGWLIFVAEQEELYVRVQNGFRKVQLEARTPLPRGTDNGSGGSGGSGGSGGSGGSGSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGGGGSGMIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 178 - DNA, 4524 bp, synthetic construct
  misc_feature 1..4524: note = Description of Artificial Sequence: Synthetic polynucleotide
  source 1..4524: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatctggtgctactaacttcagcctgctgaagcaggctggagacgtggaggagaaccctggacctggaggatccatgggtgcttcttctggtgttagactgtgggccaccagacaggccatgctgggacaagtgcacgaagtgcctgaaggctggctgatcttcgtggccgagcaagaggaactgtacgtgcgggtgcagaacggcttcagaaaggtgcagctggaagccagaacacccctgcctagaggcaccgacaatggaagcggaggttctggtggttctggcggtagcggcggcagcggaggaagtggctcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaagggaggttccggaggtggaggaggtggatcaggtatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggttccggatctggaagtggatctggtagtggttccggtagtatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagc

SEQ ID NO: 179 - protein, 1489 aa, synthetic construct
  REGION 1..1489: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGSGGSGGSGGSGGSGGSGSGLPDVASLRQQVEALQGQVQHLQAAFSQYKKVELFPNGQSGGSGGGSGGGSGMIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 180 - DNA, 4467 bp, synthetic construct
  misc_feature 1..4467: note = Description of Artificial Sequence: Synthetic polynucleotide
  source 1..4467: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatctggtgctactaacttcagcctgctgaagcaggctggagacgtggaggagaaccctggacctggaggatccatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaagggaagcggaggttctggtggttctggcggtagcggcggcagcggaggaagtggctctggactgcctgatgtggccagcctgagacagcaggttgaagccctgcaaggccaggtgcagcatctgcaggctgccttcagccagtacaagaaggtggaactgttccccaacggccagagcggaggttccggaggtggaagtggaggtggatcaggtatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggttccggatctggaagtggatctggtagtggttccggtagtatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagc

SEQ ID NO: 181 - protein, 1489 aa, synthetic construct
  REGION 1..1489: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMSGLPDVASLRQQVEALQGQVQHLQAAFSQYKKVELFPNGQSGSGGSGGSGGSGGSGGSGSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGMIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 182 - DNA, 4467 bp, synthetic construct
  misc_feature 1..4467: note = Description of Artificial Sequence: Synthetic polynucleotide
  source 1..4467: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatctggtgctactaacttcagcctgctgaagcaggctggagacgtggaggagaaccctggacctggaggatccatgtctggactgcctgatgtggccagcctgagacagcaggttgaagccctgcaaggccaggtgcagcatctgcaggctgccttcagccagtacaagaaggtggaactgttccccaacggccagagcggaagcggaggttctggtggttctggcggtagcggcggcagcggaggaagtggctcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaagggaggttccggaggtggaagtggaggtggatcaggtatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggttccggatctggaagtggatctggtagtggttccggtagtatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagc

SEQ ID NO: 183 - protein, 1576 aa, synthetic construct
  REGION 1..1576: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGMIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENGGSGGSGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 184 - DNA, 4728 bp, synthetic construct
  misc_feature 1..4728: note = Description of Artificial Sequence: Synthetic polynucleotide
  source 1..4728: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatctggtgctactaacttcagcctgctgaagcaggctggagacgtggaggagaaccctggacctggaggatccatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaagggaggttccggtggtggaagtggaggtggttccggaatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacggtggaagtggtggaagtggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactcggttccggatctggaagtggatctggtagtggttccggtagtatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagc

SEQ ID NO: 185 - protein, 1501 aa, synthetic construct
  REGION 1..1501: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGMIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENGGSGGSGGGGSGGGGSGASSGVRLWATRQAMLGQVHEVPEGWLIFVAEQEELYVRVQNGFRKVQLEARTPLPRGTDNGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 186 - DNA, 4503 bp, synthetic construct
  misc_feature 1..4503: note = Description of Artificial Sequence: Synthetic polynucleotide
  source 1..4503: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatctggtgctactaacttcagcctgctgaagcaggctggagacgtggaggagaaccctggacctggaggatccatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaagggaggttccggtggtggaagtggaggtggttccggaatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacggaggaagcggaggatctggcggaggtggtagcggcggaggcggaagtggtgcttcttctggtgttagactgtgggccaccagacaggccatgctgggacaagtgcacgaagtgcctgaaggctggctgatcttcgtggccgagcaagaggaactgtacgtgcgggtgcagaacggcttcagaaaggtgcagctggaagccagaacacccctgcctagaggcaccgacaatggttccggatctggaagtggatctggtagtggttccggtagtatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagc

SEQ ID NO: 187 - protein, 1732 aa, synthetic construct
  REGION 1..1732: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGMIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENGGGGSGGGGSSGLPDVASLRQQVEALQGQVQHLQAAFSQYKKVELFPNGQSGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGATNFSLLKQAGDVEENPGPLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 188 - DNA, 4425 bp, synthetic construct
  misc_feature 1..4425: note = Description of Artificial Sequence: Synthetic polynucleotide
  source 1..4425: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatctggtgctactaacttcagcctgctgaagcaggctggagacgtggaggagaaccctggacctggaggatccatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaagggaggttccggtggtggaagtggaggtggttccggaatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacggaggcggaggatctggcggaggcggatcttctggactgcctgatgtggccagcctgagacagcaggttgaagccctgcaaggccaggtgcagcatctgcaggctgccttcagccagtacaagaaggtggaactgttccccaacggccagagcggttccggatctggaagtggatctggtagtggttccggtagtatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagc

SEQ ID NO: 189 - protein, 1576 aa, synthetic construct
  REGION 1..1576: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGMIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 190 - DNA, 4728 bp, synthetic construct
  misc_feature 1..4728: note = Description of Artificial Sequence: Synthetic polynucleotide
  source 1..4728: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatctggtgctactaacttcagcctgctgaagcaggctggagacgtggaggagaaccctggacctggaggatccatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaagggaggttccggtggtggaagtggaggtggttccggaatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactcggttccggatctggaagtggatctggtagtggttccggtagcatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagc

SEQ ID NO: 191 - protein, 1493 aa, synthetic construct
  REGION 1..1493: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGSGGSGGSGGSGGSGGSGGSGSSDYSDLQRVKQELLEEVKKELQKVKEEIIEAFVQELRKRGSGGSGGGSGGGSGMIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 192 - DNA, 4479 bp, synthetic construct
  misc_feature 1..4479: note = Description of Artificial Sequence: Synthetic polynucleotide
  source 1..4479: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatctggtgctactaacttcagcctgctgaagcaggctggagacgtggaggagaaccctggacctggaggatccatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaagggaagcggaggaagtggtggttctggtggttctggcggtagcggcggcagcggaggttccggatctagcgactatagcgatttgcaacgggtgaaacaagaactgttggaagaagttaaaaaggagttgcagaaggtcaaggaggaaattattgaagccttcgtccaggaacttaggaaaagggggagcggaggttccggaggtggaagtggaggtggatcaggtatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggttccggatctggaagtggatctggtagtggttccggtagtatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagc

SEQ ID NO: 193 - protein, 1497 aa, synthetic construct
  REGION 1..1497: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMSSDYSDLQRVKQELLEEVKKELQKVKEEIIEAFVQELRKRGSGSGGSGGSGGSGGSGGSGGSGSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGGGSGMIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 194 - DNA, 4491 bp, synthetic construct
  misc_feature 1..4491: note = Description of Artificial Sequence: Synthetic polynucleotide
  source 1..4491: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatctggtgctactaacttcagcctgctgaagcaggctggagacgtggaggagaaccctggacctggaggatccatgtctagcgactatagcgatttgcaacgggtgaaacaagaactgttggaagaagttaaaaaggagttgcagaaggtcaaggaggaaattattgaagccttcgtccaggaacttaggaaaagggggagcggatccggaggaagtggtggttctggtggttctggcggtagcggcggcagcggaggttccggttcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggattctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaagggaggcagtggcggaggttccggaggtggaagtggaggtggatcaggtatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggttccggatctggaagtggatctggtagtggttccggtagtatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagc

SEQ ID NO: 195 - protein, 1478 aa, synthetic construct
  REGION 1..1478: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGSGSGSGSGSGSSFEMQKGGSLRPVQAQAQSDCSCSTVSPGVLAGIVMGDLVLTVLIALAVYFLGRLVPRGRGAAEAATRKQRITEGGSGGGSGGGSGGGSGSSDYSDLQRVKQELLEEVKKELQKVKEEIIEAFVQELRKRGSGSGGSGGSGGSGGSGGSGGSGMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 196 - DNA, 4434 bp, synthetic construct
  misc_feature 1..4434: note = Description of Artificial Sequence: Synthetic polynucleotide
  source 1..4434: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatctggtgctactaacttcagcctgctgaagcaggctggagacgtggaggagaaccctggacctggaggatccatgtgccaccaacagctcgtgatcagctggttctccctggtgttcctggctagccccctggtggccatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggatctggttctggttccggaagtggatctggttcagggtccagctttgaaatgcaaaaaggcggtagtctcagacctgttcaggcccaggctcagagcgattgcagctgtagcacagtgtctccaggcgtgctggccggaatcgtgatgggagatctggtgctgacagtgctgattgccctggccgtgtacttcctgggtagactggtgcctagaggaagaggcgctgccgaagccgccacaagaaagcagagaatcacagaaggcggctccggtggtggaagtggaggtggctctggtggtggcagcggatccagcgactatagcgatttgcaacgggtgaaacaagaactgttggaagaagttaaaaaggagttgcagaaggtcaaggaggaaattattgaagccttcgtccaggaacttaggaaaaggggatccggttccggaggaagtggtggttctggtggttctggcggtagcggcggcagcggaggttccggtatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggattctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaa

SEQ ID NO: 197 - protein, 1478 aa, synthetic construct
  REGION 1..1478: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGSGSGSGSGSGSSFEMQKGGSLRPVQAQAQSDCSCSTVSPGVLAGIVMGDLVLTVLIALAVYFLGRLVPRGRGAAEAATRKQRITEGGSGGGSGGGSGGGSGMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGSGGSGGSGGSGGSGGSGGSGSSDYSDLQRVKQELLEEVKKELQKVKEEIIEAFVQELRKRGS

SEQ ID NO: 198 - DNA, 4434 bp, synthetic construct
  misc_feature 1..4434: note = Description of Artificial Sequence: Synthetic polynucleotide
  source 1..4434: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatctggtgctactaacttcagcctgctgaagcaggctggagacgtggaggagaaccctggacctggaggatccatgtgccaccaacagctcgtgatcagctggttctccctggtgttcctggctagccccctggtggccatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggatctggttctggttccggtagtggatctggttcaggatccagctttgaaatgcaaaaaggcggtagtctcagacctgttcaggcccaggctcagagcgattgcagctgtagcacagtgtctccaggcgtgctggccggaatcgtgatgggagatctggtgctgacagtgctgattgccctggccgtgtacttcctgggtagactggtgcctagaggaagaggcgctgccgaagccgccacaagaaagcagagaatcacagaaggcggatccggtggtggaagtggaggtggctctggtggtggcagcggcatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggattctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaaggtagtggaggaagtggtggttctggtggttctggcggtagcggcggcagcggaggttccggatctagcgactatagcgatttgcaacgggtgaaacaagaactgttggaagaagttaaaaaggagttgcagaaggtcaaggaggaaattattgaagccttcgtccaggaacttaggaaaagggggagc

SEQ ID NO: 199 - protein, 1476 aa, synthetic construct
  REGION 1..1476: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGSGSGSGSGSGSSFEMQKGGSLTLRWELSSQPTIPIVGIIAGLVLLGAVITGAVVAAVMWRRKSSDRKGGSYTQAASSDSAQGSGGSGGGSGGGSGGGSGSSDYSDLQRVKQELLEEVKKELQKVKEEIIEAFVQELRKRGSGSGGSGGSGGSGGSGGSGGSGMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 200 - DNA, 4428 bp, synthetic construct
  misc_feature 1..4428: note = Description of Artificial Sequence: Synthetic polynucleotide
  source 1..4428: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatctggtgctactaacttcagcctgctgaagcaggctggagacgtggaggagaaccctggacctggaggatctatgtgccaccaacagctcgtgatcagctggttctccctggtgttcctggctagccccctggtggccatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggatctggttctggttccggaagtggatctggttcagggtccagctttgaaatgcaaaaaggcggtagtctgactcttaggtgggaactgagcagtcagcccactatccccattgtcggtatcattgccggactcgttttgcttggtgctgtgatcactggagcggtggtcgcggcagtgatgtggcggcgaaagtcaagcgacagaaagggcggctcctatactcaagccgcctcaagtgactctgcacagggtagcggcggctccggtggtggaagtggaggtggctctggtggtggcagcggatccagcgactatagcgatttgcaacgggtgaaacaagaactgttggaagaagttaaaaaggagttgcagaaggtcaaggaggaaattattgaagccttcgtccaggaacttaggaaaaggggatccggttccggaggaagtggtggttctggtggttctggcggtagcggcggcagcggaggttccggtatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggattctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaa

SEQ ID NO: 201 - protein, 1476 aa, synthetic construct
  REGION 1..1476: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGSGSGSGSGSGSSFEMQKGGSLTLRWELSSQPTIPIVGIIAGLVLLGAVITGAVVAAVMWRRKSSDRKGGSYTQAASSDSAQGSGGSGGGSGGGSGGGSGMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGSGGSGGSGGSGGSGGSGGSGSSDYSDLQRVKQELLEEVKKELQKVKEEIIEAFVQELRKRGS

SEQ ID NO: 202 - DNA, 4428 bp, synthetic construct
  misc_feature 1..4428: note = Description of Artificial Sequence: Synthetic polynucleotide
  source 1..4428: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatctggtgctactaacttcagcctgctgaagcaggctggagacgtggaggagaaccctggacctggaggatctatgtgccaccaacagctcgtgatcagctggttctccctggtgttcctggctagccccctggtggccatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggatctggttctggttccggaagtggatctggttcaggatccagctttgaaatgcaaaaaggcggtagtctgactcttaggtgggaactgagcagtcagcccactatccccattgtcggtatcattgccggactcgttttgcttggtgctgtgatcactggagcggtggtcgcggcagtgatgtggcggcgaaagtcaagcgacagaaagggcggctcctatactcaagccgcctcaagtgactctgcacagggtagcggcggatccggtggtggaagtggaggtggctctggtggtggcagcggcatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggattctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaaggtagtggaggaagtggtggttctggtggttctggcggtagcggcggcagcggaggttccggatctagcgactatagcgatttgcaacgggtgaaacaagaactgttggaagaagttaaaaaggagttgcagaaggtcaaggaggaaattattgaagccttcgtccaggaacttaggaaaagggggagc

SEQ ID NO: 203 - protein, 1497 aa, synthetic construct
  REGION 1..1497: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGSGSGSGSGSGSSFEMQKGGSLRPVQAQAQSDCSCSTVSPGVLAGIVMGDLVLTVLIALAVYFLGRLVPRGRGAAEAATRKQRITEGGSGGGSGGGSGGGSGGASSGVRLWATRQAMLGQVHEVPEGWLIFVAEQEELYVRVQNGFRKVQLEARTPLPRGTDNGSGGSGGSGGSGGSGGSGGSGMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 204 - DNA, 4491 bp, synthetic construct
  misc_feature 1..4491: note = Description of Artificial Sequence: Synthetic polynucleotide
  source 1..4491: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatctggtgctactaacttcagcctgctgaagcaggctggagacgtggaggagaaccctggacctggaggatccatgtgccaccaacagctcgtgatcagctggttctccctggtgttcctggctagccccctggtggccatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggatctggttctggttccggaagtggatctggttcagggtccagctttgaaatgcaaaaaggcggtagtctcagacctgttcaggcccaggctcagagcgattgcagctgtagcacagtgtctccaggcgtgctggccggaatcgtgatgggagatctggtgctgacagtgctgattgccctggccgtgtacttcctgggtagactggtgcctagaggaagaggcgctgccgaagccgccacaagaaagcagagaatcacagaaggcggctccggtggtggaagtggaggtggctctggtggtggcagcggaggtgcttcttctggtgttagactgtgggccaccagacaggccatgctgggacaagtgcacgaagtgcctgaaggctggctgatcttcgtggccgagcaagaggaactgtacgtgcgggtgcagaacggcttcagaaaggtgcagctggaagccagaacacccctgcctagaggcaccgacaatggttccggaggaagtggtggttctggtggttctggcggtagcggcggcagcggaggttccggtatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggattctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaa

SEQ ID NO: 205 - protein, 1497 aa, synthetic construct
  REGION 1..1497: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGSGSGSGSGSGSSFEMQKGGSLRPVQAQAQSDCSCSTVSPGVLAGIVMGDLVLTVLIALAVYFLGRLVPRGRGAAEAATRKQRITEGGSGGGSGGGSGGGSGMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGSGGSGGSGGSGGSGGSGGSGGASSGVRLWATRQAMLGQVHEVPEGWLIFVAEQEELYVRVQNGFRKVQLEARTPLPRGTDN

SEQ ID NO: 206 - DNA, 4491 bp, synthetic construct
  misc_feature 1..4491: note = Description of Artificial Sequence: Synthetic polynucleotide
  source 1..4491: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatctggtgctactaacttcagcctgctgaagcaggctggagacgtggaggagaaccctggacctggaggatccatgtgccaccaacagctcgtgatcagctggttctccctggtgttcctggctagccccctggtggccatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggatctggttctggttccggtagtggatctggttcaggatccagctttgaaatgcaaaaaggcggtagtctcagacctgttcaggcccaggctcagagcgattgcagctgtagcacagtgtctccaggcgtgctggccggaatcgtgatgggagatctggtgctgacagtgctgattgccctggccgtgtacttcctgggtagactggtgcctagaggaagaggcgctgccgaagccgccacaagaaagcagagaatcacagaaggcggatccggtggtggaagtggaggtggctctggtggtggcagcggcatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggattctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaaggtagtggaggaagtggtggttctggtggttctggcggtagcggcggcagcggaggttccggaggtgcttcttctggtgttagactgtgggccaccagacaggccatgctgggacaagtgcacgaagtgcctgaaggctggctgatcttcgtggccgagcaagaggaactgtacgtgcgggtgcagaacggcttcagaaaggtgcagctggaagccagaacacccctgcctagaggcaccgacaat

SEQ ID NO: 207 - protein, 1495 aa, synthetic construct
  REGION 1..1495: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGSGSGSGSGSGSSFEMQKGGSLTLRWELSSQPTIPIVGIIAGLVLLGAVITGAVVAAVMWRRKSSDRKGGSYTQAASSDSAQGSGGSGGGSGGGSGGGSGGASSGVRLWATRQAMLGQVHEVPEGWLIFVAEQEELYVRVQNGFRKVQLEARTPLPRGTDNGSGGSGGSGGSGGSGGSGGSGMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 208 - DNA, 4485 bp, synthetic construct
  misc_feature 1..4485: note = Description of Artificial Sequence: Synthetic polynucleotide
  source 1..4485: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatctggtgctactaacttcagcctgctgaagcaggctggagacgtggaggagaaccctggacctggaggatctatgtgccaccaacagctcgtgatcagctggttctccctggtgttcctggctagccccctggtggccatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggatctggttctggttccggaagtggatctggttcagggtccagctttgaaatgcaaaaaggcggtagtctgactcttaggtgggaactgagcagtcagcccactatccccattgtcggtatcattgccggactcgttttgcttggtgctgtgatcactggagcggtggtcgcggcagtgatgtggcggcgaaagtcaagcgacagaaagggcggctcctatactcaagccgcctcaagtgactctgcacagggtagcggcggctccggtggtggaagtggaggtggctctggtggtggcagcggaggtgcttcttctggtgttagactgtgggccaccagacaggccatgctgggacaagtgcacgaagtgcctgaaggctggctgatcttcgtggccgagcaagaggaactgtacgtgcgggtgcagaacggcttcagaaaggtgcagctggaagccagaacacccctgcctagaggcaccgacaatggttccggaggaagtggtggttctggtggttctggcggtagcggcggcagcggaggttccggtatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggattctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaa

SEQ ID NO: 209 - protein, 1495 aa, synthetic construct
  REGION 1..1495: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGSGSGSGSGSGSSFEMQKGGSLTLRWELSSQPTIPIVGIIAGLVLLGAVITGAVVAAVMWRRKSSDRKGGSYTQAASSDSAQGSGGSGGGSGGGSGGGSGMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGSGGSGGSGGSGGSGGSGGSGGASSGVRLWATRQAMLGQVHEVPEGWLIFVAEQEELYVRVQNGFRKVQLEARTPLPRGTDN

SEQ ID NO: 210 - DNA, 4485 bp, synthetic construct
  misc_feature 1..4485: note = Description of Artificial Sequence: Synthetic polynucleotide
  source 1..4485: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatctggtgctactaacttcagcctgctgaagcaggctggagacgtggaggagaaccctggacctggaggatctatgtgccaccaacagctcgtgatcagctggttctccctggtgttcctggctagccccctggtggccatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggatctggttctggttccggaagtggatctggttcaggatccagctttgaaatgcaaaaaggcggtagtctgactcttaggtgggaactgagcagtcagcccactatccccattgtcggtatcattgccggactcgttttgcttggtgctgtgatcactggagcggtggtcgcggcagtgatgtggcggcgaaagtcaagcgacagaaagggcggctcctatactcaagccgcctcaagtgactctgcacagggtagcggcggatccggtggtggaagtggaggtggctctggtggtggcagcggcatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggattctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaaggtagtggaggaagtggtggttctggtggttctggcggtagcggcggcagcggaggttccggaggtgcttcttctggtgttagactgtgggccaccagacaggccatgctgggacaagtgcacgaagtgcctgaaggctggctgatcttcgtggccgagcaagaggaactgtacgtgcgggtgcagaacggcttcagaaaggtgcagctggaagccagaacacccctgcctagaggcaccgacaat

SEQ ID NO: 211 - protein, 1501 aa, synthetic construct
  REGION 1..1501: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGMIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGGGGSGGGGSGASSGVRLWATRQAMLGQVHEVPEGWLIFVAEQEELYVRVQNGFRKVQLEARTPLPRGTDNGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 212 - DNA, 4503 bp, synthetic construct
  misc_feature 1..4503: note = Description of Artificial Sequence: Synthetic polynucleotide
  source 1..4503: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatctggtgctactaacttcagcctgctgaagcaggctggagacgtggaggagaaccctggacctggaggatccatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaagggaggttccggtggtggaagtggaggtggttccggaatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggcggaggtggtagcggcggaggcggaagtggtgcttcttctggtgttagactgtgggccaccagacaggccatgctgggacaagtgcacgaagtgcctgaaggctggctgatcttcgtggccgagcaagaggaactgtacgtgcgggtgcagaacggcttcagaaaggtgcagctggaagccagaacacccctgcctagaggcaccgacaatggatctggttctggaagtggatctggtagtggttccggttccatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagc

SEQ ID NO: 213 - protein, 1404 aa, synthetic construct
  REGION 1..1404: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGSGSGSGSGSGSSFEMQKLRPVQAQAQSDCSCSTVSPGVLAGIVMGDLVLTVLIALAVYFLGRLVPRGRGAAEAATRKQRITEGGSGGGSGSSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGHQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 214 - DNA, 4212 bp, synthetic construct
  misc_feature 1..4212: note = Description of Artificial Sequence: Synthetic polynucleotide
  source 1..4212: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatctggtgctactaacttcagcctgctgaagcaggctggagacgtggaggagaaccctggacctggaggatctatgtgccaccaacagctcgtgatcagctggttctccctggtgttcctggctagccccctggtggccatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggatctggttctggttccggaagtggatctggttcagggtccagctttgaaatgcaaaaactcagacctgttcaggcccaggctcagagcgattgcagctgtagcacagtgtctccaggcgtgctggccggaatcgtgatgggagatctggtgctgacagtgctgattgccctggccgtgtacttcctgggtagactggtgcctagaggaagaggcgctgccgaagccgccacaagaaagcagagaatcacagaaggcggcagcggcggaggatctggatcctcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgagaatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccatcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaa

SEQ ID NO: 215 - protein, 1481 aa, synthetic construct
  REGION 1..1481: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGMIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGGGGSGGGGSSGLPDVASLRQQVEALQGQVQHLQAAFSQYKKVELFPNGQSGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 216 - DNA, 4443 bp, synthetic construct
  misc_feature 1..4443: note = Description of Artificial Sequence: Synthetic polynucleotide
  source 1..4443: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatctggtgctactaacttcagcctgctgaagcaggctggagacgtggaggagaaccctggacctggaggatccatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaagggaggttccggtggtggaagtggaggtggttccggaatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggaggcggaggatctggcggaggcggatcttctggactgcctgatgtggccagcctgagacagcaggttgaagccctgcaaggccaggtgcagcatctgcaggctgccttcagccagtacaagaaggtggaactgttccccaacggccagagcggatctggttctggaagtggatctggtagtggttccggtagtatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagc

SEQ ID NO: 217 - protein, 1588 aa, synthetic construct
  REGION 1..1588: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGMIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENGGSGGSGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGSGSGSGSGSGSSFEMQKGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 218 - DNA, 4764 bp, synthetic construct
  misc_feature 1..4764: note = Description of Artificial Sequence: Synthetic polynucleotide
  source 1..4764: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatctggtgctactaacttcagcctgctgaagcaggctggagacgtggaggagaaccctggacctggaggatccatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaagggaggttccggtggtggaagtggaggtggttccggaatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacggtggaagtggtggaagtggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactcggatctggttctggaagtggatctggtagtggttccagctttgaaatgcaaaaaggcagcggaagtggtagtggttccatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagc

SEQ ID NO: 219 - protein, 1554 aa, synthetic construct
  REGION 1..1554: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMVGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 220 - DNA, 4662 bp, synthetic construct
  misc_feature 1..4662: note = Description of Artificial Sequence: Synthetic polynucleotide
  source 1..4662: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatctggtgctactaacttcagcctgctgaagcaggctggagacgtggaggagaaccctggacctggaggatccatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaagggaggttccggaggtggaagtggaggtggttccggagaggaatctgtcgtgcggccttccgtgtttgtggtggacggccagacagacatccccttcacaagactgggcagaagccacagacggcagagctgttctgtggccagagttggactgggactgctgctgcttctgatgggagctggactggctgtgcaaggctggtttctgctgcagctccattggagactgggcgagatggtcaccagactgcctgatggacctgccggatcttgggagcagctgatccaagagcggagaagccacgaagtgaaccctgccgcacatctgacaggcgccaattctagcctgacaggctctggtggacccctgctgtgggaaactcaactgggactcgccttcctgagaggcctgagctatcatgatggcgccctggtggttaccaaggccggctactactacatctacagcaaggtgcagctcggcggcgtgggatgtcctcttggactggcctctacaatcacccacggcctgtacaagcggacccctagataccccgaggaactggaactgctggtgtcccagcagagcccttgtggcagagccacaagcagctccagagtttggtgggacagcagctttctcggcggagtggtgcatctggaagccggcgaaaaggtggtcgtcagagtgctggatgagagactcgtgcggctgagagatggcacccggtcttacttcggcgccttcatggtgggttccggaagtggaagtggatctggtagtggttccggtagtatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagc

SEQ ID NO: 221 - protein, 1436 aa, synthetic construct
  REGION 1..1436: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGMIETYNQTSPRSAATGLPISMKARQKLQNLFINFCLILICLLLICIIVMLLLHRRLDKIEDERNLHEDFVFMKTIQRCNTGERSLSLLNCEEIKSQFEGFVKDIMLNKEETKKENSFEMQKGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 222 - DNA, 4308 bp, synthetic construct
  misc_feature 1..4308: note = Description of Artificial Sequence: Synthetic polynucleotide
  source 1..4308: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatctggtgctactaacttcagcctgctgaagcaggctggagacgtggaggagaaccctggacctggaggatccatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaagggaggttccggtggtggaagtggaggtggttccggaatgatagagacttacaaccaaacctccccaagatccgctgcaaccggcttgccaatatccatgaaggcaagacaaaaacttcagaacctgttcatcaatttctgtcttattcttatatgcttgctcctgatttgtattatcgtcatgctgcttcttcatcgccgactcgacaagatagaggacgaacgcaacttgcatgaagactttgtctttatgaaaactattcaaagatgcaacacaggtgaacgctccctgagcttgcttaactgtgaagaaataaagtcccaatttgagggttttgttaaagatataatgcttaacaaggaagagaccaaaaaagaaaattcttttgagatgcagaagggttccggaagcggaagtggatctggtagtggttccggtagtatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagc

SEQ ID NO: 223 - protein, 1395 aa, synthetic construct
  REGION 1..1395: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGDGQTDIPFTRLGRSHRRQSCSVARARQKLQNLFINFCLILICLLLICIIVMLLLQLHWRLGEMVTRLPDGPAGSWEQLIQGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 224 - DNA, 4185 bp, synthetic construct
  misc_feature 1..4185: note = Description of Artificial Sequence: Synthetic polynucleotide
  source 1..4185: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatctggtgctactaacttcagcctgctgaagcaggctggagacgtggaggagaaccctggacctggaggatccatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaagggaggttccggtggtggaagtggaggtggttccggagacggccagacagacatcccattcacaagactgggcagaagccacagacggcagagctgttctgtggccagagcaagacaaaaacttcagaacctgttcatcaatttctgtcttattcttatatgcttgctcctgatttgtattatcgtcatgctgcttctgcagctccattggagactgggcgagatggtcaccagactgcctgatggacctgccggatcttgggagcagctgatccaaggttccggaagcggaagtggatctggtagtggttccggtagtatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagc

SEQ ID NO: 225 - protein, 1450 aa, synthetic construct
  REGION 1..1450: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGSGGSGGSGGSGGSGGSGGSGSSDYSDLQRVKQELLEEVKKELQKVKEEIIEAFVQELRKRGSGGSGGGSGGGSGDGQTDIPFTRLGRSHRRQSCSVARVGLGLLLLLMGAGLAVQGWFLLQLHWRLGEMVTRLPDGPAGSWEQLIQGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 226 - DNA, 4350 bp, synthetic construct
  misc_feature 1..4350: note = Description of Artificial Sequence: Synthetic polynucleotide
  source 1..4350: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatctggtgctactaacttcagcctgctgaagcaggctggagacgtggaggagaaccctggacctggaggatccatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaagggaagcggaggaagtggtggttctggtggttctggcggtagcggcggcagcggaggttccggatctagcgactatagcgatttgcaacgggtgaaacaagaactgttggaagaagttaaaaaggagttgcagaaggtcaaggaggaaattattgaagccttcgtccaggaacttaggaaaagggggagcggaggttccggaggtggaagtggaggtggatcaggtgacggccagacagacatccccttcacaagactgggcagaagccacagacggcagagctgttctgtggccagagttggactgggactgctgctgcttctgatgggagctggactggctgtgcaaggctggtttctgctgcagctccattggagactgggcgagatggtcaccagactgcctgatggacctgccggatcttgggagcagctgatccaaggttccggatctggaagtggatctggtagtggttccggtagtatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagc

SEQ ID NO: 227 - protein, 1449 aa, synthetic construct
  REGION 1..1449: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGSGGSGGSGGSGGSGGSGGSGSGLPDVASLRQQVEALQGQVQHLQAAFSQYKKVELFPNGQSGGSGGGSGGGSGDGQTDIPFTRLGRSHRRQSCSVARVGLGLLLLLMGAGLAVQGWFLLQLHWRLGEMVTRLPDGPAGSWEQLIQGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 228 - DNA, 4347 bp, synthetic construct
  misc_feature 1..4347: note = Description of Artificial Sequence: Synthetic polynucleotide
  source 1..4347: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatctggtgctactaacttcagcctgctgaagcaggctggagacgtggaggagaaccctggacctggaggatccatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaagggaagcggaggaagtggtggttctggtggttctggcggtagcggcggcagcggaggttccggatctggactgcctgatgtggccagcctgagacagcaggttgaagccctgcaaggccaggtgcagcatctgcaggctgccttcagccagtacaagaaggtggaactgttccccaacggccagagcggaggttccggaggtggaagtggaggtggatcaggtgacggccagacagacatccccttcacaagactgggcagaagccacagacggcagagctgttctgtggccagagttggactgggactgctgctgcttctgatgggagctggactggctgtgcaaggctggtttctgctgcagctccattggagactgggcgagatggtcaccagactgcctgatggacctgccggatcttgggagcagctgatccaaggttccggatctggaagtggatctggtagtggttccggtagtatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagc

SEQ ID NO: 229 - protein, 1446 aa, synthetic construct
  REGION 1..1446: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMSGLPDVASLRQQVEALQGQVQHLQAAFSQYKKVELFPNGQSGSGGSGGSGGSGGSGGSGSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGDGQTDIPFTRLGRSHRRQSCSVARVGLGLLLLLMGAGLAVQGWFLLQLHWRLGEMVTRLPDGPAGSWEQLIQGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 230 - DNA, 4338 bp, synthetic construct
  misc_feature 1..4338: note = Description of Artificial Sequence: Synthetic polynucleotide
  source 1..4338: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatctggtgctactaacttcagcctgctgaagcaggctggagacgtggaggagaaccctggacctggaggatccatgtctggactgcctgatgtggccagcctgagacagcaggttgaagccctgcaaggccaggtgcagcatctgcaggctgccttcagccagtacaagaaggtggaactgttccccaacggccagagcggaagcggaggttctggtggttctggcggtagcggcggcagcggaggaagtggctcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaagggaggttccggaggtggaagtggaggtggatcaggtgacggccagacagacatccccttcacaagactgggcagaagccacagacggcagagctgttctgtggccagagttggactgggactgctgctgcttctgatgggagctggactggctgtgcaaggctggtttctgctgcagctccattggagactgggcgagatggtcaccagactgcctgatggacctgccggatcttgggagcagctgatccaaggttccggaagtggaagtggatctggtagtggttccggtagtatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagc

SEQ ID NO: 231 - protein, 1440 aa, synthetic construct
  REGION 1..1440: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGDGQTDIPFTRLGRSHRRQSCSVARVGLGLLLLLMGAGLAVQGWFLLQLHWRLGEMVTRLPDGPAGSWEQLIQGSGGSGGSGGSGSGLPDVASLRQQVEALQGQVQHLQAAFSQYKKVELFPNGQSGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 232 - DNA, 4320 bp, synthetic construct
  misc_feature 1..4320: note = Description of Artificial Sequence: Synthetic polynucleotide
  source 1..4320: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatctggtgctactaacttcagcctgctgaagcaggctggagacgtggaggagaaccctggacctggaggatccatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaagggaggttccggtggtggaagtggaggtggttccggagacggccagacagacatccccttcacaagactgggcagaagccacagacggcagagctgttctgtggccagagttggactgggactgctgctgcttctgatgggagctggactggctgtgcaaggctggtttctgctgcagctccattggagactgggcgagatggtcaccagactgcctgatggacctgccggatcttgggagcagctgatccaaggaagcggaggttctggtggttctggcggtagcggctctggactgcctgatgtggccagcctgagacagcaggttgaagccctgcaaggccaggtgcagcatctgcaggctgccttcagccagtacaagaaggtggaactgttccccaacggccaatccggttccggaagcggaagtggatctggtagtggttccggtagtatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagc

SEQ ID NO: 233 - protein, 1475 aa, synthetic construct
  REGION 1..1475: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGSGSGSGSGSGSSFEMQKGGSLTLRWELSSQPTIPIVGIIAGLVLLGAVITGAVVAAVMWRRKSSDRKGGSYTQAASSDSAQGSGGSGGGSGGGSGGGSGSGLPDVASLRQQVEALQGQVQHLQAAFSQYKKVELFPNGQSGSGGSGGSGGSGGSGGSGGSGMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 234 - DNA, 4425 bp, synthetic construct
  misc_feature 1..4425: note = Description of Artificial Sequence: Synthetic polynucleotide
  source 1..4425: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatctggtgctactaacttcagcctgctgaagcaggctggagacgtggaggagaaccctggacctggaggatctatgtgccaccaacagctcgtgatcagctggttctccctggtgttcctggctagccccctggtggccatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggatctggttctggttccggaagtggatctggttcagggtccagctttgaaatgcaaaaaggcggtagtctgactcttaggtgggaactgagcagtcagcccactatccccattgtcggtatcattgccggactcgttttgcttggtgctgtgatcactggagcggtggtcgcggcagtgatgtggcggcgaaagtcaagcgacagaaagggcggctcctatactcaagccgcctcaagtgactctgcacagggtagcggcggctccggtggtggaagtggaggtggctctggtggtggcagcggatccggactgcctgatgtggccagcctgagacagcaggttgaagccctgcaaggccaggtgcagcatctgcaggctgccttcagccagtacaagaaggtggaactgttccccaacggccaatccggttccggaggaagtggtggttctggtggttctggcggtagcggcggcagcggaggttccggtatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggattctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaa

SEQ ID NO: 235 - protein, 1470 aa, synthetic construct
  REGION 1..1470: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGSGSGSGSGSGSSFEMQKGGSSGLPDVASLRQQVEALQGQVQHLQAAFSQYKKVELFPNGQSGGSGGSGGGGSGGGGSLTLRWELSSQPTIPIVGIIAGLVLLGAVITGAVVAAVMWRRKSSDRKGGSYTQAASSDSAQGSGGSGGGSGGGSGGGSGMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 236 - DNA, 4410 bp, synthetic construct
  misc_feature 1..4410: note = Description of Artificial Sequence: Synthetic polynucleotide
  source 1..4410: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatctggtgctactaacttcagcctgctgaagcaggctggagacgtggaggagaaccctggacctggaggatctatgtgccaccaacagctcgtgatcagctggttctccctggtgttcctggctagccccctggtggccatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggatctggttctggttccggaagtggatctggttcaggatccagctttgaaatgcaaaaaggcggtagttctggactgcctgatgtggccagcctgagacagcaggttgaagccctgcaaggccaggtgcagcatctgcaggctgccttcagccagtacaagaaggtggaactgttccccaacggccagagcggcggcagtggcggttctggaggtggaggaagcggaggtggcggaagtctgactcttaggtgggaactgagcagtcagcccactatccccattgtcggtatcattgccggactcgttttgcttggtgctgtgatcactggagcggtggtcgcggcagtgatgtggcggcgaaagtcaagcgacagaaagggcggctcctatactcaagccgcctcaagtgactctgcacagggtagcggcggatccggtggtggaagtggaggtggctctggtggtggcagcggcatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggattctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaa

SEQ ID NO: 237 - protein, 1475 aa, synthetic construct
  REGION 1..1475: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGSGSGSGSGSGSSFEMQKGGSLTLRWELSSQPTIPIVGIIAGLVLLGAVITGAVVAAVMWRRKSSDRKGGSYTQAASSDSAQGSGGSGGGSGGGSGGGSGMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGSGGSGGSGGSGGSGGSGGSGSGLPDVASLRQQVEALQGQVQHLQAAFSQYKKVELFPNGQS

SEQ ID NO: 238 - DNA, 4425 bp, synthetic construct
  misc_feature 1..4425: note = Description of Artificial Sequence: Synthetic polynucleotide
  source 1..4425: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatctggtgctactaacttcagcctgctgaagcaggctggagacgtggaggagaaccctggacctggaggatctatgtgccaccaacagctcgtgatcagctggttctccctggtgttcctggctagccccctggtggccatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggatctggttctggttccggaagtggatctggttcaggatccagctttgaaatgcaaaaaggcggtagtctgactcttaggtgggaactgagcagtcagcccactatccccattgtcggtatcattgccggactcgttttgcttggtgctgtgatcactggagcggtggtcgcggcagtgatgtggcggcgaaagtcaagcgacagaaagggcggctcctatactcaagccgcctcaagtgactctgcacagggtagcggcggatccggtggtggaagtggaggtggctctggtggtggcagcggcatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaactaatctgaggattctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaaggtagtggaggaagtggtggttctggtggttctggcggtagcggcggcagcggaggaagtggctctggactgcctgatgtggccagcctgagacagcaggttgaagccctgcaaggccaggtgcagcatctgcaggctgccttcagccagtacaagaaggtggaactgttccccaacggccagagc

SEQ ID NO: 239 - protein, 4 aa, synthetic construct
  REGION 1..4: note = Description of Artificial Sequence: Synthetic peptide
GGSG

SEQ ID NO: 240 - protein, 20 aa, synthetic construct
  REGION 1..20: note = Description of Artificial Sequence: Synthetic peptide
  SITE 1..20: note = This sequence may encompass 1-10 "Gly Ser" repeating units
  SITE 1..20: note = See specification as filed for detailed description of substitutions and preferred embodiments
GSGSGSGSGSGSGSGSGSGS

SEQ ID NO: 241 - protein, 30 aa, synthetic construct
  REGION 1..30: note = Description of Artificial Sequence: Synthetic polypeptide
  SITE 1..30: note = This sequence may encompass 1-10 "Gly Ser Gly" repeating units
  SITE 1..30: note = See specification as filed for detailed description of substitutions and preferred embodiments
GSGGSGGSGGSGGSGGSGGSGGSGGSGGSG

SEQ ID NO: 242 - protein, 40 aa, synthetic construct
  REGION 1..40: note = Description of Artificial Sequence: Synthetic polypeptide
  SITE 1..40: note = This sequence may encompass 1-10 "Gly Gly Ser Gly" repeating units
  SITE 1..40: note = See specification as filed for detailed description of substitutions and preferred embodiments
GGSGGGSGGGSGGGSGGGSGGGSGGGSGGGSGGGSGGGSG

SEQ ID NO: 243 - DNA, 12 bp, synthetic construct
  misc_feature 1..12: note = Description of Artificial Sequence: Synthetic oligonucleotide
  source 1..12: mol_type = other DNA
aacccaggaaca

SEQ ID NO: 244 - protein, 4 aa, synthetic construct
  REGION 1..4: note = Description of Artificial Sequence: Synthetic peptide
NPGT

SEQ ID NO: 245 - protein, 513 aa, synthetic construct
  REGION 1..513: note = Synthetic Construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 246 - protein, 1408 aa, synthetic construct
  REGION 1..1408: note = Synthetic Construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGHQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGMIETYNQTSPRSAATGLPISMKARQKLQNLFINFCLILICLLLICIIVMLLLHRRLDKIEDERNLHEDFVFMKTIQRCNTGERSLSLLNCEEIKSQFEGFVKDIMLNKEETKKENSFEMQKGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 247 - protein, 1419 aa, synthetic construct
  REGION 1..1419: note = Synthetic Construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMSGLPDVASLRQQVEALQGQVQHLQAAFSQYKKVELFPNGQSGSGGSGGSGGSGGSGGSGSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGHQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGDGQTDIPFTRLGRSHRRQSCSVARVGLGLLLLLMGAGLAVQGWFLLQLHWRLGEMVTRLPDGPAGSWEQLIQGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 248 - protein, 1136 aa, synthetic construct
  REGION 1..1136: note = Synthetic Construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMSGLPDVASLRQQVEALQGQVQHLQAAFSQYKKVELFPNGQSGSGGSGGSGGSGGSGGSGSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGHQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGMIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGSGSGSGSGSGSGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 249 - protein, 1052 aa, synthetic construct
  REGION 1..1052: note = Synthetic Construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMALTFALLVALLVLSCKSSCSVGCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEGSGSGSGSGSGSGSSFEMQKLRPVQAQAQSDCSCSTVSPGVLAGIVMGDLVLTVLIALAVYFLGRLVPRGRGAAEAATRKQRITEGGSGGGSGSSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGHQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 250 - protein, 1455 aa, synthetic construct
  REGION 1..1455: note = Synthetic Construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGMIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSPWIVLKEPISVSSEQVLKFRKLNFNGEGEPEELMVDNWRPAQPLKNRQIKASFK

SEQ ID NO: 251 - protein, 1531 aa, synthetic construct
  REGION 1..1531: note = Synthetic Construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGHQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 252 - protein, 1432 aa, synthetic construct
  REGION 1..1432: note = Synthetic Construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMSGLPDVASLRQQVEALQGQVQHLQAAFSQYKKVELFPNGQSGSGGSGGSGGSGGSGGSGSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGHQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGPEAPWPPAPRARACRVLPWALVAGLLLLLLLAAACAVFLACPWAVSGARASPGSAASPRLREGPELSPDDPAGLLDLRQGMFAQLGSGSGSGSGSGSGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 253 - protein, 1288 aa, synthetic construct
  REGION 1..1288: note = Synthetic Construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMALTFALLVALLVLSCKSSCSVGIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSPGSGSGSGSGSGSGSKQEHFPDNLLPSWAITLISVNGIFVICCLTYCFAPRCRERRGSSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGHQKVVDVLDSIKTKGKSADFTNFDPRGLLPESLDYWTYPGSLTTPPLLECVT

SEQ ID NO: 254 - protein, 699 aa, synthetic construct
  REGION 1..699: note = Synthetic Construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMALTFALLVALLVLSCKSSCSVGCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 255 - protein, 1082 aa, synthetic construct
  REGION 1..1082: note = Synthetic Construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGHQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGMIETYNQTSPRSAATGLPISMKARQKLQNLFINFCLILICLLLICIIVMLLLHRRLDKIEDERNLHEDFVFMKTIQRCNTGERSLSLLNCEEIKSQFEGFVKDIMLNKEETKKENSFEMQKGSGSGSGSGSGSGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 256 - protein, 1093 aa, synthetic construct
  REGION 1..1093: note = Synthetic Construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMSGLPDVASLRQQVEALQGQVQHLQAAFSQYKKVELFPNGQSGSGGSGGSGGSGGSGGSGSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGHQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGDGQTDIPFTRLGRSHRRQSCSVARVGLGLLLLLMGAGLAVQGWFLLQLHWRLGEMVTRLPDGPAGSWEQLIQGSGSGSGSGSGSGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 257 - protein, 1136 aa, synthetic construct; identical to SEQ ID NO: 248
  REGION 1..1136: note = Synthetic Construct

SEQ ID NO: 258 - protein, 1052 aa, synthetic construct; identical to SEQ ID NO: 249
  REGION 1..1052: note = Synthetic Construct

SEQ ID NO: 259 - protein, 1129 aa, synthetic construct
  REGION 1..1129: note = Synthetic Construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGMIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGSGSGSGSGSGSGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEWIVLKEPISVSSEQVLKFRKLNFNGEGEPEELMVDNWRPAQPLKNRQIKASFK

SEQ ID NO: 260 - protein, 1205 aa, synthetic construct
  REGION 1..1205: note = Synthetic Construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGHQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSGSGSGSGSGSGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 261 - protein, 1106 aa, synthetic construct
  REGION 1..1106: note = Synthetic Construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMSGLPDVASLRQQVEALQGQVQHLQAAFSQYKKVELFPNGQSGSGGSGGSGGSGGSGGSGSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGHQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGPEAPWPPAPRARACRVLPWALVAGLLLLLLLAAACAVFLACPWAVSGARASPGSAASPRLREGPELSPDDPAGLLDLRQGMFAQLGSGSGSGSGSGSGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 262 - protein, 962 aa, synthetic construct
  REGION 1..962: note = Synthetic Construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMALTFALLVALLVLSCKSSCSVGCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEGSGSGSGSGSGSGSKQEHFPDNLLPSWAITLISVNGIFVICCLTYCFAPRCRERRGSSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGHQKVVDVLDSIKTKGKSADFTNFDPRGLLPESLDYWTYPGSLTTPPLLECVT

SEQ ID NO: 263 - protein, 997 aa, synthetic construct
  REGION 1..997: note = Synthetic Construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGSKQRGTFSEVSLAQDARQKLQNLFINFCLILICLLLICIIVMLLIPFLEQNNSSPNTRTQKSFEMQKGSGSGSGSGSGSGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 264 - protein, 1104 aa, synthetic construct
  REGION 1..1104: note = Synthetic Construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMSGLPDVASLRQQVEALQGQVQHLQAAFSQYKKVELFPNGQSGSGGSGGSGGSGGSGGSGSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGMIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGSGSGSGSGSGSGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 265 - protein, 1191 aa, synthetic construct
  REGION 1..1191: note = Synthetic Construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGMIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENGGSGGSGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGSGSGSGSGSGSGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 266 - protein, 1116 aa, synthetic construct
  REGION 1..1116: note = Synthetic Construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGMIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENGGSGGSGGGGSGGGGSGASSGVRLWATRQAMLGQVHEVPEGWLIFVAEQEELYVRVQNGFRKVQLEARTPLPRGTDNGSGSGSGSGSGSGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 267 - protein, 1090 aa, synthetic construct
  REGION 1..1090: note = Synthetic Construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGMIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENGGGGSGGGGSSGLPDVASLRQQVEALQGQVQHLQAAFSQYKKVELFPNGQSGSGSGSGSGSGSGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 268 - protein, 1191 aa, synthetic construct
  REGION 1..1191: note = Synthetic Construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGGGSGMIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGSGSGSGSGSGSGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 269 - protein, 1108 aa, synthetic construct
  REGION 1..1108: note = Synthetic Construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGSGGSGGSGGSGGSGGSGGSGSSDYSDLQRVKQELLEEVKKELQKVKEEIIEAFVQELRKRGSGGSGGGSGGGSGMIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGSGSGSGSGSGSGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 270 - protein, 1093 aa, synthetic construct
  REGION 1..1093: note = Synthetic Construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGGSMDMRVPAQLLGLLLLWLSGARCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGSGSGSGSGSGSGSSFEMQKGGSLRPVQAQAQSDCSCSTVSPGVLAGIVMGDLVLTVLIALAVYFLGRLVPRGRGAAEAATRKQRITEGGSGGGSGGGSGGGSGSSDYSDLQRVKQELLEEVKKELQKVKEEIIEAFVQELRKRGSGSGGSGGSGGSGGSGGSGGSGMSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 271 - protein, 666 aa, synthetic construct
  REGION 1..666: note = Synthetic Construct
MDMRVPAQLLGLLLLWLSGARCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGSGSGSGSGSGSGSGSGSGSGSGSGSGSGSKQEHFPDNLLPSWAITLISVNGIFVICCLTYCFAPRCRERRGSSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGSGATNFSLLKQAGDVEENPGPMGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

(19) United States
(12) Patent Application Publication
(10) Pub. No.: US 2024/0424136 A1
(43) Pub. Date: Dec. 26, 2024

(54) COMPOSITIONS AND SYSTEMS FOR REGULATION OF FUNCTION/ABUNDANCE AND DELIVERY OF POLYPEPTIDE PAYLOADS

(71) Applicant: OBSIDIAN THERAPEUTICS, INC., Cambridge, MA (US)

(72) Inventors: Dhruv Sethi, Westwood, MA (US); Sean Smith, Cambridge, MA (US); Michelle Ols, Northborough, MA (US); Dexue Sun, Cambridge, MA (US); Dan Jun Li, Cambridge, MA (US); Jan ter Meulen, Cambridge, MA (US); Michael Schebesta, Cambridge, MA (US)

(73) Assignee: OBSIDIAN THERAPEUTICS, INC., Cambridge, MA (US)

(21) Appl. No.: 18/696,810

(22) PCT Filed: Oct. 18, 2022
(86) PCT No.: PCT/US2022/046998

§ 371 Date: Mar. 28, 2024

Related U.S. Application Data
(60) Provisional application No. 63/262,646, filed on Oct. 18, 2021.
(60) Provisional application No. 63/342,443, filed on May 16, 2022.

(51) Int. Cl. A61K 48/00; A61K 38/17; A61K 38/19; A61K 47/64; C12N 15/86

(52) U.S. Cl. CPC A61K 48/005; A61K 38/1774; A61K 38/19; A61K 47/64; C12N 15/86; C12N 2740/15043

(57) ABSTRACT

Provided herein are engineered, regulatable polypeptides comprising a payload and a modulation hub with at least two drug responsive domains (DRD), wherein the modulation hub is operably linked to the payload and wherein the at least two DRDs are responsive to a ligand. Also provided are engineered monomers comprising an oligomerization domain and a payload, a DRD or both a payload and DRD, wherein the monomers are configured to assemble into oligomers by way of the oligomerization domain and the oligomers comprise at least one payload operably linked to a modulation hub. The abundance, availability and/or biological activity of the payload is regulated by interaction of the one or more DRDs and an effective amount of ligand.

### CROSS REFERENCE TO RELATED APPLICATIONS

This application claims priority to U.S. Provisional Application No. 63/262,646, filed Oct. 18, 2021 and U.S. Provisional Application No. 63/342,443, filed May 16, 2022, which are incorporated by reference herein in their entireties.

### REFERENCE TO THE SEQUENCE LISTING

The instant application contains a Sequence Listing which has been submitted electronically in XML format and is hereby incorporated by reference in its entirety. Said Sequence Listing XML, created on Oct. 18, 2022, is named 108407-1354052-October 18.xml and is 691,346 bytes in size.

### BACKGROUND

Biological products (biologics) as defined by the U.S. Food and Drug Administration include vaccines, blood and blood components, allergens, somatic cells, gene therapy, tissues, and recombinant therapeutic proteins. Cells from the same subject (autologous), from a subject of the same species (homologous or allogenic), or even from a different species (heterologous) can be administered to a subject as a biologic. In adoptive cell therapy (“ACT”), for example, T cells originating from a subject (or sometimes from a different source) are removed, genetically engineered to address the specific needs of the same or different subject, and then transferred back into the same or different subject. There are currently obstacles to widespread adoption and success of biologics such as ACT, however. Maintaining therapeutically effective levels of a biologic administered to a patient can be difficult, as the biologic may be expressed by an engineered cell at toxic levels or may be present or expressed for an unacceptably sustained duration. There is therefore a need for regulation of the biologics for optimization of therapeutic benefits and to facilitate widespread adoption of biologic therapies.

### SUMMARY

This disclosure relates to compositions and systems capable of regulating payload function, and methods of making and using the compositions and systems. Payload function can be regulated by modifying and/or modulating, among other things, abundance and/or activity of the payload. Described herein are compositions and systems capable of modifying and/or modulating payload activity and/or abundance, as well as methods of making and using the compositions and systems.

The present disclosure provides compositions, referred to as “modulation hubs,” comprising two or more drug responsive domains (“DRD”). DRDs are polypeptides that can regulate the abundance, availability, and/or activity of a payload under appropriate conditions, for example, in response to a small molecule ligand. In some embodiments, modulation hubs comprise DRDs linked in series by way of covalent bonds. In such embodiments, the DRDs are directly linked or indirectly linked, for example, through linkers or hinges. In certain embodiments, the modulation hubs comprise DRDs associated in oligomers (also referred to as multimeric fusion compositions or fusion compositions) of at least two DRD-oligomerization domain constructs, wherein the DRD-oligomerization domain constructs each comprise a DRD linked, directly or indirectly, to an oligomerization domain configured to promote oligomerization of the DRD-oligomerization domain constructs. In some embodiments, wherein the oligomerization domain is a trimerizing domain, the multimeric fusion compositions include three DRD-oligomerization domain constructs resulting in a modulation hub having at least three DRDs and three trimerizing domains. In any of the embodiments of the modulation hub, the DRDs in the modulation hub may all be the same, may all be different, or some may be the same and some may be different.

The present disclosure also provides compositions referred to as polypeptide monomers comprising an oligomerization domain configured to promote oligomerization of the polypeptide monomer; and, at least one payload having a biological activity under appropriate conditions, or at least one drug responsive domain (DRD) responsive to a ligand. In some embodiments, the regulatable polypeptide monomers comprise an oligomerization domain configured to promote oligomerization of the polypeptide, at least one payload having a biological activity level under appropriate conditions, and at least one drug responsive domain. In some such embodiments, for example those involving the same monomers, multiple monomers oligomerize to form an oligomer comprising at least two payloads operably linked to a modulation hub, which in turn comprises at least two oligomerization domains and at least two DRDs. In other embodiments, for example those involving different monomers, the multiple monomers oligomerize to form an oligomer comprising at least one payload operably linked to a modulation hub, which in turn comprises at least two oligomerization domains and at least two DRDs. In certain embodiments, regulation of payload abundance and/or function in the resultant oligomer is improved as compared to the same payload in a control construct. For example, in certain embodiments, the level of activity of the payload in the oligomer has a range spanning from a basal level of activity in the absence of ligand to a maximum activity level in the presence of a saturating amount of ligand and the basal activity level of the payload in the oligomer is less than that of the same payload in a control construct. In certain embodiments, the range of activity of the payload in the oligomer is greater than the range of activity of the same payload in a control construct. Thus, a better dose response is provided in the presence of the small molecule ligand specific for the DRD.

Also described herein are engineered, regulatable oligomers comprising at least two polypeptide monomers, wherein each monomer comprises an oligomerization domain configured to promote oligomerization of the monomers. Also described herein are engineered, regulatable oligomers comprising at least two polypeptide monomers, wherein each monomer comprises an oligomerization domain configured to promote oligomerization of the monomers, and at least one payload having a biological activity, or at least one DRD responsive to a ligand, or at least one payload having a biological activity and at least one DRD responsive to a ligand, provided that the oligomer comprises at least one payload and at least two DRDs. In some embodiments, the oligomer comprises at least two payloads and at least two DRDs. In some embodiments, the at least two payloads and the at least two DRDs are present in a 1:1 ratio.

In some embodiments, the engineered, regulatable oligomer comprises at least two polypeptide monomers, wherein each monomer comprises an oligomerization domain, at least one payload, and at least one DRD, such that the oligomer comprises at least two payloads operably linked to at least two DRDs. The oligomer can be formed by the same or different monomers, and each of those monomers can have one or more DRDs and/or one or more payloads. If a monomer has more than one DRD and/or more than one payload, the multiple DRDs and multiple payloads in the oligomer will be the same if the oligomer is formed from all the same monomers and all DRDs and all payloads in the monomer are the same. Alternatively, the multiple DRDs and multiple payloads in the oligomer will be different if the oligomer is formed from different monomers having different payloads and DRDs or the oligomer is formed from the same monomers, but each monomer has multiple different payloads and multiple different DRDs. In certain embodiments, the oligomer may have multiples of the same payload and multiple different DRDs or multiple different payloads and multiple of the same DRD.

These engineered, regulatable oligomers are optionally hetero-oligomers comprising either different DRDs, different payloads, or different DRDs and different payloads. In some hetero-oligomer embodiments, the oligomerization domain of one of the monomers is a hetero-oligomerization domain. In certain hetero-oligomer embodiments, the engineered, regulatable oligomer comprises at least two polypeptide monomers constituting a first, a second, and a third polypeptide monomer, wherein the first and second polypeptide monomers are the same, wherein each of the first and second polypeptide monomers comprises an oligomerization domain and a DRD, and wherein the third polypeptide monomer comprises a hetero-oligomerization domain and a payload.

In embodiments of the regulatable, engineered oligomers, regulation of function of a payload in the oligomer is improved as compared to the same payload in a control construct. For example, in certain embodiments, the biological activity of a payload in the engineered, regulatable oligomer has a range spanning from a basal level of activity in the absence of ligand to a maximum activity in the presence of a saturating amount of ligand and the basal activity level of the payload in the oligomer is less than that of the same payload in a control construct. In some embodiments, the range of biological activity of a payload in the oligomer is greater than the range of the same payload in a control construct.

Also provided are compositions referred to as engineered, regulatable polypeptides with at least one payload having a biological activity level and a modulation hub having at least two drug responsive domains (DRD), wherein the modulation hub is operably linked to the payload and wherein the at least two DRDs are responsive to a ligand. The biological activity of the payload is regulated by interaction of the at least two DRDs of the modulation hub with an effective amount of the ligand under appropriate conditions. In embodiments, regulation of function of the at least one payload within the polypeptide is improved as compared to the same payload in a control construct. For example, in some such embodiments, the activity of the at least one payload ranges from a basal activity level in the absence of the ligand to a maximum activity in the presence of a saturating amount of the ligand, and the basal activity of the at least one payload is lower than that of the same payload in a control construct. As another example, the range of activity level and the dose-response of the at least one payload in the engineered, regulatable polypeptide is optionally greater than that of the same payload in a control construct.

Provided herein are expressible nucleic acid constructs and vectors that encode one or more engineered, regulatable polypeptide monomers, which, when expressed, are capable of assembling into oligomers. Also provided are pluralities of nucleic acid constructs encoding the same or different engineered, regulatable polypeptide monomers such that, when expressed, assemble into oligomers, wherein the oligomers comprise at least one payload operably linked to a modulation hub comprising at least two DRDs. In embodiments the oligomers comprise at least two payloads operably linked to a modulation hub comprising at least two DRDs, wherein the ratio of payload:DRD is 1:1. The disclosure also provides nucleic acid constructs encoding engineered, regulatable polypeptides.

Cells containing one or more expressible nucleic acid constructs or vectors are also provided. Optionally, the cell is a human cell, for example, an immune cell.

The disclosure also provides systems for modifying and/or regulating (hereinafter “regulating”) the function of a payload, comprising a ligand and any of the referenced polypeptides, oligomers, nucleic acid constructs, vectors, or cells.

Disclosed herein are methods of regulating the function of a payload. In some embodiments, the method of regulation comprises engineering a cell to express a payload operably linked to a modulation hub comprising two or more DRDs. In such embodiments, the level of activity of the payload in the absence of ligand is reduced as compared to a control cell, for example, a cell engineered to express the payload independent of a DRD. Optionally, the method comprises contacting a cell engineered to express a payload operably linked to a modulation hub comprising two or more DRDs responsive to a ligand with an effective amount of the ligand such that the activity level of the payload is increased relative the basal activity level. Optionally, the two or more DRDs are responsive to the same ligand and the method comprises alternatively contacting the cell with a first selected dose of a ligand and a second selected dose of the same ligand, wherein the first and second selected dose of the ligand results in a selected activity level of the payload, the first and second selected dose of the ligand may be near zero and up to a saturating dose and may be the same or different. In embodiments wherein the modulation hub comprises a first DRD responsive to a first ligand and a second DRD responsive to a second ligand, and further wherein the first DRD is different from the second DRD and the first ligand is different from the second ligand, these embodiments optionally comprise contacting the hub with a selected amount of the first ligand and a selected amount of the second ligand, wherein the first ligand is different from the second ligand.

Disclosed herein are methods of controlling the dose or duration of administration of a payload to a subject, comprising administering to the subject one or more nucleic acid constructs, vectors, or cells as described herein. The method optionally further includes administering to the subject a selected amount of a ligand to deliver a selected activity of the payload to the subject thereby controlling the activity level of the payload administered by way of the one or more nucleic acid constructs, vectors or cells.

Also disclosed herein are methods of regulating expression of a downstream target protein or peptide of a gene editing process. Such methods can comprise engineering a cell to express an engineered, regulatable oligomer or polypeptide comprising a payload such as a CAS9 protein or transcription factor protein involved in controlling a gene editor, wherein the payload is operably linked to a modulation hub. In such methods, a nucleic acid construct or vector described herein is administered to a cell (in vivo, ex vivo, or in vitro) or a cell comprising the nucleic acid or vector is administered to a subject to regulate expression of the target protein or peptide.

The identified embodiments are exemplary only and are therefore non-limiting. The details of one or more non-limiting embodiments of the invention are set forth in the accompanying drawing and the description below. Other embodiments of the invention should be apparent to those of ordinary skill in the art after consideration of the present disclosure.

### BRIEF DESCRIPTION OF THE DRAWINGS

FIG. 1 schematically illustrates polypeptide constructs useful in understanding the scope of the disclosure. FIG. 1A depicts a construct, which produces a control composition, a non-oligomerized polypeptide. FIG. 1B depicts an embodiment of a construct according to this disclosure that comprises a trimerizing oligomerization domain, which produces monomers that self-assemble into a trimerized regulatable payload composition. FIG. 1C depicts the engineered regulatable payload composition that results from association of the monomers of FIG. 1B. FIG. 1D depicts illustrative examples of engineered regulatable payload compositions having a modulation hub with two DRDs operably connected to a payload in accordance with certain embodiments of the present disclosure. The embodiment of FIG. 1D lacks a transmembrane region, thus showing an embodiment for an intracellular polypeptide.

FIG. 2 schematically illustrates various construct monomers, each having a payload operably connected to an oligomerization domain and DRD. When expressed, the construct monomers self-assemble via the oligomerization domains and form a multimeric regulatable polypeptide (an engineered regulatable polypeptide) according to embodiments of the present disclosure. The degree of multimerization is generally determined by the choice of oligomerization domain. Thus, for example, constructs having CD40L extracellular domain (ECD), Collagen 18, langerin, collectin 7, DAP12, and 4-1BBl oligomerization domains tend to form trimerized regulatable payload compositions, whereas those with VASP oligomerization domains tend to form tetrameric regulatable payload compositions, and those with phospholamban oligomerization domains tend to form pentameric regulatable payload compositions.

FIG. 3 shows schematics of the polypeptide components encoded in constructs: IL12-217 (SEQ ID NOs: 125 and 126), IL12-223 (SEQ ID NOs: 127 and 128), IL12-241-252 (SEQ ID NOs: 129-152), IL12-254-262 (SEQ ID NOs: 153-170), CD19-IL12-192 (SEQ ID NOs: 123 and 124), CD19-IL12-297-316 (SEQ ID NOs: 171-210), and CD19-IL12-319-332 (SEQ ID NOs: 211-238). Although the schematics of CD19-IL12-192 (SEQ ID NOs: 123 and 124), CD19-IL12-297-316, and CD19-IL12-319-332 do not show the CD19-CAR sequence located at the beginning of the construct, it is present separated by a P2A sequence and included in the sequence listing for each construct. IL12-217 through IL12-262 include mCherry separated by a P2A sequence in the construct, whereas IL12-192 and IL12-297 through IL12-332 include CD19-CAR at the beginning of the construct. These additional domains are present and included in the sequence listing for each construct. Certain of the constructs encode control compositions. Control compositions referred to as “control constructs” are those that encode polypeptides and oligomers that lack an oligomerization domain and/or lack multiple DRDs (e.g., IL12-217 and IL12-223) as defined in the definitions section of this specification. Control compositions that are referred to as “DRD controls” are those lacking a functional DRD. An example of a DRD control is a construct with a wild type (wt) DRD such as CA2(wt). Other constructs described herein are polypeptides with multiple DRDs and polypeptide monomers, which can self-assemble into regulatable oligomers according to embodiments of this disclosure. Other constructs encode polypeptide monomers which can self-assemble into regulatable polypeptides according to embodiments of this disclosure.

FIG. 4 shows the representative procedure for in vitro characterization and/or validation of CA2 regulatable membrane-bound IL12 (“mbIL12”) expression in T cells. On day 0, peripheral blood T cells were stimulated with Dynabeads (T-expander CD3/CD28). On day 1, cells were transduced with lentiviral vectors. On day 6, the cells were assessed for transduction efficiency using flow cytometry. As a readout of cell transduction, CD19-CAR expression was detected using a CD19-Fc reagent followed by a fluorescently labeled anti-human Fc antibody. On day 8, a normalized number of transduced (CAR+) cells were plated on a 96-well plate and treated with either acetazolamide (ACZ) or an equivalent volume of DMSO vehicle control in the absence or presence of antigen re-stimulation. Stimulation conditions were either human Immunocult soluble CD3/CD28 reagent (StemCell Technologies, catalogue number 10971), K562 cells (Lozzio and Lozzio (1975) Blood 45:321-334) stably expressing the CAR antigen, CD19, at an effector to target (E:T) ratio of 1:1, or parental control K562 cells. On day 9, mbIL12 expression on transduced T-cells was analyzed.

FIG. 5A-C provides flow cytometry graphs showing surface expression (abundance) of IL12 in HEK cells transfected with control and inventive constructs, demonstrating regulation of IL12 function in HEK cells engineered according to embodiments of the disclosure. The constructs include mCherry (not shown).

FIG. 6 is a graph showing geometric Mean Fluorescent Intensity (“MFI”) indicative of IL12 surface expression (abundance) in Jurkat cells transduced with control (217) and inventive constructs (241, 243, 245, 247, 248, 254, 256, and 258, which include mCherry), demonstrating regulation of IL12 function in Jurkat cells according to embodiments of this disclosure.

FIG. 7 is a graph showing that the level of IL12p70 secreted from T-cells transduced with control (192 and 223) and inventive constructs (241, 245, 247, 256, and 260) is consistent with results in Jurkat cells of FIG. 6, demonstrating regulation of IL12 according to embodiments of this disclosure. Approximately equally transduced cells (15-25%) were plated in equal numbers. Constructs include mCherry (not shown), except for CD19 IL12-192.

FIG. 8 is a graph showing geometric MFI indicative of IL12 abundance on the surface of CAR-T cells transduced with control (192) and inventive constructs (241, 245, 247, 256, 260), demonstrating regulation of IL12 function and lower basal off-state levels of the inventive as compared to control constructs. Constructs include mCherry (not shown).

FIG. 9 provides schematic illustrations of polypeptide monomer constructs in accordance with embodiments of the disclosure. CD19-IL12-302-306 (SEQ ID NOs: 181-189) and CD19-IL12-319-329 (SEQ ID NOs: 211-231). Although contained within the test constructs, the CD19-CAR sequence is not shown in the FIG. 9. TM, transmembrane domain. Cyto, cytoplasmic tail.

FIG. 10A shows the MFI representative of membrane-bound IL12 expression in activated T cells transduced with inventive (CD19-IL12-302-306 and CD19-IL12-319-329) and control (CD19-IL12-192, empty vector (EV), untransduced (UT)) constructs, demonstrating lower basal off-states for the cells engineered with inventive as compared to control constructs. The results are shown for CD19-CAR T cells.

FIG. 10B shows the MFI representative of membrane-bound IL12 abundance (determined by CD19-CAR positivity) in non-activated CAR T cells transduced with the same constructs as shown in FIG. 10A, again demonstrating lower basal off-states for cells engineered with inventive as compared to control constructs.

FIG. 11A is a graph of the abundance of IL12 shed into 200 μl of media over 20 hours from 40,000 activated CD-19 CAR+ engineered T cells of FIG. 10A, demonstrating regulation was largely maintained and comparable to the results of FIG. 10A.

FIG. 11B is a graph of the abundance of IL12 shed into 200 μl of media over 20 hours from 40,000 CD-19 non-activated CAR+ of FIG. 10B, demonstrating regulation was largely maintained and comparable to the results of FIG. 10B.

FIG. 12 illustrates a procedure for testing IL12 abundance and activity by measuring phosphorylation of STAT4.

FIG. 13A shows the MFI detected from pSTAT expression in NK bystander assays as described in Example 7, and suggests that regulation of the shed portion of constructs in accordance with embodiments of the disclosure is functionally significant.

FIG. 13B shows the standard curve of pSTAT MFI values in relation to IL12 concentrations.

FIG. 14 schematically illustrates additional embodiments of polypeptide monomer constructs each comprising a payload, a DRD and an oligomerization domain, which may self-assemble into oligomers according to this disclosure.

FIG. 15 are stylized illustrations of regulatable payload compositions (showing IL12 as an exemplary payload or indicating Protein of Interest (POI) as a generic payload and showing CA2 as the exemplary DRDs or DRD1-3 as generic DRDs) that are produced by cells engineered to express nucleic acid constructs in accordance with embodiments of the disclosure.

FIG. 16 illustrates exemplary stylized nucleic acid constructs comprising a payload and a modulation hub comprising more than one DRD.

FIG. 17 is an example of a monomer construct that assembles into a regulatable oligomer and the corresponding regulatable oligomer in accordance with embodiments of the invention.

FIG. 18 schematically illustrates a method of making engineered regulatable constructs of this disclosure by selecting DRD/ligand pairs, designing the polypeptide or oligomer for proper function, providing expression control, and by providing for effective delivery to a target cell. FIG. 18 discloses SEQ ID NOS 243 and 244, respectively, in order of appearance.

FIG. 19 shows an exemplary process of designing engineered, regulatable polypeptides that include a membrane tethered cytokine with a sufficient dynamic range.

FIG. 20 schematically illustrates exemplary monomeric constructs for creating a heterotrimer having a single payload and multiple DRDs. Each construct includes DAP12 and NKG2C hetero-oligomerization domain.

FIG. 21A shows histograms of cellular IL12 levels in the presence of acetazolamide (+) or DMSO (−). Cells were transduced with the nucleic acids encoding the monomers depicted in FIG. 21B.

FIG. 21B is a schematic depicting the assembly and the oligomeric states using the monomers depicted in FIG. 20 (upper panel) and exemplary heterodimers (IL12-267 is a control construct, having only one DRD). IL12-266 and 268 show engineered, regulatable homodimers (IL12-266) and heterotrimers (IL12-268) with modulation hubs comprising multiple DRDs.

FIG. 21C shows fold change in the IL12 geometric MFI after addition of drug to each of IL12-266, 267 and 268. The transduced cells show a pattern of increasing fold regulation as they progress from monomer, dimer through to trimer.

FIG. 22 is a graph showing surface IL23 expression on CD19 CAR+ cells plotted using Prism Software.

FIG. 23 is graph showing IFNα expression on CD19 CAR+ cells plotted using Prism Software.

FIG. 24 is a graph showing IL2 expression on CD19 CAR+ cells plotted using Prism Software.

FIG. 25 shows the schematic showing the experimental design for determining IL18 expression.

FIG. 26 is a schematic showing the experimental design for determining IFNγ levels.

FIG. 27 is a graph showing IL18 expression in unactivated T cells transformed with non-regulated IL18 constructs.

FIG. 28 is a graph showing IL18 expression in activated T cells transformed with non-regulated IL18 constructs.

FIG. 29 is a graph showing interferon gamma (IFNγ) expression in untreated T cells transformed with non-regulated IL18 constructs.

FIG. 30 is a graph showing interferon gamma (IFNγ) expression in IL15 treated T cells transformed with non-regulated IL18 constructs.

FIG. 31 is a graph showing IL18 expression in unactivated T cells transformed with regulated IL18 constructs.

FIG. 32 is a graph showing IL18 expression in activated T cells transformed with regulated IL18 constructs.

FIG. 33 is a graph showing IL18 expression on day 8 in unactivated T cells transformed with regulated IL18 constructs.

FIG. 34 is a graph showing IL18 expression on day 10 in unactivated T cells transformed with regulated IL18 constructs.

FIG. 35 is a graph showing IL18 expression on day 8 in activated T cells transformed with regulated IL18 constructs.

FIG. 36 is a graph showing IFNγ expression T cells treated with 20 ng/ml IL15 and transformed with regulated IL18 constructs.

FIG. 37 is a graph showing IFNγ expression T cells treated with 20 ng/ml IL15 and transformed with regulated IL18 constructs.

FIG. 38 is a bar graph showing IL18 expression in T cells transformed with regulated IL18 constructs in the presence and absence of acetazolamide (ACZ).

FIG. 39 is a bar graph showing geometric mean fluorescence intensity (GMFI) for T cells expressing membrane-bound IL18 in the presence and absence of ACZ.

FIG. 40 is a bar graph showing IL18 expression in T cells transformed with regulated IL18 constructs in the presence and absence of ACZ.

FIG. 41 is a graph showing IL18 expression and shedding in cells untransformed (UT) or transformed with IL18-064 in the presence and absence of ACZ. The first three columns are unactivated cells and the last three columns are activated cells. In unactivated cells with IL18-064 treated with ACZ, there was a 1.7-fold increase in shed of IL18 treated with ACZ. In activated cells with IL18-064 treated with ACZ, there was a 3.1-fold increase in IL18 expression and 2.1-fold increase in IL18 shedding.

FIG. 42 is a graph showing IL18 expression and shedding in cells untransformed (UT) or transformed with IL18-066 in the presence and absence of ACZ. The first three columns are unactivated cells and the last three columns are activated cells. In unactivated cells with IL18-066 treated with ACZ, there was a 2.0-fold increase in shedding of IL18 treated with ACZ. In activated cells with IL18-066 treated with ACZ, there was a 3.1-fold increase in IL18 expression and 2.6-fold increase in IL18 shed.

FIG. 43 is a graph showing IL18 expression and shedding in cells untransformed (UT) or transformed with IL18-069 in the presence and absence of ACZ. The first three columns are unactivated cells and the last three columns are activated cells. In activated cells with IL18-069 treated with ACZ, there was a 1.9-fold increase in IL18 expression and 1.3-fold increase in IL18 shed.

FIG. 44 is a graph showing IL18 expression and shedding in cells untransformed (UT) or transformed with IL18-070 in the presence and absence of ACZ. The first three columns are unactivated cells and the last three columns are activated cells. In activated cells with IL18-070 treated with ACZ, there was a 1.8-fold increase in IL18 expression and 1.1-fold increase in IL18 shed.

FIG. 45 is a graph showing IL18 expression and shedding in cells untransformed (UT) or transformed with IL18-071 in the presence and absence of ACZ. The first three columns are unactivated cells and the last three columns are activated cells. In activated cells with IL18-071 treated with ACZ, there was a 1.8-fold increase in IL18 expression and 1.2-fold increase in IL18 shed.

FIG. 46 is a bar graph showing IL18 expression in T cells transformed with IL18-048 construct and treated with 20 ng/ml IL15 in the presence and absence of ACZ.

FIG. 47 is a bar graph showing IL18 expression in T cells transformed with IL18-064 construct and treated with 20 ng/ml IL15 in the presence and absence of ACZ.

FIG. 48 is a bar graph showing IL18 expression in T cells transformed with IL18-066 construct and treated with 20 ng/ml IL15 in the presence and absence of ACZ.

FIG. 49 is a bar graph showing IL18 expression in T cells transformed with IL18-069 construct and treated with 20 ng/ml IL15 in the presence and absence of ACZ.

FIG. 50 is a bar graph showing IL18 expression in T cells transformed with IL18-070 construct and treated with 20 ng/ml IL15 in the presence and absence of ACZ.

FIG. 51 is a bar graph showing IL18 expression in T cells transformed with IL18-071 construct and treated with 20 ng/mL IL15 in the presence and absence of ACZ.

FIG. 52 is a bar graph showing IL18 expression and shedding in T cells transformed with the IL18-064 construct in the presence and absence of ACZ.

FIG. 53 is a bar graph showing IL18 expression and shedding in T cells transformed with the IL18-066 construct in the presence and absence of ACZ.

FIG. 54 is a bar graph showing IL18 expression and shedding in T cells transformed with the IL18-048 construct in the presence and absence of ACZ.

FIG. 55 are graphs showing IL18 expression by Jurkat cells in the presence and absence of ACZ.

FIG. 56 is a graph showing surface recognition of IL12 in resting T cells.

FIG. 57 is a graph showing shedding of IL12 in resting T cells transformed with the control and experimental IL12 constructs.

FIG. 58 is a graph showing shed IL12 activity on NK cells.

FIG. 59 is a graph showing surface recognition of IL12 in activated T cells stimulated with CD3/CD28.

FIG. 60 is a graph showing shedding of IL12 in activated T cells transformed with the control and experimental IL12 constructs.

FIG. 61 is a graph showing shed IL12 activity on NK cells.

FIG. 62 is a schematic showing the experimental design for the in vivo studies of IL12 regulation.

FIG. 63A-63C are graphs showing ACZ regulates mbIL12 on CD19-CARTs in vivo.

FIG. 64 are graphs showing oligomerizing constructs shed IL12 in the plasma was regulated by ACZ with a non-zero off-state.

FIG. 65 is a schematic showing scRaji solid tumor model study design.

FIG. 66 is a graph showing tumor growth in control groups CAR alone or empty vector (EV).

FIG. 67 is a graph showing tumor growth in mice given CAR-T cells with or without ACZ.

FIG. 68 is a graph showing infusion of IL12-Expressing CAR-T Cells shows delayed yet robust regulated anti-tumor activity at suboptimal doses of CAR-T cells.

FIG. 69A and FIG. 69B are graphs showing modulation hubs allow ACZ-driven control of IL12 in the plasma.

FIG. 70A and FIG. 70B are graphs showing mbIL12 modulation hubs allow control of systemic IFNγ. Of note, by day 14 IFNγ levels return to baseline for all mbIL12 constructs except those turned ON using ACZ

FIG. 71A and FIG. 71B are graphs showing mbIL12 modulation hubs allow control of IFNγ in the tumor (Day 7 post ACT).

FIG. 72A and FIG. 72B are graphs showing mbIL12 modulation hubs enhances localization of IFNγ production to the tumor versus the circulation. Higher IFNγ ratio in the tumor for regulated vs constitutive mbIL12 is likely due to the activation dependence of IL12 regulation.

FIG. 73 is a graph showing ON-state IL12 leads to increased activation of dendritic cells.

FIG. 74 is a graph showing ON-state IL12 leads to increased activation of MHCII Monocytes.

FIG. 75 is a graph showing the percentage of tumor-associated macrophages which are reduced by IL12.

FIG. 76 is a graph showing the percentage of TAMs with TAM phenotypes, which are reduced by IL12.

FIG. 77 is a graph showing IL12 skews TAMs to an M1-like phenotype.

FIG. 78 are graphs showing PD-L1 is more elevated with secreted IL12 than mbIL12 across multiple cell types and tissues.

FIG. 79 are flow cytometry read-outs showing IL12 expression in Jurkat cells transduced with IL12 constructs in the presence or absence of VDF 10 μM for 24 hours.

FIG. 80 are images showing percent of Jurkat cells expression IL12 in the presence or absence of VDF 10 μM for 24 hours.

FIG. 81 are graphs showing geometric MFI indicative of IFNα abundance on the surface of tumor infiltrating lymphocytes.

FIG. 82 is a graph showing regulation of IFNα with ACZ.

FIG. 83 is a schematic showing the T-cell transduction experiment using constructs containing IL15 and IFNα.

FIG. 84 is a graph showing the enrichment of mbIL15 on the surface of T cells over time. Pattern bars refer to +ACZ and solid bars refer to +Veh. The first bar set (solid bars then pattern bars) is for cells containing IFNα-003, the next bar set is for cells containing IFNα-007, the third bar set is for cells containing IFNα-015, the fourth bar set is for cells containing IFNα-016 and the fifth and final bar set is for cells containing IFNα-018.

FIG. 85 is a graph showing IFNα is regulated approximately 20-fold in IFNα-016 and -018 constructs. Pattern bars refer to +ACZ and solid bars refer to +Veh. The first bar set (solid bars then pattern bars) is for cells containing IFNα-003, the next bar set is for cells containing IFNα-007, the third bar set is for cells containing IFNα-015, the fourth bar set is for cells containing IFNα-016, the fifth bar set is for cells containing IFNα-018, and the sixth and final bar set are for control cells with empty vector.

FIG. 86 is a schematic showing Jurkat cell transduction experimental layout.

FIG. 87 is a bar graph showing percentage of IL15 expressing cells after transduction with IL18 constructs. 300 μl virus generated % 70 to % 90 transduced cells, 100 μl virus generated % 40 to % 65 transduced cells and 33 μl virus generated % 7 to % 33 transduced cells.

FIG. 88 are graphs showing membrane-bound IL18 regulation was observed with VEGFR containing constructs.

FIG. 89 is a bar graph showing 10-fold regulation of membrane IL18 (geometric MFI) was observed in IL18-080 construct. Bars refer to left y axes, showing mbIL18 expression. Bottom numbers show mbIL18 fold change. Circles refer to right y axes, showing released IL18 in media. Top numbers show shed IL18 fold change.

FIG. 90 regulation of percent IL18 on the cell membrane was more pronounced in IL18-080 construct. Numbers show mbIL18 percent fold change. IL18-081 and -082 show very high fold-change that is due to very low value of percent IL18 in off-state.

FIG. 91 is an image showing small scale virus transduction in Jurkat cells for SOX2 constructs.

### DETAILED DESCRIPTION

The present disclosure provides compositions and systems for regulatory control of a payload (“regulation compositions and systems”). Regulation compositions according to this disclosure include modulation hubs, wherein a modulation hub, comprises at least two drug responsive domains (DRDs) responsive to a ligand; and, at least one payload operably linked to the modulation hub. Regulation compositions also include engineered, regulatable polypeptide monomers (or polypeptide monomers, or simply, monomers); engineered, regulatable oligomers (or regulatable oligomers, or simply, oligomers); and engineered, regulatable polypeptides (or regulated polypeptides, or simply, polypeptides). The regulatable oligomers are formed from the regulatable monomers, and the regulatable oligomers and regulatable polypeptides comprise at least one payload operably linked to a modulation hub. Regulation systems according to this disclosure comprise the regulation compositions and a stabilizing ligand.

In some embodiments, this disclosure serves to enable regulation of a biologically active payload. For example, this disclosure describes improved drug responsive domain (DRD)-based regulation of payloads by improving control over the off-state of the payload (i.e., reducing the abundance or availability of the payload in the absence of ligand) and/or optimizing its range of regulation as compared to DRD-regulation systems comprising a single DRD.

Without meaning to be limited by theory, DRDs are thought to be unstable polypeptides that degrade in the absence of their corresponding stabilizing ligand (also referred to as the paired ligand or ligand), but whose stability is rescued by binding to the stabilizing ligand. Because binding of the ligand to the DRD is reversible, later removal of the ligand results in the DRD unfolding, becoming unstable, and ultimately being tagged for degradation by the ubiquitin-proteasome system (“UPS”). Accordingly, it is believed that when a DRD is operably linked to a payload, the entire construct (i.e., DRD plus payload) itself is rendered unstable and degraded by the UPS. However, in the presence of the paired ligand, the construct is stabilized, and the payload remains available for use. Further, it is believed that the conditional nature of DRD stability allows a rapid and non-perturbing switch from stable polypeptide to unstable UPS substrate, and may facilitate regulation of a payload's activity level, and/or modulation of a payload's activity level. The underlying discovery that led to the improved regulatory control is that multiple DRDs associated together in a modulation hub result in a payload having a lower off-state as compared to the same payload operably linked to a single DRD. That is, in the absence of ligand, increasing the density of DRDs (for example with multiple DRDs) is thought to reduce the abundance or availability of a payload as compared to a payload that is operably linked to only a single DRD.

Payloads should be understood to include one or more polypeptides having one or more functions, such as one or more biological activities, desired to be regulated. Payloads include multiple classes of therapeutically important polypeptides (proteins and peptides) such as Type I/II membrane proteins, cytokines, intracellular proteins, secreted proteins, CAS9 proteins, and transcription factor proteins.

Because the abundance and availability of a payload are related to the activity of a payload, for purposes of this disclosure, the terms “abundance,” “availability,” “activity,” and the phrase “abundance and/or activity” (and similarly “level of abundance,” “level of availability,” “level of activity,” and “level of abundance and/or activity”) are used interchangeably throughout this disclosure and are generally referred to as “activity,” unless explicitly stated otherwise or nonsensical in context. Further, measurements of abundance or availability are used as a proxy for activity level and may be used herein to reflect the activity level. Consequently, changes in the abundance or availability of a payload in the presence of an effective amount of ligand as compared to in the absence of ligand optionally serves as a proxy for measuring changes in activity level.

### Engineered, Regulatable Polypeptides

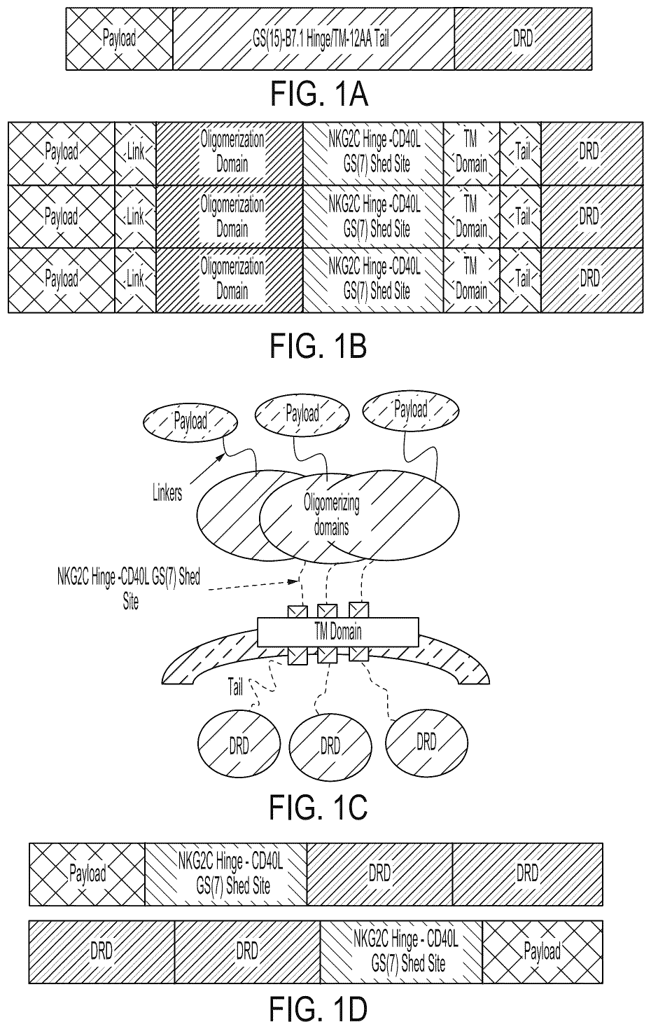

Provided herein are engineered, regulatable polypeptides having at least one payload and a modulation hub with at least two drug responsive domains (DRD), wherein the modulation hub is operably linked to the payload and wherein the at least two DRDs are responsive to a ligand. The at least two DRDs can be the same or different, and optionally the least two DRDs are responsive to the same ligand. For example, in certain embodiments, the modulation hub comprises three DRDs, wherein the three DRDs are responsive to the same ligand. The at least one payload has a biological activity, and the biological activity of the payload is regulated by interaction of the two or more DRDs (e.g., 2, 3, 4, or more, which are responsive to the same or different ligands) with an effective amount of the ligand. In the case of multiple different DRDs responsive to different ligands, it is understood that reference to “the ligand” means “the ligands.” The activity level of the at least one payload ranges from a basal activity level in the absence of the ligand to a maximum activity level in the presence of a saturating amount of the ligand, and the basal activity level of the payload in the regulatable polypeptide is lower than that of the same payload in a control construct. Saturating amount of the ligand refers to any amount of ligand at or above the amount that results in the maximum abundance and/or activity of the payload. By way of example, the activity level of the at least one payload ranges from a basal activity level in the absence of the ligand to a maximum activity level in the presence of a saturating amount of the ligand, and the range of activity level of the payload in the regulatable polypeptide is greater than that of the same payload in a control construct. In some embodiments, the at least one payload is multiple payloads (e.g., two payloads or three payloads, which are the same as or different from each other). Thus, for example, in some embodiments, the at least one payload is three payloads and the at least two DRDs is three DRDs, wherein the three payloads are the same and the three DRDs are responsive to the same ligand. In certain embodiments, the at least one payload is one payload and the at least two DRDs is three DRDs responsive to the same ligand. FIG. 1D depicts illustrative examples of engineered regulatable polypeptides having a modulation hub with two DRDs operably connected to a payload.

### Polypeptide Monomers, and Engineered, Regulatable Oligomers

Provided herein are engineered polypeptide monomers and engineered, regulatable oligomers of at least two engineered polypeptide monomers. Oligomers comprise at least one payload operably linked to a modulation hub comprising two or more DRDs. The payload has a biological activity, and the biological activity of the payload is regulated by interaction of the two or more DRDs (e.g., 2, 3, 4, or more, which are responsive to the same or different ligands) and an effective amount of the ligand.

In some embodiments, the monomers comprise at least one payload, an oligomerization domain (e.g., a dimerization domain, a trimerization domain, a tetramerization domain, a pentamerization domain, or a hexamerization domain) configured to promote oligomerization of the monomers, and at least one DRD responsive to a ligand, wherein the at least one payload is operably linked to the at least one DRD. In certain embodiments, the monomers comprise an oligomerization domain; and, at least one payload, or at least one DRD, or at least one payload and at least one DRD, provided that when at least two or more monomers oligomerize to form an oligomer, the resultant oligomer comprises at least one payload operably linked to a modulation hub comprising at least two DRDs.

Oligomers according to this disclosure are a multimeric association of at least two engineered, regulatable polypeptide monomers. The monomers of the oligomer associate through an oligomerization domain. The oligomer, for example, can be a dimer resulting from association of monomers via a dimerization domain; or it can be a trimer resulting from association of monomers via a trimerization domain; or it can be a tetramer resulting from association of monomers via a tetramerizing domain; or it can be a pentamer resulting from association of monomers via a pentamerizing domain; or it can be a hexamer resulting from association of monomers via a hexamerizing domain. In each case, the oligomer may result from monomers that are the same or different. Accordingly, the payload or payloads of each of the monomers is the same or different and the DRDs of each of the monomers is the same or different. Thus, by way of example, the oligomer can be an association of three monomers, wherein each monomer comprises the same payload, oligomerization domain, and DRD and consequently the oligomer comprises a first, second, and third payload that are the same and a modulation hub comprising a first, second, and third DRD that are responsive to the same ligand.

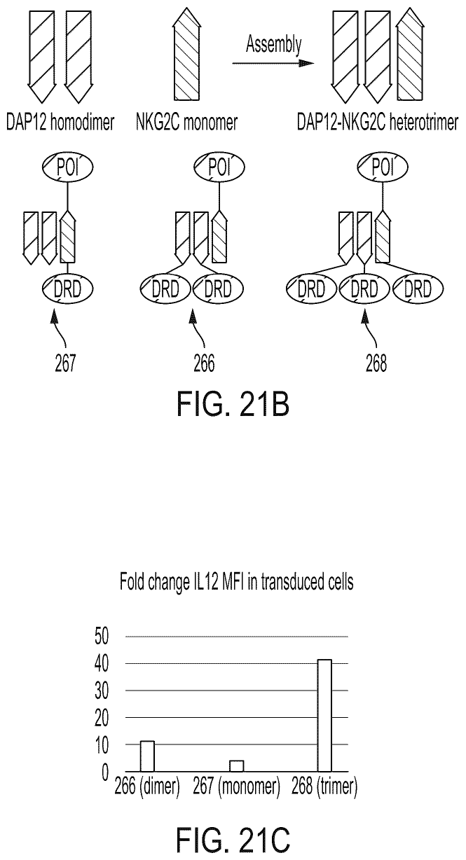

Thus, provided herein is also an engineered, regulatable oligomer comprising at least two polypeptide monomers, wherein each monomer comprises an oligomerization domain and at least one payload or one DRD, wherein the oligomer comprises at least one payload operably linked to at least two DRDs, wherein the at least one payload has a biological activity, wherein the at least two DRDs are responsive to a ligand. Optionally, the oligomer is a hetero-oligomer wherein the at least two polypeptide monomers of the hetero-oligomer each comprise an oligomerization domain and wherein in some embodiments the oligomerization domain of one of the polypeptide monomers comprises a hetero-oligomerization domain. Optionally the DRDs in the hetero-oligomer are the same or different. Optionally, the at least two DRDs are responsive to the same ligand. By way of example, the engineered, regulatable hetero-oligomer comprises a first, a second, and a third polypeptide monomer, wherein the first and second polypeptide monomers are the same, wherein each of the first and second polypeptide monomers comprises a DRD, and wherein the third polypeptide monomer comprises a hetero-oligomerization domain and a payload. FIG. 21B depicts hetero-trimers comprising one payload and two, or three DRDs as well as a control construct with only one DRD. In certain hetero-oligomer embodiments, each monomer comprises the same oligomerization domain but include different payloads and/or DRDs. Hetero-oligomeric constructs can similarly include a first and second monomers, each including a DRD and payload, and a third monomer including a DRD without a payload. The engineered, regulatable hetero-oligomer, however, can have a 1:1 payload:DRD ratio.

Multiple payloads in an oligomer can be the same or different, but in embodiments, each of the payload(s) has a biological activity under appropriate conditions. In some such embodiments, the level of activity of each payload in the oligomer has a range spanning from a basal level of activity in the absence of ligand to a maximum activity in the presence of a saturating amount of ligand, and the basal activity of each payload of the oligomer is less than that of the same payload in a control construct. In some embodiments, the range of activity of each payload in the oligomer is greater than that of the same payload in a control construct. See, e.g., examples 3-5 below.

Also provided are oligomers comprising at least two polypeptide monomers, wherein each monomer comprises at least one payload, an oligomerization domain, and one DRD, wherein the oligomer comprises at least two payloads operably linked to at least two DRDs, wherein the at least two payloads have a biological activity level, wherein the DRDs are responsive to a ligand, and wherein, in some embodiments, the at least two payloads and the at least two DRDs are present in a 1:1 ratio. In certain embodiments, the payload is present in a an oligomer in a 1:2, a 1:3, a 2:3, a 1:4, a 3:4, a 1:5, a 2:5, a 3:5 or a 4:5 ratio of payload to DRD or in a 2:1, 3:1, 3:2, 4:1, 4:3, 5:1, 5:2, 5:3 or 5:4 ratio of DRD to payload (provided that the oligomer always comprises at least two DRDs). In some embodiments, the at least two DRDs may be the same or different, the at least one payload is multiple payloads, which may be the same or different, or the multiple DRDs may be different but responsive to the same ligand. The oligomer can include, for example, a modulation hub comprising a first, second, and third DRD, which are responsive to the same or different ligands, and a first, second, and third payload that are the same or different. In some embodiments, the first, second, and third DRD are responsive to the same ligand and the first, second and third payloads are the same. The level of activity of each payload in the oligomer has a range spanning from a basal level of activity in the absence of ligand to a maximum activity in the presence of a saturating amount of ligand and wherein the basal activity of each payload in the oligomer is less than that of the same payload in a control construct. Optionally, the range of activity level of the payload in the oligomer is greater than that of the same payload in a control construct.

FIG. 1 depicts example polypeptide constructs useful in understanding the scope of the disclosure are provided. FIG. 1A depicts a control construct having a payload, hinge region, and DRD. FIG. 1B depicts a polypeptide monomer construct according to this disclosure that comprises an oligomerization domain, which produces monomers that self-assemble into an oligomer according to this disclosure. FIG. 1C depicts the engineered, regulatable oligomer that results from association of the monomers of FIG. 1B.

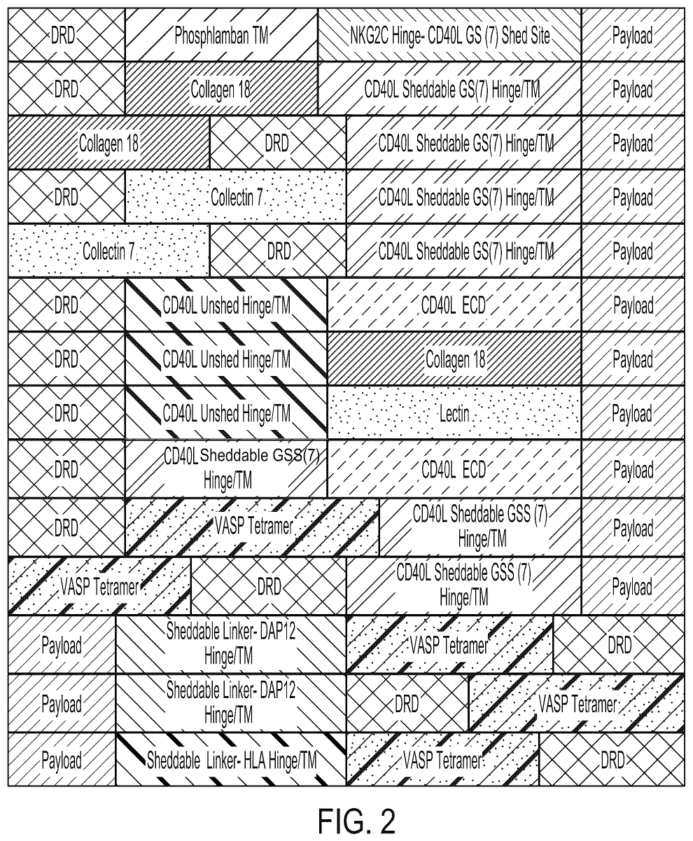

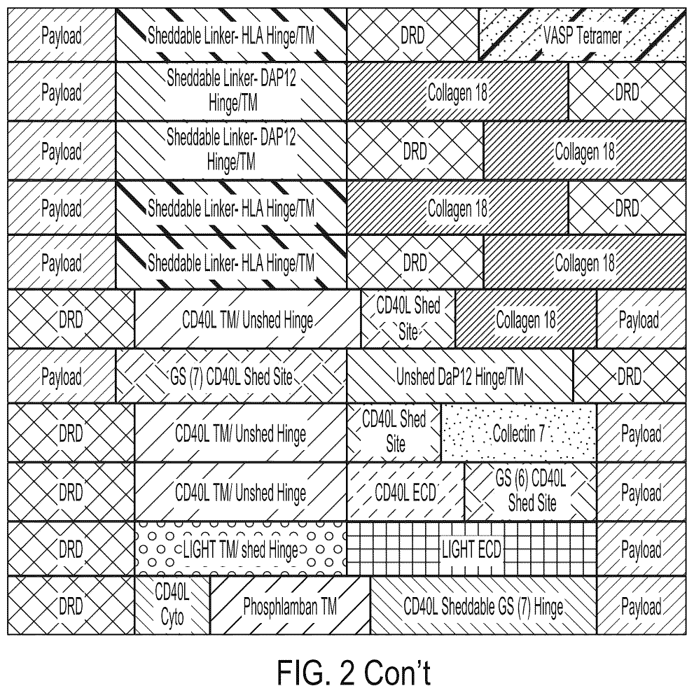

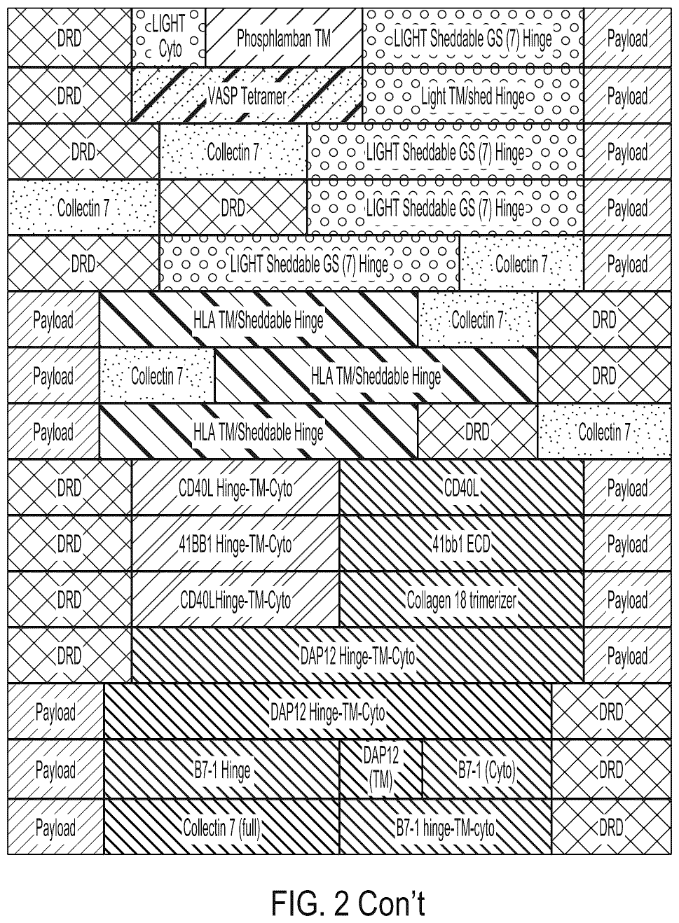

FIG. 2 shows schematics of various polypeptide monomer constructs, each having a payload operably connected to an oligomerization domain and DRD. When expressed, the polypeptide monomers self-assemble via the oligomerization domains and form a multimeric regulatable polypeptide (an engineered regulatable payload composition, which when formed from monomers is also referred to as an oligomer) according to embodiments of the present disclosure. The degree of multimerization is generally determined by the choice of oligomerization domain. Thus, for example, constructs having CD40L ECD, Collagen 18, Lectin, DAP12, and 41BBl oligomerization domains tend to form trimerized regulatable payload compositions, whereas those with VASP oligomerization domains tend to form tetrameric regulatable payload compositions, and those with phospholamban oligomerization domains tend to form pentameric regulatable payload compositions.

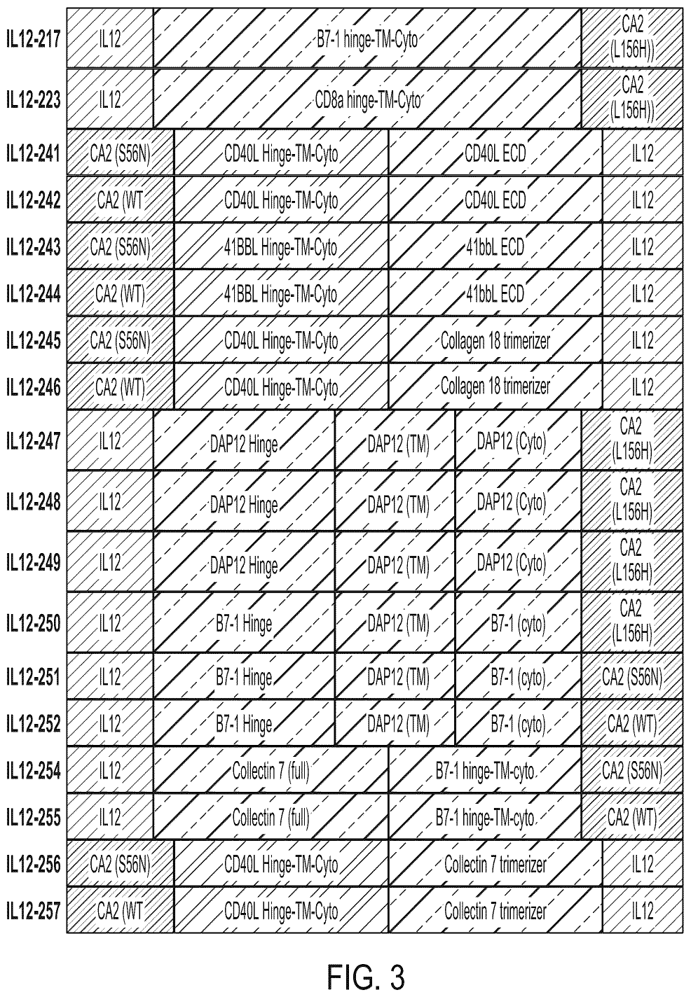

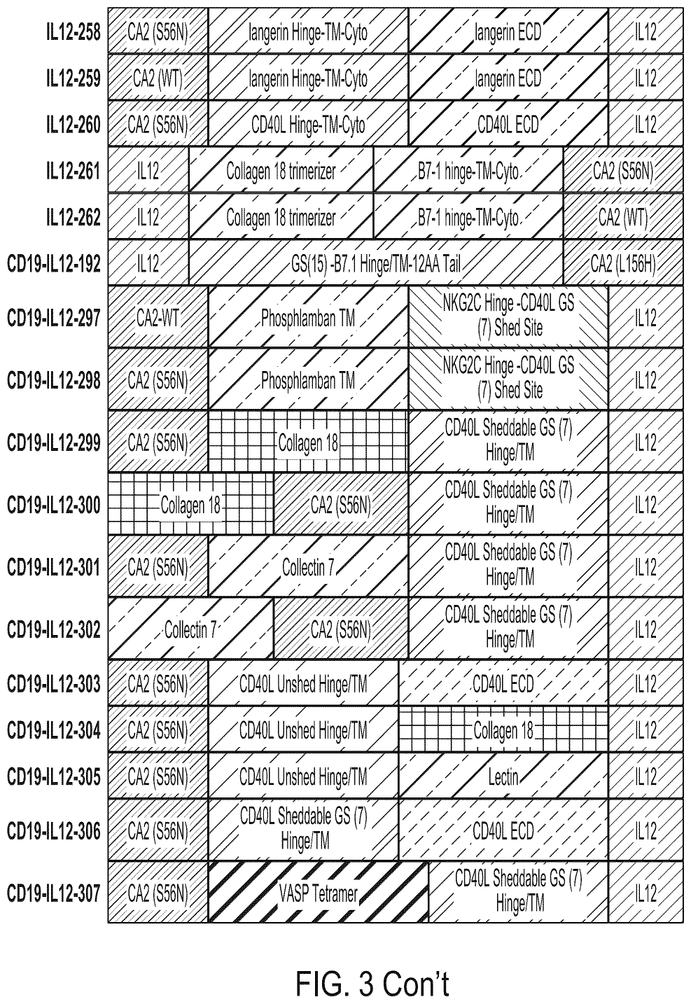

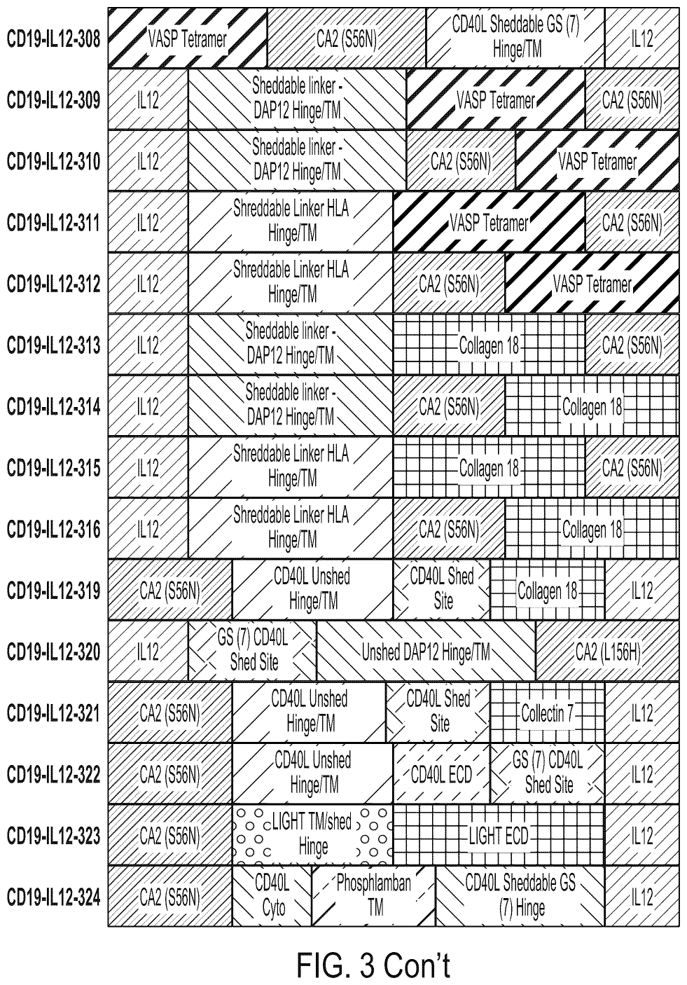

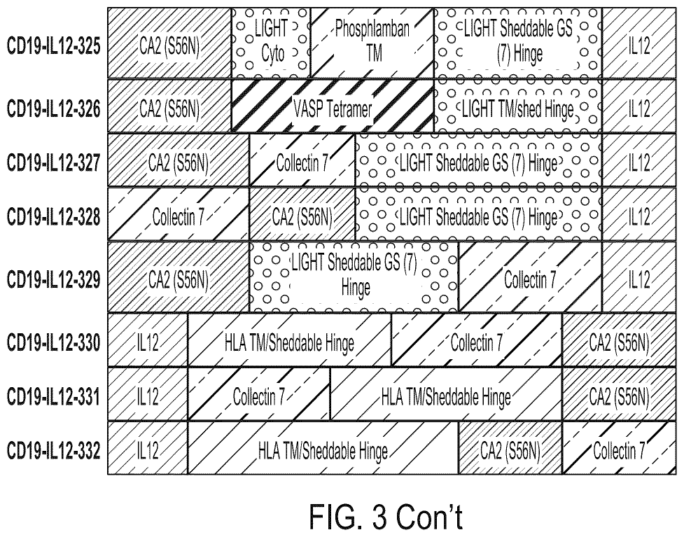

FIG. 3 shows schematics of the polypeptide components of constructs IL12-217 (SEQ ID NOs: 125 and 126), IL12-223 (SEQ ID NOs: 127 and 128), IL12-241-262 (SEQ ID NOs: 129-170), CD19-IL12-192 (SEQ ID NOs: 123 and 124), CD19-IL12-297-316 (SEQ ID NOs: 171-210), and CD19-IL12-319-332 (SEQ ID NOs: 211-238). Certain of the constructs are control constructs for regulatable compositions and lack the oligomerization domain (e.g., IL12-217, IL12-223, and CD19-IL12-192) resulting in a payload operably connected to only a single DRD, others are control constructs for DRDs and include the wild type CA2 polypeptide in place of a mutated CA2 (242, 246, 249, 252, 255, 257, 259, 262, 297), whereas other constructs (e.g., IL12-241) are polypeptide monomers that can self-assemble into regulatable payload compositions according to embodiments of this disclosure.

### Components of Monomers and Regulatable Polypeptides

Example components (building blocks) of the engineered monomers which associate to form regulatable, engineered oligomers, and of the engineered, regulatable polypeptides described herein are referenced throughout this disclosure and provided below. As described herein, monomers include at least an oligomerization domain and: a payload, or DRD, or a payload and a DRD. As also described herein oligomers and polypeptides include one or more payloads and a modulation hub comprising at least two DRDs. The monomers, oligomers, and polypeptides optionally include one or more additional components such as linkers, hinges (e.g., sheddable and non-sheddable hinges), tails (e.g., cytoplasmic tails), and transmembrane domains. As described below in the section related to methods of making, one of skill in the art may select from the various components using guideposts provided below to achieve a desired outcome (e.g., location of the oligomerization domain, payload, or DRD relative the cell in which it is expressed, whether or not the payload is secreted from the cell or tethered to the membrane, or the desired activity of the payload or payloads).

### Modulation Hubs

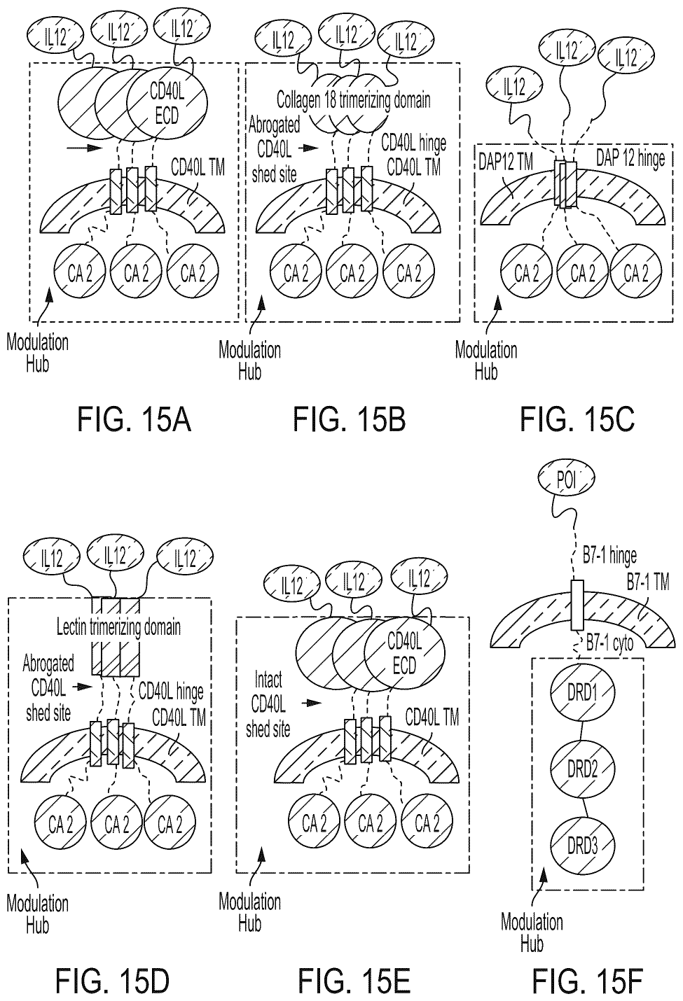

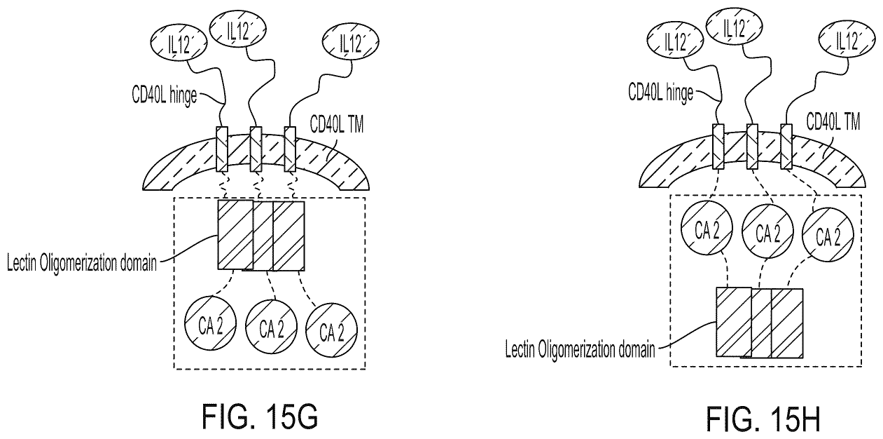

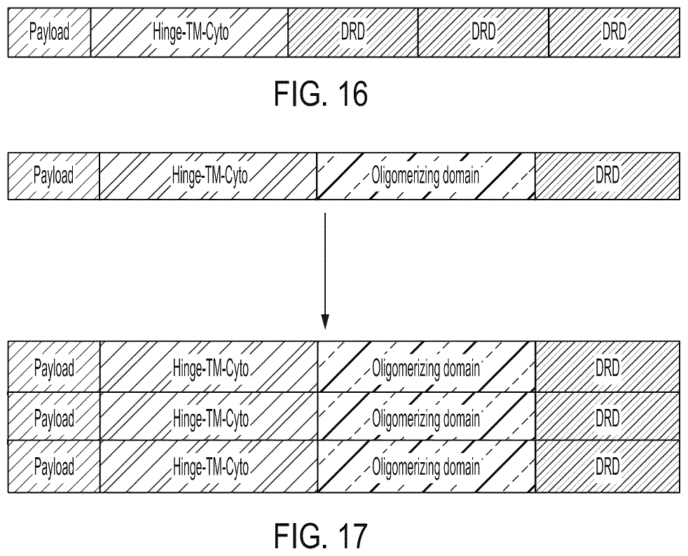

In general, modulation hubs are polypeptides designed to facilitate regulation of a characteristic (e.g., abundance or activity) of a target payload to which it is operably linked and whose foundational building blocks are DRDs. Modulation hubs according to this disclosure comprise at least two DRDs, and in some embodiments such hubs may be formed upon oligomerization of at least two polypeptide monomers as described herein. Thus, in some embodiments, at least two DRDs are linked together via non-covalent bonds formed through oligomerization, optionally by oligomerization domains associated with each DRD as illustrated in FIG. 15A-E. In some embodiments, the at least two DRDs are linked, optionally through a linker, via covalent bonds, as illustrated in FIG. 15F and FIG. 16.

### DRDs

DRDs interact with a ligand such that, when the DRD is operatively linked to a payload, it confers ligand-dependent reversible regulation of a characteristic of the payload (for example, activity). Although referred to as drug responsive domains, the ligand to which a DRD is responsive need not be a drug. Suitable DRDs (and their paired ligands), which may be referred to as destabilizing domains or ligand binding domains, are also known in the art. See, e.g., U.S. Pat. Nos. 9,487,787 and 10,137,180, U.S. Publication Nos.: 2019/0192691; 2020/0101142; 2020/0172879; 2021/0069248, and U.S. patent application Ser. Nos. 17,251,635; and 17/288,373, and WO2018/161000; WO2018/231759; WO2019/241315; U.S. Pat. Nos. 8,173,792; 8,530,636; WO2018/237323; WO2017/181119; US2017/0114346; US2019/0300864; WO2017/156238; Miyazaki et al., J Am Chem Soc, 134:3942 (2012); Banaszynski et al. (2006) Cell 126:995-1004; Stankunas, K. et al. (2003) Mol. Cell 12:1615-1624; Banaszynski et al. (2008) Nat. Med. 14:1123-1127; Iwamoto et al. (2010) Chem. Biol. 17:981-988; Armstrong et al. (2007) Nat. Methods 4:1007-1009; Madeira da Silva et al. (2009) Proc. Natl. Acad. Sci. USA 106:7583-7588; Pruett-Miller et al. (2009) PLOS Genet. 5: e1000376; and Feng et al. (2015) Elife 4: e10606, the contents of each of which are hereby incorporated by reference in their entirety.

Certain of these and other example DRDs suitable for use with modulation hubs according to this disclosure are also provided elsewhere in this specification.

The DRDs, by way of example, can be chosen from FKBP (SEQ ID NO:93), ecDHFR (SEQ ID NO:18), hDHFR (SEQ ID NO:37), ER (SEQ ID NO:96), PDE5 full length (SEQ ID NO:95), PDE5 ligand binding domain (SEQ ID NO:94) and CA2 (SEQ ID NO:1) or a portion of any of the foregoing that maintains DRD function or an amino acid sequence having at least 85%, 86%, 87%, 88%, 89%, 90%, 91%, 92%, 93%, 94%, 95%, 96%, 97%, 98%, 99% or 100% identity to SEQ ID NOs: 1, 18, 37, 93, 94, 95, or 96 or the DRD functional portion thereof. One or more mutations (including truncations, substitutions, and deletions) in the amino acid sequence of FKBP, ecDHFR, hDHFR, ER, PDE5, and CA2, for example, can be advantageous to further destabilize the DRD.

Numerous DRD are described herein, but one of skill in the art can identify additional DRDs suitable for use with modulation hubs and in regulatable compositions according to this disclosure. By way of example, DRDs can be identified using library screening and structure-guided engineering to select the optimal DRD variant with sufficient instability in the absence of the ligand and sufficient stability in the presence of the ligand. A variant library can be generated using random mutagenesis screening by transducing cells (e.g., Jurkat cells) with mutant DRD candidates. To produce an enriched library, cells with the desired characteristics (low basal activity/expression and high dynamic range) are selected by testing polypeptide abundance across a range of concentrations of ligand. Single cell clones are then produced and characterized to identify candidate DRDs.

The DRDs described herein are responsive to a paired ligand (also referred to as a “stabilizing ligand” or simply as a “ligand.” Optionally, the DRDs are responsive to a paired ligand that is a small molecule drug, such as an FDA-approved small molecule. However, one of skill in the art can select the DRD and its paired ligand to meet the specific needs of the system. Examples of DRD/ligand pairs are shown in Table 1.

TABLE 1
Listing of DRD and exemplary ligands

| | SEQ ID | |
|---|---|---|
| DRD Polypeptide | NO: | Exemplary Ligands |
| E. coli dihydrofolate | 18 | Methotrexate (MTX) |
| reductase (ecDHFR) | | Trimethoprim (TMP) |
| (Uniprot ID: P0ABQ4) | | |
| Human dihydrofolate | 37 | Methotrexate (MTX) |
| reductase (hDHFR) | | Trimethoprim (TMP) |
| (Uniprot ID: P00374) | | |
| Human FKBP | 93 | Shield-1 |
| (FK506 binding protein) | | |
| (Uniprot ID: P62942) | | |
| Phosphodiesterase 5 (PDE5), | 94 | Sildenafil; |
| ligand binding domain | | Vardenafil; |
| (Uniprot ID: Uniprot ID O76074) | | Tadalafil |
| Phosphodiesterase 5 (PDE5), | 95 | Sildenafil; |
| full-length | | Vardenafil; |
| (Uniprot ID: Uniprot ID O76074) | | Tadalafil |
| Carbonic anhydrase II (CA2) | 1 | Celecoxib |
| (Uniprot ID: P00918) | | Acetazolamide |
| Human estrogen receptor (ER) | 96 | Bazedoxifene |
| Uniprot ID: P03372.2) | | Raloxifene |

Optionally, a DRD of the present disclosure may be derived from a carbonic anhydrase, which is a member of a superfamily of metalloenzymes. For example, human carbonic anhydrase (hCA2) can be adapted for use as a DRD. A DRD of the present disclosure may be derived from amino acids 1-260 of CA2 (Uniprot ID: P00918). Optionally, DRDs are derived from CA2 comprising amino acids 2-260 of the parent CA2 sequence (e.g., amino acids 2-260). This is referred to herein as a CA2 M1del mutation (CA2; SEQ ID NO: 5). Optionally, a DRD of the present disclosure comprises a region of or the whole human carbonic anhydrase 2, and further comprises one or more mutations relative to the full-length sequence selected from M1del, L156H, and S56N. Optionally, the DRD is selected from the group consisting of SEQ ID NOs: 1, 3, 5, 7 and 9.

### Oligomerization Domains

For purposes of this disclosure, oligomerization domains are sequences that promote self-assembly of monomers into oligomers, for example, by non-covalent bonding. Such domains may be found in nature and may be adapted for use in forming oligomers including a payload and modulation hub as described herein. Examples of oligomerization domains include, but are not limited to, phospholamban, collectin, collagen, VASP, CD40L, TNFSF (e.g., TNFSF14, also referred to herein as LIGHT and 4-1BBL), langerin, DAP12, and NKG2C. Optionally, the oligomerization domain is a phospholamban transmembrane domain, collagen 18, avidin, streptavidin, CD40L extra cellular domain (ECD), langerin ECD, 4-1BBL ECD, VASP tetramer, DAP12, LIGHT extracellular domain (ECD), collectin 7, fungal lectin (5xzk), aspartate transcarbomylase (1EKX), 4-OT trimer, RH3 designed coiled coil (1TGG), HIV-1 gp41, FIV dUTP pyrophosphate, SIV gp41, the Ebola virus gp-2, HTLV-1 gp-21 domain, a foldon domain, a GCN4 domain, a T4 fibritin domain, the yeast heat shock transcription factor, and human collagen VIII, Collagen 18, Collagen 15 and hnRNP. Certain oligomerization domains do not oligomerize with themselves and only oligomerize with a different oligomerization domain, such as NKG2C. Such oligomerization domains are referred to herein as hetero-oligomerization domains.

FIG. 15A-E provide non-limiting examples of implementations of oligomerization domains as described herein. Specifically, in the example embodiments of FIGS. 15A and E, the ECD motif of CD40L (SEQ. ID NO. 15) corresponds to the oligomerization domain. In FIG. 15B, the Collagen 18 trimerizing motif (SEQ. ID NO. 19) corresponds to the oligomerization domain. In FIG. 15C, the DAP12 dimerizing/trimerizing motif (SEQ. ID NO. 79) corresponds to the oligomerization domain. In FIG. 15D, the collectin 7 trimerizing motif (SEQ. ID NO. 23) corresponds to the oligomerization domain. Although FIG. 15 illustrates the use of trimerizing motifs as oligomerization domains, any polypeptide motif that facilitates self-assembly or associates into larger constructs, may be used as the basis of an oligomerization domain in accordance with this disclosure, including dimerizing motifs or motifs that result in four or five or six or more polypeptide motifs assembling together. For example, the VASP motif illustrates an example of a motif suitable for use as an oligomerization domain, which would promote self-assembly into a tetramer. As another example, the phospholamban motif is an example of a motif suitable for use as an oligomerization domain which would result in self-assembly into a pentameric modulation hub.

Thus, although the example modulation hubs depicted in FIG. 16 all comprise three DRDs, modulation hubs according to this disclosure may comprise two or more, three or more, four or more, five or more, six or more, etc. DRDs. Further the DRDs forming a modulation hub may be the same or different. Thus, for example, in FIG. 15F, DRD1, DRD2 and DRD3 can each be the same DRD, or each of DRD1, DRD2 and DRD3 may be a different DRD, or two of the DRDs (for example DRD1 and DRD2) may be the same DRD whereas DRD3 may be a different DRD. FIG. 17 shows a schematic construct of a monomer (top) with a payload, hinge, transmembrane region, cytoplasmic tail, oligomerization domain, and the oligomer resulting from self-assembly of the monomers. Any DRD may be used to form a modulation hub and certain non-limiting examples of suitable DRDs are referenced throughout this application including in patents and applications referenced in this specification.

### Payloads

By way of example, the payload can be any polypeptide having a desired biological function. Such payloads can be modified polypeptides such as glycosylated polypeptides or lipopeptides, which upon expression are modified in a cell by constitutive enzymatic activity or by over-expression of selected enzymes. Payloads include multiple classes of therapeutically important polypeptides or any active portion thereof. For example, payloads include Type I/II membrane proteins such as CD40L, 4-1BBL, and CAR or active portions thereof. Payloads also include cytokines such as IL15, IL12, IL1B, IL2, IL7, IL18, IL21, IL23, IL36, TNFα, IFNγ, IFNα, IFNβ, etc., including membrane-tethered forms thereof or active portions thereof. Payloads also include intracellular polypeptides such as dnSHP2, T7, RNA polymerase, Cas9, or active portions thereof. Payloads include secreted proteins such as VEGF-trap, and native cytokines, or active portions thereof. And payloads further include transcription factors such as Foxp3, c-Myc, STAT5, and c-Jun, or active portions thereof, or constitutively active versions thereof. Other examples of suitable payloads include cytokine receptors, T-cell receptors (TCR), chimeric antigen receptors (CAR), immunomodulatory proteins in addition to those already exemplified, or any active portion thereof. The payload can also be a gene editing polypeptide or transcription factor in addition to those previously exemplified. The payload can also be a combination of polypeptides having a desired combination of actions, or active portions thereof.

In some embodiments, payloads are therapeutic agents chosen from a cancer therapeutic agent, a therapeutic agent for an autoimmune disease, an immunotherapeutic agent, an anti-inflammatory agent, an anti-pathogenic agent, a gene therapy agent, or combinations thereof. The immunotherapeutic agent may be an antibody or fragments and variants thereof, a TCR, a CAR, a chimeric switch receptor, an antagonist of a co-inhibitory molecule, an agonist of a co-stimulatory molecule, a cytokine, a mutated version of a cytokine possessing altered receptor binding properties (also called a mutein), a cytokine receptor, a chemokine, a chemokine receptor, a metabolic factor, a coagulation factor, an enzyme, a homing receptor, a kinase, a phosphatase, a dominant negative version of a phosphatase (such as SHP-1 or SHP-2), a dominant negative signaling molecule or receptor (such as a dominant negative Fas Receptor), a dominant negative transcription factor, and a safety switch.

In some embodiments, payloads of the present disclosure may be cytokines, and fragments, variants, analogs and derivatives thereof, including but not limited to interleukins, tumor necrosis factors (TNFs), interferons (IFNs), TGFβ, and chemokines. The interleukins may be chosen from IL15, IL12, IL1β, IL2, IL7, IL18, IL21, IL23, IL36, and variants thereof including membrane-bound, secreted, fusion polypeptide, or cytokine mutants with altered receptor binding properties (such as muteins), and bicistronic forms of the interleukins, and combinations thereof.

For example, IL12 may include both p35 and p40 subunits encoded by a single nucleic acid that produces a single chain polypeptide. The single chain polypeptide may be generated by placing the p35 subunit at the N terminus or the C terminus of the single chain polypeptide. Similarly, the p40 subunit may be at the N terminus or C terminus of the single chain polypeptide. By way of another example, the payload may be a bicistronic IL12 containing p40 and p35 subunits.

In some embodiments, the payload can be an active portion or variant of a polypeptide with a desired biological function, so long as the payload retains the desired biological function. By way of example, the payload may be the p40 subunit of IL12 or the p35 subunit of IL12 or a variant of IL12 that promotes NK cell survival, regulates NK cell and T cell activation and proliferation, and/or supports NK cell development from hematopoietic stem cells.

In some embodiments, payloads of the present disclosure may be chimeric antigen receptors (CARs) comprising an extracellular targeting domain (e.g., a scFv that recognizes a specific tumor antigen or other tumor cell-surface molecules), a transmembrane domain/region, and an intracellular signaling/activation domain (e.g., the signal region of CD3ζ, and/or one or more costimulatory signaling domains, such as those from CD28, 4-1BB (CD137) and OX-40 (CD134)).

In some embodiments, payloads can be selected that reduce immune responses in a subject. For example, the payload can be an anti-cytokine, such as neutralizing antibodies to tumor necrosis factor (TNF)-α or an interleukin. In some embodiments, payloads of the present disclosure target B-cell depletion, such as neutralizing antibodies to CD20, CD22, CD28, CTLA-4, and B-lymphocyte stimulator (BLyS).

In some embodiments, payloads can also be contractile proteins (e.g., actin and myosin), enzymes (e.g., lactase and pepsin), hormones (e.g., insulin, oxytocin, and somatotropin), structural proteins (e.g., keratin, collagen, and elastin), storage proteins (e.g., ovalbumin and ferritin), transport proteins (e.g., hemoglobin), membrane-bound proteins (e.g., class I, II, or III transmembrane proteins; receptors, transporters, and the like).

In some embodiments, payloads of the present disclosure may be one or more components of a gene editing system. In such embodiments, the oligomer or engineered, regulatable polypeptide regulates activity of the gene editing system and consequently expression of a downstream target protein. A target protein, as used herein, refers to a protein selected for gene editing, including, for example, a protein having a genetic mutation that results in a deleterious effect in a subject or in a cell. For example, payloads of the present disclosure may be a Cas protein (CRISPR-associated protein), including Cas9 and Cas12. The Cas protein may be altered or otherwise modified. For example, the Cas protein may be a deadCas9. Optionally, the Cas9 protein is an enzymatically active Cas9 protein, a Cas9 protein wild-type protein, a Cas9 protein nickase or a nuclease null or nuclease deficient Cas9 protein. Such payloads optionally include nucleases (e.g., Zinc finger nuclease, TALEN (Transcription activator-like effector-based nucleases), or meganucleases) and/or recombinases, such as a Cre recombinase.

Payloads useful in the present disclosure also include polypeptides involved in nucleic acid synthesis and replication, for example, DNA and RNA polymerases, transcription factors, primases, helicases, RNases, ligases, topoisomerases, endonucleases, IRES, and telomerases.

### Linkers

Linker sequences (linkers) are known in the art and are described in references cited herein. Linkers include, for example, GS linkers, GSG linkers, and GGSG (SEQ ID NO: 239) linkers. These linkers are repeats of the subunit one or more times. Thus, a GS linker is a GSn linker (SEQ ID NO:240) where n is a numerical number being 1, 2, 3, 4, 5, 6, 7, 8, 9, 10 or more. Similarly, a GSG linker is a GSGn linker (SEQ ID NO:241) wherein n is a numerical number being 1, 2, 3, 4, 5, 6, 7, 8, 9, 10, or more. A GGSG linker (SEQ ID NO: 239) is a GGSGn linker (SEQ ID NO:242) where n is a numerical number being 1, 2, 3, 4, 5, 6, 7, 8, 9, 10 or more.

### Hinges and Transmembrane Domains

A hinge sequence is a short sequence of amino acids that facilitates flexibility between connected components. The hinge sequence can be any suitable sequence derived or obtained from any suitable molecule. The hinge sequence may be derived from all or part of an immunoglobulin (e.g., IgG1, IgG2, IgG3, IgG4) hinge region, i.e., the sequence that falls between the CH1 and CH2 domains of an immunoglobulin (e.g., an IgG4 Fc hinge), or the extracellular regions of type 1 membrane proteins such as CD8a CD4, CD28 and CD7, which may be a wild type sequence or a derivative thereof. Some hinge regions include an immunoglobulin CH3 domain or both a CH3 domain and a CH2 domain. In some embodiments, the hinge is derived from a transmembrane domain.

Transmembrane domains, useful in the engineered regulatable polypeptide constructs of the present disclosure can include, for example, a MHC1 transmembrane domain, a CD8α transmembrane domain, a B7-1 transmembrane domain, a CD4 transmembrane domain, a CD28 transmembrane domain, a CTLA-4 transmembrane domain, a PD-1 transmembrane domain, or a human IgG4 Fc region.

### Intracellular/Cytoplasmic or Transmembrane Tails

Optionally, the herein provided monomers or polypeptide constructs comprise an intracellular/cytoplasmic or transmembrane tail. Optionally, the intracellular/cytoplasmic or transmembrane tail is a CD8, CD40L, LIGHT, NKG2C, or B7.1 intracellular tail. The absence of a transmembrane region in a construct can be, for example, designed for a secreted payload or a payload with intracellular or intranuclear activity.

### Tags

Optionally, the monomers or polypeptides described herein include a tag. Such a tag allows for isolation or detection of the monomer or polypeptide or for isolation or detection of the payload. Such tags optionally include fluorescent proteins (e.g., green fluorescent protein), His-tag, HA-tag, Myc tag, FLAG tag, mCherry, CD20, CD34, nerve growth factor receptor (NGFR), truncated NGFR (tNGRR), epidermal growth factor (EGFR), or a truncated EGFR (tEGFR).

### Ligands

Ligands may be any agent that binds to the DRDs of the modulation hubs of the engineered, regulatable polypeptides or oligomers described herein, an effective amount of which results in a measurable change in a modification of a characteristic (e.g., abundance, availability, activity) of a payload operably linked to the DRD. In some embodiments, ligands may be synthetic molecules. In some embodiments, stabilizing ligands of the present disclosure may be small molecule compounds. Stabilizing ligands are optionally small molecule therapeutic drugs previously approved by a regulatory agency, such as the U.S. Food and Drug Administration (FDA). Examples of stabilizing ligands and their corresponding DRDs suitable for use in the modulation hubs described herein are shown in Table 1, in patents and applications referenced above in the section exemplifying suitable DRDs, and in U.S. Pat. No. 9,487,787 filed Mar. 33, 2012, U.S. Pat. No. 10,137,180 filed Sep. 6, 2013, PCT Application No. PCT/US2018/037005, filed Jun. 12, 2018, PCT Application No. PCT/US2019/036654 filed Jun. 12, 2019, PCT Application No. PCT/US2019/057698 filed Oct. 23, 2019, PCT Application No. PCT/US2020/021596 filed Mar. 6, 2020, and U.S. application Ser. No. 16/558,224 filed Sep. 2, 2019, the disclosures of all of the aforereferenced applications are incorporated herein by reference in their entireties.

### Nucleic Acids, Vectors, and Cells

Provided herein are expressible nucleic acid constructs encoding one or more engineered, regulatable polypeptide monomers or regulatable polypeptides as described herein. In embodiments, the nucleic acid constructs encoding a monomer include an oligomerization domain and at least one DRD and/or at least one payload and optionally comprise additional components, such as hinges, linkers, transmembrane domains, tags, and intracellular/cytoplasmic and transmembrane tails as described herein. In certain embodiments, a single nucleic acid construct comprises nucleic acid sequences that encode all components of a monomer as described herein. For example, the nucleic acid construct encodes at least a payload, a DRD, and an oligomerization domain. When expressed multiple times, the monomers can assemble into oligomers, for example, dimers, trimers, tetramers, pentamers or hexamers depending on the oligomerization domain and by way of the oligomerization domain. Optionally, a single nucleic acid construct encodes multiple copies of the same or different monomers, which, when expressed assemble into an oligomer. Optionally, certain nucleic acid constructs encode an oligomerization domain and at least one payload, and certain nucleic acid constructs encode an oligomerization domain and at least one DRD, such that when expressed they assemble into an oligomer comprising at least one payload and at least two DRDs.

In embodiments, the nucleic acid constructs encoding regulatable polypeptides include at least one payload and at least one modulation hub comprising at least two DRDs. The nucleic acid constructs encoding regulatable polypeptides may include additional components such as hinges, linkers, transmembrane domains, tags, and intracellular/cytoplasmic and transmembrane tails as described herein.

The nucleic acid constructs encoding monomers and regulatable polypeptides optionally also encode additional components such as signal sequences and cleavage sites including shedding domains. The constructs optionally further comprise a promoter sequence and other regulatory elements (enhancers, translational control elements (e.g., IRES), and elements that control half-life).

Further provided is a plurality of nucleic acid constructs, each construct encoding one or more monomers or polypeptides described herein. Such a plurality of constructs, upon expression, optionally provide for monomers that oligomerize. The constructs of the plurality can be the same or different. Thus, the nucleic acids of the plurality may encode the same polypeptides or monomers or may encode polypeptides or monomers comprising different DRDs, payloads, and/or additional components.

Also provided herein are vectors for expressing one or more of the nucleic acids. Such a vector can be chosen from viral vectors and non-viral vectors, plasmids, cosmids, and artificial chromosomes. By way of example, the vector can be a viral vector, such as a lentiviral vector, a retroviral vector, an adenoviral vector, or an adeno-associated viral vector. The vector optionally comprises nucleic acid sequences that encode transposases and/or nucleases. Non-viral vector examples include physical vectors such as electroporation and chemical vectors such as lipid nanoparticles.

Cells containing one or more nucleic acid constructs or vectors as described herein are provided. The cell provides an expression system or a therapeutic target for the monomers, oligomers, or polypeptides described herein. Suitable cells include immune cells or stem cells. Optionally, the immune cells are primary human T cells, such as T cells derived from human peripheral blood mononuclear cells (PBMC), PBMC collected after stimulation with G-CSF, bone marrow, or umbilical cord blood. In embodiments, the immune cells are tumor infiltrating lymphocytes (TILs), for example collected from a tumor. The immune effector cells may also be NK cells, αβ Tcells, iNKT cells, γδT cells, macrophages, B cells, dendritic cells, myeloid derived progenitor cells, eosinophils, basophils, neutrophils, or Tregs. Optionally, the stem cells are hematopoietic stem cells, human embryonic stem cells, or iPSCs. The cells provided herein are optionally mammalian cells, or, more specifically, human cells.

### Methods of Making

The present disclosure provides methods of making nucleic acid constructs and vectors encoding monomers and polypeptides of this disclosure, methods of making the monomers and polypeptides of this disclosure by expression of nucleic acid constructs, and methods of making cells comprising the nucleic acid constructs or vectors described herein.

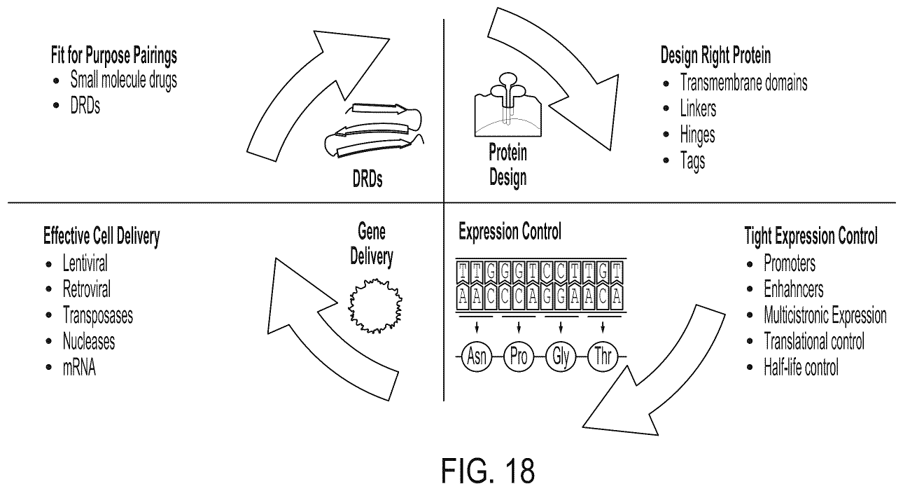

FIG. 18 provides an embodiment of a process for designing nucleic acid constructs and vectors encoding monomers or polypeptides of this invention and thereafter delivering the constructs or vectors for expression in a cell. As shown, a DRD/ligand pair is chosen for regulation of the desired payload. Optionally, a DRD is chosen having a ligand pair that is an FDA-approved small molecule drug. In some embodiments, the DRD/FDA-approved small molecule drug combination is chosen such that it is clinically tractable (e.g., the ligand is capable of regulating the abundance of or regulating the activity of the payload within the approved FDA dose range of the ligand). Thereafter, the nucleic acid or vector is designed to encode the building blocks/components of the polypeptide or monomers described herein, which components include in the case of polypeptides at least a payload and two of the chosen DRDs, and in the case of a monomer, at least an oligomerization domain and at least one of the chosen DRDs, or at least one payload, or at least one payload and at least one of the chosen DRDs. The nucleic acid or vector may be further designed to encode additional building block/components such as transmembrane domains, linkers, hinges, and tags. One of skill in the art, using this disclosure, can select the appropriate components and order them within the construct to achieve the desired outcome. For example, the order of building blocks influences whether the DRD regulates the payload at the N- or C-terminus.

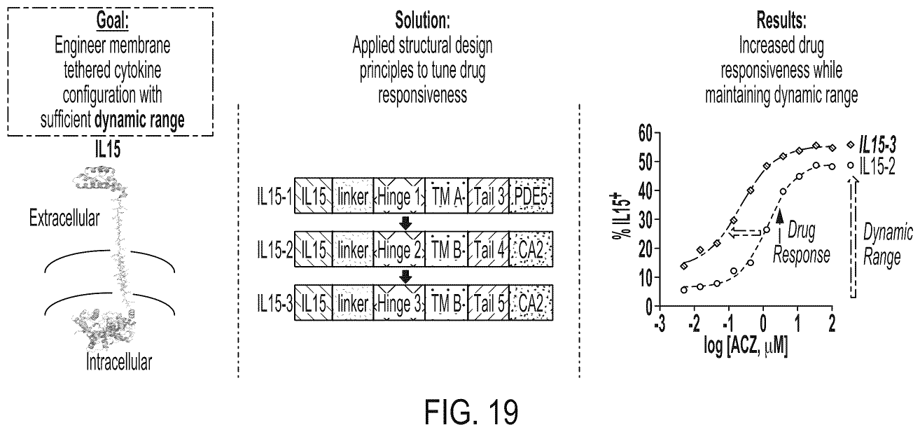

As another example, linker and linker length may influence “constitutive” activity level (i.e., basal activity in the absence of ligand) and in embodiments, the specific linker and length is chosen to maximize the on state (e.g., maximum activity level) while maintaining low basal activity level and ligand (e.g., drug) responsiveness. As yet another example, the specific hinge may allow for conformational changes and thereby influence ligand responsiveness and is thus chosen to result in a sufficient dynamic range to obtain a desired range of payload abundance and biologic activity (i.e., an acceptable payload activity range that corresponds to variation in ligand from zero or minimal to maximum saturation). By way of example, as shown in FIG. 19, if the goal is to create a membrane-tethered cytokine like IL15 with a sufficient dynamic range, the polypeptide optionally includes from the N-terminus the payload (IL15), a linker, a hinge, a transmembrane region, a tail, and a DRD.

In some embodiments, the nucleic acid sequences encoding polypeptides or monomers may be engineered to include one or more additional components, such that, when expressed in a cell, result in the payload localizing internal to the cell membrane (optionally, tethered to the membrane or released into the cytoplasm) and, in certain embodiments, external to the cell membrane (optionally, tethered to the membrane or released extracellularly (i.e., shed or cleaved off of the membrane). Similarly, the nucleic acid constructs are optionally configured to encode polypeptide monomers that oligomerize by way of association of an intracellular, transmembrane, or extracellular oligomerization domain. Thus, all or a portion of the modulation hub or oligomerization domain can be engineered to reside intracellularly, within the cell membrane, or on the surface of the cell (e.g., tethered to the cell surface). See FIG. 15.

In some embodiments, the nucleic acid sequences encoding polypeptide monomer constructs or regulatable payload compositions may be engineered to include one or more of these additional components such that, when expressed in a cell, the regulatable payload composition is engineered with a signal sequence to transport part or all of the engineered regulatable polypeptide construct into the secretory pathway.

After designing the construct for proper function including localization, appropriate components such as promoters, enhancers, multicistronic expression, translation control and half-life control elements are selected to achieve the desired control of payload abundance or activity. Additionally, nucleic acid constructs are designed for cistronic or multicistronic expression as required for the desired expression of various engineered components.

Additionally, vehicles/vectors are chosen and designed to deliver the nucleic acid constructs into the desired cell. For example, the vehicles may be chosen from those previously described including viral vectors (such as lentiviral vectors, retroviral vectors, and adeno-associated vectors), plasmids, cosmids, and artificial chromosomes. Such vectors can be designed to encode transposases, nucleases, and elements that control translation (e.g., IRES). The choice of vector may also influence the choice of various building block components. For example, vectors which demand smaller constructs may require using smaller DRDs.

Cells for engineering can be isolated from any biological sample, including for example, blood (e.g., umbilical cord blood or peripheral blood), bone marrow, fetal tissue (human embryonic stem cells), or tumors. Cells can be modified cells, such as a stem cell modified to be pluripotent (e.g., an induced pluripotent stem cell (iPSC)) or CAR T cells, prior to transduction with the described nucleic acids and vectors.

By way of example, isolated T cells or tumor infiltrating lymphocytes (TILs) can be isolated from a biological sample, transduced, and, optionally, expanded in culture. Such expansion can be performed, for example, by contacting the transduced cell with IL2, feeder cells, recombinant antigen, or an antibody that stimulates cell expansion.

The cell to which the nucleic acid is delivered is selected based, at least in part, on the ability of the cell to allow expression of the polypeptides or monomers disclosed herein and to allow payload activity in a sufficient dynamic range. Optionally, the cell expresses little or no payload in the absence of the provided nucleic acid or vector. In certain embodiments, one of skill in the art would select a cell in need of an increase in payload activity or abundance in a cell that expresses the payload. In certain embodiments, one of skill in the art would select a cell in need of gene editing. In certain embodiments the cell is selected as an effector cell, for example, an immune effector cell.

A person of ordinary skill in the art applying knowledge from this disclosure can build a variety of monomers, polypeptides, and oligomers, as well as nucleic acids and vectors encoding them within scope of this disclosure beyond those explicitly exemplified herein. For example as described, modulation hubs, payloads, DRDs, oligomerization domains, and additional components such as linkers, tails, transmembrane domains, signal sequences, hinges, and the like can be selected to create a variety of polypeptides, oligomers, and nucleic acids encoding them upon considering such factors as desired therapeutic outcome, whether the payload is membrane-bound or secreted, whether the payload acts intracellularly or extracellularly, and whether the oligomerization domain should be located intracellularly, extracellularly, or transmembrane. Accordingly, the example embodiments are non-limiting and help provide guidance for a person of skill in the art to implement other embodiments within the scope of this disclosure.

### Methods of Use

Disclosed herein are methods of regulating payload function, for example methods of regulating abundance, availability and/or activity (“activity”) of a payload. In some embodiments, the method of regulation is a method of modifying the activity of the payload comprising engineering a cell to express a payload operably linked to a modulation hub comprising two or more DRDs responsive to a ligand. In some such embodiments, the activity of the payload (e.g., corresponding to the abundance and/or availability of the payload) is reduced as compared to the activity of a payload in a control cell, for example, a cell engineered to express the payload independent of a DRD.

Optionally, the payload has a biological activity level ranging from a basal activity level in the absence of ligand to a maximum activity in the presence of a saturating amount of ligand, and the method is a method of modulating the activity of a payload comprising contacting a cell engineered to express a payload operably linked to a modulation hub with an effective amount of ligand such that the activity of the payload is increased relative to the basal activity level. In some embodiments, the method comprises contacting the cell with a selected amount of ligand, wherein the selected amount of ligand results in a selected activity level of the payload. In certain embodiments, the method comprises alternatively contacting the cell with varying selected amounts of ligand, to achieve varying selected activity levels ranging from the basal level to the maximum level.

The contacting step can occur in vitro, ex vivo, or in vitro. The contacting step is optionally performed to achieve a continuous selected activity of the payload (i.e., to achieve a continuous on-state of the payload) or to achieve intermittent activity of the payload (i.e., to provide a pulsed delivery of the payload between an on-state and an off-state). “Off-state” means the payload activity is the basal activity level. “On-state” means a selected activity level in the presence of an effective amount of ligand, which is greater than the off-state. Continuous activity of the payload can be achieved by continuous contact of the DRDs with an effective amount of ligand or by providing a subsequent contacting step or steps of the DRDs with the ligand, wherein the subsequent contacting step or steps is/are performed before the activity level of the payload from the previous contacting step reaches the basal activity level. Each contacting step can be varied with regard to the amount of ligand such that, when more ligand is used, more payload activity results, and, when less ligand is used, less payload activity results. Thus, the amount of ligand can be varied with subsequent contacting steps to tune up or tune down the amount and/or activity of the payload over time. Each contacting step can also be varied with respect to frequency in order to achieve a desired pattern of activity level. Because the modulation hubs as described herein result in a lower off-state and optionally a wider dynamic range of activity for a payload as compared to the same payload in a control construct, the activity of the payload can be regulated from a lower off-state and optionally varied over a greater range as compared to a payload in a control construct.

Also disclosed are methods of delivering a payload to a subject, for example a therapeutically effective payload to a subject in need thereof, whereby a nucleic acid construct or a vector as described herein is administered to the subject. The method results in expression, for example in target cells of the subject, of one or more polypeptides or monomers, which monomers self-assemble to form oligomers as described herein. The methods may further comprise controlling the dose or duration of administration of a payload to a subject. For example, the method optionally further comprises administering to the subject a selected amount of paired ligand to deliver a selected activity of the payload to the subject. The ligand can be delivered to achieve continuous or intermittent payload activity in the subject. Continuous payload activity may be a substantially consistent level of activity, or the level of activity may be modulated. Intermittent activity, between the off-state and on-state includes modulating activity between the off-state and a substantially consistent on-state, or between the off-state and varying on-state activity levels. A higher dose or longer duration of administration of the ligand is administered when more activity of the payload is desired, and reduction or elimination of the ligand dose is chosen when less activity is desired. The dose and duration of ligand administration and the resulting activity of the payload may be selected to avoid unacceptable or undesired side effects or toxicity in the subject. Dosages of ligand and schedules for administering the dosages of ligand may be determined empirically by one skilled in the art based on the amount of resulting payload, the activity of the payload, or based on one or more signs of the effect of the payload activity. The ranges for administration of the ligand range from any amount above zero to a saturating dose and the resulting payload activity ranges from a basal level to a maximal level, optionally with a sufficient dynamic range that allows for the desired dose-response to the ligand and concomitant activity range for the payload (e.g., for a given ligand and payload, the range of difference in off-state and maximum payload activity would result from at least a 10 fold range of ligand). This sufficient dynamic range allows for fine tuning and a dose response curve that is not unacceptably steep. In embodiments, the dosage or frequency of administration of ligand and resulting abundance and activity of payload is chosen to avoid, mitigate against, or limit unacceptable or undesired adverse side effects and will vary with the age, condition, and/or sex of the subject, and type of condition being treated, the extent of the condition, or, and whether other therapeutic agents are included in the treatment regimen. Guidance can be found in the literature for appropriate dosages for given classes of ligands.

By way of further example, for a subject with cancer, a nucleic acid construct or vector according to this disclosure is provided to a subject, wherein the nucleic acid construct or vector encodes a payload operably linked to a modulation hub and wherein the payload targets a tumor cell or an immune cell that in turn targets the tumor cell. Such a method optionally comprises administering to the subject a nucleic acid construct or vector according to this disclosure encoding a payload operably linked to a modulation hub, wherein the payload is an immune checkpoint inhibitor, a cytokine, CAR, or TCR.

Also provided herein are methods of delivering a payload operably linked to a modulation hub to a subject by administering to the subject a cell containing a nucleic acid or vector described herein. Such a method can further comprise isolating cells from a subject, transducing the isolated cells with the nucleic acids or vectors encoding the monomers (which when expressed assemble into regulatable oligomers) or regulatable polypeptides described in this disclosure, expanding the cells in vitro, and providing the cells to the same or different subject. The payload is selected to treat the subject receiving the cells. The subject may have cancer, an autoimmune disease, a genetic mutation, a deficiency in an essential polypeptide or the like. The transduced cells can be, by way of example, immune effector cell (e.g., an NK cell, iNKT, αβ T cell, γδ T cell, tumor infiltrating lymphocyte (TILs), macrophages, B cells), or stem cells). The cells can be isolated from the same subject (autologous source) that receives the transduced cells or the cells can be isolated from a different subject (e.g., an allogeneic source). By way of example, when the subject has cancer, a T cell or TIL can be isolated from the subject and engineered to express a cytokine, a CAR, and/or a TCR. For example, the cell can be a CD19 CAR-T cell. The cell is optionally expanded, and the cell or the expanded population of cells is administered to the same or different subject. In certain embodiments, the T-cells, CAR-T cells, NK cells, or TILs, are administered in an amount from about 1000 cells/injection to up to about 10 billion cells/injection, such as 1×10¹⁰, 1×10⁹, 1×10⁸, 1×10⁷, 5×10⁷, 1×10⁶, 5×10⁶, 1×10⁵, 5×10⁵, 1×10⁴, 5×10⁴, 1×10³, 5×10³ cells per injection, or any ranges between any two of the numbers, end points inclusive. Optionally, from 1×10⁸ to 1×10¹⁰ cells are administered to the subject. Optionally, the cells are administered one, two, three, or four times as needed.

In the treatment methods, the ligand dosage regimen including the selected amount of ligand for administration to the subject and frequency of administration of the selected amount of ligand is chosen to result in regulation of the payload and/or a desired outcome for the subject. The subject is optionally monitored for the outcome. Thus, for example, the number of malignant cells in a sample, the circulating tumor DNA in a sample, or the size of a solid tumor upon imaging can be detected. If the desired end point is achieved (e.g., showing successful treatment of cancer), the ligand can be reduced or discontinued so as to reduce or eliminate the payload, for example to reduce the abundance, availability and/or activity of the payload below a pre-determined threshold to eliminate or mitigate against unwanted or undesired side effects. Similarly, if the subject develops a cytokine storm, an allergic reaction, or other adverse effect from the payload, the ligand can be reduced or discontinued. Also disclosed herein are methods of regulating expression of a downstream target protein of a gene editing process. In some embodiments, such method comprises engineering a cell to express an oligomer or engineered, regulatable polypeptide comprising a payload such as a CAS9 protein or transcription factor protein operably linked to a modulation hub. In certain embodiments, for a subject with a genetic mutation, a nucleic acid construct or vector according to this disclosure is provided to deliver a payload that provides a nucleic acid editing polypeptide or system operably linked to a modulating hub. Target cells in the subject are transduced with the nucleic acid construct or vector to allow the gene editing payload to modify the nucleic acid (e.g., genomic DNA or RNA) of the transduced cell. The activity level of the payload and therefore expression of the target protein can be regulated by administration of ligand to the subject. Systems for nucleic acid editing are known in the art, such as CRISPR/Cas 9 systems, TALENs, retrotransposons, and the like. The nucleic acid construct or vector can be engineered to encode one or more components of any one of the systems, such that one or more nucleic acids in the transduced cell of the subject is edited to provide a desired nucleic acid modification.

### Definitions

The terms “about” and “approximate,” when used to refer to a measurable value such as an amount, concentration, dose, time, temperature, activity, level, number, frequency, percentage, dimension, size, weight, position, length and the like, is meant to account for variations due to experimental error, which could encompass variations of ±15%, ±10%, ±5%, ±1%, ±0.5%, or even ±0.1% of the specified amount, concentration, dose, time, temperature, activity, level, number, frequency, percentage, dimension, size, weight, position, length and the like. All measurements or numbers are implicitly understood to be modified by the word about, even if the measurement or number is not explicitly modified by the word about. In instances in which the terms “about” and “approximate” are used in connection with the location or position of regions within a reference polypeptide, these terms encompass variations of ±up to 20 amino acid residues, ±up to 15 amino acid residues, ±up to 10 amino acid residues, ±up to 5 amino acid residues, ±up to 4 amino acid residues, ±up to 3 amino acid residues, ±up to 2 amino acid residues, or even ±1 amino acid residue.

Reference is made herein to a “basal activity level.” Basal level as used herein can be zero, near zero, or any amount in the absence of exogenous ligand. Basal activity may occur because of endogenous levels of the same or different ligand or may occur because of a resting level of payload production in the absence of exogenous ligand.

Reference to “biological activity” is understood to mean under appropriate conditions even if not so stated.

As used herein “contacting” is understood to mean providing an agent (such as a ligand) to a target (such as a DRD) such that the agent and target may come into contact with one another. For example, contacting includes providing a ligand in vitro to a cell (e.g. when the DRD is located extracellularly or on the cell surface). As another example, contacting also includes providing the ligand to a cell, wherein the DRD is located intracellularly, such that the ligand reaches the cytoplasm. Similarly, a cell can be contacted in vivo, by administering a ligand to a subject such that the ligand reaches a cell or DRD.

As used herein, the term “control construct” refers to a construct similar to the test engineered, regulatable oligomer or engineered regulatable polypeptide (“test construct”) except the control construct lacks an oligomerization domain and has only a single DRD.

“DRD” is understood to mean a domain responsive to a ligand, even if not so stated.

A “hetero-oligomerization domain” as described herein is a first oligomerization domain on a first monomer that promotes oligomerization through a second oligomerization domain on a second monomer, wherein the second oligomerization domain is different than the first and wherein the first oligomerization domain does not promote oligomerization with an oligomerization domain that is the same.

The terms “ligand,” “paired ligand,” and “stabilizing ligand” are used interchangeably and mean the same thing when used in reference to a drug responsive domain (“DRD”) and/or modulation hub.

As used herein, “operably linked” means that, in the presence of a paired ligand, the modulation hub or DRD is linked to the payload directly or indirectly so as to alter a measurable characteristic of the payload (e.g., alters the level of activity of the payload as compared to the level of activity in the absence of the paired ligand). In some embodiments, the measured level of amount and/or activity of the payload increases in the presence of an effective amount of ligand as compared to the measured level of expression or activity in the absence of ligand. An effective amount of ligand means the amount of ligand needed to see an increase in the measure of the amount or activity of the payload. In some embodiments, the effective amount is not so great as to produce unacceptable toxicity or off-target effects. Optionally, the measurable characteristic is a therapeutic outcome, an amount of the payload in a sample, or a biological activity level of the payload (for which measuring the amount of payload can serve as a proxy).

The term “payload” refers to the agent whose abundance, activity, availability, expression, function or other characteristic is desired to be regulated by a DRD and/or modulation hub.

Wherever the phrase “linked” or “bound” or the like is used, the phrase “directly or indirectly” is understood to follow unless explicitly stated otherwise or nonsensical in context. Thus, the phrase “a DRD linked to another DRD” or “two linked DRDs” means in both cases that a first DRD is directly or indirectly linked to a second DRD. For example, “two linked DRDs” and the like covers the situation wherein two DRDs are not directly connected to one another but rather are in association with one another because each is connected to an oligomerization domain, and the respective oligomerization domains are linked, e.g., via non-covalent bonds.

The details of one or more embodiments of the present disclosure are set forth in the description and accompanying drawings. It is to be understood that other embodiments may be utilized and structural or process changes made without departing from the scope of the disclosure. In other words, illustrative embodiments and aspects are described below. But it will be appreciated that in the development of any such actual embodiment, numerous implementation-specific decisions may be made to achieve the developer's specific goals, such as compliance with system-related and business-related constraints, which may vary from one implementation to another. Moreover, it will be appreciated that such development effort might be complex and time-consuming but would nevertheless be a routine undertaking for those of ordinary skill in the art having the benefit of this disclosure.

Publications cited herein and the material for which they are cited are hereby specifically incorporated by reference in their entireties.

The examples below are intended to further illustrate certain aspects of the methods and compositions described herein and are not intended to limit the scope of the claims.

### EXAMPLES

### Example 1. Cloning of IL12-X and CD19-IL12-X Constructs

IL12-(241-252 and 254-262) and CD19-IL12-(297-316, 319-332) plasmids were each constructed in a pELNS vector (a third-generation self-inactivating lentiviral expression vector) using standard molecular biology techniques. Gene fragments (Gblocks or strings DNA) encoding IL12, Glycine-serine linkers, various hinges, transmembrane domains and cytoplasmic tails were purchased from Integrated DNA Technologies or Thermo-fisher scientific. The gene fragments were inserted into the pELNS vector and placed under the control of the EF1a promoter using Gibson assembly (NEBuilder Hifi). The assembled plasmid was transformed into E. coli (NEB stable) for amplification and sequence confirmed before proceeding with virus production.

### Example 2. Regulation of IL12 from Tandem IL12 mCherry Constructs in HEK Cells

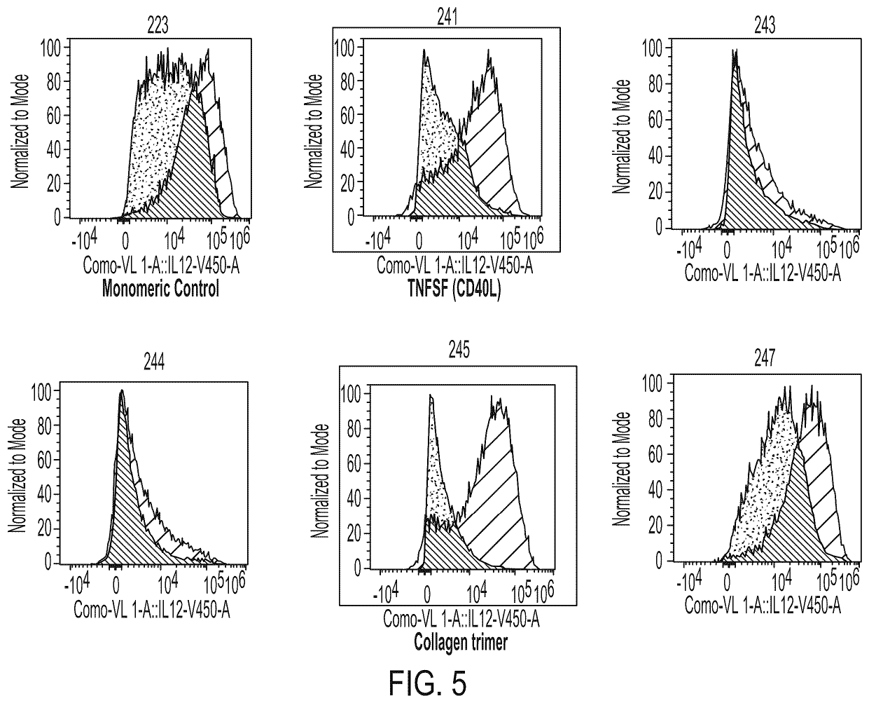

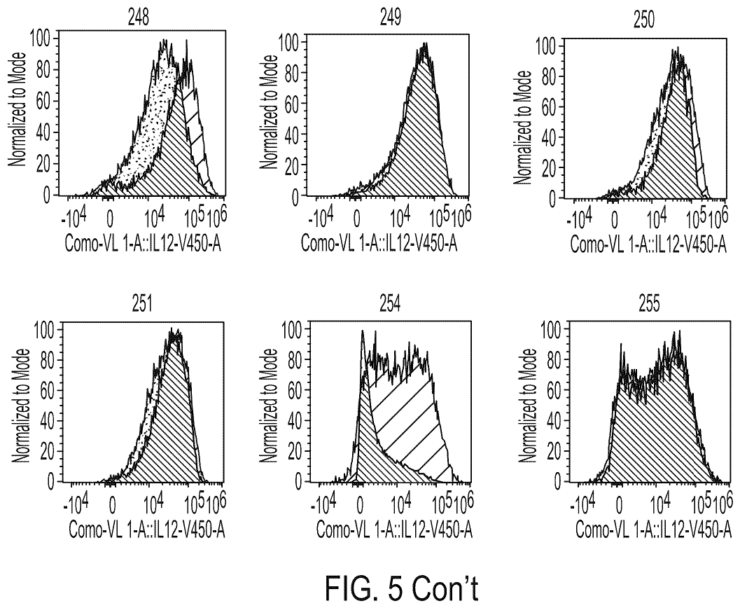

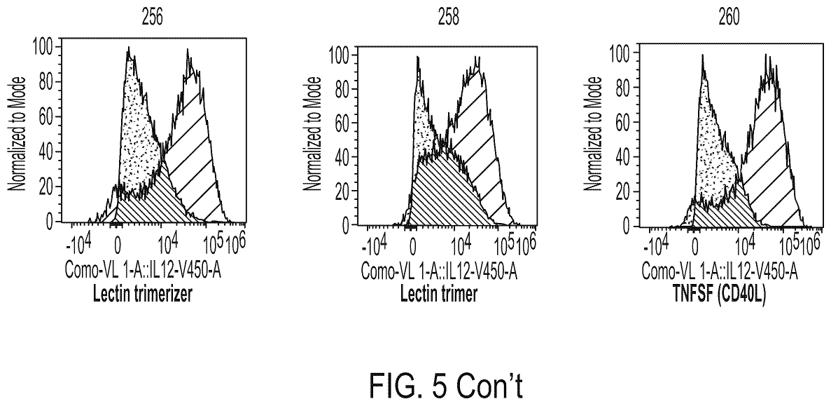

HEK-293 and T cells were cultured in standard media (DMEM (Fisher Scientific Cat #11-960-044) with 10% FBS (Fisher Scientific Cat #10-082-147) and 1% P/S (ThermoFisher Cat #15140122). HEK293 and T cells were transiently transfected using manufacturer's protocol with Lipofectamine 2000 and 1 μg plasmid DNA each of: IL12-223, 241, 243, 244, 245, 247, 248, 249, 250, 251, 254, 255, 256, 258 and 260. 100 μM acetazolamide was added one day after transfection, and the cells were further cultured for 1 more day. mCherry was used as a transfection marker and anti-IL12p40/70-V450 Cat #561380 BD was used to stain for IL12. The flow cytometry graphs in FIG. 5A, FIG. 5B, and FIG. 5C show the surface abundance of IL12 in HEK cells in the presence or absence of ACZ. IL12-223 which was configured without an oligomerization motif showed higher basal payload abundance on cell surface than constructs IL12-241, 245, 254, 256, 258 and 260 which were configured with trimerization motifs.

### Example 3. Regulation of IL12 from Tandem IL12 mCherry Constructs in Jurkat Cells

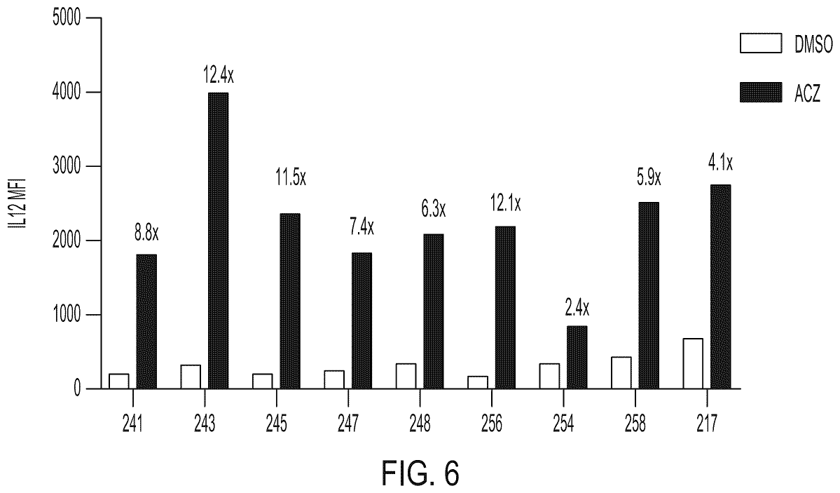

Jurkat, Clone E6-1, cells were cultured in standard media (RPMI+ GlutaMAX Supplement: Life Technologies Cat #61870127, Fetal Bovine Serum (FBS) Life Technologies Cat #10-082-147). Jurkat cells were transduced with lentivirus produced with each of IL12-217, -241, -243, -245, -247, -248, -254, -256, and -258. The components of these constructs are depicted in FIG. 3. Jurkat cells were transduced to 10-25% positive as determined by using mCherry as a transduction marker. Five days post transduction, the cells were treated with 100 μM acetazolamide or DMSO and the cells were further cultured for 1 more day. mCherry was used as a transduction marker and anti-IL12p40/70-V450 Cat #561380 BD was used to stain for IL12. FIG. 6 shows geometric MFI indicative of IL12 construct expression on mCherry positive Jurkat cells in the presence and absence of ACZ. Note the cells transduced with trimerized constructs (IL12-245, 247, 248, 254, 256) have low IL12 expression (off-state) in the absence of ACZ. The numbers above the ACZ treated cells represent high fold change between on and off-state. MFI was calculated on cells within the mCherry gate.

### Example 4. Regulation of Shed IL12 from Tandem IL12 mCherry Constructs in Primary Human T Cells

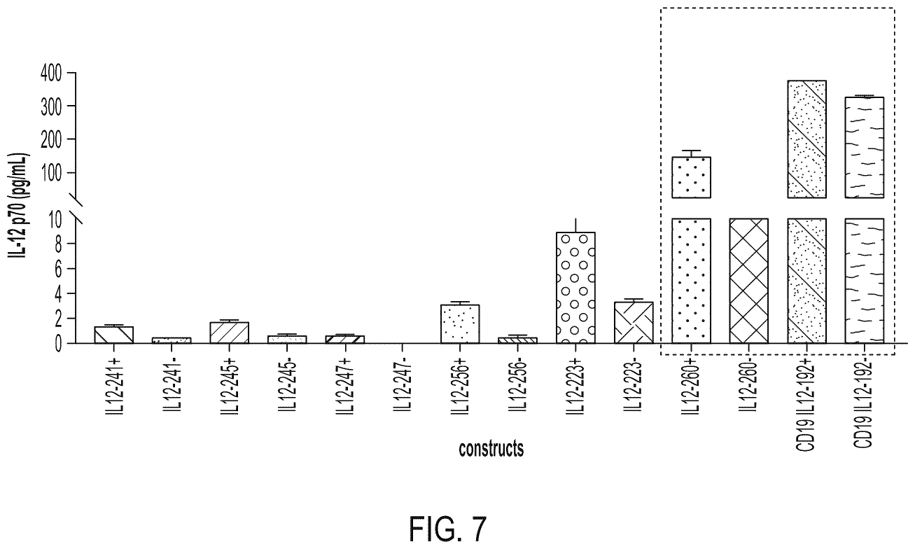

On day 0, primary human T cells were stimulated with Dynabeads (T-expander CD3/CD28) at a 3:1 bead:cell ratio in media containing 10% fetal bovine serum (FBS). The next day, lentivirus produced with constructs (IL12-241, 245, 247, 256, 223, 260 and CD19-IL12-192) and controls were added in the presence of reduced serum (5% FBS). The components of these constructs are depicted in FIG. 3. On day 2, the cells were diluted 1:2 with fresh 10% FBS media. On day 5, the cells were analyzed for mCherry transduction and replated to account for differences in transduction efficiency, media was replaced, and cells were treated with 100 μM acetazolamide or DMSO. Cytokine that had accumulated for 24 hr in the culture supernatants (from 100,000 cells per 200 μL media) were measured using human IL12p70 (and/or human interferon-gamma) MSD V-plex assay kits (Meso Scale Discovery). FIG. 7 shows the level of IL12p70 secreted from T cells transduced with different constructs. The same constructs tested in Jurkat cells produced similar results in the transduced T cells. Note that only the IL12 that possessed a hinge with a defined shedding mechanism resulted in an amplified dynamic range for regulation of the shed IL12 fraction in the cell supernatants.

### Example 5. Regulation of Membrane-Bound IL12 Constructs in Primary Human T Cells

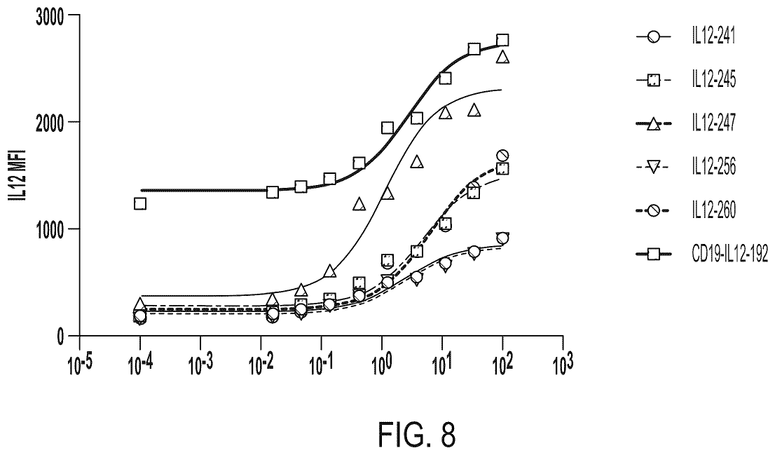

On day 0, primary human T cells were stimulated with Dynabeads (T-expander CD3/CD28) at a 3:1 bead:cell ratio in media containing 10% fetal bovine serum (FBS). The next day, lentivirus produced with constructs expressing mCherry and membrane-bound trimerizing flexi-IL12 (IL12-241, 245, 247, 256, 260, and CD19-IL12-192) and controls were added in the presence of reduced serum (5% FBS). On day 2, the cells were diluted 1:2 with fresh 10% FBS media. On day 5, the cells were analyzed for mCherry transduction media was replaced and cells were treated with 100 μM acetazolamide or DMSO. Twenty-four hours later T cells were analyzed by FACs. mCherry was used as a transduction marker and anti-IL12p40/70-V450 Cat #561380 BD was used to stain for IL12. Alternatively, cells were expanded for a total of 10-11 days and then frozen in liquid nitrogen. Next, T cells were thawed and counted. 1-2×10⁵ cells were plated per well of a 96-well V-bottom plate, re-stimulated with soluble CD3/CD28 Immunocult reagent (Stem Cell Technologies) and treated with a dose response of acetazolamide ranging from 0-100 μM. After overnight incubation, transduction efficiency was analyzed by flow cytometry using mCherry. Surface expressed IL12 was detected with an anti-IL12p70 antibody (BD). FIG. 8 shows the geometric MFI of surface IL12p70 expression on mCherry or CAR+ cells. Ligand concentration dose response curves were fit using Prism Software.

TABLE 2
Fold-change in surface IL12 abundance after construct transduction.

| Construct | Fold change | EC50 (μM) |
|---|---|---|
| IL12-241 | 5.3 | 2.6 |
| IL12-245 | 8.3 | 4.6 |
| IL12-247 | 8.8 | 1.1 |
| IL12-256 | 5.6 | 2.4 |
| IL12-260 | 9.8 | 6.2 |
| CD19-IL12-192 | 2.2 | 2.7 |

### Example 6. Measuring Membrane-Bound IL12 on T Cells after Construct Transduction

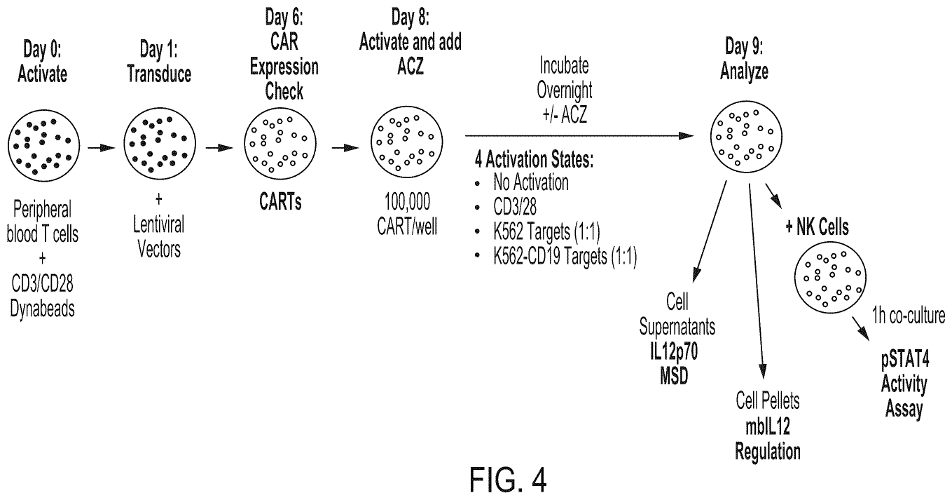

See FIG. 4 for an overview of the testing schematic. On day 0, primary human T cells were stimulated with Dynabeads (T-expander CD3/CD28) at a 3:1 bead:cell ratio in media containing 10% fetal bovine serum (FBS). The next day, any one of the constructs from CD19-IL12-192 (SEQ ID NO:124) and CD19-IL12 (297-316 or 319-332) (SEQ ID NO: 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232) were packaged as lentivirus.

TABLE 3
Amino acid and nucleic sequence IDs of CA2 variants, IL12, and tested constructs

| Amino | Nucleic | | |
|---|---|---|---|
| acid SEQ | Acid | | |
| ID NO: | SEQ ID: | Name | Description |
| 1 | 2 | CA2-WT | Human wildtype CA2 |
| 3 | 4 | Mdel1-CA2 | CA2 wildtype sequence with Mdel1 |
| 5 | 6 | L156H | L156H mutation in SEQ ID NO: 3 |
| 7 | 8 | CA2-S56N | S56N mutation in SEQ ID NO: 1 |
| 9 | 10 | Mdel1-CA2- | S56N mutation in SEQ ID NO: 3 |
| | | S56N | |
| 117 | 118 | IL12 | Human IL12; Flexi-IL12, P40 and P35 subunits |
| | | | are linked through a (G₄S)₃ linker |
| 121 | 122 | CD19-063 | |
| 123 | 124 | CD19-IL12- | CD19CAR-P2A-IL12-(GS)15-B7-1hinge-TM- |
| | | 192 | 12aa cyto-CA2(L156H) |
| 125 | 126 | IL12-217 | IL12-(GS)15-B7-1hinge-TM-cyto- |
| | | | CA2(L156H)-P2A-mCherry |
| 127 | 128 | IL12-223 | IL12-(GS)15-CD8ahinge-TM-cyto- |
| | | | CA2(L156H)-P2A-mCherry |
| 129 | 130 | IL12-241 | CA2(S56N)-(GGSG)3-CD40L cyto- |
| | | | CD40LTM-CD40hinge-CD40L-abrogatedshed |
| | | | site-CD40L ECD-(GS)7-IL12-P2A-mCherry |
| 131 | 132 | IL12-242 | CA2(WT)-(GGSG)3-CD40L cyto-CD40LTM- |
| | | | CD40Lhinge-CD40L-abrogated shed site- |
| | | | CD40L ECD-(GS)7-IL12-P2A-mCherry |
| 133 | 134 | IL12-243 | CA2(S56N)-(GGSG)2-4-1BBL cyto-4-1BBL |
| | | | TM-4-1BBLhinge-4-1BBL ECD-(GS)7-IL12- |
| | | | P2A-mCherry |
| 135 | 136 | IL12-244 | CA2(WT)-(GGSG)2-4-1BBL cyto-4-1BBL |
| | | | TM-4-1BBLhinge-41BBL ECD-(GS)7-IL12- |
| | | | PA2-mCherry |
| 137 | 138 | IL12-245 | CA2(S56N)-linker-CD40L cyto-CD40L TM- |
| | | | hinge(nonshed)-linker-collagen 18 trimer- |
| | | | linker-IL12-P2A-mCherry |
| 139 | 140 | IL12-246 | CA2(WT)-linker-CD40cyto-TM- |
| | | | hinge(nonshed)-linker-collagen 18 trimer- |
| | | | linker-IL12-P2A-mCherry |
| 141 | 142 | IL12-247 | P40sp-IL12-linker-DAP12hinge-DAP12TM- |
| | | | DAP12cyto-CA2(L156H)-P2A-mCherry |
| 143 | 144 | IL12-248 | P40sp-IL12-linker-DAP12hinge-DAP12TM- |
| | | | DAP12cyto-CA2(S56N)-P2A-mCherry |
| 145 | 146 | IL12-249 | P40sp-IL12-linker-DAP12hinge-DAP12TM- |
| | | | DAP12cyto-CA2(WT)-P2A-mCherry |
| 147 | 148 | IL12-250 | P40sp-IL12-linker-B7-1hinge-DAP12TM-B7- |
| | | | 1cyto-CA2(L156H)-P2A-mCherry |
| 149 | 150 | IL12-251 | P40sp-IL12-linker-B7-1hinge-DAP12TM-B7- |
| | | | 1cyto-CA2(S56N)-P2A-mCherry |
| 151 | 152 | IL12-252 | P40sp-IL12-linker-B7-1hinge-DAP12TM-B7- |
| | | | 1cyto-CA2(WT)-P2A-mCherry |
| 153 | 154 | IL12-254 | P40sp-IL12-linker-collectin7-linker-B7- |
| | | | 1hinge-TM-Cyto-CA2(S56N)-P2A-mCherry |
| 155 | 156 | IL12-255 | P40sp-IL12-linker-collectin7-linker-B7- |
| | | | 1hinge-TM-Cyto-CA2(WT)-P2A-mCherry |
| 157 | 158 | IL12-256 | CA2(S56N)-linker-cd40Lcyto-TM- |
| | | | hinge(nonshed)-G4S-trimerizing domain |
| | | | (collectin 7)-(GS)7-IL12-P2A-mCherry |
| 159 | 160 | IL12-257 | CA2(WT)-linker-cd40Lcyto-TM- |
| | | | hinge(nonshed)-G4S-trimerizing domain |
| | | | (collectin 7)-(GS)7-IL12-P2A-mCherry |
| 161 | 162 | IL12-258 | CA2(S56N)-Langerin cyto-langerinTM- |
| | | | langerin hinge-langerinECD-(GS)7-IL12-P2A- |
| | | | mCherry |
| 163 | 164 | IL12-259 | CA2(WT)-Langerin cyto-langerin TM-langerin |
| | | | hinge-langerinECD-(GS)7-IL12-P2A-mCherry |
| 165 | 166 | IL12-260 | CA2(S56N)-(GGSG)3-CD40L cyto- |
| | | | CD40LTM-CD40Lhinge-CD40Lshed site- |
| | | | CD40L ECD-(GS)7-IL12-P2A-mCherry |
| 167 | 168 | IL12-261 | P40sp-IL12-linker-collagen 18 trimer-linker- |
| | | | B7-1hinge-TM-Cyto-CA2(S56N)-P2A- |
| | | | mCherry |
| 169 | 170 | IL12-262 | P40sp-IL12-linker-collagen 18 trimer-linker- |
| | | | B7-1hinge-TM-Cyto-CA2(WT)-P2A-mCherry |
| 171 | 172 | CD19-IL12- | CD19-CAR-P2A-CA2-(GGSG)3-NKG2Ccyto- |
| | | 297 | Phospholamban cyto PhospholambanTM- |
| | | | NKG2C hinge (GGS)₂ (GS)7-IL12 |
| 173 | 174 | CD19-IL12- | CD19-CAR-P2A-CA2-(GGSG)3-NKG2Ccyto- |
| | | 298 | Phospholamban cyto-Phospholamban TM- |
| | | | NKG2Chinge-CD40Lshedsite-(GS)7-IL12 |
| 175 | 176 | CD19-IL12- | CD19-CAR-P2A-CA2(S56N)-(GSG)6- |
| | | 299 | collagen18trimer-(GGSG)3-CD40Lcyto-TM- |
| | | | hinge(shed)-(GS)7-IL12 |
| 177 | 178 | CD19-IL12- | CD19-CAR-P2A-collagen 18trimer-(GSG)6- |
| | | 300 | S56N-(GGSG)3-CD40Lcyto-TM-hinge(shed)- |
| | | | (GS)7-IL12 |
| 179 | 180 | CD19-IL12- | CD19CAR-P2A-CA2(S56N)-(GSG)6- |
| | | 301 | trimerizing domain (collectin 7)-(GGSG)3- |
| | | | CD40Lcyto-TM-hinge(shed)-(GS)7-IL12 |
| 181 | 182 | CD19-IL12- | CD19CAR-P2A-trimerizing domain (collectin |
| | | 302 | 7)-(GSG)6-S56N-(GGSG)3-CD40Lcyto-TM- |
| | | | hinge(shed)-(GS)7-IL12 |
| 183 | 184 | CD19-IL12- | CD19CAR-P2A-CA2(S56N)-CD40Lcyto-TM- |
| | | 303 | hinge-(non-shed)-CD40L ECD-IL12 |
| 185 | 186 | CD19-IL12- | CD19CAR-P2A-S56N-linker-cd40Lcyto-TM- |
| | | 304 | hinge(nonshed)-linker-collagen trimer-linker- |
| | | | IL12 |
| 187 | 188 | CD19-IL12- | CD19CAR-P2A-CA2(S56N)-linker- |
| | | 305 | cd40Lcyto-TM-hinge(nonshed)-G4S- |
| | | | trimerizing domain (collectin 7)-(GS)7-IL12 |
| 189 | 190 | CD19-IL12- | CD19CAR-P2A CA2(S56N)-(GGSG)3-CD40L |
| | | 306 | cyto-CD40LTM-CD40Lhinge-CD40Lshed site- |
| | | | CD40L ECD-(GS)7-IL12 |
| 191 | 192 | CD19-IL12- | CD19CAR-P2A-CA2(S56N)-(GSG)7- |
| | | 307 | VASPtetramer-(GGSG)3-CD40Lcyto-TM- |
| | | | hinge(shed)-(GS)7-IL12 |
| 193 | 194 | CD19-IL12- | CD19CAR-P2A VASPtetramer-(GSG)7-S56N- |
| | | 308 | (GGSG)4-CD40Lcyto-TM-hinge(shed)-(GS)7- |
| | | | IL12 |
| 195 | 196 | CD19-IL12- | CD19CAR-P2A-P40sp-IL12-linker- |
| | | 309 | DAP12hinge-DAP12TM-DAP12cyto- |
| | | | (GGSG)4-VASPtetramer-linker-CA2(S56N) |
| 197 | 198 | CD19-IL12- | CD19CAR-P2A-P40sp-IL12-linker- |
| | | 310 | DAP12hinge-DAP12TM-DAP12cyto- |
| | | | (GGSG)4-CA2(S56N)-(GSG)7-VASPtetramer |
| 199 | 200 | CD19-IL12- | CD19CAR-P2A-P40sp-IL12-linker-hlaahinge- |
| | | 311 | hlaaTM-hlaacyto-(GGSG)4-VASPtetramer- |
| | | | linker-CA2(S56N) |
| 201 | 202 | CD19-IL12- | CD19CAR-P2A-P40sp-IL12-linker-hlaahinge- |
| | | 312 | hlaaTM-hlaacyto-(GGSG)4-CA2(S56N)- |
| | | | (gsg)7-VASPtetramer |
| 203 | 204 | CD19-IL12- | CD19CAR-P2A-P40sp-IL12-linker- |
| | | 313 | DAP12hinge-DAP12TM-DAP12cyto- |
| | | | (GGSG)4-collagen18trimer-linker-CA2(S56N) |
| 205 | 206 | CD19-IL12- | CD19CAR-P2A-P40sp-IL12-linker- |
| | | 314 | DAP12hinge-DAP12TM-DAP12cyto- |
| | | | (GGSG)4-CA2(S56N)-(gsg)7-collagen18 |
| | | | trimer |
| 207 | 208 | CD19-IL12- | CD19CAR-P2A-P40sp-IL12-linker-hlaahinge- |
| | | 315 | hlaaTM-hlaacyto-(GGSG)4-collagen18trimer- |
| | | | linker-CA2(S56N) |
| 209 | 210 | CD19-IL12- | CD19CAR-P2A-P40sp-IL12-linker-hlaahinge- |
| | | 316 | hlaaTM-hlaacyto-(GGSG)4-CA2(S56N)- |
| | | | (gsg)7-collagen18trimer |
| 211 | 212 | CD19-IL12- | CD19CAR-P2A-CA2(S56N)-linker-cd40cyto- |
| | | 319 | TM-hinge(shed)-linker-collagen trimer-linker- |
| | | | IL12 |
| 213 | 214 | CD19-IL12- | CD19CAR-P2A-P40sp-IL12-linker-CD40L |
| | | 320 | shed site-DAP12hingeDAP12TM-DAP12cyto- |
| | | | CA2(L156H) |
| 215 | 216 | CD19-IL12- | CD19CAR-P2A-CA2(S56N)-linker-cd40cyto- |
| | | 321 | TM-hinge(shed)-linker-collectin7 trimerizer- |
| | | | linker-IL12 |
| 217 | 218 | CD19-IL12- | CD19CAR-P2A-CA2(S56N)-linker- |
| | | 322 | CD40L(non-shedding)-(GS)6-Shedding-(GS)4- |
| | | | IL12 |
| 219 | 220 | CD19-IL12- | CD19CAR-P2A-CA2(S56N)-(GGSG)3-Light- |
| | | 323 | (gs)7-IL12 |
| 221 | 222 | CD19-IL12- | CD19CAR-P2A-CA2(S56N)-(GGSG)3- |
| | | 324 | CD40Lcyto-phospholamban cyto-TM- |
| | | | CD40Lhinge-(GS)7-IL12 |
| 223 | 224 | CD19-IL12- | CD19CAR-P2A-CA2(S56N)-(GGSG)3-Light |
| | | 325 | cyto-phospholamban cyto-tm-LIGHT hinge- |
| | | | (GS)7-IL12 |
| 225 | 226 | CD19-IL12- | CD19CAR-P2A-CA2(S56N)-(GSG)7- |
| | | 326 | VASPtetramer(GGSG)3-light(cyto-TM-hinge)- |
| | | | GS7-IL12 |
| 227 | 228 | CD19-IL12- | CD19CAR-P2A-CA2(S56N)-(GSG)7- |
| | | 327 | collectin7-(GGSG)3-LIGHT(cyto-TM-hinge)- |
| | | | GS7-IL12 |
| 229 | 230 | CD19-IL12- | CD19CAR-P2A-collectin7-(GSG)6- |
| | | 328 | CA2(S56N)-(GGSG)3-LIGHT(cyto-TM- |
| | | | hinge)-GS7-IL12 |
| 231 | 232 | CD19-IL12- | CD19CAR-P2A-CA2(S56N-(GGSG)3-LIGHT |
| | | 329 | (cyto-TM-hinge)-(GSG)4-collectin7-GS7-IL12 |
| 233 | 234 | CD19-IL12- | CD19CAR-P2A-P40sp-IL12-linker-hlaahinge- |
| | | 330 | hlaaTM-hlaacyto-(GGSG)4-collectin7-linker- |
| | | | CA2(S56N) |
| 235 | 236 | CD19-IL12- | CD19CAR-P2A-P40sp-IL12-linkershed- |
| | | 331 | collectin7-linker-hlaahinge-hlaaTM-hlaacyto- |
| | | | (GGSG)4-CA2(S56N) |
| 237 | 238 | CD19-IL12- | CD19CAR-P2A-P40sp-IL12-linkershed- |
| | | 332 | hlaahinge-hlaaTM-hlaacyto-(GGSG)4- |
| | | | CA2(S56N) linker collectin7 |

On day 2, the cells were diluted 1:2 with fresh 10% FBS media. On day 3 the cells were expanded 1:3 into with fresh medium. On day 6, the cells were assessed for transduction efficiency using flow cytometry. As a readout of cell transduction, CD19-CAR expression was detected using a CD19-Fc reagent followed by a fluorescently labeled anti-human Fc antibody. The Dynabeads were removed using magnetic selection and the cells split 1:2 into fresh medium. On day 8, a normalized number of transduced (CAR+) cells were plated on a 96-well plate and treated with either 20 μM ACZ or an equivalent volume of DMSO vehicle control in the absence or presence of antigen re-stimulation. Stimulation conditions were either human Immunocult soluble CD3/CD28 reagent (StemCell Technologies, catalogue number 10971), K562 cells stably expressing the CAR antigen CD19 at a E:T ratio of 1:1, or parental control K562 cells. After overnight incubation, mbIL12 abundance on transduced T-cells was analyzed by flow cytometry using CD19-Fc followed by a fluorescently labeled anti-human Fc antibody to detect surface CAR expression, anti-IL12p70, anti-CD45, anti-CD3, anti-CD4, and anti-CD8 antibodies. The geometric mean fluorescent intensities (GMFIs) of IL12p70 in transduced cells were normalized against the gMFIs of mbIL12 from identically treated (ligand+antigen restimulation) untransduced cells with values close to 1 reflecting an undetectable amount of mbIL12. Table 4 shows the fold-change of mbIL12 within a restimulation condition was calculated as the gMFI of mbIL12 with ligand added divided by the gMFI without ligand for a given construct.

TABLE 4
Expression constructs and their fold
expression change in transfected T cells

| | | | Off- | Fold | Fold |
|---|---|---|---|---|---|
| Construct | Oligomerization | Shedding | state | MFI | MSD |
| no. | domain location | linker | MFI | change | change |
| CD19-IL12- | Penta/TM | Mono | 1.8 | 4.9 | 2.5 |
| 298 | | | | | |
| CD19-IL12- | Trimer/Intra | Mono | 1.3 | 3.0 | 3.1 |
| 301 | | | | | |
| CD19-IL12- | Trimer/Intra | Mono | 0.4 | 30.4 | 13.2 |
| 302 | | | | | |
| CD19-IL12- | Trimer/Extra | None | 1.4 | 16.7 | 1.4 |
| 303 | | | | | |
| CD19-IL12- | Trimer/Extra | None | 0.8 | 33.4 | 1.1 |
| 304 | | | | | |
| CD19-IL12- | Trimer/Extra | None | 0.7 | 48.4 | 3.4 |
| 305 | | | | | |
| CD19-IL12- | Trimer/Extra | Trimer | 1 | 16.9 | 3.1 |
| 306 | | | | | |
| CD19-IL12- | Trimer/Intra | Mono | 1.1 | 2.9 | 5.4 |
| 307 | | | | | |
| CD19-IL12- | Mixed | Mono | 2.4 | 2.5 | 1.3 |
| 309 | | | | | |
| CD19-IL12- | Trimer/Extra | Trimer | 0.6 | 27.9 | 2.7 |
| 319 | | | | | |
| CD19-IL12- | Trimer/TM | Mono | 1.1 | 20 | 9.7 |
| 320 | | | | | |
| CD19-IL12- | Trimer/Extra | Trimer | 0.3 | 84.5 | 12.3 |
| 321 | | | | | |
| CD19-IL12- | Trimer/Extra | Mono | 0.8 | 26.4 | 11.9 |
| 322 | | | | | |
| CD19-IL12- | Trimer/Extra | Trimer | 1.9 | 7.5 | 3.0 |
| 323 | | | | | |
| CD19-IL12- | Penta/TM | Mono | 1.8 | 16.2 | 7.7 |
| 324 | | | | | |
| CD19-IL12- | Penta/TM | Mono | 3.8 | 5.8 | 2.6 |
| 325 | | | | | |
| CD19-IL12- | Tetra/Intra | Mono | 1.3 | 11.8 | 6.2 |
| 326 | | | | | |
| CD19-IL12- | Trimer/Intra | Mono | 2.1 | 5.5 | 2.8 |
| 327 | | | | | |
| CD19-IL12- | Trimer/Intra | Mono | 0.7 | 13.8 | 9.3 |
| 328 | | | | | |
| CD19-IL12- | Trimer/Extra | Trimer | 0.3 | 122.3 | 10.8 |
| 329 | | | | | |

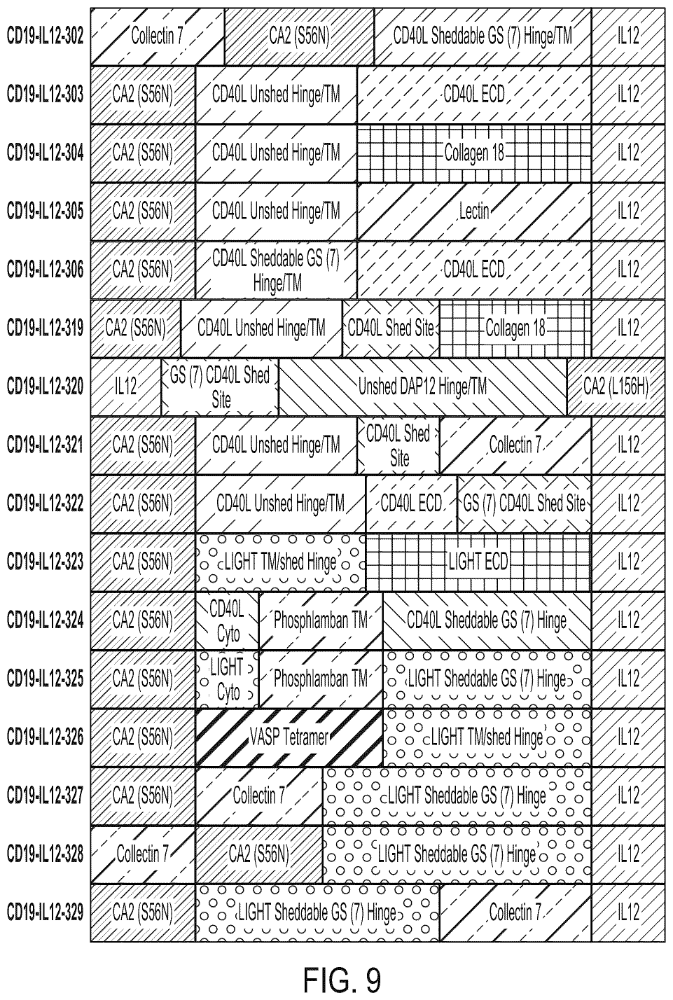

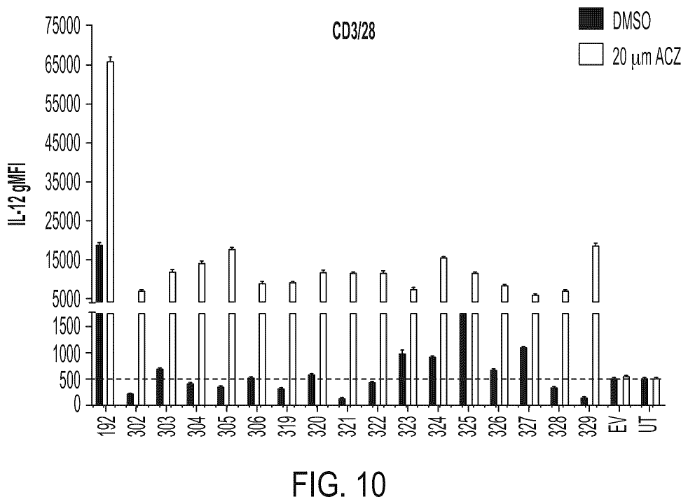

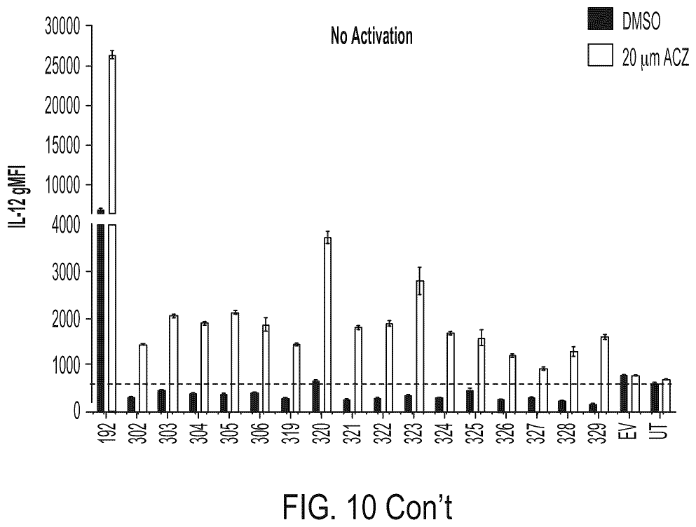

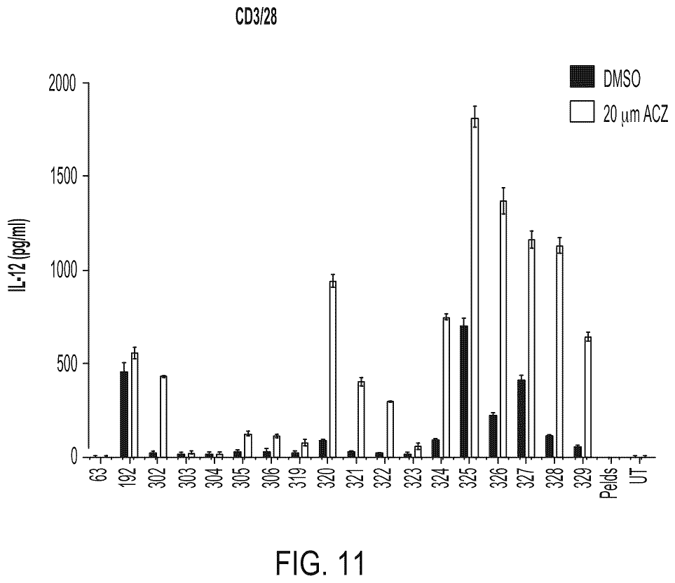

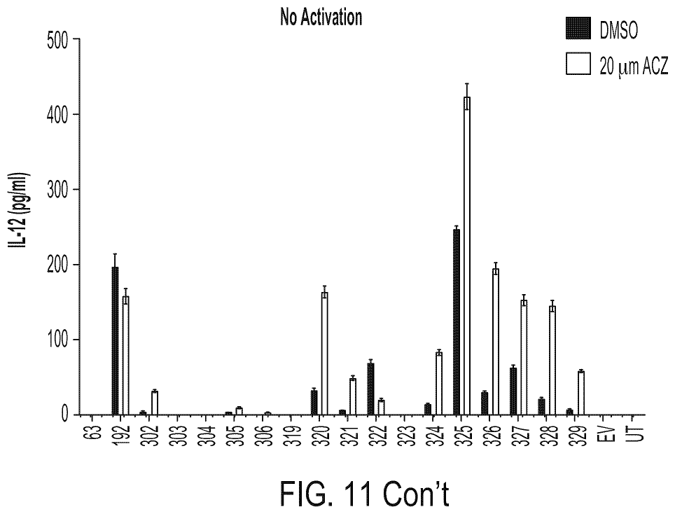

Cytokine that accumulated in the overnight culture supernatants from 40,000 transduced cells (CD19-CAR+) per 200 μL media were measured using human IL12p70 and/or human interferon-gamma MSD V-plex assay kits (Meso Scale Discovery). Fold-change of IL12 within a restimulation condition was calculated as the concentration of IL12 with ligand added divided by the concentration without ligand for a given construct. All constructs transduced and expanded efficiently in primary human T-cells. FIG. 3, FIG. 9 and Table 3 provide a list and schematic of all constructs generated with oligomerization domains. A subset of constructs (FIG. 9 and Table 3) were moved forward into assessment of expression and activity in T-cells based on achieving a low off-state and high regulation in HEK cells and T-cells. All constructs containing oligomerization domains reduced off-state (no-ligand) abundance of mbIL12 (FIG. 10A and FIG. 10B) when compared to a similar construct without oligomerization domains (construct CD19-IL12-192). This regulation was largely maintained when looking at the fraction of IL12 shed into the supernatant (FIG. 11A and FIG. 11B).

### Example 7. Regulation of IL12 Activity Assessed Using STAT4 Phosphorylation on Bystander Natural Killer Cells

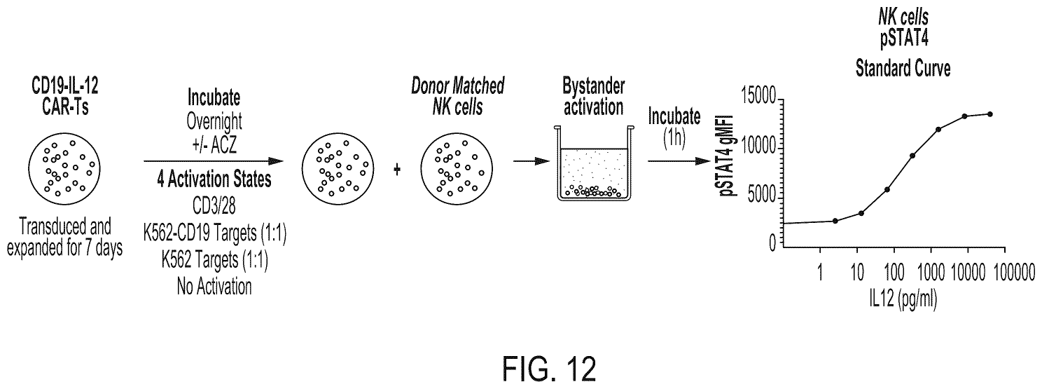

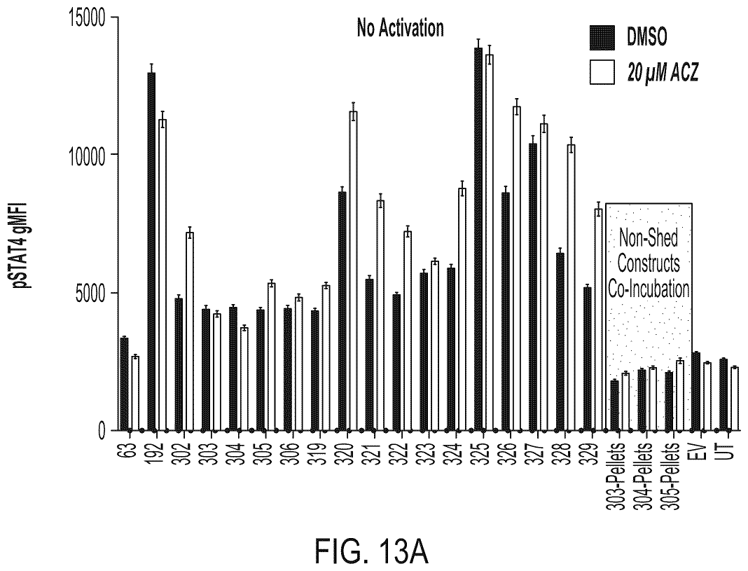

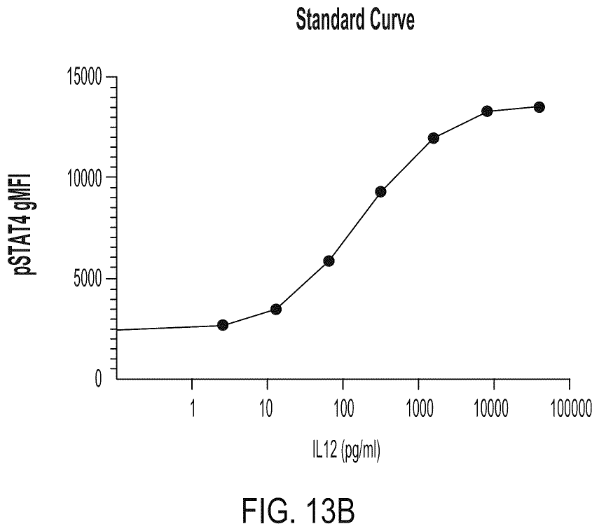

FIG. 12 describes the procedures for testing IL12 engagement and function with its receptors by measuring phosphorylation of STAT4. Supernatants (100 μL) from cells plated on day 8 and harvested on day 9 were added to 40,000 natural killer (NK) cells (in 100 μL of media) derived from the peripheral blood of matched donors. The NK cells were thawed from liquid nitrogen and allowed to rest overnight before beginning the experiment. After 1 hour, the NK cells were then stained with anti-CD56, anti-CD16, anti-CD45, and anti-CD3 and assessed for STAT4 phosphorylation. All constructs transduced and expanded efficiently in primary human T-cells (as described in Example 6). A list of constructs generated with CD19-CAR oligomerization domains are shown in FIG. 2C-FIG. 2E. Construct CD19-IL12-192 does not contain an oligomerizing domain. Other controls include CD19-CAR only (063), empty vector (EV), and untransduced (UT) cells. For non-shed constructs (303-305), transduced cells were co-incubated with donor-matched NK cells (pellets). All other samples tested were supernatants of cultured T cells. Regulation of pSTAT4 as shown in FIG. 13A suggests that the regulation of the shed portion is functionally significant. FIG. 13B shows standard curve representing pSTAT MFI in relation to IL12 concentration.

### Example 8. Oligomerization in Cytoplasmic Settings: Trimerization and Tetramerization of FoxP3

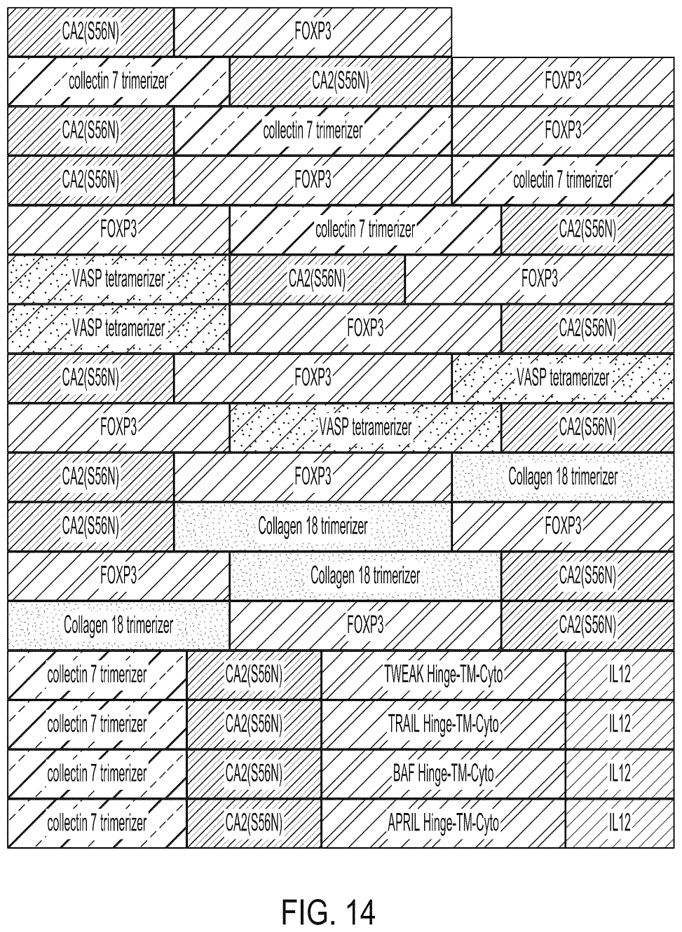

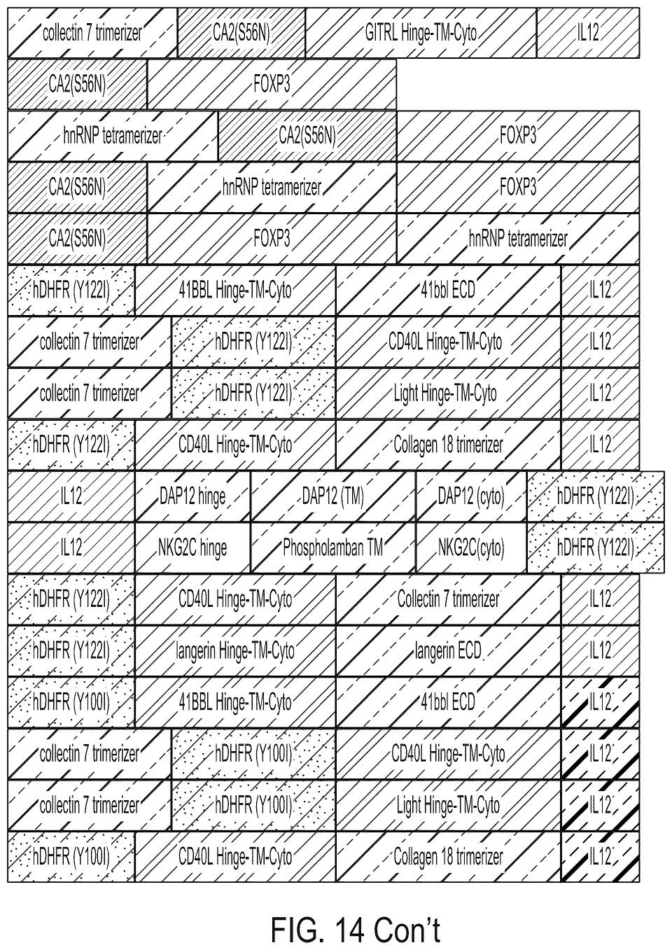

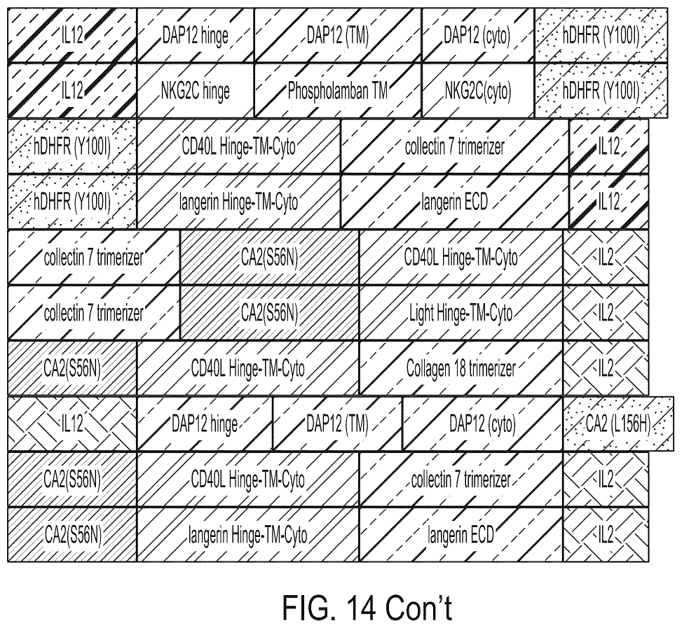

HEK-293T cells are cultured in standard media (DMEM (Fisher Scientific Cat #11-960-044) with 10% FBS (Fisher Scientific Cat #10-082-147) and 1% penicillin/streptomycin (P/S, ThermoFisher Cat #15140122). HEK293T cells are transiently transfected using manufacturer's protocol with Lipofectamine 2000 and 1 μg plasmid DNA each of FOXP3-X constructs (wherein X is varied) as shown in FIG. 14. 100 μM acetazolamide are added one day after transfection, and the cells are further cultured for 1 more day. mCherry is used as a transfection marker and after checking expression level by FACs, equal number of transfected cells are pelleted and lysed using T-PER Tissue protein extraction reagent (ThermoFisher cat #78510). Lysate is run on NuPAGE 4-12% Bis-Tris gel (ThermoFisher cat #NP0321BOX) and is transferred onto a nitrocellulose membrane (iBLOT2 NC regular stacks (ThermoFisher IB23001)). FoxP3 is detected using anti-FOXP3 monoclonal antibody (ThermoFisher Cat #MA5-16222) and IRDye 800CW Goat anti-Mouse IgG (LI-COR P/N 926-32210).

### Example 9. Multiple TNFSF Cytoplasmic Tail-TM-Hinge Combinations

Multiple TNFSF cytoplasmic tail-TM-hinge combinations (from TWEAK, TRAIL, BAFF, GITRL and APRIL) trimerized using collectin 7 trimerizing domain are used to fine tune regulation of IL12. HEK-293T cells are cultured in standard medium (DMEM (Fisher Scientific, Cat #11-960-044) with 10% FBS (Fisher Scientific, Cat #10-082-147) and 1% P/S (ThermoFisher Cat #15140122). HEK293T cells are transiently transfected using manufacturer's protocol with Lipofectamine 2000 and 1 μg plasmid DNA each of IL12-X (wherein X is varied) as shown in FIG. 14. 100 μM acetazolamide is added one day after transfection, and the cells are further cultured for 1 more day. mCherry is used as a transfection marker and anti-IL12p40/70-V450 Cat #561380 BD is used to stain for IL12.

### Example 10. Use of Other Oligomerization Domains Such as Tetramerization Domain from hnRNP

HEK-293T cells are cultured in standard media (DMEM FisherScientific Cat #11-960-044) with 10% FBS (FisherScientific Cat #10-082-147) and 1%

P/S (ThermoFisher Cat #15140122). HEK293T cells are transiently transfected using manufacturer's protocol with Lipofectamine 2000 and 1 μg plasmid DNA each of FOXP3-X (where X is varied) as shown in FIG. 14. 100 μM acetazolamide are added one day after transfection, and the cells are further cultured for 1 more day. mCherry is used as a transfection marker, and, after checking expression level by FACs, an equal number of transfected cells are pelleted and lysed using T-PER Tissue protein extraction reagent (ThermoFisher cat #78510). Lysate is run on NuPAGE 4-12% Bis-Tris gel (ThermoFisher cat #NP0321BOX) and is transferred onto a nitrocellulose membrane (iBLOT2 NC regular stacks (ThermoFisher IB23001)). FoxP3 is detected using anti-FOXP3 monoclonal antibody (ThermoFisher Cat #MA5-16222) and IRDye 800CW Goat anti-Mouse IgG (LI-COR P/N 926-32210).

### Example 11. hDHFR-IL12 and hDHFR-IL2 Constructs to Show Benefit of Trimerization with Existing Oligomerization Domains and an Alternate DRD (hDHFR)

HEK-293T cells are cultured in standard media (DMEM (Fisher Scientific, Cat #11-960-044) with 10% FBS (Fisher Scientific, Cat #10-082-147) and 1% P/S (ThermoFisher Cat #15140122). HEK293T cells are transiently transfected using the manufacturer's protocol with Lipofectamine 2000 and 1 μg plasmid DNA each of IL12-X (wherein X is varied) as shown in FIG. 14. 100 μM acetazolamide are added one day after transfection, and the cells are further cultured for 1 more day. mCherry is used as a transfection marker and anti-IL12p40/70-V450 Cat #561380 BD is used to stain for IL12.

In a similar experiment, HEK-293T cells are cultured in standard media (DMEM (Fisher Scientific, Cat #11-960-044) with 10% FBS (Fisher Scientific, Cat #10-082-147) and 1% P/S (ThermoFisher Cat #15140122). HEK293T cells are transiently transfected using the manufacturer's protocol with Lipofectamine 2000 and 1 μg plasmid DNA each of IL2-X (wherein X is varied) as shown in FIG. 14. 100 μM acetazolamide is added one day after transfection, and the cells are further cultured for 1 more day. mCherry is used as a transfection marker and anti-IL2 antibody Biolegend Cat #500311 is used to stain for IL2.

### Example 12. Trimerization Motif with IL2 Payloads

HEK-293T cells are cultured in standard media (DMEM (Fisher Scientific, Cat #11-960-044) with 10% FBS (Fisher Scientific, Cat #10-082-147) and 1% P/S (ThermoFisher Cat #15140122). HEK293T cells are transiently transfected using manufacturer's protocol with Lipofectamine 2000 and 1 μg plasmid DNA each of IL2-X (wherein X is varied. 100 μM acetazolamide are added one day after transfection, and the cells are further cultured for 1 more day. mCherry is used as a transfection marker and anti-IL2 antibody (Biolegend Cat #500311) is used to stain for IL2.

### Example 13. Trimerization Motif with IL15 Payloads

HEK-293T cells are cultured in standard media (DMEM (V Cat #11-960-044) with 10% FBS (V Cat #10-082-147) and 1% P/S (ThermoFisher Cat #15140122). HEK293T cells are transiently transfected using manufacturer's protocol with Lipofectamine 2000 and 1 μg plasmid DNA each of IL15-X (wherein X is varied). 100 μM acetazolamide are added one day after transfection, and the cells are further cultured for 1 more day. mCherry is used as a transfection marker and IL15Ra-Fc-biotin (Cat #ILA-82F4 Acrobiosystems) followed by Streptavidin BV421 (Biolegend, Cat #405225) to stain for IL15.

### Example 14. Use of Heterotrimerization for Regulating a Single Copy of Payload

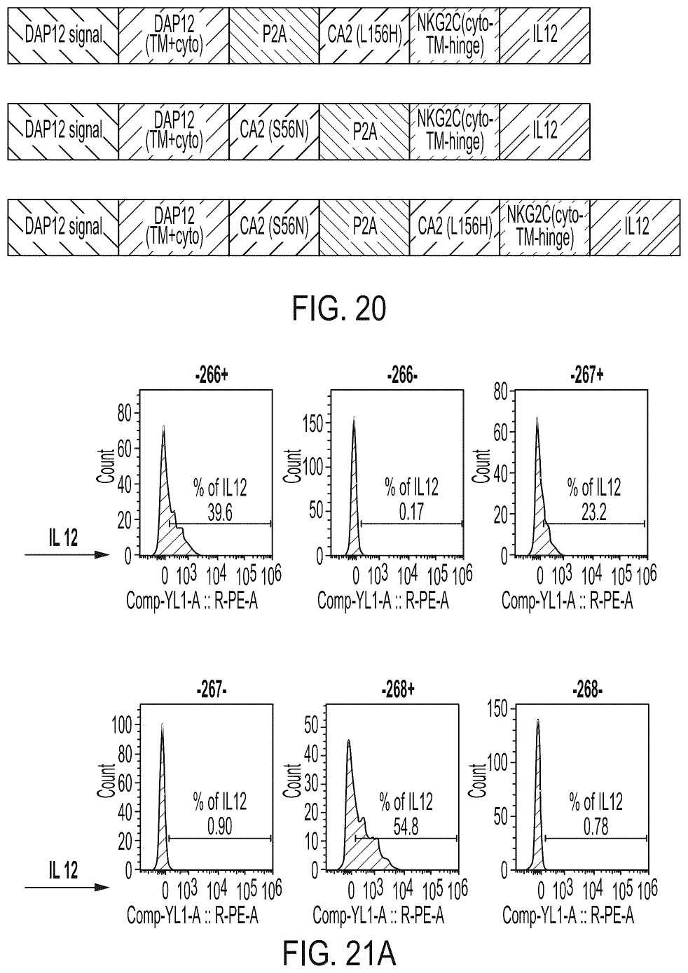

Jurkat cells were transduced to 10-15% positive as determined by using RQR8 as a transduction marker with lentivirus generated using constructs IL12-266, 267 and 268 shown in FIG. 20. Five days post transduction, the cells were treated with 100 μM acetazolamide or DMSO and the cells were further cultured for 1 more day. RQR8 was detected using anti-CD34 clone QBEND10 (R&D cat #FAB7227A) and IL12 using anti-IL12p40/70-V450 (BD Cat #561380). FIG. 21A shows histograms showing IL12 levels in the presence and absence of acetazolamide. FIG. 21B is a schematic showing the assembly and the oligomeric states of DRDs in IL12-266, 267 and 268. FIG. 21C shows fold change in the IL12 geometric MFI after addition of drug to each of IL12-266, 267 and 268. The transduced cells show a pattern of increasing fold regulation as they progress from monomer, dimer through to trimer.

### Example 15. Regulation of Membrane-Bound IL23 Constructs in Primary Human CAR-T Cells

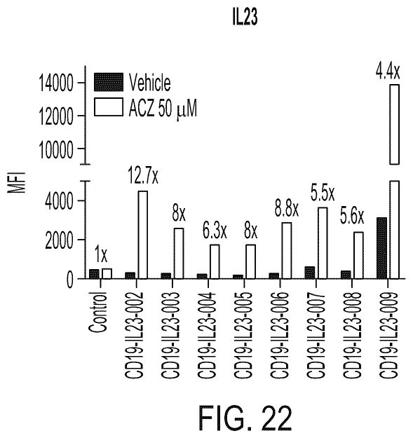

On day 0, primary human T cells were stimulated with Dynabeads (T-expander CD3/CD28) at a 3:1 bead:cell ratio in media containing 10% fetal bovine serum (FBS). The next day, lentivirus produced with constructs expressing CD19 CAR and membrane-bound oligomerizing flexi-IL23 (CD19-IL23-002, CD19-IL23-003, CD19-IL23-004, CD19-IL23-005, CD19-IL23-006, CD19-IL23-007, CD19-IL23-008) and control constructs (CD19-IL23-001, which expresses secreted flexi-IL23; CD19-IL23-009, which expresses flexi-IL23 without any oligomerizing modulation hub) were added in the presence of reduced serum (5% FBS). On day 2, the cells were diluted 1:2 with fresh 10% FBS media. On day 5, the cells were analyzed for CD19 CAR expression using CD19-Fc (R&D Systems, Minneapolis, MN; Part #9269-CD-050) followed by anti-human Alexa 647 anti-Human IgG (Jackson ImmunoResearch, West Grove, PA; Part #109-607-003). Media was replaced and cells were treated with 100 μM acetazolamide or DMSO. Twenty-four hours later T cells were analyzed by FACs. CD19 CAR was used as a transduction marker and anti-IL12/IL23p40-PE (Biolegend, San Diego, CA; Cat #501807) was used to stain for IL23 expression. The Geometric MFI of surface IL23p40 expression on CAR+ cells was plotted using Prism Software. The results are shown in FIG. 22. IL23 abundance was analyzed in the presence and absence of ACZ. The cells transduced with some oligomerized constructs have low IL23 abundance (off-state) in the absence of ACZ. The numbers above the ACZ treated cells represent fold change between on and off-state. Oligomerized constructs with multiple DRDs reduced basal abundance and enhanced fold change between on and off-states for IL23.

The following constructs were used in this example:

| Name | Description |
|---|---|
| CD19-IL23-001 | CD19CAR-p2a-p40sp-IL23 |
| SEQ ID NO: 245 | |

MCHQQLVISWFSLVFLASPLVAIWELKKDVYVVELDWYPDAPGEMVVLTCDTPEED
GITWTLDQSSEVLGSGKTLTIQVKEFGDAGQYTCHKGGEVLSHSLLLLHKKEDGIWS
TDILKDQKEPKNKTFLRCEAKNYSGRFTCWWLTTISTDLTFSVKSSRGSSDPQGVTC
GAATLSAERVRGDNKEYEYSVECQEDSACPAAEESLPIEVMVDAVHKLKYENYTSS
FFIRDIIKPDPPKNLQLKPLKNSRQVEVSWEYPDTWSTPHSYFSLTFCVQVQGKSKRE
KKDRVFTDKTSATVICRKNASISVRAQDRYYSSSWSEWASVPCSGGGGSGGGGSGG
GGSRAVPGGSSPAWTQCQQLSQKLCTLAWSAHPLVGHMDLREEGDEETTNDVPHIQ
CGDGCDPQGLRDNSQFCLQRIHQGLIFYEKLLGSDIFTGEPSLLPDSPVGQLHASLLG
LSQLLQPEGHHWETQQIPSLSPSQPWQRLLLRFKILRSLQAFVAVAARVFAHGAATL
SP

| Name | Description |
|---|---|
| CD19-IL23-002 | CD19CAR-p2a-CA2(L156H)-(GGSG)3-CD40Lcyto- |
| (SEQ ID | phospholambancyto-tm-CD40Lhinge(shed)-(GS)7-IL23 |
| NO: 246) | |

MALPVTALLLPLALLLHAARPDIQMTQTTSSLSASLGDRVTISCRASQDISKYLNWY
QQKPDGTVKLLIYHTSRLHSGVPSRFSGSGSGTDYSLTISNLEQEDIATYFCQQGNTLP
YTFGGGTKLEITGGGGSGGGGSGGGGSEVKLQESGPGLVAPSQSLSVTCTVSGVSLP
DYGVSWIRQPPRKGLEWLGVIWGSETTYYNSALKSRLTIIKDNSKSQVFLKMNSLQT
DDTAIYYCAKHYYYGGSYAMDYWGQGTSVTVSSTTTPAPRPPTPAPTIASQPLSLRP
EACRPAAGGAVHTRGLDFACDIYIWAPLAGTCGVLLLSLVITLYCKRGRKKLLYIFK
QPFMRPVQTTQEEDGCSCRFPEEEEGGCELRVKFSRSADAPAYKQGQNQLYNELNL
GRREEYDVLDKRRGRDPEMGGKPRRKNPQEGLYNELQKDKMAEAYSEIGMKGERR
RGKGHDGLYQGLSTATKDTYDALHMQALPPRGSGATNFSLLKQAGDVEENPGPGG
SSHHWGYGKHNGPEHWHKDFPIAKGERQSPVDIDTHTAKYDPSLKPLSVSYDQATS
LRILNNGHAFNVEFDDSQDKAVLKGGPLDGTYRLIQFHFHWGSLDGQGSEHTVDKK
KYAAELHLVHWNTKYGDFGKAVQQPDGLAVLGIFLKVGSAKPGHQKVVDVLDSIK
TKGKSADFTNFDPRGLLPESLDYWTYPGSLTTPPLLECVTWIVLKEPISVSSEQVLKF
RKLNFNGEGEPEELMVDNWRPAQPLKNRQIKASFKGGSGGGSGGGSGMIETYNQTS
PRSAATGLPISMKARQKLQNLFINFCLILICLLLICIIVMLLLHRRLDKIEDERNLHEDF
VFMKTIQRCNTGERSLSLLNCEEIKSQFEGFVKDIMLNKEETKKENSFEMQKGSGSGS
GSGSGSGSIWELKKDVYVVELDWYPDAPGEMVVLTCDTPEEDGITWTLDQSSEVLG
SGKTLTIQVKEFGDAGQYTCHKGGEVLSHSLLLLHKKEDGIWSTDILKDQKEPKNKT
FLRCEAKNYSGRFTCWWLTTISTDLTFSVKSSRGSSDPQGVTCGAATLSAERVRGDN
KEYEYSVECQEDSACPAAEESLPIEVMVDAVHKLKYENYTSSFFIRDIIKPDPPKNLQ
LKPLKNSRQVEVSWEYPDTWSTPHSYFSLTFCVQVQGKSKREKKDRVFTDKTSATVI
CRKNASISVRAQDRYYSSSWSEWASVPCSGGGGSGGGGSGGGGSRAVPGGSSPAWT
QCQQLSQKLCTLAWSAHPLVGHMDLREEGDEETTNDVPHIQCGDGCDPQGLRDNS
QFCLQRIHQGLIFYEKLLGSDIFTGEPSLLPDSPVGQLHASLLGLSQLLQPEGHHWETQ
QIPSLSPSQPWQRLLLRFKILRSLQAFVAVAARVFAHGAATLSP

| Name | Description |
|---|---|
| CD19-IL23-003 | CD19CAR-p2a-collectin7-(GSG)6-CA2(L156H)- |
| (SEQ ID | (GGSG)3-light(cyto-TM-hinge)-(GS)7-IL23 |
| NO: 247) | |

MALPVTALLLPLALLLHAARPDIQMTQTTSSLSASLGDRVTISCRASQDISKYLNWY
QQKPDGTVKLLIYHTSRLHSGVPSRFSGSGSGTDYSLTISNLEQEDIATYFCQQGNTLP
YTFGGGTKLEITGGGGSGGGGSGGGGSEVKLQESGPGLVAPSQSLSVTCTVSGVSLP
DYGVSWIRQPPRKGLEWLGVIWGSETTYYNSALKSRLTIIKDNSKSQVFLKMNSLQT
DDTAIYYCAKHYYYGGSYAMDYWGQGTSVTVSSTTTPAPRPPTPAPTIASQPLSLRP
EACRPAAGGAVHTRGLDFACDIYIWAPLAGTCGVLLLSLVITLYCKRGRKKLLYIFK
QPFMRPVQTTQEEDGCSCRFPEEEEGGCELRVKFSRSADAPAYKQGQNQLYNELNL
GRREEYDVLDKRRGRDPEMGGKPRRKNPQEGLYNELQKDKMAEAYSEIGMKGERR
RGKGHDGLYQGLSTATKDTYDALHMQALPPRGSGATNFSLLKQAGDVEENPGPGG
SMSGLPDVASLRQQVEALQGQVQHLQAAFSQYKKVELFPNGQSgsggsggsggsggsggsg
SHHWGYGKHNGPEHWHKDFPIAKGERQSPVDIDTHTAKYDPSLKPLSVSYDQATSL
RILNNGHAFNVEFDDSQDKAVLKGGPLDGTYRLIQFHFHWGSLDGQGSEHTVDKKK
YAAELHLVHWNTKYGDFGKAVQQPDGLAVLGIFLKVGSAKPGHQKVVDVLDSIKT
KGKSADFTNFDPRGLLPESLDYWTYPGSLTTPPLLECVTWIVLKEPISVSSEQVLKFR
KLNFNGEGEPEELMVDNWRPAQPLKNRQIKASFKGGSGGGSGGGSGDGQTDIPFTR
LGRSHRRQSCSVARVGLGLLLLLMGAGLAVQGWFLLQLHWRLGEMVTRLPDGPAG
SWEQLIQGSGSGSGSGSGSGSIWELKKDVYVVELDWYPDAPGEMVVLTCDTPEEDG
ITWTLDQSSEVLGSGKTLTIQVKEFGDAGQYTCHKGGEVLSHSLLLLHKKEDGIWST
DILKDQKEPKNKTFLRCEAKNYSGRFTCWWLTTISTDLTFSVKSSRGSSDPQGVTCG
AATLSAERVRGDNKEYEYSVECQEDSACPAAEESLPIEVMVDAVHKLKYENYTSSFF
IRDIIKPDPPKNLQLKPLKNSRQVEVSWEYPDTWSTPHSYFSLTFCVQVQGKSKREKK
DRVFTDKTSATVICRKNASISVRAQDRYYSSSWSEWASVPCSGGGGSGGGGSGGGG
SRAVPGGSSPAWTQCQQLSQKLCTLAWSAHPLVGHMDLREEGDEETTNDVPHIQCG
DGCDPQGLRDNSQFCLQRIHQGLIFYEKLLGSDIFTGEPSLLPDSPVGQLHASLLGLSQ
LLQPEGHHWETQQIPSLSPSQPWQRLLLRFKILRSLQAFVAVAARVFAHGAATLSP

| Name | Description |
|---|---|
| CD19-IL23-004 | CD19CAR-p2a-trimerizing domain (collectin 7)-(GSG)6- |
| (SEQ ID | CA2(L156H)-(ggsg)3-CD40Lcyto-TM-hinge(shed)-(GS)7-IL23 |
| NO: 248) | |

MALPVTALLLPLALLLHAARPDIQMTQTTSSLSASLGDRVTISCRASQDISKYLNWY
QQKPDGTVKLLIYHTSRLHSGVPSRFSGSGSGTDYSLTISNLEQEDIATYFCQQGNTLP
YTFGGGTKLEITGGGGSGGGGSGGGGSEVKLQESGPGLVAPSQSLSVTCTVSGVSLP
DYGVSWIRQPPRKGLEWLGVIWGSETTYYNSALKSRLTIIKDNSKSQVFLKMNSLQT
DDTAIYYCAKHYYYGGSYAMDYWGQGTSVTVSSTTTPAPRPPTPAPTIASQPLSLRP
EACRPAAGGAVHTRGLDFACDIYIWAPLAGTCGVLLLSLVITLYCKRGRKKLLYIFK
QPFMRPVQTTQEEDGCSCRFPEEEEGGCELRVKFSRSADAPAYKQGQNQLYNELNL
GRREEYDVLDKRRGRDPEMGGKPRRKNPQEGLYNELQKDKMAEAYSEIGMKGERR
RGKGHDGLYQGLSTATKDTYDALHMQALPPRGSGATNFSLLKQAGDVEENPGPGG
SMSGLPDVASLRQQVEALQGQVQHLQAAFSQYKKVELFPNGQSgsggsggsggsggsggsg
SHHWGYGKHNGPEHWHKDFPIAKGERQSPVDIDTHTAKYDPSLKPLSVSYDQATSL
RILNNGHAFNVEFDDSQDKAVLKGGPLDGTYRLIQFHFHWGSLDGQGSEHTVDKKK
YAAELHLVHWNTKYGDFGKAVQQPDGLAVLGIFLKVGSAKPGHQKVVDVLDSIKT
KGKSADFTNFDPRGLLPESLDYWTYPGSLTTPPLLECVTWIVLKEPISVSSEQVLKFR
KLNFNGEGEPEELMVDNWRPAQPLKNRQIKASFKggsgggsgggsgMIETYNQTSPRSAA
TGLPISMKIFMYLLTVFLITQMIGSALFAVYLHRRLDKIEDERNLHEDFVFMKTIQRC
NTGERSLSLLNCEEIKSQFEGFVKDIMLNKEETKKENSFEMQKGSGSGSGSGSGSGSC
DLPQTHSLGSRRTLMLLAQMRKISLFSCLKDRHDFGFPQEEFGNQFQKAETIPVLHE
MIQQIFNLFSTKDSSAAWDETLLDKFYTELYQQLNDLEACVIQGVGVTETPLMKEDS
ILAVRKYFQRITLYLKEKKYSPCAWEVVRAEIMRSFSLSTNLQESLRSKE

| Name | Description |
|---|---|
| CD19-IL23-005 | CD19CAR-p2a-p40sp-IL23-linker-CD40L shed site- |
| (SEQ ID | DAP12hingeDAP12TMDAP12cyto-CA2(L156H) |
| NO: 249) | |

MALPVTALLLPLALLLHAARPDIQMTQTTSSLSASLGDRVTISCRASQDISKYLNWY
QQKPDGTVKLLIYHTSRLHSGVPSRFSGSGSGTDYSLTISNLEQEDIATYFCQQGNTLP
YTFGGGTKLEITGGGGSGGGGSGGGGSEVKLQESGPGLVAPSQSLSVTCTVSGVSLP
DYGVSWIRQPPRKGLEWLGVIWGSETTYYNSALKSRLTIIKDNSKSQVFLKMNSLQT
DDTAIYYCAKHYYYGGSYAMDYWGQGTSVTVSSTTTPAPRPPTPAPTIASQPLSLRP
EACRPAAGGAVHTRGLDFACDIYIWAPLAGTCGVLLLSLVITLYCKRGRKKLLYIFK
QPFMRPVQTTQEEDGCSCRFPEEEEGGCELRVKFSRSADAPAYKQGQNQLYNELNL
GRREEYDVLDKRRGRDPEMGGKPRRKNPQEGLYNELQKDKMAEAYSEIGMKGERR
RGKGHDGLYQGLSTATKDTYDALHMQALPPRGSGATNFSLLKQAGDVEENPGPGG
SMALTFALLVALLVLSCKSSCSVGCDLPQTHSLGSRRTLMLLAQMRKISLFSCLKDR
HDFGFPQEEFGNQFQKAETIPVLHEMIQQIFNLFSTKDSSAAWDETLLDKFYTELYQQ
LNDLEACVIQGVGVTETPLMKEDSILAVRKYFQRITLYLKEKKYSPCAWEVVRAEIM
RSFSLSTNLQESLRSKEGSGSGSGSGSGSGSSFEMQKLRPVQAQAQSDCSCSTVSPGV
LAGIVMGDLVLTVLIALAVYFLGRLVPRGRGAAEAATRKQRITEGGSGGGSGSSHH
WGYGKHNGPEHWHKDFPIAKGERQSPVDIDTHTAKYDPSLKPLSVSYDQATSLRILN
NGHAFNVEFDDSQDKAVLKGGPLDGTYRLIQFHFHWGSLDGQGSEHTVDKKKYAA
ELHLVHWNTKYGDFGKAVQQPDGLAVLGIFLKVGSAKPGHQKVVDVLDSIKTKGK
SADFTNFDPRGLLPESLDYWTYPGSLTTPPLLECVTWIVLKEPISVSSEQVLKFRKLNF
NGEGEPEELMVDNWRPAQPLKNRQIKASFK

| Name | Description |
|---|---|
| CD19-IL23-006 | CD19CAR-p2a-(GSG)6-CA2(S56N)-(ggsg)3-CD40Lcyto-TM- |
| (SEQ ID | hinge(shed)-(GS)7-IL23 (control typeII, shed) |
| NO: 250) | |

MALPVTALLLPLALLLHAARPDIQMTQTTSSLSASLGDRVTISCRASQDISKYLNWY
QQKPDGTVKLLIYHTSRLHSGVPSRFSGSGSGTDYSLTISNLEQEDIATYFCQQGNTLP
YTFGGGTKLEITGGGGSGGGGSGGGGSEVKLQESGPGLVAPSQSLSVTCTVSGVSLP
DYGVSWIRQPPRKGLEWLGVIWGSETTYYNSALKSRLTIIKDNSKSQVFLKMNSLQT
DDTAIYYCAKHYYYGGSYAMDYWGQGTSVTVSSTTTPAPRPPTPAPTIASQPLSLRP
EACRPAAGGAVHTRGLDFACDIYIWAPLAGTCGVLLLSLVITLYCKRGRKKLLYIFK
QPFMRPVQTTQEEDGCSCRFPEEEEGGCELRVKFSRSADAPAYKQGQNQLYNELNL
GRREEYDVLDKRRGRDPEMGGKPRRKNPQEGLYNELQKDKMAEAYSEIGMKGERR
RGKGHDGLYQGLSTATKDTYDALHMQALPPRGSGATNFSLLKQAGDVEENPGPGG
SSHHWGYGKHNGPEHWHKDFPIAKGERQSPVDIDTHTAKYDPSLKPLSVSYDQATN
LRILNNGHAFNVEFDDSQDKAVLKGGPLDGTYRLIQFHFHWGSLDGQGSEHTVDKK
KYAAELHLVHWNTKYGDFGKAVQQPDGLAVLGIFLKVGSAKPGLQKVVDVLDSIK
TKGKSADFTNFDPRGLLPESLDYWTYPGSLTTPPLLECVTWIVLKEPISVSSEQVLKF
RKLNFNGEGEPEELMVDNWRPAQPLKNRQIKASFKggsgggsgggsgMIETYNQTSPRSA
ATGLPISMKIFMYLLTVFLITQMIGSALFAVYLHRRLDKIEDERNLHEDFVFMKTIQR
CNTGERSLSLLNCEEIKSQFEGFVKDIMLNKEETKKENSFEMQKGSGSGSGSGSGSGS
IWELKKDVYVVELDWYPDAPGEMVVLTCDTPEEDGITWTLDQSSEVLGSGKTLTIQ
VKEFGDAGQYTCHKGGEVLSHSLLLLHKKEDGIWSTDILKDQKEPKNKTFLRCEAK
NYSGRFTCWWLTTISTDLTFSVKSSRGSSDPQGVTCGAATLSAERVRGDNKEYEYSV
ECQEDSACPAAEESLPIEVMVDAVHKLKYENYTSSFFIRDIIKPDPPKNLQLKPLKNSR
QVEVSWEYPDTWSTPHSYFSLTFCVQVQGKSKREKKDRVFTDKTSATVICRKNASIS
VRAQDRYYSSSWSEWASVPCSGGGGSGGGGSGGGGSRAVPGGSSPAWTQCQQLSQ
KLCTLAWSAHPLVGHMDLREEGDEETTNDVPHIQCGDGCDPQGLRDNSQFCLQRIH
QGLIFYEKLLGSDIFTGEPSLLPDSPVGQLHASLLGLSQLLQPEGHHWETQQIPSLSPS
QPWQRLLLRFKILRSLQAFVAVAARVFAHGAATLSP
WIVLKEPISVSSEQVLKFRKLNFNGEGEPEELMVDNWRPAQPLKNRQIKASFK

| Name | Description |
|---|---|
| CD19-IL23-007 | CA2(L156H)-(GGSG)3-41BBL cyto (21 aa)-41BBL TM- |
| (SEQ ID | 41BBLhinge-41BBL ECD-(GS)7-IL23 |
| NO: 251) | |

MALPVTALLLPLALLLHAARPDIQMTQTTSSLSASLGDRVTISCRASQDISKYLNWY
QQKPDGTVKLLIYHTSRLHSGVPSRFSGSGSGTDYSLTISNLEQEDIATYFCQQGNTLP
YTFGGGTKLEITGGGGSGGGGSGGGGSEVKLQESGPGLVAPSQSLSVTCTVSGVSLP
DYGVSWIRQPPRKGLEWLGVIWGSETTYYNSALKSRLTIIKDNSKSQVFLKMNSLQT
DDTAIYYCAKHYYYGGSYAMDYWGQGTSVTVSSTTTPAPRPPTPAPTIASQPLSLRP
EACRPAAGGAVHTRGLDFACDIYIWAPLAGTCGVLLLSLVITLYCKRGRKKLLYIFK
QPFMRPVQTTQEEDGCSCRFPEEEEGGCELRVKFSRSADAPAYKQGQNQLYNELNL
GRREEYDVLDKRRGRDPEMGGKPRRKNPQEGLYNELQKDKMAEAYSEIGMKGERR
RGKGHDGLYQGLSTATKDTYDALHMQALPPRGSGATNFSLLKQAGDVEENPGPGG
SSHHWGYGKHNGPEHWHKDFPIAKGERQSPVDIDTHTAKYDPSLKPLSVSYDQATS
LRILNNGHAFNVEFDDSQDKAVLKGGPLDGTYRLIQFHFHWGSLDGQGSEHTVDKK
KYAAELHLVHWNTKYGDFGKAVQQPDGLAVLGIFLKVGSAKPGHQKVVDVLDSIK
TKGKSADFTNFDPRGLLPESLDYWTYPGSLTTPPLLECVTWIVLKEPISVSSEQVLKF
RKLNFNGEGEPEELMVDNWRPAQPLKNRQIKASFKGGSGGGSGGGSGPEAPWPPAP
RARACRVLPWALVAGLLLLLLLAAACAVFLACPWAVSGARASPGSAASPRLREGPE
LSPDDPAGLLDLRQGMFAQLVAQNVLLIDGPLSWYSDPGLAGVSLTGGLSYKEDTK
ELVVAKAGVYYVFFQLELRRVVAGEGSGSVSLALHLQPLRSAAGAAALALTVDLPP
ASSEARNSAFGFQGRLLHLSAGQRLGVHLHTEARARHAWQLTQGATVLGLFRVTPE
IPAGLPSPRSEGSGSGSGSGSGSGSIWELKKDVYVVELDWYPDAPGEMVVLTCDTPE
EDGITWTLDQSSEVLGSGKTLTIQVKEFGDAGQYTCHKGGEVLSHSLLLLHKKEDGI
WSTDILKDQKEPKNKTFLRCEAKNYSGRFTCWWLTTISTDLTFSVKSSRGSSDPQGV
TCGAATLSAERVRGDNKEYEYSVECQEDSACPAAEESLPIEVMVDAVHKLKYENYT
SSFFIRDIIKPDPPKNLQLKPLKNSRQVEVSWEYPDTWSTPHSYFSLTFCVQVQGKSK
REKKDRVFTDKTSATVICRKNASISVRAQDRYYSSSWSEWASVPCSGGGGSGGGGS
GGGGSRAVPGGSSPAWTQCQQLSQKLCTLAWSAHPLVGHMDLREEGDEETTNDVP
HIQCGDGCDPQGLRDNSQFCLQRIHQGLIFYEKLLGSDIFTGEPSLLPDSPVGQLHASL
LGLSQLLQPEGHHWETQQIPSLSPSQPWQRLLLRFKILRSLQAFVAVAARVFAHGAA
TLSP

| Name | Description |
|---|---|
| CD19-IL23-008 | CD19CAR-p2a-trimerizing domain (collectin 7)-(GSG)6- |
| (SEQ ID | CA2(L156H)-(ggsg)3-41BBLcyto(21 aa)-TM-hinge-(GS)7-IL23 |
| NO: 252) | |

MALPVTALLLPLALLLHAARPDIQMTQTTSSLSASLGDRVTISCRASQDISKYLNWY
QQKPDGTVKLLIYHTSRLHSGVPSRFSGSGSGTDYSLTISNLEQEDIATYFCQQGNTLP
YTFGGGTKLEITGGGGSGGGGSGGGGSEVKLQESGPGLVAPSQSLSVTCTVSGVSLP
DYGVSWIRQPPRKGLEWLGVIWGSETTYYNSALKSRLTIIKDNSKSQVFLKMNSLQT
DDTAIYYCAKHYYYGGSYAMDYWGQGTSVTVSSTTTPAPRPPTPAPTIASQPLSLRP
EACRPAAGGAVHTRGLDFACDIYIWAPLAGTCGVLLLSLVITLYCKRGRKKLLYIFK
QPFMRPVQTTQEEDGCSCRFPEEEEGGCELRVKFSRSADAPAYKQGQNQLYNELNL
GRREEYDVLDKRRGRDPEMGGKPRRKNPQEGLYNELQKDKMAEAYSEIGMKGERR
RGKGHDGLYQGLSTATKDTYDALHMQALPPRGSGATNFSLLKQAGDVEENPGPGG
SMSGLPDVASLRQQVEALQGQVQHLQAAFSQYKKVELFPNGQSgsggsggsggsggsggsg
SHHWGYGKHNGPEHWHKDFPIAKGERQSPVDIDTHTAKYDPSLKPLSVSYDQATSL
RILNNGHAFNVEFDDSQDKAVLKGGPLDGTYRLIQFHFHWGSLDGQGSEHTVDKKK
YAAELHLVHWNTKYGDFGKAVQQPDGLAVLGIFLKVGSAKPGHQKVVDVLDSIKT
KGKSADFTNFDPRGLLPESLDYWTYPGSLTTPPLLECVTWIVLKEPISVSSEQVLKFR
KLNFNGEGEPEELMVDNWRPAQPLKNRQIKASFKggsgggsgggsgPEAPWPPAPRARAC
RVLPWALVAGLLLLLLLAAACAVFLACPWAVSGARASPGSAASPRLREGPELSPDDP
AGLLDLRQGMFAQLGSGSGSGSGSGSGSIWELKKDVYVVELDWYPDAPGEMVVLT
CDTPEEDGITWTLDQSSEVLGSGKTLTIQVKEFGDAGQYTCHKGGEVLSHSLLLLHK
KEDGIWSTDILKDQKEPKNKTFLRCEAKNYSGRFTCWWLTTISTDLTFSVKSSRGSSD
PQGVTCGAATLSAERVRGDNKEYEYSVECQEDSACPAAEESLPIEVMVDAVHKLKY
ENYTSSFFIRDIIKPDPPKNLQLKPLKNSRQVEVSWEYPDTWSTPHSYFSLTFCVQVQ
GKSKREKKDRVFTDKTSATVICRKNASISVRAQDRYYSSSWSEWASVPCSGGGGSG
GGGSGGGGSRAVPGGSSPAWTQCQQLSQKLCTLAWSAHPLVGHMDLREEGDEETT
NDVPHIQCGDGCDPQGLRDNSQFCLQRIHQGLIFYEKLLGSDIFTGEPSLLPDSPVGQL
HASLLGLSQLLQPEGHHWETQQIPSLSPSQPWQRLLLRFKILRSLQAFVAVAARVFA
HGAATLSP

| Name | Description |
|---|---|
| CD19-IL23-009 | CD19CAR-p2a-p40sp-Il23-linker(GS)7-B7-1 hingeTM-tail- |
| (SEQ ID | CA2(L156H)(control regulated construct, with B7-1 hinge) |
| NO: 253) | |

MALPVTALLLPLALLLHAARPDIQMTQTTSSLSASLGDRVTISCRASQDISKYLNWY
QQKPDGTVKLLIYHTSRLHSGVPSRFSGSGSGTDYSLTISNLEQEDIATYFCQQGNTLP
YTFGGGTKLEITGGGGSGGGGSGGGGSEVKLQESGPGLVAPSQSLSVTCTVSGVSLP
DYGVSWIRQPPRKGLEWLGVIWGSETTYYNSALKSRLTIIKDNSKSQVFLKMNSLQT
DDTAIYYCAKHYYYGGSYAMDYWGQGTSVTVSSTTTPAPRPPTPAPTIASQPLSLRP
EACRPAAGGAVHTRGLDFACDIYIWAPLAGTCGVLLLSLVITLYCKRGRKKLLYIFK
QPFMRPVQTTQEEDGCSCRFPEEEEGGCELRVKFSRSADAPAYKQGQNQLYNELNL
GRREEYDVLDKRRGRDPEMGGKPRRKNPQEGLYNELQKDKMAEAYSEIGMKGERR
RGKGHDGLYQGLSTATKDTYDALHMQALPPRGSGATNFSLLKQAGDVEENPGPGG
SMALTFALLVALLVLSCKSSCSVGIWELKKDVYVVELDWYPDAPGEMVVLTCDTPE
EDGITWTLDQSSEVLGSGKTLTIQVKEFGDAGQYTCHKGGEVLSHSLLLLHKKEDGI
WSTDILKDQKEPKNKTFLRCEAKNYSGRFTCWWLTTISTDLTFSVKSSRGSSDPQGV
TCGAATLSAERVRGDNKEYEYSVECQEDSACPAAEESLPIEVMVDAVHKLKYENYT
SSFFIRDIIKPDPPKNLQLKPLKNSRQVEVSWEYPDTWSTPHSYFSLTFCVQVQGKSK
REKKDRVFTDKTSATVICRKNASISVRAQDRYYSSSWSEWASVPCSGGGGSGGGGS
GGGGSRAVPGGSSPAWTQCQQLSQKLCTLAWSAHPLVGHMDLREEGDEETTNDVP
HIQCGDGCDPQGLRDNSQFCLQRIHQGLIFYEKLLGSDIFTGEPSLLPDSPVGQLHASL
LGLSQLLQPEGHHWETQQIPSLSPSQPWQRLLLRFKILRSLQAFVAVAARVFAHGAA
TLSPGSGSGSGSGSGSGSKQEHFPDNLLPSWAITLISVNGIFVICCLTYCFAPRCRERR
GSSHHWGYGKHNGPEHWHKDFPIAKGERQSPVDIDTHTAKYDPSLKPLSVSYDQAT
SLRILNNGHAFNVEFDDSQDKAVLKGGPLDGTYRLIQFHFHWGSLDGQGSEHTVDK
KKYAAELHLVHWNTKYGDFGKAVQQPDGLAVLGIFLKVGSAKPGHQKVVDVLDSI
KTKGKSADFTNFDPRGLLPESLDYWTYPGSLTTPPLLECVT

### Example 16. Regulation of Membrane-Bound IFNα Constructs in Primary Human CAR-T Cells

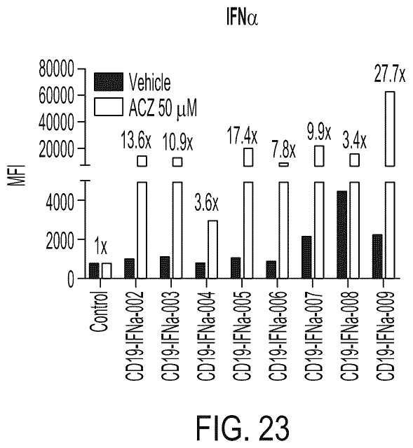

On day 0, primary human T cells were stimulated with Dynabeads (T-expander CD3/CD28) at a 3:1 bead:cell ratio in media containing 10% fetal bovine serum (FBS). The next day, lentivirus produced with constructs expressing CD19 CAR and membrane-bound oligomerizing IFNα (CD19-IFNα-002, CD19-IFNα-003, CD19-IFNα-004, CD19-IFNα-005, CD19-IFNα-006, CD19-IFNα-007, CD19-IFNα-008), and control constructs (CD19-IFNα-001, which expresses secreted IFNα; CD19-IFNα-009, which expresses monomeric IFNα without any oligomerizing modulation hub) were added in the presence of reduced serum (5% FBS). On day 2, the cells were diluted 1:2 with fresh 10% FBS media. On day 5, the cells were analyzed for CD19 CAR expression using CD19-Fc (R&D Systems; Part #9269-CD-050) followed by anti-human Alexa 647 anti-Human IgG (Jackson ImmunoResearch; Part #109-607-003). Media was replaced and cells were treated with 100 μM acetazolamide or DMSO. Twenty-four hours later T cells were analyzed by FACs. CD19 CAR was used as a transduction marker and anti-IFNα PE (BD Biosciences; Cat #560097) was used to stain for IFNα expression. The Geometric MFI of surface IFNα expression on CAR+ cells was plotted using Prism Software. The results are shown in FIG. 23. IFNα abundance was analyzed in the presence and absence of ACZ. The cells transduced with some oligomerized constructs have low IFNα abundance (off-state) in the absence of ACZ. The numbers above the ACZ treated cells represent fold change between on and off-state. Oligomerized constructs with multiple DRDs reduced basal abundance and enhanced fold change between on and off-states for IFNα. Of note CD19-IFNα-009 which is configured as a non-oligomerizing construct becomes robustly abundant on the cell surface after stabilization with ACZ, however, it does not possess a low basal abundance similar to the oligomerizing constructs.

The following constructs were used in this example:

| Name | Description |
|---|---|
| CD19-IFNα-001 | CD19CAR-p2a-IFNαsp-IFNα |
| (SEQ ID | |
| NO: 254) | |

MALPVTALLLPLALLLHAARPDIQMTQTTSSLSASLGDRVTISCRASQDISKYLNWY
QQKPDGTVKLLIYHTSRLHSGVPSRFSGSGSGTDYSLTISNLEQEDIATYFCQQGNTLP
YTFGGGTKLEITGGGGSGGGGSGGGGSEVKLQESGPGLVAPSQSLSVTCTVSGVSLP
DYGVSWIRQPPRKGLEWLGVIWGSETTYYNSALKSRLTIIKDNSKSQVFLKMNSLQT
DDTAIYYCAKHYYYGGSYAMDYWGQGTSVTVSSTTTPAPRPPTPAPTIASQPLSLRP
EACRPAAGGAVHTRGLDFACDIYIWAPLAGTCGVLLLSLVITLYCKRGRKKLLYIFK
QPFMRPVQTTQEEDGCSCRFPEEEEGGCELRVKFSRSADAPAYKQGQNQLYNELNL
GRREEYDVLDKRRGRDPEMGGKPRRKNPQEGLYNELQKDKMAEAYSEIGMKGERR
RGKGHDGLYQGLSTATKDTYDALHMQALPPRGSGATNFSLLKQAGDVEENPGPGG
SMALTFALLVALLVLSCKSSCSVGCDLPQTHSLGSRRTLMLLAQMRKISLFSCLKDR
HDFGFPQEEFGNQFQKAETIPVLHEMIQQIFNLFSTKDSSAAWDETLLDKFYTELYQQ
LNDLEACVIQGVGVTETPLMKEDSILAVRKYFQRITLYLKEKKYSPCAWEVVRAEIM
RSFSLSTNLQESLRSKE

| Name | Description |
|---|---|
| CD19-IFNα-002 | CD19CAR-p2a-CA2(L156H)-(GGSG)3-CD40Lcyto- |
| (SEQ ID | phospholambancyto-tm-CD40Lhinge(shed)-(GS)7-IFNα |
| NO: 255) | |

MALPVTALLLPLALLLHAARPDIQMTQTTSSLSASLGDRVTISCRASQDISKYLNWY
QQKPDGTVKLLIYHTSRLHSGVPSRFSGSGSGTDYSLTISNLEQEDIATYFCQQGNTLP
YTFGGGTKLEITGGGGSGGGGSGGGGSEVKLQESGPGLVAPSQSLSVTCTVSGVSLP
DYGVSWIRQPPRKGLEWLGVIWGSETTYYNSALKSRLTIIKDNSKSQVFLKMNSLQT
DDTAIYYCAKHYYYGGSYAMDYWGQGTSVTVSSTTTPAPRPPTPAPTIASQPLSLRP
EACRPAAGGAVHTRGLDFACDIYIWAPLAGTCGVLLLSLVITLYCKRGRKKLLYIFK
QPFMRPVQTTQEEDGCSCRFPEEEEGGCELRVKFSRSADAPAYKQGQNQLYNELNL
GRREEYDVLDKRRGRDPEMGGKPRRKNPQEGLYNELQKDKMAEAYSEIGMKGERR
RGKGHDGLYQGLSTATKDTYDALHMQALPPRGSGATNFSLLKQAGDVEENPGPGG
SSHHWGYGKHNGPEHWHKDFPIAKGERQSPVDIDTHTAKYDPSLKPLSVSYDQATS
LRILNNGHAFNVEFDDSQDKAVLKGGPLDGTYRLIQFHFHWGSLDGQGSEHTVDKK
KYAAELHLVHWNTKYGDFGKAVQQPDGLAVLGIFLKVGSAKPGHQKVVDVLDSIK
TKGKSADFTNFDPRGLLPESLDYWTYPGSLTTPPLLECVTWIVLKEPISVSSEQVLKF
RKLNFNGEGEPEELMVDNWRPAQPLKNRQIKASFKGGSGGGSGGGSGMIETYNQTS
PRSAATGLPISMKARQKLQNLFINFCLILICLLLICIIVMLLLHRRLDKIEDERNLHEDF
VFMKTIQRCNTGERSLSLLNCEEIKSQFEGFVKDIMLNKEETKKENSFEMQKGSGSGS
GSGSGSGSCDLPQTHSLGSRRTLMLLAQMRKISLFSCLKDRHDFGFPQEEFGNQFQK
AETIPVLHEMIQQIFNLFSTKDSSAAWDETLLDKFYTELYQQLNDLEACVIQGVGVTE
TPLMKEDSILAVRKYFQRITLYLKEKKYSPCAWEVVRAEIMRSFSLSTNLQESLRSKE

| Name | Description |
|---|---|
| CD19-IFNα-003 | CD19CAR-p2a-collectin7-(GSG)6-CA2(L156H)- |
| (SEQ ID | (GGSG)3-light(cyto-TM-hinge)-(GS)7-IFNα |
| NO: 256) | |

MALPVTALLLPLALLLHAARPDIQMTQTTSSLSASLGDRVTISCRASQDISKYLNWY
QQKPDGTVKLLIYHTSRLHSGVPSRFSGSGSGTDYSLTISNLEQEDIATYFCQQGNTLP
YTFGGGTKLEITGGGGSGGGGSGGGGSEVKLQESGPGLVAPSQSLSVTCTVSGVSLP
DYGVSWIRQPPRKGLEWLGVIWGSETTYYNSALKSRLTIIKDNSKSQVFLKMNSLQT
DDTAIYYCAKHYYYGGSYAMDYWGQGTSVTVSSTTTPAPRPPTPAPTIASQPLSLRP
EACRPAAGGAVHTRGLDFACDIYIWAPLAGTCGVLLLSLVITLYCKRGRKKLLYIFK
QPFMRPVQTTQEEDGCSCRFPEEEEGGCELRVKFSRSADAPAYKQGQNQLYNELNL
GRREEYDVLDKRRGRDPEMGGKPRRKNPQEGLYNELQKDKMAEAYSEIGMKGERR
RGKGHDGLYQGLSTATKDTYDALHMQALPPRGSGATNFSLLKQAGDVEENPGPGG
SMSGLPDVASLRQQVEALQGQVQHLQAAFSQYKKVELFPNGQSgsggsggsggsggsggsg
SHHWGYGKHNGPEHWHKDFPIAKGERQSPVDIDTHTAKYDPSLKPLSVSYDQATSL
RILNNGHAFNVEFDDSQDKAVLKGGPLDGTYRLIQFHFHWGSLDGQGSEHTVDKKK
YAAELHLVHWNTKYGDFGKAVQQPDGLAVLGIFLKVGSAKPGHQKVVDVLDSIKT
KGKSADFTNFDPRGLLPESLDYWTYPGSLTTPPLLECVTWIVLKEPISVSSEQVLKFR
KLNFNGEGEPEELMVDNWRPAQPLKNRQIKASFKGGSGGGSGGGSGDGQTDIPFTR
LGRSHRRQSCSVARVGLGLLLLLMGAGLAVQGWFLLQLHWRLGEMVTRLPDGPAG
SWEQLIQGSGSGSGSGSGSGSCDLPQTHSLGSRRTLMLLAQMRKISLFSCLKDRHDF
GFPQEEFGNQFQKAETIPVLHEMIQQIFNLFSTKDSSAAWDETLLDKFYTELYQQLND
LEACVIQGVGVTETPLMKEDSILAVRKYFQRITLYLKEKKYSPCAWEVVRAEIMRSF
SLSTNLQESLRSKE

| Name | Description |
|---|---|
| CD19-IFNα-004 | CD19CAR-p2a-trimerizing domain (collectin 7)-(GSG)6- |
| (SEQ ID | CA2(L156H)-(ggsg)3-CD40Lcyto-TM-hinge(shed)-(GS)7-IFNα |
| NO: 257) | |

MALPVTALLLPLALLLHAARPDIQMTQTTSSLSASLGDRVTISCRASQDISKYLNWY
QQKPDGTVKLLIYHTSRLHSGVPSRFSGSGSGTDYSLTISNLEQEDIATYFCQQGNTLP
YTFGGGTKLEITGGGGSGGGGSGGGGSEVKLQESGPGLVAPSQSLSVTCTVSGVSLP
DYGVSWIRQPPRKGLEWLGVIWGSETTYYNSALKSRLTIIKDNSKSQVFLKMNSLQT
DDTAIYYCAKHYYYGGSYAMDYWGQGTSVTVSSTTTPAPRPPTPAPTIASQPLSLRP
EACRPAAGGAVHTRGLDFACDIYIWAPLAGTCGVLLLSLVITLYCKRGRKKLLYIFK
QPFMRPVQTTQEEDGCSCRFPEEEEGGCELRVKFSRSADAPAYKQGQNQLYNELNL
GRREEYDVLDKRRGRDPEMGGKPRRKNPQEGLYNELQKDKMAEAYSEIGMKGERR
RGKGHDGLYQGLSTATKDTYDALHMQALPPRGSGATNFSLLKQAGDVEENPGPGG
SMSGLPDVASLRQQVEALQGQVQHLQAAFSQYKKVELFPNGQSgsggsggsggsggsggsg
SHHWGYGKHNGPEHWHKDFPIAKGERQSPVDIDTHTAKYDPSLKPLSVSYDQATSL
RILNNGHAFNVEFDDSQDKAVLKGGPLDGTYRLIQFHFHWGSLDGQGSEHTVDKKK
YAAELHLVHWNTKYGDFGKAVQQPDGLAVLGIFLKVGSAKPGHQKVVDVLDSIKT
KGKSADFTNFDPRGLLPESLDYWTYPGSLTTPPLLECVTWIVLKEPISVSSEQVLKFR
KLNFNGEGEPEELMVDNWRPAQPLKNRQIKASFKggsgggsgggsgMIETYNQTSPRSAA
TGLPISMKIFMYLLTVFLITQMIGSALFAVYLHRRLDKIEDERNLHEDFVFMKTIQRC
NTGERSLSLLNCEEIKSQFEGFVKDIMLNKEETKKENSFEMQKGSGSGSGSGSGSGSC
DLPQTHSLGSRRTLMLLAQMRKISLFSCLKDRHDFGFPQEEFGNQFQKAETIPVLHE
MIQQIFNLFSTKDSSAAWDETLLDKFYTELYQQLNDLEACVIQGVGVTETPLMKEDS
ILAVRKYFQRITLYLKEKKYSPCAWEVVRAEIMRSFSLSTNLQESLRSKE

| Name | Description |
|---|---|
| CD19-IFNα-005 | CD19CAR-p2a-IFNαsp-IFNα-linker-CD40L shed site- |
| (SEQ ID | DAP12hingeDAP12TMDAP12cyto-CA2(L156H) |
| NO: 258) | |

MALPVTALLLPLALLLHAARPDIQMTQTTSSLSASLGDRVTISCRASQDISKYLNWY
QQKPDGTVKLLIYHTSRLHSGVPSRFSGSGSGTDYSLTISNLEQEDIATYFCQQGNTLP
YTFGGGTKLEITGGGGSGGGGSGGGGSEVKLQESGPGLVAPSQSLSVTCTVSGVSLP
DYGVSWIRQPPRKGLEWLGVIWGSETTYYNSALKSRLTIIKDNSKSQVFLKMNSLQT
DDTAIYYCAKHYYYGGSYAMDYWGQGTSVTVSSTTTPAPRPPTPAPTIASQPLSLRP
EACRPAAGGAVHTRGLDFACDIYIWAPLAGTCGVLLLSLVITLYCKRGRKKLLYIFK
QPFMRPVQTTQEEDGCSCRFPEEEEGGCELRVKFSRSADAPAYKQGQNQLYNELNL
GRREEYDVLDKRRGRDPEMGGKPRRKNPQEGLYNELQKDKMAEAYSEIGMKGERR
RGKGHDGLYQGLSTATKDTYDALHMQALPPRGSGATNFSLLKQAGDVEENPGPGG
SMALTFALLVALLVLSCKSSCSVGCDLPQTHSLGSRRTLMLLAQMRKISLFSCLKDR
HDFGFPQEEFGNQFQKAETIPVLHEMIQQIFNLFSTKDSSAAWDETLLDKFYTELYQQ
LNDLEACVIQGVGVTETPLMKEDSILAVRKYFQRITLYLKEKKYSPCAWEVVRAEIM
RSFSLSTNLQESLRSKEGSGSGSGSGSGSGSSFEMQKLRPVQAQAQSDCSCSTVSPGV
LAGIVMGDLVLTVLIALAVYFLGRLVPRGRGAAEAATRKQRITEGGSGGGSGSSHH
WGYGKHNGPEHWHKDFPIAKGERQSPVDIDTHTAKYDPSLKPLSVSYDQATSLRILN
NGHAFNVEFDDSQDKAVLKGGPLDGTYRLIQFHFHWGSLDGQGSEHTVDKKKYAA
ELHLVHWNTKYGDFGKAVQQPDGLAVLGIFLKVGSAKPGHQKVVDVLDSIKTKGK
SADFTNFDPRGLLPESLDYWTYPGSLTTPPLLECVTWIVLKEPISVSSEQVLKFRKLNF
NGEGEPEELMVDNWRPAQPLKNRQIKASFK

| Name | Description |
|---|---|
| CD19-IFNα-006 | CD19CAR-p2a-(GSG)6-CA2(S56N)-(ggsg)3-CD40Lcyto-TM- |
| (SEQ ID | hinge(shed)-(GS)7-IFNα(control typeII, shed) |
| NO: 259) | |

MALPVTALLLPLALLLHAARPDIQMTQTTSSLSASLGDRVTISCRASQDISKYLNWY
QQKPDGTVKLLIYHTSRLHSGVPSRFSGSGSGTDYSLTISNLEQEDIATYFCQQGNTLP
YTFGGGTKLEITGGGGSGGGGSGGGGSEVKLQESGPGLVAPSQSLSVTCTVSGVSLP
DYGVSWIRQPPRKGLEWLGVIWGSETTYYNSALKSRLTIIKDNSKSQVFLKMNSLQT
DDTAIYYCAKHYYYGGSYAMDYWGQGTSVTVSSTTTPAPRPPTPAPTIASQPLSLRP
EACRPAAGGAVHTRGLDFACDIYIWAPLAGTCGVLLLSLVITLYCKRGRKKLLYIFK
QPFMRPVQTTQEEDGCSCRFPEEEEGGCELRVKFSRSADAPAYKQGQNQLYNELNL
GRREEYDVLDKRRGRDPEMGGKPRRKNPQEGLYNELQKDKMAEAYSEIGMKGERR
RGKGHDGLYQGLSTATKDTYDALHMQALPPRGSGATNFSLLKQAGDVEENPGPGG
SSHHWGYGKHNGPEHWHKDFPIAKGERQSPVDIDTHTAKYDPSLKPLSVSYDQATN
LRILNNGHAFNVEFDDSQDKAVLKGGPLDGTYRLIQFHFHWGSLDGQGSEHTVDKK
KYAAELHLVHWNTKYGDFGKAVQQPDGLAVLGIFLKVGSAKPGLQKVVDVLDSIK
TKGKSADFTNFDPRGLLPESLDYWTYPGSLTTPPLLECVTWIVLKEPISVSSEQVLKF
RKLNFNGEGEPEELMVDNWRPAQPLKNRQIKASFKggsgggsgggsgMIETYNQTSPRSA
ATGLPISMKIFMYLLTVFLITQMIGSALFAVYLHRRLDKIEDERNLHEDFVFMKTIQR
CNTGERSLSLLNCEEIKSQFEGFVKDIMLNKEETKKENSFEMQKGSGSGSGSGSGSGS
CDLPQTHSLGSRRTLMLLAQMRKISLFSCLKDRHDFGFPQEEFGNQFQKAETIPVLHE
MIQQIFNLFSTKDSSAAWDETLLDKFYTELYQQLNDLEACVIQGVGVTETPLMKEDS
ILAVRKYFQRITLYLKEKKYSPCAWEVVRAEIMRSFSLSTNLQESLRSKEWIVLKEPIS
VSSEQVLKFRKLNFNGEGEPEELMVDNWRPAQPLKNRQIKASFK

| Name | Description |
|---|---|
| CD19-IFNα-007 | CA2(L156H)-(GGSG)3-41BBL cyto (21 aa)- |
| (SEQ ID NO: | 41BBL TM-41BBLhinge-41BBL ECD-(GS)7-IFNα |
| 260) | |

MALPVTALLLPLALLLHAARPDIQMTQTTSSLSASLGDRVTISCRASQDISKYLNWY
QQKPDGTVKLLIYHTSRLHSGVPSRFSGSGSGTDYSLTISNLEQEDIATYFCQQGNTLP
YTFGGGTKLEITGGGGSGGGGSGGGGSEVKLQESGPGLVAPSQSLSVTCTVSGVSLP
DYGVSWIRQPPRKGLEWLGVIWGSETTYYNSALKSRLTIIKDNSKSQVFLKMNSLQT
DDTAIYYCAKHYYYGGSYAMDYWGQGTSVTVSSTTTPAPRPPTPAPTIASQPLSLRP
EACRPAAGGAVHTRGLDFACDIYIWAPLAGTCGVLLLSLVITLYCKRGRKKLLYIFK
QPFMRPVQTTQEEDGCCRFPEEEEGGCELRVKFSRSADAPAYKQGQNQLYNELNL
GRREEYDVLDKRRGRDPEMGGKPRRKNPQEGLYNELQKDKMAEAYSEIGMKGERR
RGKGHDGLYQGLSTATKDTYDALHMQALPPRGSGATNFSLLKQAGDVEENPGPGG
SSHHWGYGKHNGPEHWHKDFPIAKGERQSPVDIDTHTAKYDPSLKPLSVSYDQATS
LRILNNGHAFNVEFDDSQDKAVLKGGPLDGTYRLIQFHFHWGSLDGQGSEHTVDKK
KYAAELHLVHWNTKYGDFGKAVQQPDGLAVLGIFLKVGSAKPGHQKVVDVLDSIK
TKGKSADFTNFDPRGLLPESLDYWTYPGSLTTPPLLECVTWIVLKEPISVSSEQVLKF
RKLNFNGEGEPEELMVDNWRPAQPLKNRQIKASFKGGSGGGSGGGSGPEAPWPPAP
RARACRVLPWALVAGLLLLLLLAAACAVFLACPWAVSGARASPGSAASPRLREGPE
LSPDDPAGLLDLRQGMFAQLVAQNVLLIDGPLSWYSDPGLAGVSLTGGLSYKEDTK
ELVVAKAGVYYVFFQLELRRVVAGEGSGSVSLALHLQPLRSAAGAAALALTVDLPP
ASSEARNSAFGFQGRLLHLSAGQRLGVHLHTEARARHAWQLTQGATVLGLFRVTPE
IPAGLPSPRSEGSGSGSGSGSGSGSCDLPQTHSLGSRRTLMLLAQMRKISLFSCLKDRH
DFGFPQEEFGNQFQKAETIPVLHEMIQQIFNLFSTKDSSAAWDETLLDKFYTELYQQL
NDLEACVIQGVGVTETPLMKEDSILAVRKYFQRITLYLKEKKYSPCAWEVVRAEIMR
SFSLSTNLQESLRSKE

| Name | Description |
|---|---|
| CD19-IFNα-008 | CD19CAR-p2a-trimerizing domain (collectin 7)-(GSG)6- |
| (SEQ ID | CA2(L156H)-(ggsg)3-41BBLcyto (21 aa)-TM-hinge-(GS)7-IFNα |
| NO: 261) | |

MALPVTALLLPLALLLHAARPDIQMTQTTSSLSASLGDRVTISCRASQDISKYLNWY
QQKPDGTVKLLIYHTSRLHSGVPSRFSGSGSGTDYSLTISNLEQEDIATYFCQQGNTLP
YTFGGGTKLEITGGGGSGGGGSGGGGSEVKLQESGPGLVAPSQSLSVTCTVSGVSLP
DYGVSWIRQPPRKGLEWLGVIWGSETTYYNSALKSRLTIIKDNSKSQVFLKMNSLQT
DDTAIYYCAKHYYYGGSYAMDYWGQGTSVTVSSTTTPAPRPPTPAPTIASQPLSLRP
EACRPAAGGAVHTRGLDFACDIYIWAPLAGTCGVLLLSLVITLYCKRGRKKLLYIFK
QPFMRPVQTTQEEDGCSCRFPEEEEGGCELRVKFSRSADAPAYKQGQNQLYNELNL
GRREEYDVLDKRRGRDPEMGGKPRRKNPQEGLYNELQKDKMAEAYSEIGMKGERR
RGKGHDGLYQGLSTATKDTYDALHMQALPPRGSGATNFSLLKQAGDVEENPGPGG
SMSGLPDVASLRQQVEALQGQVQHLQAAFSQYKKVELFPNGQSgsggsggsggsggsggsg
SHHWGYGKHNGPEHWHKDFPIAKGERQSPVDIDTHTAKYDPSLKPLSVSYDQATSL
RILNNGHAFNVEFDDSQDKAVLKGGPLDGTYRLIQFHFHWGSLDGQGSEHTVDKKK
YAAELHLVHWNTKYGDFGKAVQQPDGLAVLGIFLKVGSAKPGHQKVVDVLDSIKT
KGKSADFTNFDPRGLLPESLDYWTYPGSLTTPPLLECVTWIVLKEPISVSSEQVLKFR
KLNFNGEGEPEELMVDNWRPAQPLKNRQIKASFKggsgggsgggsgPEAPWPPAPRARAC
RVLPWALVAGLLLLLLLAAACAVFLACPWAVSGARASPGSAASPRLREGPELSPDDP
AGLLDLRQGMFAQLGSGSGSGSGSGSGSCDLPQTHSLGSRRTLMLLAQMRKISLFSC
LKDRHDFGFPQEEFGNQFQKAETIPVLHEMIQQIFNLFSTKDSSAAWDETLLDKFYTE
LYQQLNDLEACVIQGVGVTETPLMKEDSILAVRKYFQRITLYLKEKKYSPCAWEVV
RAEIMRSFSLSTNLQESLRSKE

| Name | Description |
|---|---|
| CD19-IFNα-009 | CD19CAR-p2a-IFNαsp-IFNα-linker(GS)7-B7-1 hingeTM-tail- |
| (SEQ ID | CA2(L156H)(control regulated construct, with B7-1 hinge) |
| NO: 262) | |

MALPVTALLLPLALLLHAARPDIQMTQTTSSLSASLGDRVTISCRASQDISKYLNWY
QQKPDGTVKLLIYHTSRLHSGVPSRFSGSGSGTDYSLTISNLEQEDIATYFCQQGNTLP
YTFGGGTKLEITGGGGSGGGGSGGGGSEVKLQESGPGLVAPSQSLSVTCTVSGVSLP
DYGVSWIRQPPRKGLEWLGVIWGSETTYYNSALKSRLTIIKDNSKSQVFLKMNSLQT
DDTAIYYCAKHYYYGGSYAMDYWGQGTSVTVSSTTTPAPRPPTPAPTIASQPLSLRP
EACRPAAGGAVHTRGLDFACDIYIWAPLAGTCGVLLLSLVITLYCKRGRKKLLYIFK
QPFMRPVQTTQEEDGCSCRFPEEEEGGCELRVKFSRSADAPAYKQGQNQLYNELNL
GRREEYDVLDKRRGRDPEMGGKPRRKNPQEGLYNELQKDKMAEAYSEIGMKGERR
RGKGHDGLYQGLSTATKDTYDALHMQALPPRGSGATNFSLLKQAGDVEENPGPGG
SMALTFALLVALLVLSCKSSCSVGCDLPQTHSLGSRRTLMLLAQMRKISLFSCLKDR
HDFGFPQEEFGNQFQKAETIPVLHEMIQQIFNLFSTKDSSAAWDETLLDKFYTELYQQ
LNDLEACVIQGVGVTETPLMKEDSILAVRKYFQRITLYLKEKKYSPCAWEVVRAEIM
RSFSLSTNLQESLRSKEGSGSGSGSGSGSGSKQEHFPDNLLPSWAITLISVNGIFVICCL
TYCFAPRCRERRGSSHHWGYGKHNGPEHWHKDFPIAKGERQSPVDIDTHTAKYDPS
LKPLSVSYDQATSLRILNNGHAFNVEFDDSQDKAVLKGGPLDGTYRLIQFHFHWGSL
DGQGSEHTVDKKKYAAELHLVHWNTKYGDFGKAVQQPDGLAVLGIFLKVGSAKPG
HQKVVDVLDSIKTKGKSADFTNFDPRGLLPESLDYWTYPGSLTTPPLLECVT

### Example 17. Regulation of IL2 from Tandem CD19CAR-p2a-IL2 Constructs in Jurkat Cells

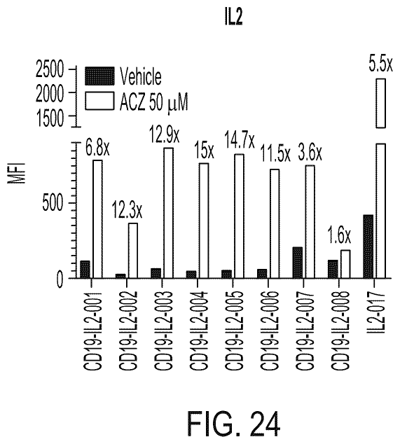

Jurkat, Clone E6-1, cells were cultured in standard media (RPMI+ GlutaMAX Supplement (Life Technologies; Cat #61870127), Fetal Bovine Serum (FBS) (Life Technologies; Cat #10-082-147)). Jurkat cells were transduced with lentivirus produced with each of CD19CAR-IL2-001, CD19CAR-IL2-002, CD19CAR-IL2-003, CD19CAR-IL2-004, CD19CAR-IL2-005, CD19CAR-IL2-006, CD19CAR-IL2-007, CD19CAR-IL2-008 and IL2-017. Jurkat cells were transduced to 10-25% positive as determined by using CD19 CAR as a transduction marker. CD19 CAR expression was assayed using CD19-Fc (R&D Systems; Part #9269-CD-050) followed by anti-human Alexa 647 anti-Human IgG (Jackson ImmunoResearch; Part #109-607-003). Five days post transduction, the cells were treated with 100 μM acetazolamide or DMSO, and the cells were further cultured for 1 more day. PE Anti-human IL2 antibody (Biolegend; Cat #500307) was used to stain for IL2 expression. FIG. 24 shows geometric MFI indicative of IL2 abundance on CD19CAR positive Jurkat cells in the presence and absence of ACZ. Note the cells transduced with some oligomerized constructs (CD19CAR-IL2-001, CD19CAR-IL2-002, CD19CAR-IL2-003, CD19CAR-IL2-004, CD19CAR-IL2-005, CD19CAR-IL2-006) have low IL2 abundance (off-state) in the absence of ACZ, whereas oligomerizing concepts investigated in CD19CAR-IL2-007, CD19CAR-IL2-008 did not result in significant improvement compared to IL2-017. which is a non-oligomerizing DRD regulated construct. The numbers above the ACZ treated cells represent fold change between on and off-state. MFI was calculated on cells within the CD19CAR positive gate. Oligomerized constructs with multiple DRDs reduced basal abundance and enhanced fold change between on and off-states for IL2.

The following constructs were used in this example:

Name
CD19-IL2-001 (SEQ ID NO: 263)
MALPVTALLLPLALLLHAARPDIQMTQTTSSLSASLGDRVTISCRASQDISKYLNWY
QQKPDGTVKLLIYHTSRLHSGVPSRFSGSGSGTDYSLTISNLEQEDIATYFCQQGNTLP
YTFGGGTKLEITGGGGSGGGGSGGGGSEVKLQESGPGLVAPSQSLSVTCTVSGVSLP
DYGVSWIRQPPRKGLEWLGVIWGSETTYYNSALKSRLTIIKDNSKSQVFLKMNSLQT
DDTAIYYCAKHYYYGGSYAMDYWGQGTSVTVSSTTTPAPRPPTPAPTIASQPLSLRP
EACRPAAGGAVHTRGLDFACDIYIWAPLAGTCGVLLLSLVITLYCKRGRKKLLYIFK
QPFMRPVQTTQEEDGCSCRFPEEEEGGCELRVKFSRSADAPAYKQGQNQLYNELNL
GRREEYDVLDKRRGRDPEMGGKPRRKNPQEGLYNELQKDKMAEAYSEIGMKGERR
RGKGHDGLYQGLSTATKDTYDALHMQALPPRGSGATNFSLLKQAGDVEENPGPGG
SMSHHWGYGKHNGPEHWHKDFPIAKGERQSPVDIDTHTAKYDPSLKPLSVSYDQAT
NLRILNNGHAFNVEFDDSQDKAVLKGGPLDGTYRLIQFHFHWGSLDGQGSEHTVDK
KKYAAELHLVHWNTKYGDFGKAVQQPDGLAVLGIFLKVGSAKPGLQKVVDVLDSI
KTKGKSADFTNFDPRGLLPESLDYWTYPGSLTTPPLLECVTWIVLKEPISVSSEQVLK
FRKLNFNGEGEPEELMVDNWRPAQPLKNRQIKASFKGGSGGGSGGGSGSKQRGTFS
EVSLAQDARQKLQNLFINFCLILICLLLICIIVMLLIPFLEQNNSSPNTRTQKSFEMQKG
SGSGSGSGSGSGSAPTSSSTKKTQLQLEHLLLDLQMILNGINNYKNPKLTRMLTFKFY
MPKKATELKHLQCLEEELKPLEEVLNLAQSKNFHLRPRDLISNINVIVLELKGSETTF
MCEYADETATIVEFLNRWITFCQSIISTLT
Name
CD19-IL2-002 (SEQ ID NO: 264)
MALPVTALLLPLALLLHAARPDIQMTQTTSSLSASLGDRVTISCRASQDISKYLNWY
QQKPDGTVKLLIYHTSRLHSGVPSRFSGSGSGTDYSLTISNLEQEDIATYFCQQGNTLP
YTFGGGTKLEITGGGGSGGGGSGGGGSEVKLQESGPGLVAPSQSLSVTCTVSGVSLP
DYGVSWIRQPPRKGLEWLGVIWGSETTYYNSALKSRLTIIKDNSKSQVFLKMNSLQT
DDTAIYYCAKHYYYGGSYAMDYWGQGTSVTVSSTTTPAPRPPTPAPTIASQPLSLRP
EACRPAAGGAVHTRGLDFACDIYIWAPLAGTCGVLLLSLVITLYCKRGRKKLLYIFK
QPFMRPVQTTQEEDGCSCRFPEEEEGGCELRVKFSRSADAPAYKQGQNQLYNELNL
GRREEYDVLDKRRGRDPEMGGKPRRKNPQEGLYNELQKDKMAEAYSEIGMKGERR
RGKGHDGLYQGLSTATKDTYDALHMQALPPRGSGATNFSLLKQAGDVEENPGPGG
SMSGLPDVASLRQQVEALQGQVQHLQAAFSQYKKVELFPNGQSGSGGSGGSGGSG
GSGGSGSHHWGYGKHNGPEHWHKDFPIAKGERQSPVDIDTHTAKYDPSLKPLSVSY
DQATNLRILNNGHAFNVEFDDSQDKAVLKGGPLDGTYRLIQFHFHWGSLDGQGSEH
TVDKKKYAAELHLVHWNTKYGDFGKAVQQPDGLAVLGIFLKVGSAKPGLQKVVD
VLDSIKTKGKSADFTNFDPRGLLPESLDYWTYPGSLTTPPLLECVTWIVLKEPISVSSE
QVLKFRKLNFNGEGEPEELMVDNWRPAQPLKNRQIKASFKGGSGGGSGGGSGMIET
YNQTSPRSAATGLPISMKIFMYLLTVFLITQMIGSALFAVYLHRRLDKIEDERNLHED
FVFMKTIQRCNTGERSLSLLNCEEIKSQFEGFVKDIMLNKEETKKENSFEMQKGSGSG
SGSGSGSGSAPTSSSTKKTQLQLEHLLLDLQMILNGINNYKNPKLTRMLTFKFYMPK
KATELKHLQCLEEELKPLEEVLNLAQSKNFHLRPRDLISNINVIVLELKGSETTFMCE
YADETATIVEFLNRWITFCQSIISTLT
Name
CD19-IL2-003 (SEQ ID NO: 265)
MALPVTALLLPLALLLHAARPDIQMTQTTSSLSASLGDRVTISCRASQDISKYLNWY
QQKPDGTVKLLIYHTSRLHSGVPSRFSGSGSGTDYSLTISNLEQEDIATYFCQQGNTLP
YTFGGGTKLEITGGGGSGGGGSGGGGSEVKLQESGPGLVAPSQSLSVTCTVSGVSLP
DYGVSWIRQPPRKGLEWLGVIWGSETTYYNSALKSRLTIIKDNSKSQVFLKMNSLQT
DDTAIYYCAKHYYYGGSYAMDYWGQGTSVTVSSTTTPAPRPPTPAPTIASQPLSLRP
EACRPAAGGAVHTRGLDFACDIYIWAPLAGTCGVLLLSLVITLYCKRGRKKLLYIFK
QPFMRPVQTTQEEDGCSCRFPEEEEGGCELRVKFSRSADAPAYKQGQNQLYNELNL
GRREEYDVLDKRRGRDPEMGGKPRRKNPQEGLYNELQKDKMAEAYSEIGMKGERR
RGKGHDGLYQGLSTATKDTYDALHMQALPPRGSGATNFSLLKQAGDVEENPGPGG
SMSHHWGYGKHNGPEHWHKDFPIAKGERQSPVDIDTHTAKYDPSLKPLSVSYDQAT
NLRILNNGHAFNVEFDDSQDKAVLKGGPLDGTYRLIQFHFHWGSLDGQGSEHTVDK
KKYAAELHLVHWNTKYGDFGKAVQQPDGLAVLGIFLKVGSAKPGLQKVVDVLDSI
KTKGKSADFTNFDPRGLLPESLDYWTYPGSLTTPPLLECVTWIVLKEPISVSSEQVLK
FRKLNFNGEGEPEELMVDNWRPAQPLKNRQIKASFKGGSGGGSGGGSGMIETYNQT
SPRSAATGLPISMKIFMYLLTVFLITQMIGSALFAVYLHRRLDKIEDERNLHEDFVFM
KTIQRCNTGERSLSLLNCEEIKSQFEGFVKDIMLNKEETKKENGGSGGSGDQNPQIAA
HVISEASSKTTSVLQWAEKGYYTMSNNLVTLENGKQLTVKRQGLYYIYAQVTFCSN
REASSQAPFIASLCLKSPGRFERILLRAANTHSSAKPCGQQSIHLGGVFELQPGASVFV
NVTDPSQVSHGTGFTSFGLLKLGSGSGSGSGSGSGSAPTSSSTKKTQLQLEHLLLDLQ
MILNGINNYKNPKLTRMLTFKFYMPKKATELKHLQCLEEELKPLEEVLNLAQSKNFH
LRPRDLISNINVIVLELKGSETTFMCEYADETATIVEFLNRWITFCQSIISTLT
Name
CD19-IL2-004 (SEQ ID NO: 266)
MALPVTALLLPLALLLHAARPDIQMTQTTSSLSASLGDRVTISCRASQDISKYLNWY
QQKPDGTVKLLIYHTSRLHSGVPSRFSGSGSGTDYSLTISNLEQEDIATYFCQQGNTLP
YTFGGGTKLEITGGGGSGGGGSGGGGSEVKLQESGPGLVAPSQSLSVTCTVSGVSLP
DYGVSWIRQPPRKGLEWLGVIWGSETTYYNSALKSRLTIIKDNSKSQVFLKMNSLQT
DDTAIYYCAKHYYYGGSYAMDYWGQGTSVTVSSTTTPAPRPPTPAPTIASQPLSLRP
EACRPAAGGAVHTRGLDFACDIYIWAPLAGTCGVLLLSLVITLYCKRGRKKLLYIFK
QPFMRPVQTTQEEDGCSCRFPEEEEGGCELRVKFSRSADAPAYKQGQNQLYNELNL
GRREEYDVLDKRRGRDPEMGGKPRRKNPQEGLYNELQKDKMAEAYSEIGMKGERR
RGKGHDGLYQGLSTATKDTYDALHMQALPPRGSGATNFSLLKQAGDVEENPGPGG
SMSHHWGYGKHNGPEHWHKDFPIAKGERQSPVDIDTHTAKYDPSLKPLSVSYDQAT
NLRILNNGHAFNVEFDDSQDKAVLKGGPLDGTYRLIQFHFHWGSLDGQGSEHTVDK
KKYAAELHLVHWNTKYGDFGKAVQQPDGLAVLGIFLKVGSAKPGLQKVVDVLDSI
KTKGKSADFTNFDPRGLLPESLDYWTYPGSLTTPPLLECVTWIVLKEPISVSSEQVLK
FRKLNFNGEGEPEELMVDNWRPAQPLKNRQIKASFKGGSGGGSGGGSGMIETYNQT
SPRSAATGLPISMKIFMYLLTVFLITQMIGSALFAVYLHRRLDKIEDERNLHEDFVFM
KTIQRCNTGERSLSLLNCEEIKSQFEGFVKDIMLNKEETKKENGGSGGSGGGGSGGG
GSGASSGVRLWATRQAMLGQVHEVPEGWLIFVAEQEELYVRVQNGFRKVQLEART
PLPRGTDNGSGSGSGSGSGSGSAPTSSSTKKTQLQLEHLLLDLQMILNGINNYKNPKL
TRMLTFKFYMPKKATELKHLQCLEEELKPLEEVLNLAQSKNFHLRPRDLISNINVIVL
ELKGSETTFMCEYADETATIVEFLNRWITFCQSIISTLT
Name
CD19-IL2-005 (SEQ ID NO: 267)
MALPVTALLLPLALLLHAARPDIQMTQTTSSLSASLGDRVTISCRASQDISKYLNWY
QQKPDGTVKLLIYHTSRLHSGVPSRFSGSGSGTDYSLTISNLEQEDIATYFCQQGNTLP
YTFGGGTKLEITGGGGSGGGGSGGGGSEVKLQESGPGLVAPSQSLSVTCTVSGVSLP
DYGVSWIRQPPRKGLEWLGVIWGSETTYYNSALKSRLTIIKDNSKSQVFLKMNSLQT
DDTAIYYCAKHYYYGGSYAMDYWGQGTSVTVSSTTTPAPRPPTPAPTIASQPLSLRP
EACRPAAGGAVHTRGLDFACDIYIWAPLAGTCGVLLLSLVITLYCKRGRKKLLYIFK
QPFMRPVQTTQEEDGCSCRFPEEEEGGCELRVKFSRSADAPAYKQGQNQLYNELNL
GRREEYDVLDKRRGRDPEMGGKPRRKNPQEGLYNELQKDKMAEAYSEIGMKGERR
RGKGHDGLYQGLSTATKDTYDALHMQALPPRGSGATNFSLLKQAGDVEENPGPGG
SMSHHWGYGKHNGPEHWHKDFPIAKGERQSPVDIDTHTAKYDPSLKPLSVSYDQAT
NLRILNNGHAFNVEFDDSQDKAVLKGGPLDGTYRLIQFHFHWGSLDGQGSEHTVDK
KKYAAELHLVHWNTKYGDFGKAVQQPDGLAVLGIFLKVGSAKPGLQKVVDVLDSI
KTKGKSADFTNFDPRGLLPESLDYWTYPGSLTTPPLLECVTWIVLKEPISVSSEQVLK
FRKLNFNGEGEPEELMVDNWRPAQPLKNRQIKASFKGGSGGGSGGGSGMIETYNQT
SPRSAATGLPISMKIFMYLLTVFLITQMIGSALFAVYLHRRLDKIEDERNLHEDFVFM
KTIQRCNTGERSLSLLNCEEIKSQFEGFVKDIMLNKEETKKENGGGGSGGGGSSGLPD
VASLRQQVEALQGQVQHLQAAFSQYKKVELFPNGQSGSGSGSGSGSGSGSAPTSSST
KKTQLQLEHLLLDLQMILNGINNYKNPKLTRMLTFKFYMPKKATELKHLQCLEEEL
KPLEEVLNLAQSKNFHLRPRDLISNINVIVLELKGSETTFMCEYADETATIVEFLNRWI
TFCQSIISTLT
Name
CD19-IL2-006 (SEQ ID NO: 268)
MALPVTALLLPLALLLHAARPDIQMTQTTSSLSASLGDRVTISCRASQDISKYLNWY
QQKPDGTVKLLIYHTSRLHSGVPSRFSGSGSGTDYSLTISNLEQEDIATYFCQQGNTLP
YTFGGGTKLEITGGGGSGGGGSGGGGSEVKLQESGPGLVAPSQSLSVTCTVSGVSLP
DYGVSWIRQPPRKGLEWLGVIWGSETTYYNSALKSRLTIIKDNSKSQVFLKMNSLQT
DDTAIYYCAKHYYYGGSYAMDYWGQGTSVTVSSTTTPAPRPPTPAPTIASQPLSLRP
EACRPAAGGAVHTRGLDFACDIYIWAPLAGTCGVLLLSLVITLYCKRGRKKLLYIFK
QPFMRPVQTTQEEDGCSCRFPEEEEGGCELRVKFSRSADAPAYKQGQNQLYNELNL
GRREEYDVLDKRRGRDPEMGGKPRRKNPQEGLYNELQKDKMAEAYSEIGMKGERR
RGKGHDGLYQGLSTATKDTYDALHMQALPPRGSGATNFSLLKQAGDVEENPGPGG
SMSHHWGYGKHNGPEHWHKDFPIAKGERQSPVDIDTHTAKYDPSLKPLSVSYDQAT
NLRILNNGHAFNVEFDDSQDKAVLKGGPLDGTYRLIQFHFHWGSLDGQGSEHTVDK
KKYAAELHLVHWNTKYGDFGKAVQQPDGLAVLGIFLKVGSAKPGLQKVVDVLDSI
KTKGKSADFTNFDPRGLLPESLDYWTYPGSLTTPPLLECVTWIVLKEPISVSSEQVLK
FRKLNFNGEGEPEELMVDNWRPAQPLKNRQIKASFKGGSGGGSGGGSGMIETYNQT
SPRSAATGLPISMKIFMYLLTVFLITQMIGSALFAVYLHRRLDKIEDERNLHEDFVFM
KTIQRCNTGERSLSLLNCEEIKSQFEGFVKDIMLNKEETKKENSFEMQKGDQNPQIAA
HVISEASSKTTSVLQWAEKGYYTMSNNLVTLENGKQLTVKRQGLYYIYAQVTFCSN
REASSQAPFIASLCLKSPGRFERILLRAANTHSSAKPCGQQSIHLGGVFELQPGASVFV
NVTDPSQVSHGTGFTSFGLLKLGSGSGSGSGSGSGSAPTSSSTKKTQLQLEHLLLDLQ
MILNGINNYKNPKLTRMLTFKFYMPKKATELKHLQCLEEELKPLEEVLNLAQSKNFH
LRPRDLISNINVIVLELKGSETTFMCEYADETATIVEFLNRWITFCQSIISTLT
Name
CD19-IL2-007 (SEQ ID NO: 269)
MALPVTALLLPLALLLHAARPDIQMTQTTSSLSASLGDRVTISCRASQDISKYLNWY
QQKPDGTVKLLIYHTSRLHSGVPSRFSGSGSGTDYSLTISNLEQEDIATYFCQQGNTLP
YTFGGGTKLEITGGGGSGGGGSGGGGSEVKLQESGPGLVAPSQSLSVTCTVSGVSLP
DYGVSWIRQPPRKGLEWLGVIWGSETTYYNSALKSRLTIIKDNSKSQVFLKMNSLQT
DDTAIYYCAKHYYYGGSYAMDYWGQGTSVTVSSTTTPAPRPPTPAPTIASQPLSLRP
EACRPAAGGAVHTRGLDFACDIYIWAPLAGTCGVLLLSLVITLYCKRGRKKLLYIFK
QPFMRPVQTTQEEDGCCRFPEEEEGGCELRVKFSRSADAPAYKQGQNQLYNELNL
GRREEYDVLDKRRGRDPEMGGKPRRKNPQEGLYNELQKDKMAEAYSEIGMKGERR
RGKGHDGLYQGLSTATKDTYDALHMQALPPRGSGATNFSLLKQAGDVEENPGPGG
SMSHHWGYGKHNGPEHWHKDFPIAKGERQSPVDIDTHTAKYDPSLKPLSVSYDQAT
NLRILNNGHAFNVEFDDSQDKAVLKGGPLDGTYRLIQFHFHWGSLDGQGSEHTVDK
KKYAAELHLVHWNTKYGDFGKAVQQPDGLAVLGIFLKVGSAKPGLQKVVDVLDSI
KTKGKSADFTNFDPRGLLPESLDYWTYPGSLTTPPLLECVTWIVLKEPISVSSEQVLK
FRKLNFNGEGEPEELMVDNWRPAQPLKNRQIKASFKGSGGSGGSGGSGGSGGSGGS
GSSDYSDLQRVKQELLEEVKKELQKVKEEIIEAFVQELRKRGSGGSGGGSGGGSGMI
ETYNQTSPRSAATGLPISMKIFMYLLTVFLITQMIGSALFAVYLHRRLDKIEDERNLHE
DFVFMKTIQRCNTGERSLSLLNCEEIKSQFEGFVKDIMLNKEETKKENSFEMQKGSGS
GSGSGSGSGSAPTSSSTKKTQLQLEHLLLDLQMILNGINNYKNPKLTRMLTFKFYMP
KKATELKHLQCLEEELKPLEEVLNLAQSKNFHLRPRDLISNINVIVLELKGSETTFMC
EYADETATIVEFLNRWITFCQSIISTLT
Name
CD19-IL2-008 (SEQ ID NO: 270)
MALPVTALLLPLALLLHAARPDIQMTQTTSSLSASLGDRVTISCRASQDISKYLNWY
QQKPDGTVKLLIYHTSRLHSGVPSRFSGSGSGTDYSLTISNLEQEDIATYFCQQGNTLP
YTFGGGTKLEITGGGGSGGGGSGGGGSEVKLQESGPGLVAPSQSLSVTCTVSGVSLP
DYGVSWIRQPPRKGLEWLGVIWGSETTYYNSALKSRLTIIKDNSKSQVFLKMNSLQT
DDTAIYYCAKHYYYGGSYAMDYWGQGTSVTVSSTTTPAPRPPTPAPTIASQPLSLRP
EACRPAAGGAVHTRGLDFACDIYIWAPLAGTCGVLLLSLVITLYCKRGRKKLLYIFK
QPFMRPVQTTQEEDGCSCRFPEEEEGGCELRVKFSRSADAPAYKQGQNQLYNELNL
GRREEYDVLDKRRGRDPEMGGKPRRKNPQEGLYNELQKDKMAEAYSEIGMKGERR
RGKGHDGLYQGLSTATKDTYDALHMQALPPRGSGATNFSLLKQAGDVEENPGPGG
SMDMRVPAQLLGLLLLWLSGARCAPTSSSTKKTQLQLEHLLLDLQMILNGINNYKN
PKLTRMLTFKFYMPKKATELKHLQCLEEELKPLEEVLNLAQSKNFHLRPRDLISNINV
IVLELKGSETTFMCEYADETATIVEFLNRWITFCQSIISTLTGSGSGSGSGSGSGSSFEM
QKGGSLRPVQAQAQSDCSCSTVSPGVLAGIVMGDLVLTVLIALAVYFLGRLVPRGR
GAAEAATRKQRITEGGSGGGSGGGSGGGSGSSDYSDLQRVKQELLEEVKKELQKVK
EEIIEAFVQELRKRGSGSGGSGGSGGSGGSGGSGGSGMSHHWGYGKHNGPEHWHK
DFPIAKGERQSPVDIDTHTAKYDPSLKPLSVSYDQATNLRILNNGHAFNVEFDDSQD
KAVLKGGPLDGTYRLIQFHFHWGSLDGQGSEHTVDKKKYAAELHLVHWNTKYGDF
GKAVQQPDGLAVLGIFLKVGSAKPGLQKVVDVLDSIKTKGKSADFTNFDPRGLLPES
LDYWTYPGSLTTPPLLECVTWIVLKEPISVSSEQVLKFRKLNFNGEGEPEELMVDNW
RPAQPLKNRQIKASFK
Name
IL2-017 (SEQ ID NO: 271)
MDMRVPAQLLGLLLLWLSGARCAPTSSSTKKTQLQLEHLLLDLQMILNGINNYKNP
KLTRMLTFKFYMPKKATELKHLQCLEEELKPLEEVLNLAQSKNFHLRPRDLISNINVI
VLELKGSETTFMCEYADETATIVEFLNRWITFCQSIISTLTGSGSGSGSGSGSGSGSGS
GSGSGSGSGSGSKQEHFPDNLLPSWAITLISVNGIFVICCLTYCFAPRCRERRGSSHHW
GYGKHNGPEHWHKDFPIAKGERQSPVDIDTHTAKYDPSLKPLSVSYDQATSLRILNN
GHAFNVEFDDSQDKAVLKGGPLDGTYRLIQFHFHWGSLDGQGSEHTVDKKKYAAE
LHLVHWNTKYGDFGKAVQQPDGLAVLGIFLKVGSAKPGLQKVVDVLDSIKTKGKS
ADFTNFDPRGLLPESLDYWTYPGSLTTPPLLECVTWIVLKEPISVSSEQVLKFRKLNF
NGEGEPEELMVDNWRPAQPLKNRQIKASFKGSGATNFSLLKQAGDVEENPGPMGTS
LLCWMALCLLGADHADACPYSNPSLCSGGGGSELPTQGTFSNVSTNVSPAKPTTTAC
PYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVV

### Example 18. Oligomerizing IL18 Constructs

TABLE 5
Constructs Used in Example 18

| | |
|---|---|
| Thy1.2-001 | Thy1.2 only |
| IL15-448 | Thy1.2—P2A—mbIL15 |
| IL18-013 | mbIL18 (B7.1 TM) |
| IL18-015 | secIL15—P2A—Thy1.2 |
| IL18-016 | mbIL18(CD40LTM-shed)—P2A—Thy1.2 |
| IL18-017 | mbIL18(CD40LTM-non shed)—P2A—Thy1.2 |
| IL18-018 | mbIL18(CD40LTM-shed)—P2A—mbIL15— |
| | P2A—Thy1.2 |
| IL18-019 | mbIL18(CD40LTM-non shed)—P2A— |
| | mbIL15—P2A—Thy1.2 |
| IL18-030 | secIL18—P2A—mbIL15—P2A—Thy1.2 |
| IL18-033 | mbIL18 (Col7/CD40LTM)CA2(L156H)— |
| | P2A—Thy1.2 |
| IL18-037 | mbIL18 (B7.1TM) CA2(L156H)—P2A— |
| | Thy1.2 |
| IL18-038 | mbIL18 (B7.1TM) ctdegron CA2(L156H)— |
| | P2A—Thy1.2 |
| IL18-039 | mbIL18 B7.1(hinge-TM-ERlinker)CA2(L156H)— |
| | P2A—Thy1.2 |
| IL18-040 | mbIL18(phospholambdaTM(shed))CA2(L156H)— |
| | P2A—Thy1.2 |
| IL18-041 | mbIL18(Col7 Light TM)CA2(L156H)—P2A— |
| | Thy1.2 |
| IL18-042 | mbIL18 (B7.1TM) CA2(L156H) (improved |
| | shed)—P2A—Thy1.2 |
| IL18-043 | mbIL18 (B7.1TM) ctdegron CA2(L156H) |
| | (improved shed)—P2A—Thy1.2 |
| IL18-044 | mbIL18 B7.1(hinge-TM-ERlinker)CA2(L156H) |
| | (improved she)—P2A—Thy1.2 |
| IL18-047 | pELNS-Kv7.4-CA2(L156H)-CD40LTMhinge- |
| | mbhIL18(shed)-P2a-Thy1.2 |
| IL18-048 | pELNS-CA2(S56N)-(GGSG)3-41BBL-(GS)7- |
| | IL18-P2A-Thy1.2 |
| IL18-049 | pELNS-IL18cs2-P2A-Thy1.2 |
| IL18-050 | pELNS-collectin7-CA2(L156H)- |
| | CD40LTMhinge (Shed)-mbhIL18cs2-P2a- |
| | Thy1.2 |
| IL18-051 | pELNS-CD40LTM-hinge-mbhIL18cs2(non- |
| | shed)-p2a-Thy1.2 |
| IL18-052 | pELNS-LKC-IL18cs2-B7-1(cyto-TM-tail)- |
| | CA2(L156H)-P2a-Thy1.2 |
| IL18-053 | mbIL18 (B7.1TM) CA2(L156H)—P2A— |
| | mbIL15—P2A—Thy1.2 |
| IL18-054 | mbIL15—P2A—mbIL18 (B7.1TM) |
| | CA2(L156H)—P2A—Thy1.2 |
| IL18-055 | mbIL18 (B7.1TM) CA2(L156H) (improved |
| | shed)—P2A mbIL15—P2A—Thy1.2 |
| IL16-056 | mbIL15—P2A—mbIL18 (B7.1TM) |
| | CA2(L156H) (improved shed)—P2A—Thy1.2 |
| IL18-057 | pELNS-LKC-IL18-CD4hinge-TM- |
| | CYTO + linker-CA2-linker-collectin7-P2A- |
| | QCD4 |
| IL18-058 | pELNS-DAP12sp-DAP12hinge_TM_cyto- |
| | CA2DRD(L156H)-p2a-NKG2C(cyto-TM- |
| | hinge)-IL18-P2A-QCD4 |
| IL18-059 | pELNS-LKCsp-IL18-GS(10)-DAP12-hinge- |
| | TM-Cyto-CA2(L156H)-p2a-QCD4 |
| IL18-060 | pELNS-CA2(L156H)-IL18sp-IL18-p2a-QCD4 |
| IL18-061 | pELNS-Collectin7-(linker 21aa)-CA2(L156H)- |
| | IL18sp-IL18-p2a-QCD4 |
| IL18-062 | pELNS-LKCsp-IL18-GS(10)-EphA2-hinge- |
| | TM-Cyto-CA2(L156H)-p2a-QCD4 |
| IL18-063 | pELNS-LKCsp-IL18-GS(10)-FGFR3-hinge- |
| | TM-Cyto-CA2(L156H)-p2a-QCD4 |
| IL18-064 | pELNS-LKCsp-IL18-GS(10)-VEGFR2-hinge- |
| | TM-Cyto-CA2(L156H)-p2a-QCD4 |
| IL18-065 | pELNS-LKCsp-IL18-GS(10)-ERBB2-hinge- |
| | TM-Cyto-CA2(L156H)-p2a-QCD4 |
| IL18-066 | pELNS-LKC-IL18-EPOR-hinge-TM- |
| | CYTO + linker-CA2(L156H)-linker-collectin7- |
| | P2A-QCD4 |
| IL18-067 | pELNS-LKC-IL18-B7-1-hinge-TM- |
| | CYTO + linker-CA2(L156H)-linker-collectin7- |
| | P2A-QCD4 |
| IL18-068 | pELNS-LKC-IL18-linker-collectin7-linker- |
| | EPOR-hinge-TM-CYTO + linker-CA2(L156H)- |
| | P2A-QCD4 |
| IL18-069 | pELNS-LKC-IL18-linker-collectin7-linker-B7- |
| | 1-hinge-TM-CYTO + linker-CA2(L156H)-P2A- |
| | QCD4 |
| IL18-070 | pELNS-LKC-IL18-linker-collectin7-linker- |
| | CD4hinge-TM-CYTO + linker-CA2-P2A-QCD4 |
| IL18-071 | pELNS-LKC-IL18-linker-collectin7FL-linker- |
| | B7-1-hinge-TM-CYTO + linker-CA2(L156H)- |
| | P2A-QCD4 |

### T Cell Transduction

On day 1, primary human T cells were stimulated with Dynabeads (T-expander CD3/CD28) at a 3:1 bead:cell ratio in media containing 10% fetal bovine serum (FBS). The next day, lentiviral vectors were used to transduce the T cells with IL18 oligomerizing constructs. On day 3, the cells were diluted 1:2 with fresh 10% FBS media. On day 4, cells were split 1:4 with fresh 10% FBS media.

On day 6, cells were de-beaded and re-plated at 1×10⁶ cells/mL. The next day cells were left unstimulated or activated with soluble CD3/CD28 (StemCell Technologies) and were treated with either 50 μM acetazolamide (ACZ) or DMSO. After 24 hours, anti-IL18 (Clone 159-12B, MBL Int Cat #D045-3) and anti-Rat IgG2a PE-conjugated Biolegend, Cat #407508) was used to detect membrane-bound IL18 by flow cytometry. Cytokines that had accumulated for 24 hours in the culture supernatants (from 200,000 cells per 200 μL media) were measured using human IL18 MSD V-plex assay kits (Meso Scale Discovery).

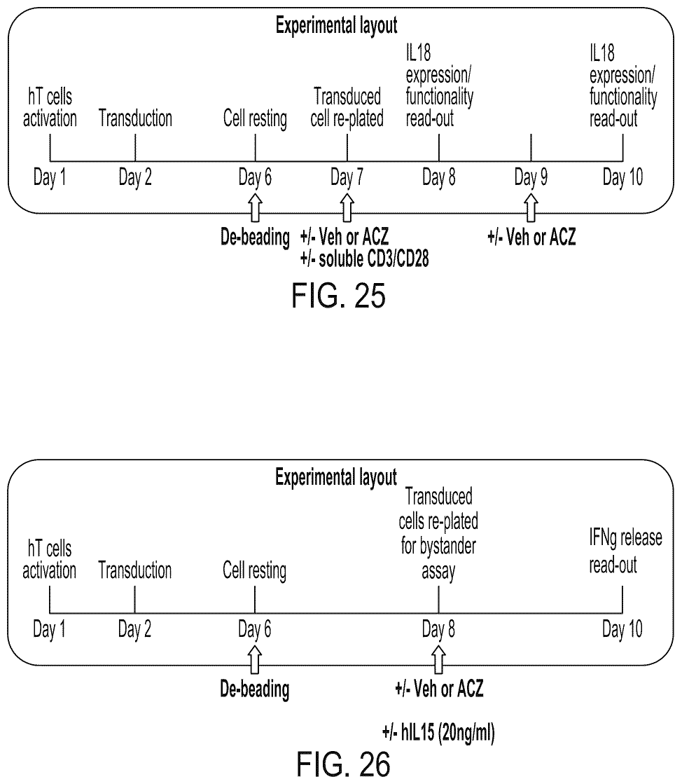

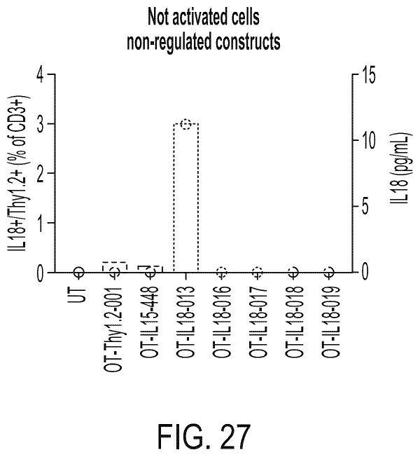

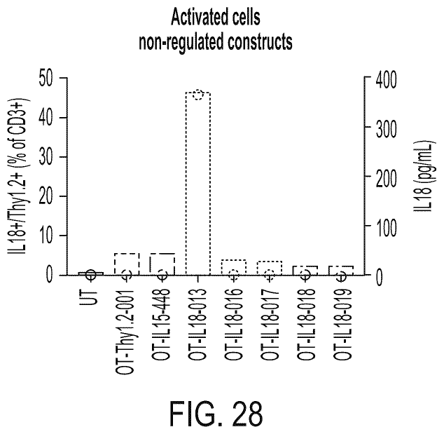

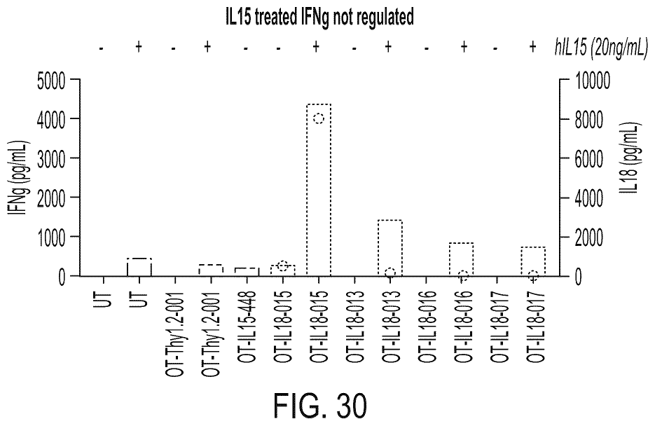

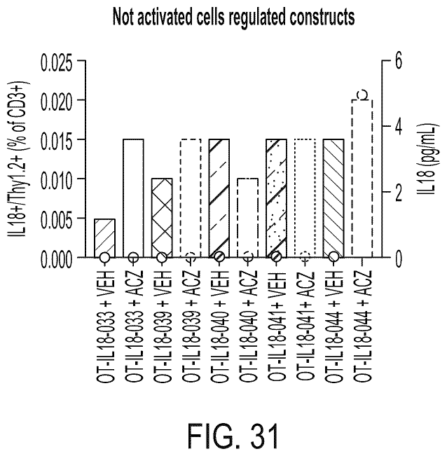

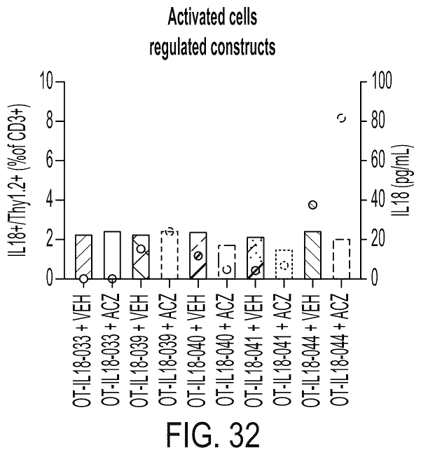

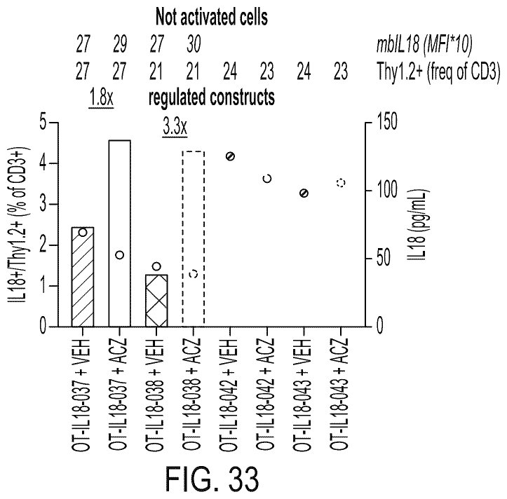

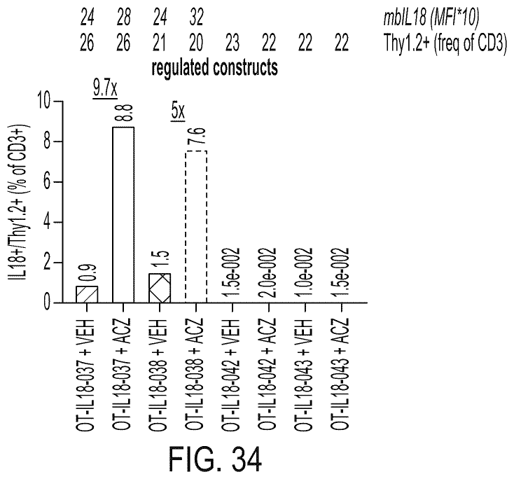

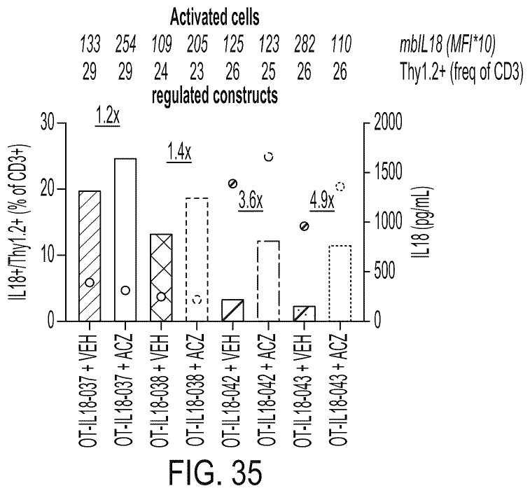

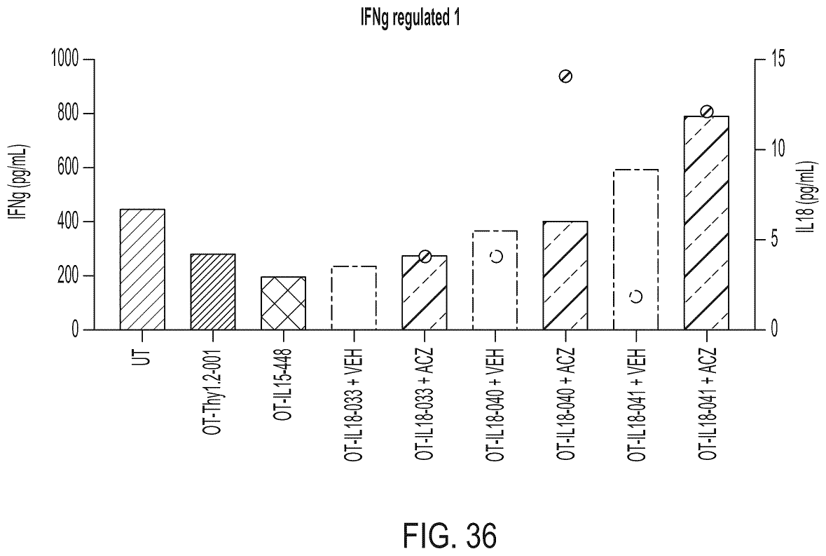

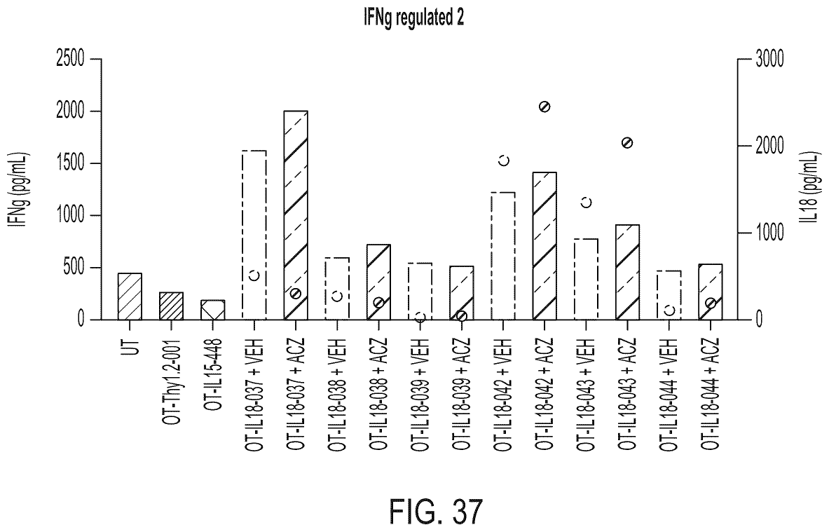

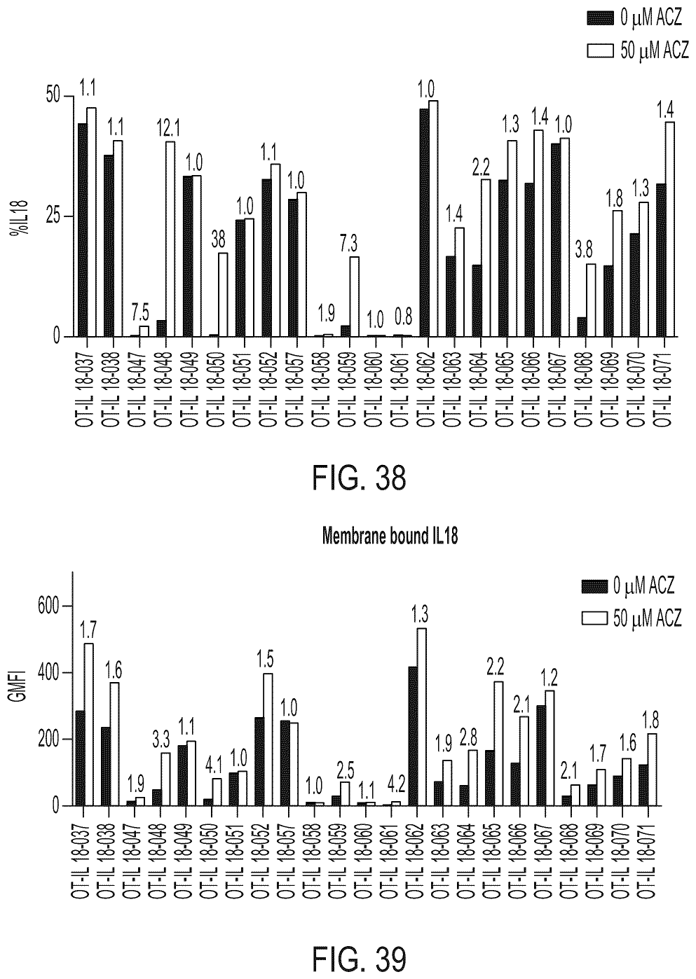

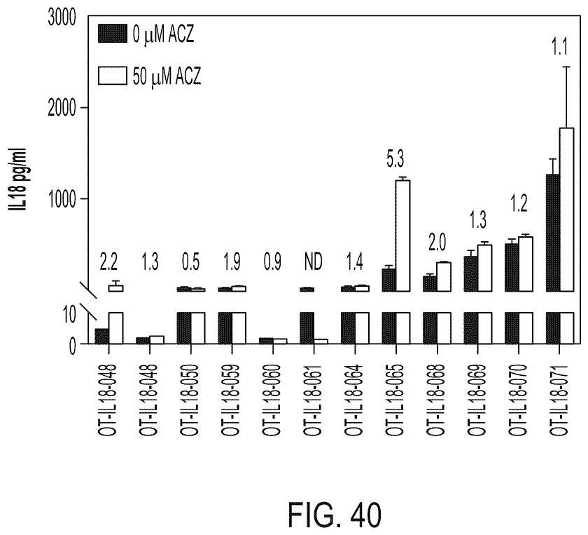

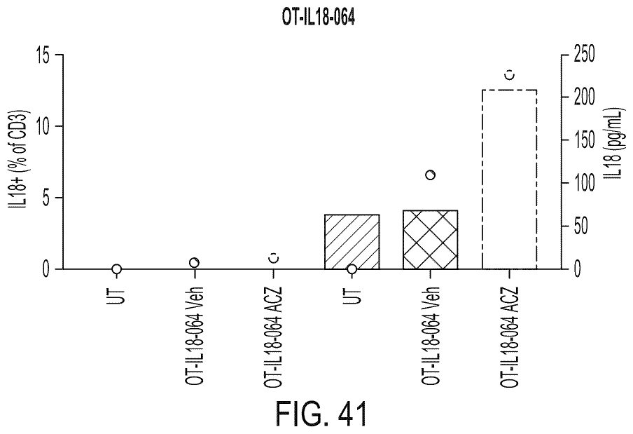

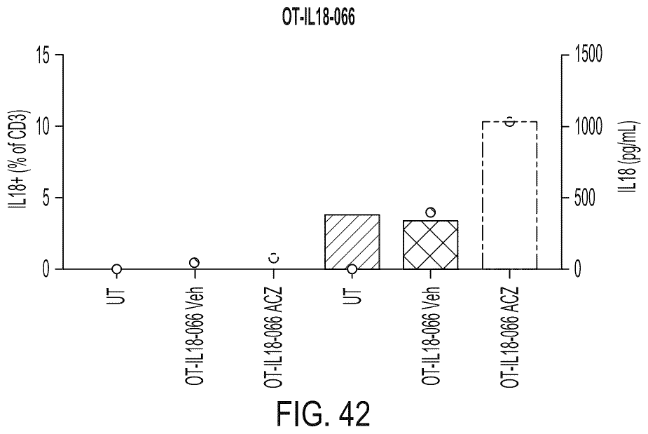

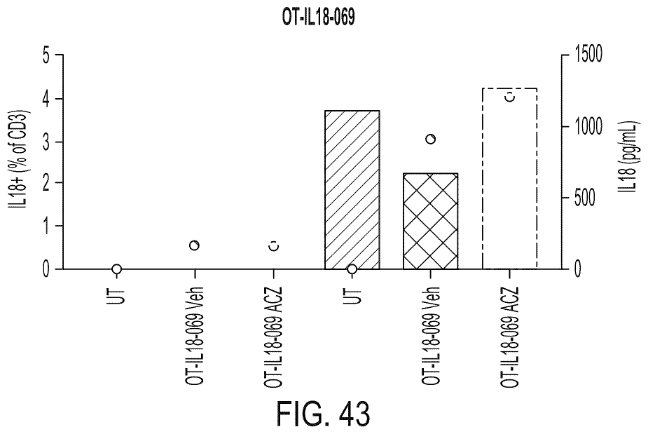

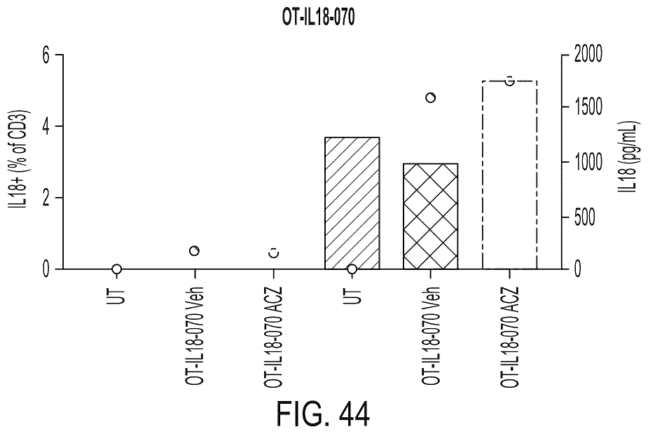

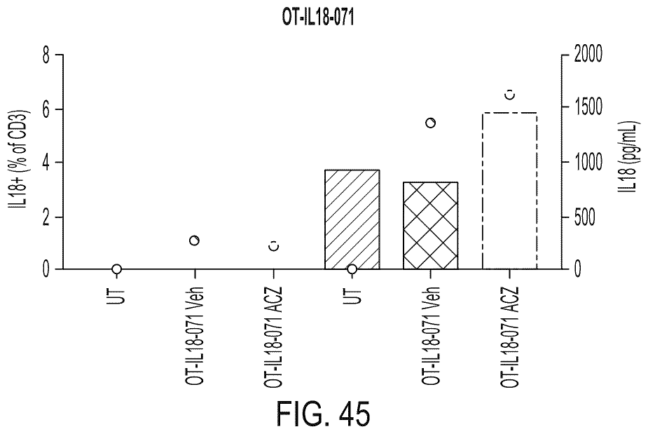

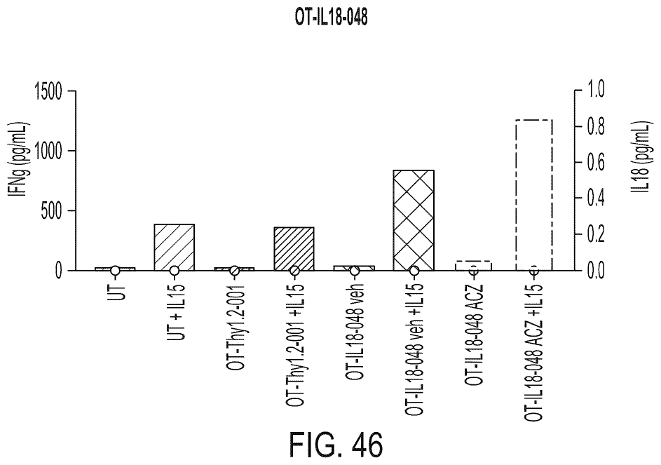

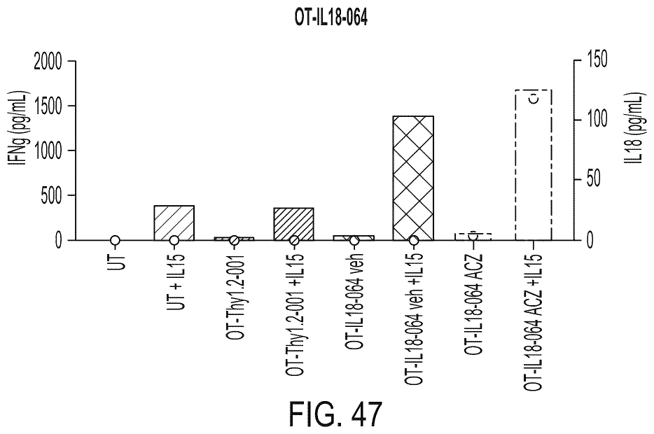

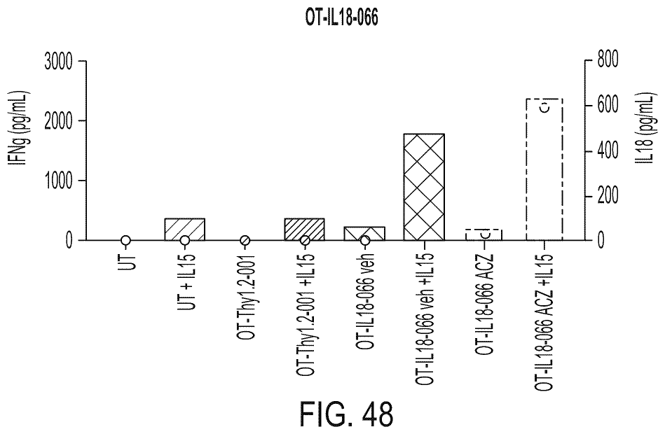

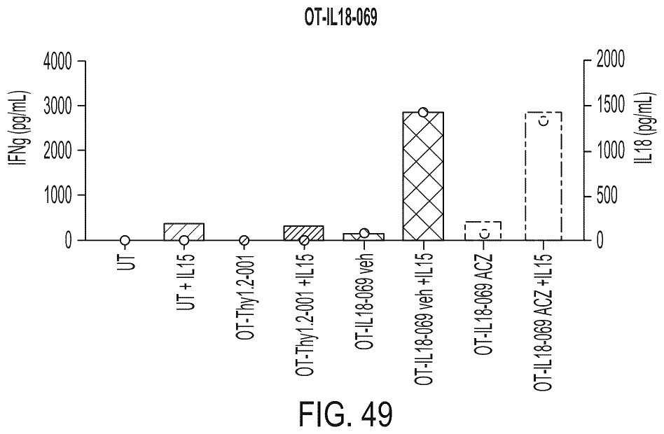

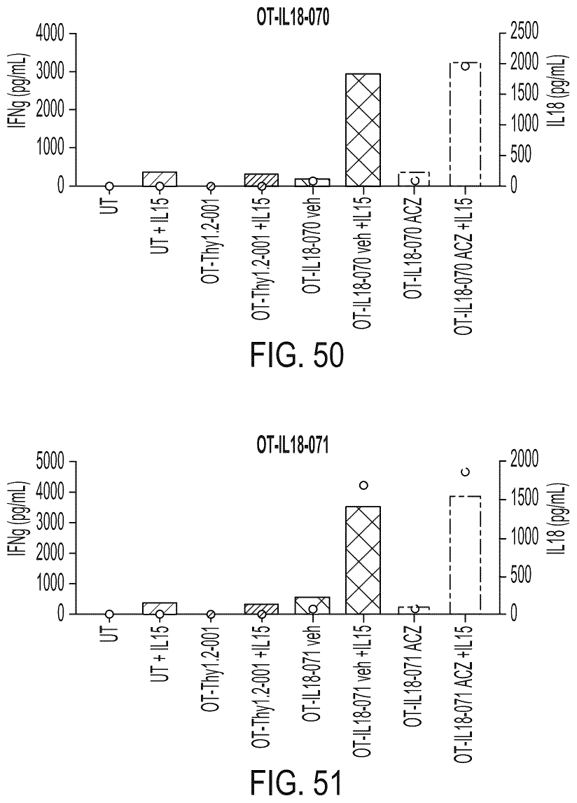

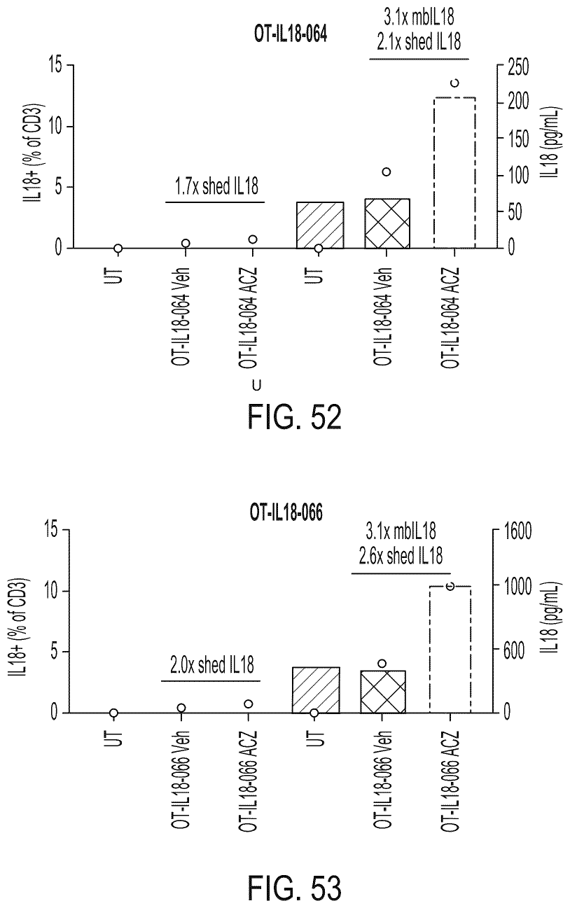

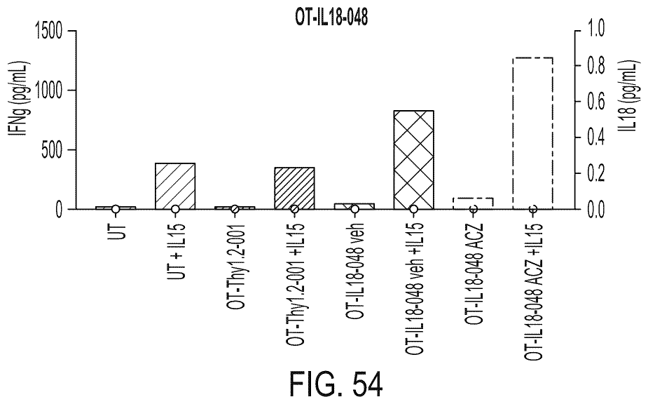

FIG. 25 is a schematic showing the experimental design for determining IL18 expression, and FIG. 26 is a schematic showing the experimental design for determining IFNγ release. FIGS. 27, 28, 31-32, 33-35, 38-45, 52-53 show the frequency of IL18-expressing T cells in the presence and absence of ACZ. For FIGS. 38-40, the numbers above the ACZ-treated cells represent fold change between on and off-state in the presence or absence of ACZ. In FIGS. 27-33, 35-37, and 41-54, figures with open circles refer to the left y-axes and show IL18 released into the supernatant. The IL18 constructs containing a hinge with a defined shedding-site resulted in an amplified dynamic range for regulation of the shed IL18 fraction in the cell supernatants.

### Antigen-Independent Interferon-Gamma Release

On day 1, primary human T cells were stimulated with Dynabeads (T-expander CD3/CD28) at a 3:1 bead:cell ratio in medium containing 10% fetal bovine serum (FBS). The next day, lentiviral vectors were used to transduce the T cells with IL18 oligomerizing constructs. On day 3, the cells were diluted 1:2 with fresh 10% FBS medium. On day 4, cells were split 1:4 with fresh 10% FBS medium.

On day 6, cells were de-beaded and re-plated at 1×10⁶ cells/mL and rested for 48 hours. On day 8, cells were re-plated at 200,000 cells per 200 μL medium and treated or untreated with recombinant 20 ng/ml of human IL15 (Biolegend, Cat #570304). Interferon (IFN)-γ and IL18 that had accumulated for 48 hours in the culture supernatants was measured using customized human IFNγ/IL18 MSD V-plex assay kits (Meso Scale Discovery).

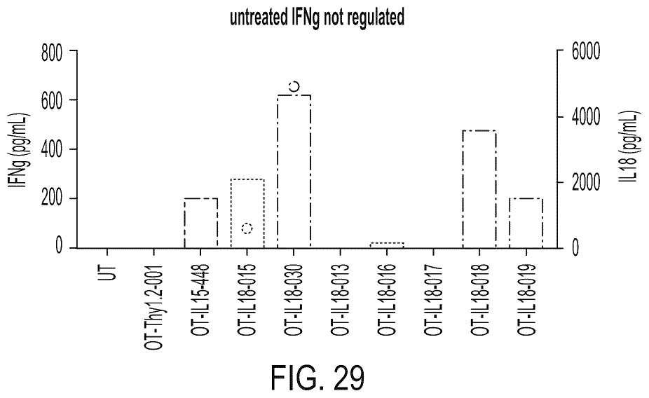

FIGS. 29, 30, 36, 37, 46-51, and 54 are graphs showing the amount of IFNγ released from transduced T cells treated or untreated with recombinant IL15. Circles refer to the right y-axes and show the amount of IL18 released into the supernatant.

### Jurkat Transduction

Jurkat, Clone E6-1, cells (ATCC) were cultured in standard media (RPMI+ GlutaMAX Supplement: Life Technologies Cat #61870036, Fetal Bovine Serum (FBS) Life Technologies Cat #A38400-01). Jurkat cells were transduced with lentiviral vectors containing IL18 oligomerizing constructs. Five days post transduction, the cells were treated with 50 μM acetazolamide or DMSO, and the cells were further cultured for 1 more day. Anti-IL18 (Clone 159-12B, MBL Int Cat #D045-3) and anti-Rat IgG2a PE-conjugated (Biolegend, Cat #407508) were used to detect membrane-bound I-18 by flow cytometry.

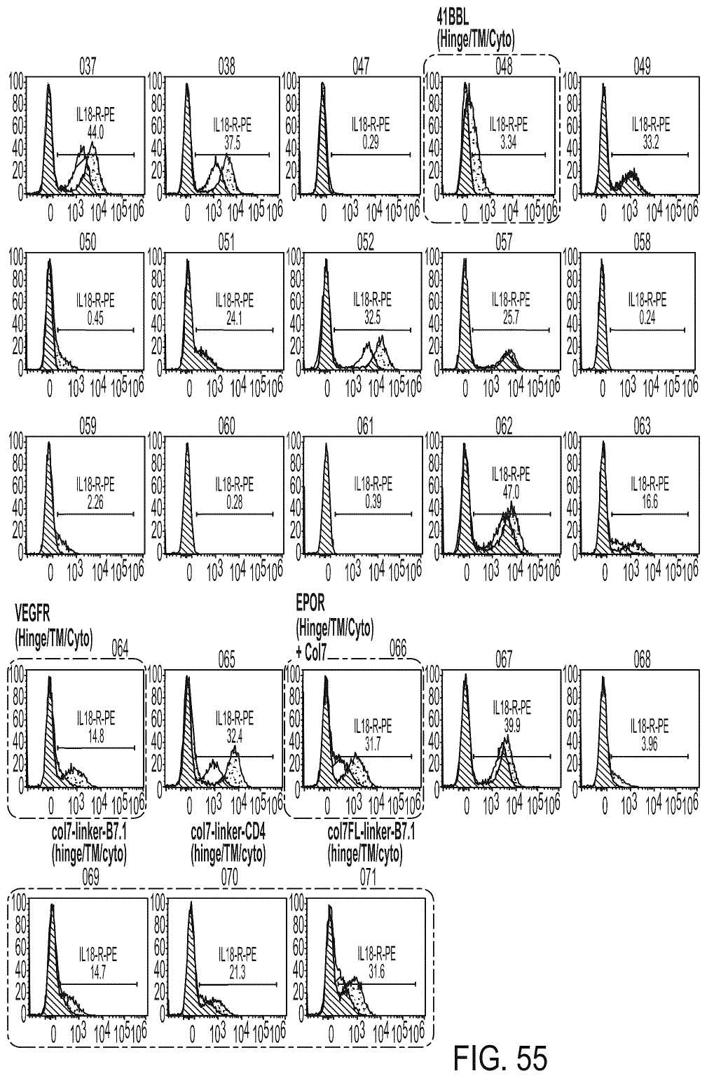

FIG. 55 shows flow cytometry histograms indicating change in mbIL18 expression levels on Jurkat cells in the presence and absence of ACZ. The numbers above the ACZ-treated cells represent fold change between IL18 levels observed in the presence versus absence of ACZ (the on versus the off-state). MFI was calculated on cells within the mCherry gate.

In summary, mbIL18 synergizes with mbIL15 or recombinant IL15 to induce IFNγ release in T cells. IL18 constructs IL18-037 and IL18-042 show 2 to 3-fold regulation and ability to synergize with recombinant IL15 to induce IFNγ release in T cells. The IL18-042 construct shows IL18 activity also in media collected from transduced activated cells, supporting potential pleiotropic effect.

### Example 19. Pharmacologically Controlled Expression of Membrane-Bound IL12 Results in T-Cell Therapy with Enhanced Potency in Preclinical Solid Tumor Models

TABLE 6
Constructs Used in Example 19

| CD19-063 | CD19-CAR only control |
|---|---|
| CD19-IL12-192 | CD19-CAR-P2A-IL12—(GS)15—B7.1 |
| | Hinge—B7.1 TM/Tail—CA2(L156H) |
| CD19-IL12-302 | CD19-CAR-P2A-Collectin 7—CA2 (S56N)— |
| | CCD40L TM—Shed CD40L Hinge—IL12 |
| CD19-IL12-333 | CD19-CAR-P2A-Collectin 7—CA2 (L156H)— |
| | CD40L TM—Shed CD40L Hinge—IL12 |
| CD19-IL12-345 | CD19-CAR-P2A-Collectin 7— |
| | CA2 (L156H)—CD40L TM—Shed |
| | CD40L Hinge—Codon Depotimized IL12 |

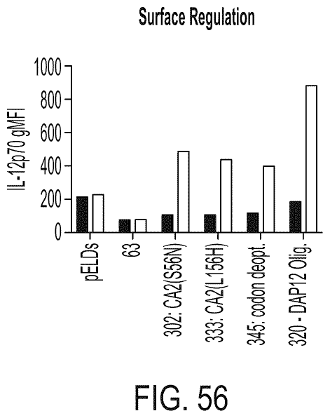

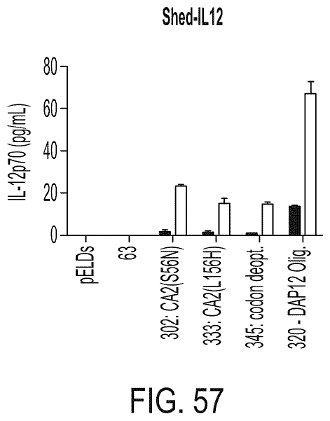

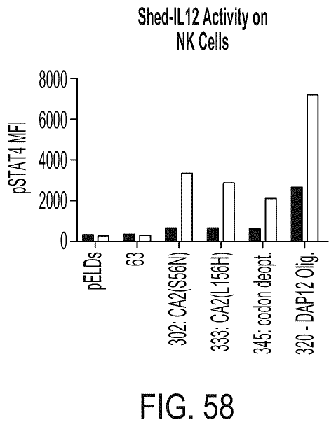

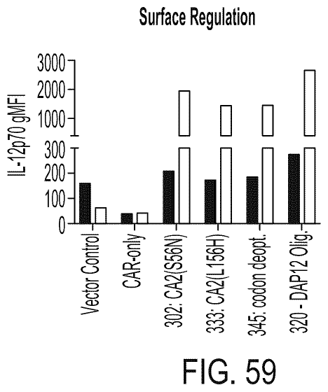

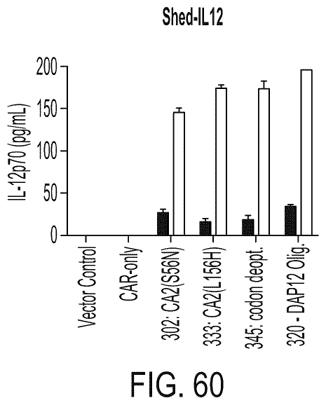

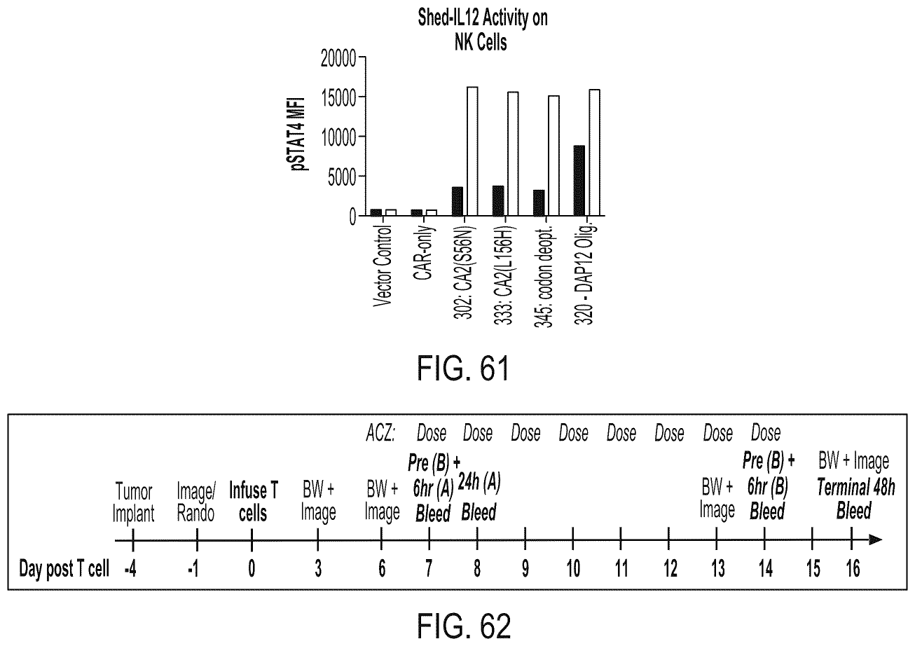

Constructs 333 and 345 were characterized in vitro. FIGS. 56-61 show geometric MFI indicative of IL12 abundance on the surfaces of CAR-T cells transduced with control (192) and IL12 constructs (302, 333, 345), demonstrating regulation of IL12 function and lower basal off-state levels of the IL12 constructs containing the collectin 7 oligomerization domain as compared to control monomeric constructs. Also shown are shedding levels of IL12 detected using Meso-scale discovery (MSD) assays and activity of the shedding component on NK cells. FIGS. 56-58 are graphs showing in resting T cells that collectin-7 trimerizing constructs have a low off-state with regulation of shed-IL12 levels (FIG. 57) that demonstrate functional activity on bystander natural killer (NK) cells (FIG. 58). FIGS. 59-61 are graphs showing that, in activated T cells stimulated with CD3/CD28 for 20 hours, IL12 constructs containing collectin-7 trimerizing domains show a low basal off-state with regulated shed-IL12 levels (FIG. 60) and in vitro functional activity (phosphorylation of STAT4) against bystander NK cells (FIG. 61).

For in vivo studies, NOD SCID gamma mice were implanted intravenously with 100,000 cells from the CD19+ Raji Burkitt's lymphoma cell line stably expressing luciferase to track tumor growth. The mice were randomized into treatment groups for equal tumor burden three (3) days post tumor implantation. Four days post tumor implantation, different cell doses of CD19-CAR-T cells bearing various IL12 constructs were treated via adoptive cell transfer (ACT). Mice were bled 7 days post ACT to provide a baseline of cytokine levels and cell expansion before dosing with ACZ with oral gavage. ACZ dosing and bleeds continued as described in FIG. 62 until the end of the study on day 16 post ACT. IL12 was regulated on the surface of CD19+ cells, with constructs 302, 333, and 345 showing a greater ability to regulate the surface abundance than control monomeric construct 192. Similarly, IL12 in the plasma was regulated with the 302, 333 and 345 constructs, but not control construct showing up to a 54× regulation using ACZ.

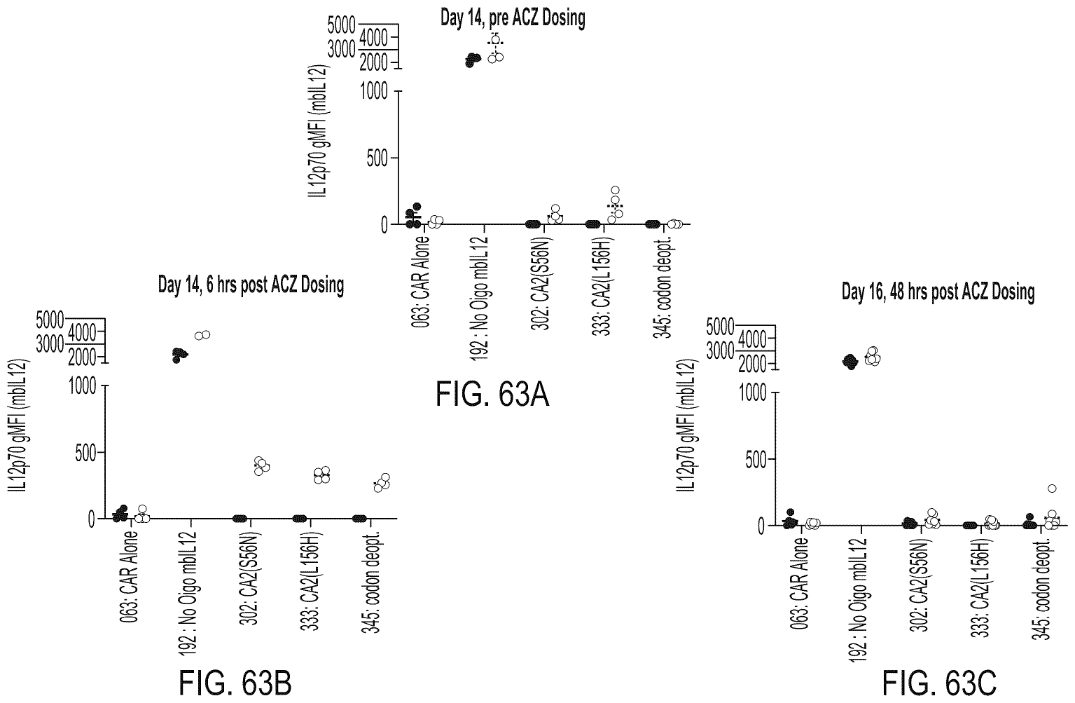

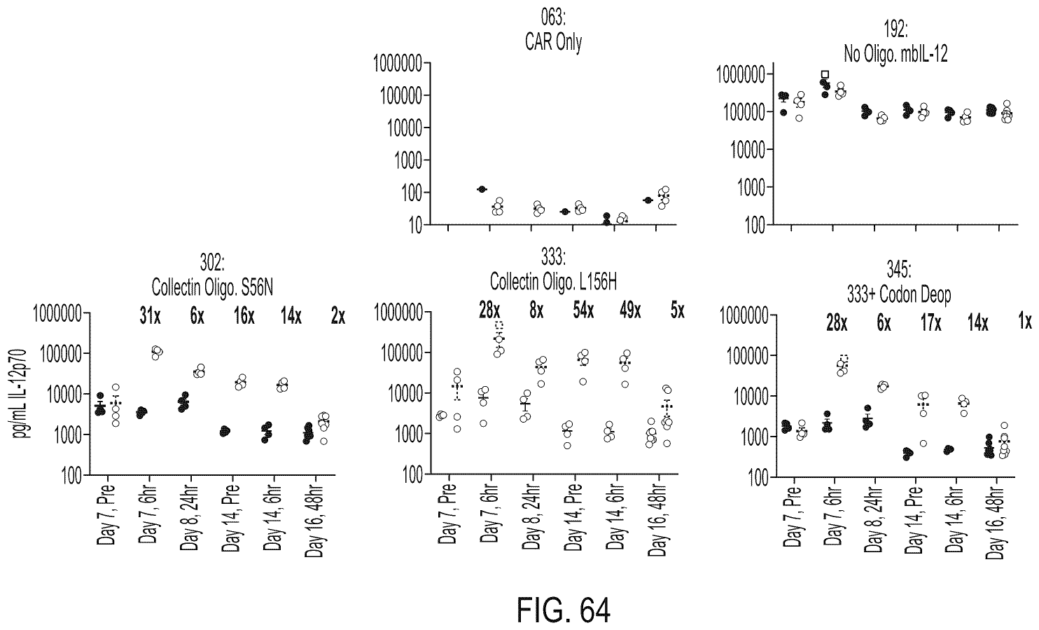

As shown in FIGS. 63 and 64, IL12 is regulated in vivo. FIG. 63 shows that IL12 is regulated on the surface and FIG. 64 shows that it is also regulated in the plasma. FIG. 63 shows that IL12 was regulated on the surface of CD19+ cells, with constructs 302, 333, and 345 showing a greater ability to regulate the surface abundance than control monomeric construct 192. Similarly, FIG. 64 shows that IL12 in the plasma was regulated with the 302, 333 and 345 constructs, but not the CAR-only (063 construct) or monomeric control (192 construct) showing up to a 54× regulation using ACZ.

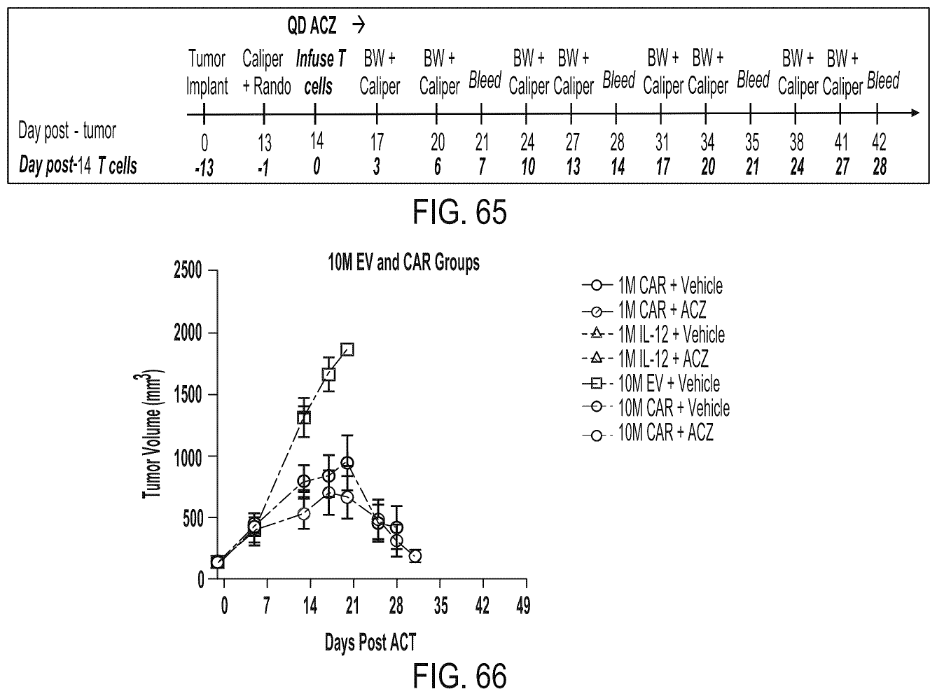

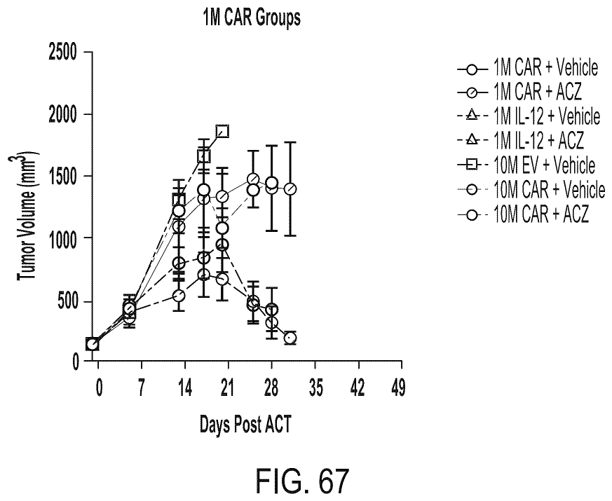

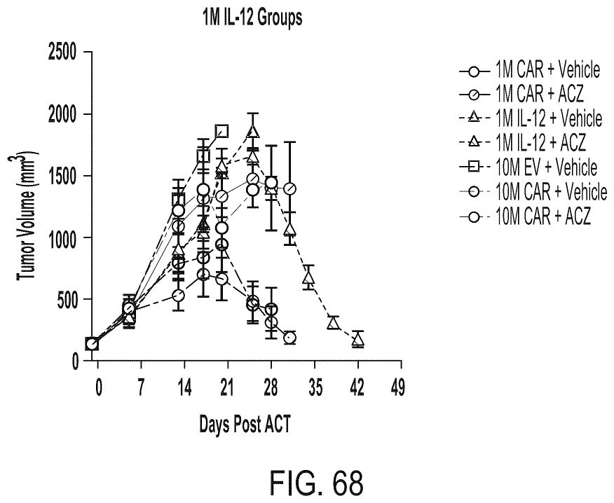

For the scRaji study, NOD SCID gamma mice were implanted subcutaneously with 5 million Raji cells—a CD19+ Burkitt's lymphoma cell line. The mice were randomized into treatment groups for equal tumor volume (measured using digital calipers) 13 days post tumor implantation. Fourteen (14) days post tumor implantation, CD19-CAR-T cells modified with control constructs 063 (CAR-Alone), EV (Empty vector) or with construct 345 were dosed for adoptive cell transfer (ACT). Mice received continuous daily dosing of ACZ or vehicle starting on the same day as ACT. Mice were bled and measured as described in the study design (FIG. 65). Cell expansion and phenotype in the blood was measured using flow cytometry and cytokine levels in the plasma determined using Meso Scale Discovery assays. Mice were euthanized upon reaching endpoints of tumor volume (2000 mm3), hind limb paralysis, or evidence of graft verses host disease. Animals dosed with CAR-T cells containing construct 345 showed efficacy in the ON state and ability to control large solid tumors. Subcutaneous Raji tumors were measured twice weekly using digital calipers. Mice treated with CAR-T cells modified with construct 345 showed ACZ regulated efficacy (in terms of tumor burden) at doses 10× lower than those modified with the CAR-only control construct 063 (FIGS. 66-68). FIGS. 66-68 show infusion of modulation hub IL12-expressing CAR-T cells (construct 345) provided regulated anti-tumor activity at suboptimal doses of CAR-T cells.

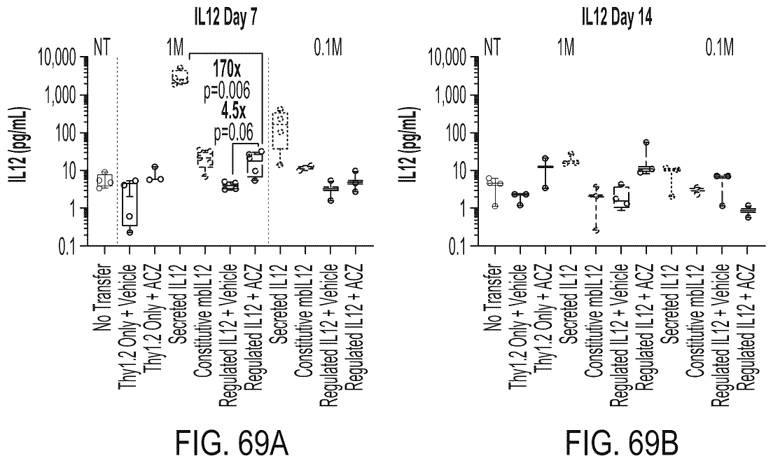

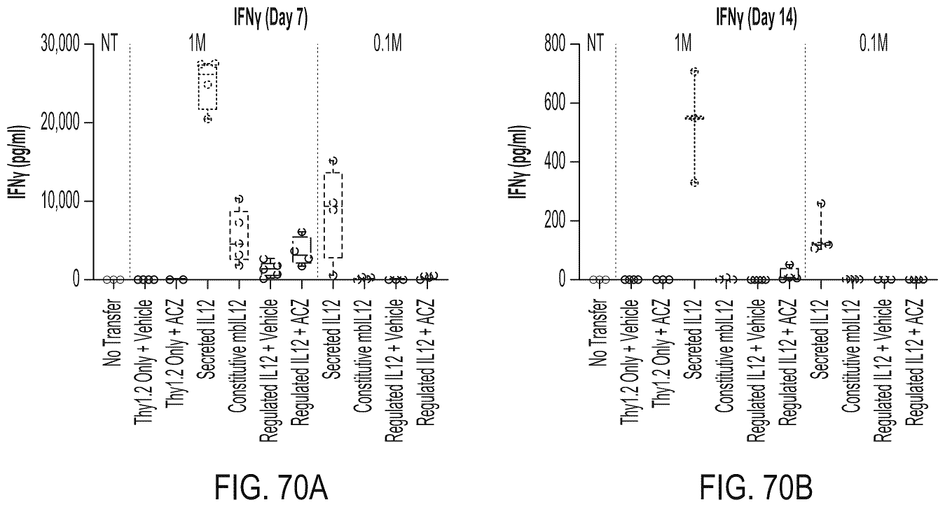

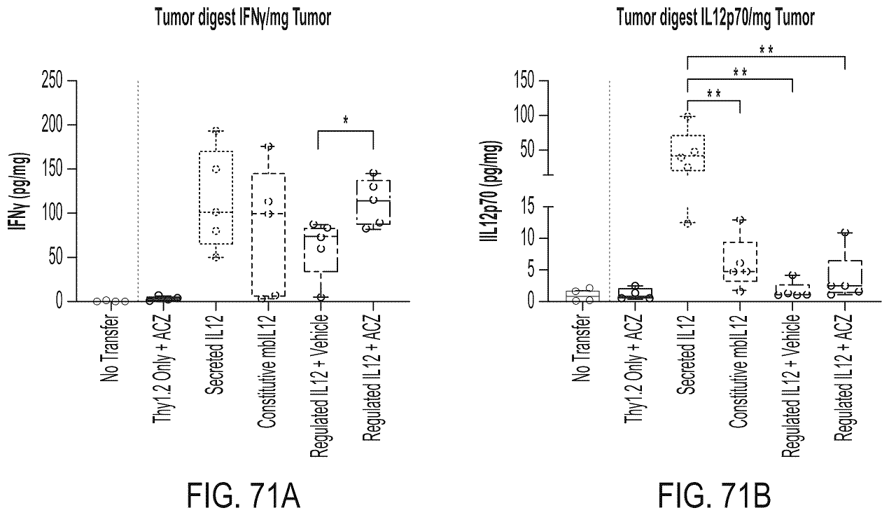

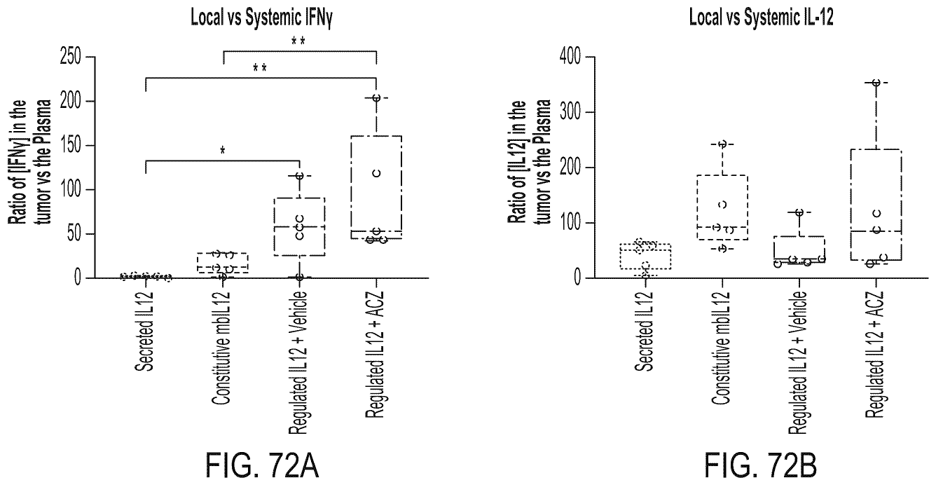

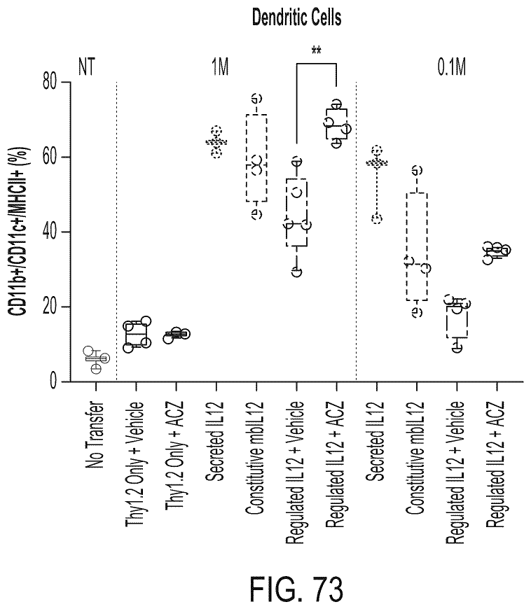

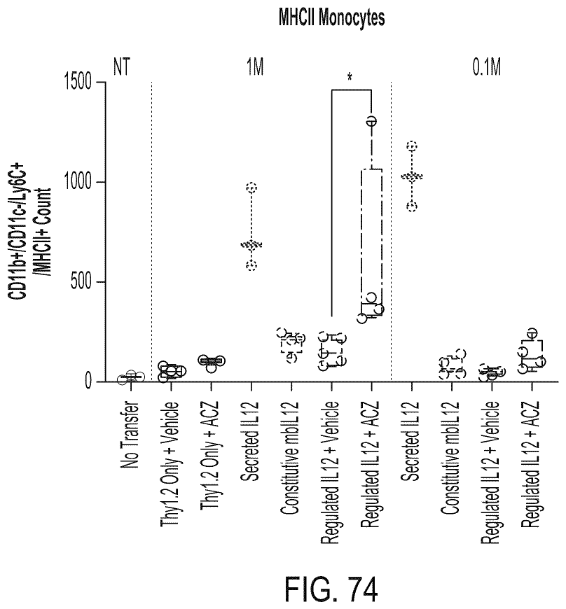

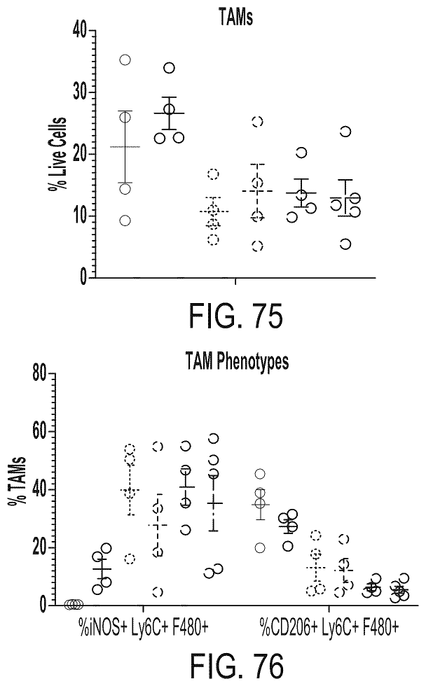

For the PMEL study (FIGS. 69-78), C57BL/6 mice were implanted subcutaneously with 500,000 B16-F10 (PMEL antigen positive) melanoma cells. The mice were randomized into treatment groups for equal tumor burden (measured using digital calipers) 13 days post tumor implantation and the endogenous immune system depleted using intravenous Cytoxan. Fourteen (14) days post tumor implantation, PMEL antigen specific T-cells with or without IL12 armoring were treated via adoptive cell transfer (ACT) with surrogate murine IL12 constructs. Mice received continuous daily dosing of ACZ or vehicle starting on day of ACT. Mice were bled and measured as shown in FIG. 65. Cell expansion and phenotype in the blood were measured using flow cytometry and cytokine levels were determined using MSD assays. On day 7 post ACT, tumors, spleens, and tumor draining lymph nodes (TDLNs) were harvested, digested and analyzed using flow cytometry. Cytokine levels in the tumor after digestion were determined using Meso Scale Discovery assays. Regulated mbIL12 construct, which is the surrogate murine analogue of human construct 333, demonstrated IL12 and IFNγ localization to the tumor and regulation in the plasma (FIGS. 69-72). Modulation hub IL12 constructs enabled regulation of IL12 in the plasma and the tumor. In animals receiving Pmels cells engineered with modulation hub IL12 constructs, IL12 and IFNγ levels displayed a greater degree of localization (greater ratio of tumor versus plasma cytokines) than with non-regulated (lacking the DRD CA2) secreted or membrane-bound IL12. This ACZ-driven regulation of IL12 using modulation hub IL12 constructs had functional impacts in terms of regulating the frequency of circulating dendritic cells (FIG. 73), the phenotype of activated monocytes in circulation (FIG. 74) and showing secretion of IL12 and IFNγ levels in the tumor but not systemically (FIGS. 69-72). As shown in FIGS. 73 and 74, MHC II monocytes on day 14 post ACT from the study showed that the ACZ-induced regulation of IL12 with constructs having the modulation hubs led to regulated phenotypes (MHCII+ activated monocytes) in the circulation. As shown in FIGS. 75-78, phenotypes of the myeloid population in the tumor and systemically in animals receiving Pmel cells transduced with modulation hub IL12 constructs show the same M1 to M2 and phenotype switch in the tumor microenvironment as animals receiving Pmel cells transduced with the secreted, nonregulated IL12 construct. These phenotype changes were more localized (occurring more at the tumor site than within the spleen or lymph node) for the modulation hub IL12 than secreted or constitutive mbIL12 as evidenced by PD-L1 upregulation on myeloid cell populations in the spleen and TDLN, but a similar degree of PD-L1 upregulation in the spleen.

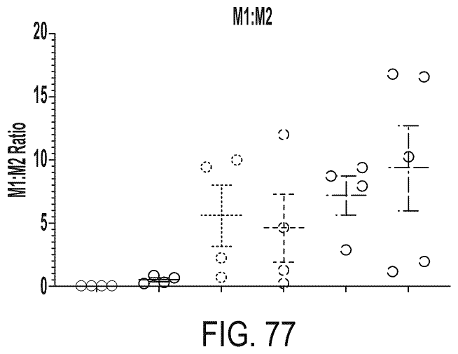

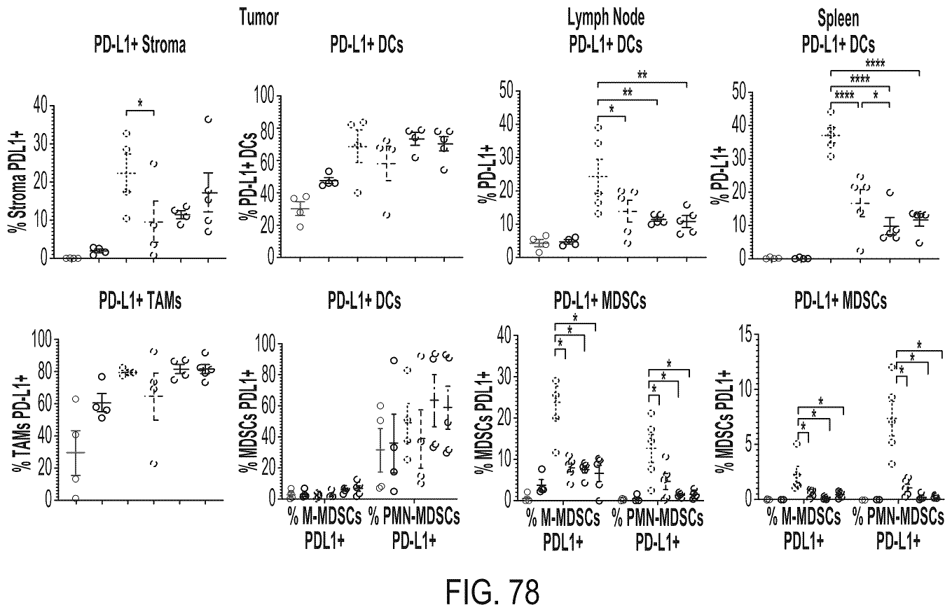

To summarize, IL12 was mostly localized in the tumor using membrane-bound regulated constructs, whereas regulated IL12 and IFNγ were observed systemically. In the blood, it was found that the regulated modulation hub IL12 had functional impacts. The on-state IL12 levels (in the plasma and the tumor) produced by adoptively transferred Pmel cells engineered with modulation hub IL12 constructs were closer to IL12 levels in animals receiving Pmel cells expressing constitutive mbIL12 constructs than IL12 levels in animals in which modulation hub IL12 was left in the off-state (no doses of ACZ). Likewise, phenotypes such as MHCII expression on circulating monocytes were regulated by ACZ in animals receiving Pmels cells engineered with modulation hub IL12 (FIG. 74). Functional impacts were also observed in the tumor microenvironment (TME). For example, modulation hub IL12 promoted switching of the M1-M2 phenotype of macrophages to a similar extent as nonregulated, secreted IL12 (FIG. 77). However, nonregulated, secreted IL12 produced different effects systemically than modulation hub IL12. For example, nonregulated, secreted IL12 led to significantly greater PD-L1 expression on dendritic cells and myeloid derived suppressor cells (MDSC) in the spleen and lymph nodes than modulation hub IL12 (FIG. 78).

### Example 20. IL12 Constructs with PDE5

TABLE 7
Constructs Used in Example 20

| | |
|---|---|
| IL12-229 | pELNS-IL12-(GS)15-sCD8hinge-(B7-1TM)- |
| | TYCFAPRCRERRRGS- |
| | hPDE5[R732L]-p2A-mCherry |
| IL12-235 | pELNS-IL12-(GS)15-CD8ahinge-CD8TM- |
| | LYCNHRNRRRVCKGS- |
| | hPDE5[R732L]-p2A-mCherry |
| IL12-274 | pElLNS-hPDE5(R732L)-(GGSG)3- |
| | CD40L(non-shedding)-(GS)7-IL12-P2A-mCherry |
| IL12-275 | pELNS-IL12Flex-(GS)8-DAP12(Hinge + TM + Cyto)- |
| | hPDE5(R732L)-P2A-mCherry |
| IL12-276 | pELDS-hPDE5(R732L)—(GGSG)3-CD40L- |
| | collectin7a-(GS)7-IL12-P2A-mCherry |

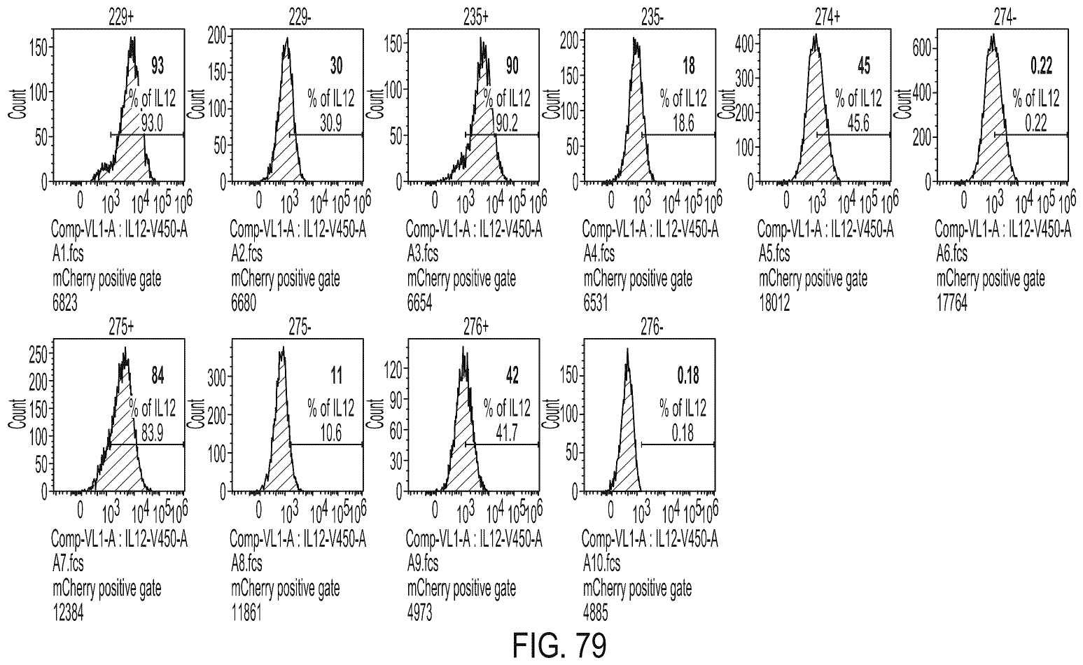

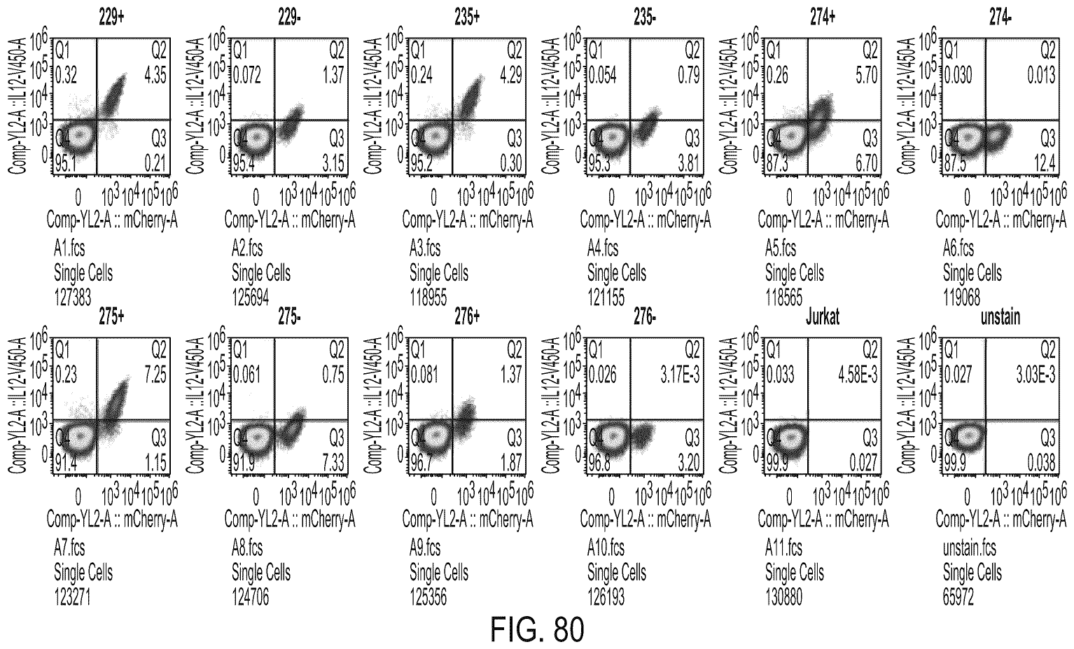

Three oligomerization concepts were tested with PDE5 as the DRD. Two control constructs IL12-229 and IL12-235 were included in the test. In IL12-274, IL12 was directly fused with CD40L and the natural shedding site of CD40L was abrogated. IL12-275 uses the DAP12 transmembrane hinge. IL12-276 was oligomerized with a collectin 7 helix trimer, except the collectin 7 was placed intracellularly. Flow cytometry analysis shown in FIGS. 79 and 80 demonstrate that performance is similar to oligomerized CA2 DRD-IL12 fusions, with a reduction in basal abundance.

### Example 21. Regulation of IFNα in Tumor Infiltrating Lymphocytes in Combination with Constitutive Membrane-Bound IL15 (mbIL15)

TABLE 8
Constructs Used in Example 21

| IL15-292 | mbIL15 |
|---|---|
| IFNα-003 | pELNS-CA2(L156H)-(GGSG)3- |
| | CD40Lcyto-phospholambancyto-tm- |
| | CD40Lhinge(shed)-(GS)7-IFNα- |
| | P2AmbIL15_B7-1-P2A-CD4Q |
| IFNα-007 | pELNS-collectin7-(GSG)6-CA2(L156H)- |
| | (ggsg)3-CD40Lcyto-TM-hinge(shed)- |
| | (GS)7-IFNα-IL15(292)-CD4Q (from CD19-IFNα-004) |
| IFNα-015 | pELNS-IFNα-P2A-IL15(292) |
| IFNα-016 | pELNS-collectin7-(GSG)6-CA2(L156H)- |
| | (GGSG)3-light(cyto-TM-hinge)- |
| | GS7-IFNα-P2A-IL15(292) |
| IFNα-018 | pELNS-mbIL15_B7-P2A-collectin7- |
| | (GSG)6-CA2(L156H)-(GGSG)3- |
| | light(cyto-TM-hinge)-GS7-IFNα |

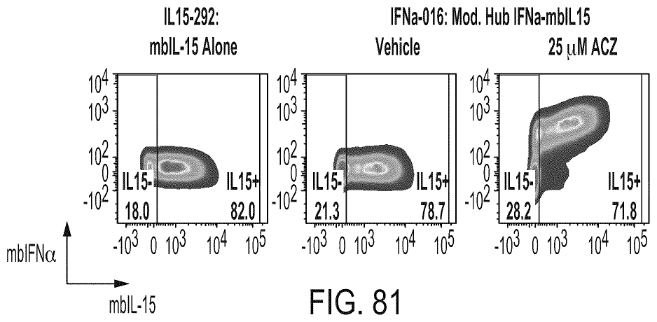

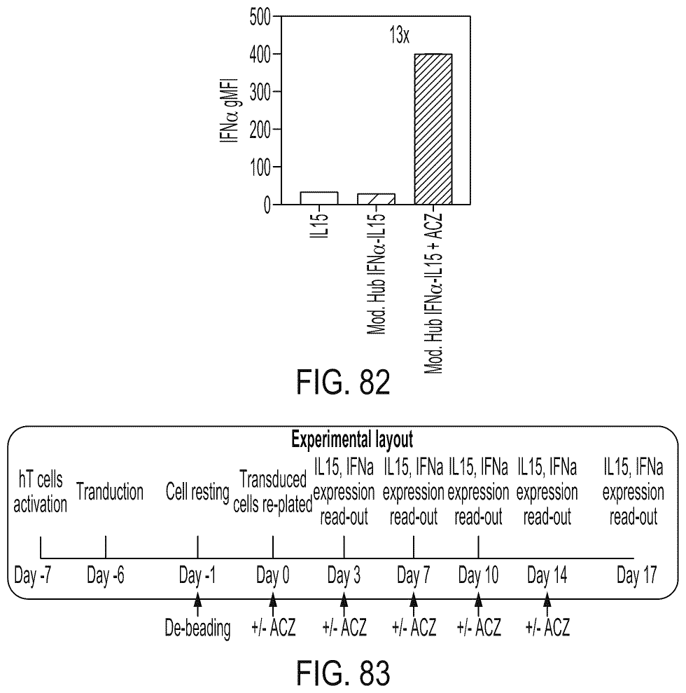

Tumor infiltrating lymphocytes (TILs) were isolated from tumors, expanded, and transduced with a control construct (Construct 292) containing only membrane-bound IL15 (mbIL15) and a construct containing both constitutive mbIL15 and a CA2-modulation hub regulated form of IFNα (Construct 016). Transduced cells (500,000) were transferred into a GREX device and allowed to expand for 2 weeks according to a rapid expansion protocol with or without 25 μM ACZ. At the end of that 2 weeks, the TILs were analyzed for surface abundance of IFNα using flow cytometry (BD Biosciences Cat #560097). The results are shown in FIGS. 81 and 82. Graphs showing geometric median fluorescence intensity (geoMFI) indicative of membrane-bound IFNα abundance on the surface of tumor infiltrating lymphocytes transduced with control (Construct 292) and IFNα experimental (Construct 016) constructs, demonstrating regulation of IFNα with ACZ added throughout the rapid expansion protocol. Regulation is shown on the final day of TIL expansion.

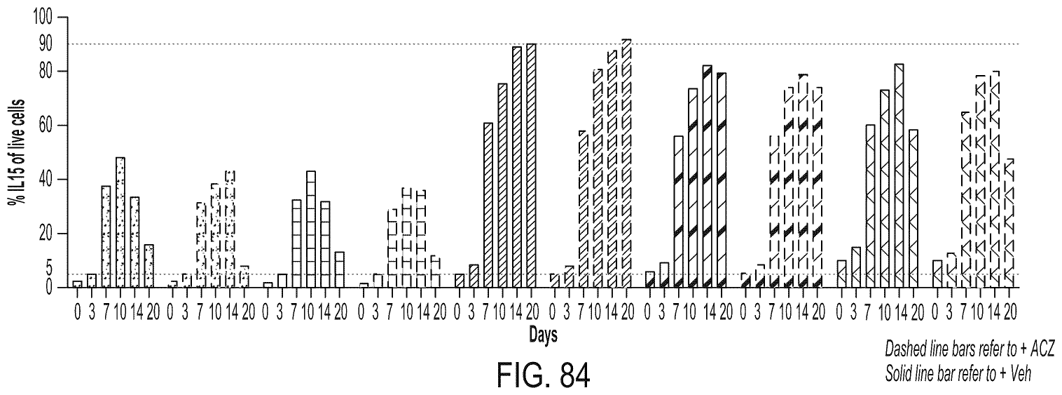

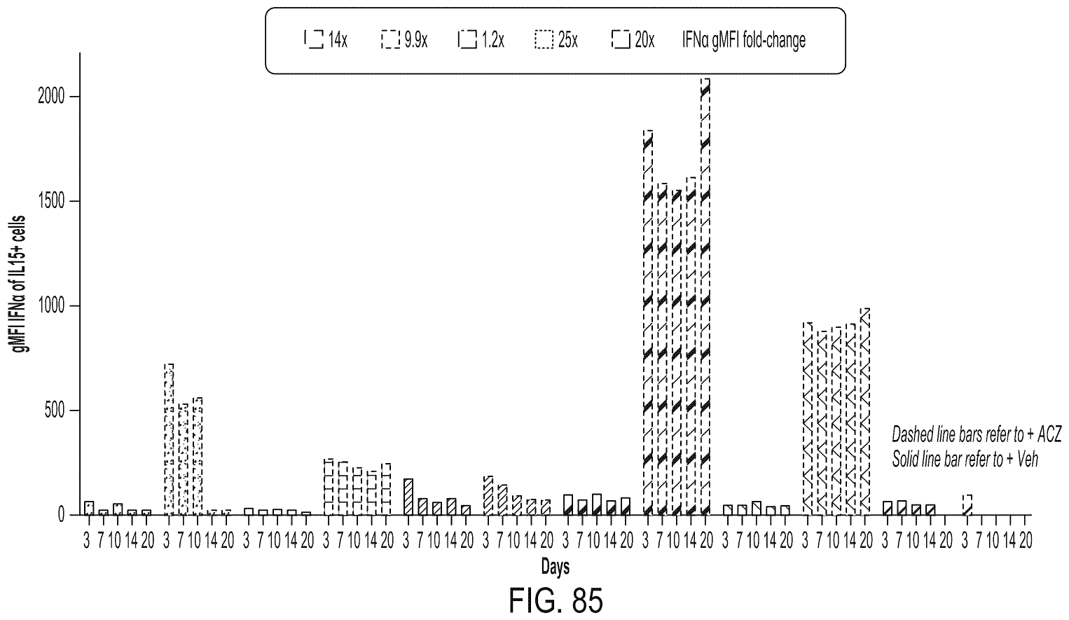

For FIGS. 83-85, on day 7, primary human T cells were stimulated with Dynabeads (T-expander CD3/CD28) at a 3:1 bead:cell ratio in medium containing 10% fetal bovine serum (FBS). The next day, lentiviral vectors were used to transduce the T cells with IL15-IFNα oligomerizing constructs. On day 5, the cells were diluted 1:2 with fresh 10% FBS medium. On day 4, cells were split 1:2 with fresh 10% FBS medium. On day 1, cells were de-beaded and re-plated at 1×10⁶ cells/mL and rested for 24 hours. On day 0, cells were re-plated at 500,000 cells per 1000 μL medium and treated or untreated with 50 μM of ACZ. The expression of membrane-bound IL15 and membrane-bound IFNα was measured at day 3, 7, 10, 14 and 20. As shown in FIGS. 83-85, mbIL15 expressing cells accumulated over time and IFNα was regulated approximately 20-fold in IFNα-016 and -018 constructs.

In another round of experiments, human TILs were expanded and engineered with lentiviral vectors to express IL15 with IFNα, IL18 (IL1 family member) (see Example 18) or TNFSF-X (TNF superfamily member). Expanded TILs were immunophenotyped and assessed for polyfunctionality by flow cytometry after CD3/CD28 stimulation. Engineered TILs were transferred into NSG mice to assess antigen-independent TIL persistence in the absence of exogenous IL2. Cytokines modified with our carbonic anhydrase 2 (CA2)-based drug responsive domain (DRD) were evaluated for control of protein levels with the CA2 ligand, acetazolamide (ACZ). Cytokine expression was evaluated in flow cytometry and Meso Scale Discovery assays.

Engineered TILs expressing both IL15 and either IFNα, IL18 or TNFSF-X showed similar fold expansion, immunophenotype, and polyfunctionality in vitro as TILs expressing only IL15. Combination cytokine-expressing TILs showed similar in vivo antigen-independent persistence in the absence of IL2 as TILs engineered with only IL15. As compared to control cells, sub-optimal cell doses of T cells expressing both IL15 and either IFNα or IL18 showed improved efficacy and TME remodeling, whereas combining IL15 with TNFSF-X resulted in significant tumor growth arrest of B16 melanoma tumors without escape.

IL15 drives expansion and persistence of cytoTIL15TM cells without IL2, and adding pleotropic and highly immune-stimulatory members of the IFN, IL1, or TNF families provides enhanced efficacy for patients with solid tumors marked by an immunosuppressive TME.

### Example 22. Jurkat Transduced IL18 Constructs

TABLE 9
Constructs used in Example 22.

| | |
|---|---|
| IL18-072 | CA2(L156H)-4-1BBL (cyto-TM-hinge)-collectin7trimer- |
| | CD40Lshedsite-IL18-p2a-IL15(292) |
| IL18-073 | Collectin7-CA2(L156H)-4-1BBL (cyto-TM-hinge)- |
| | CD40Lshedsite-IL18-p2a-IL15(292) |
| IL18-074 | CA2(L156H)-CD70L (cyto-TM-hinge)-collectin7trimer- |
| | CD40Lshedsite-IL18-p2a-IL15(292) |
| IL18-075 | Collectin7-CA2(L156H)-CD70L (cyto-TM-hinge)- |
| | CD40Lshedsite-IL18-p2a-IL15(292) |
| IL18-076 | CA2(L156H)-CD30L (cyto-TM-hinge)-collectin7trimer- |
| | CD40Lshedsite-IL18-p2a-IL15(292) |
| IL18-077 | Collectin7-CA2(L156H)-CD30L (cyto-TM-hinge)- |
| | CD40Lshedsite-IL18-p2a-IL15(292) |
| IL18-078 | LKCsp-IL18-GS(10)-VEGFR2[hinge-TM(3T-->C)-Cyto]- |
| | CA2(L156H)-p2a-mbIL15 |
| IL18-079 | LKCsp-IL18-GS(10)-VEGFR2[hinge-TM(6E-->C)-Cyto]- |
| | CA2(L156H)-p2a—mbIL15 |
| IL18-080 | LKCsp-IL18-GS(10)-VEGFR2[hinge-TM(31V-->C)-Cyto]- |
| | CA2(L156H)-p2a—mbIL15 |
| IL18-081 | LKCsp-IL18-GS(10)-VEGFR2[hinge-TM(11V-->E)-Cyto]- |
| | CA2(L156H)-p2a—mbIL15 |
| IL18-082 | LKCsp-IL18-GS(10)-VEGFR2[hinge-TM(12G-->E, |
| | 19F-->E)-Cyto]-CA2(L156H)-p2a—mbIL15 |
| IL18-083 | LKCsp-IL18-GS(10)-VEGFR2[wt]-CA2(L156H)- |
| | p2a—mbIL15 |

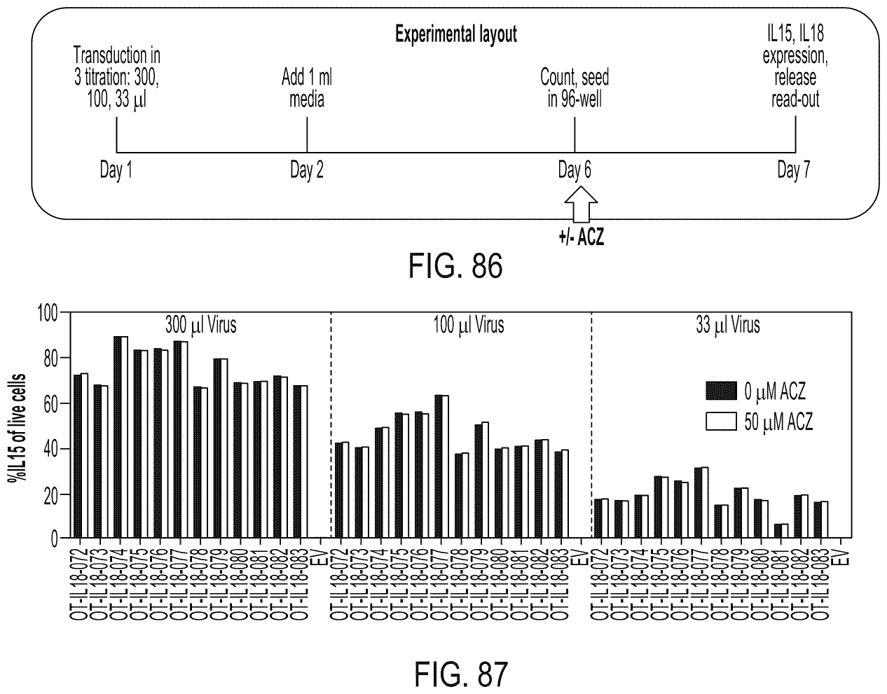

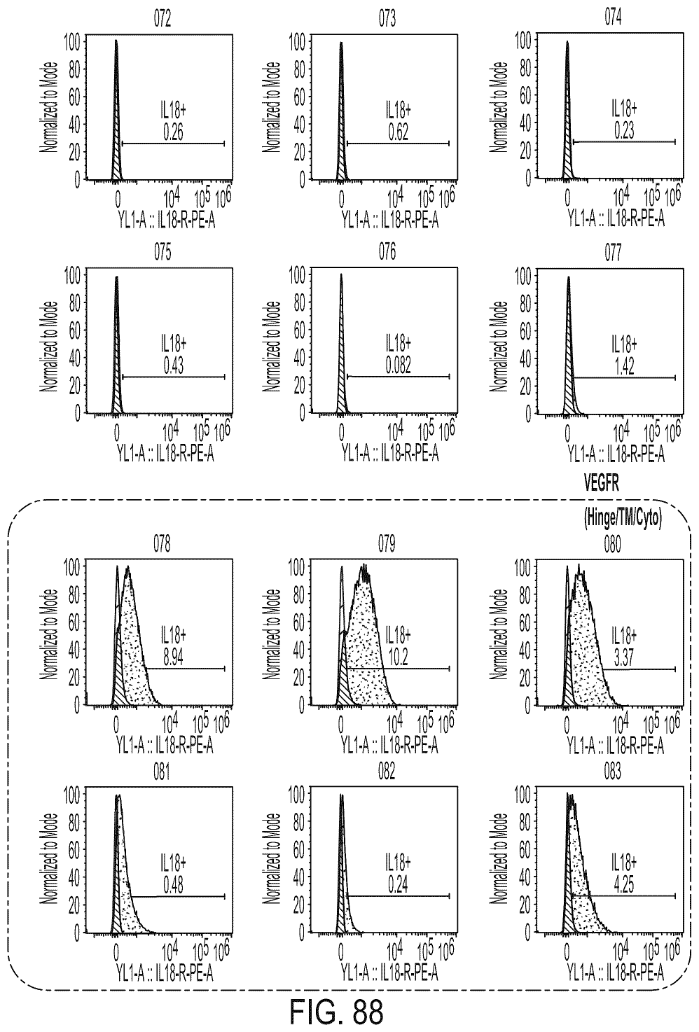

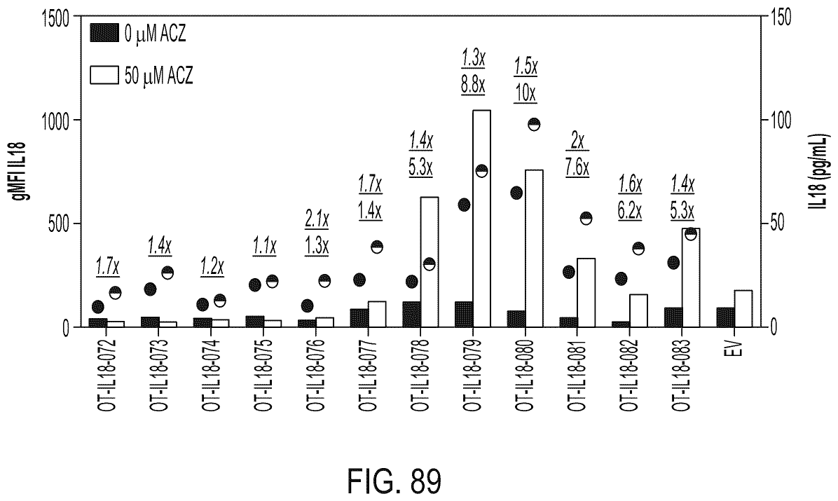

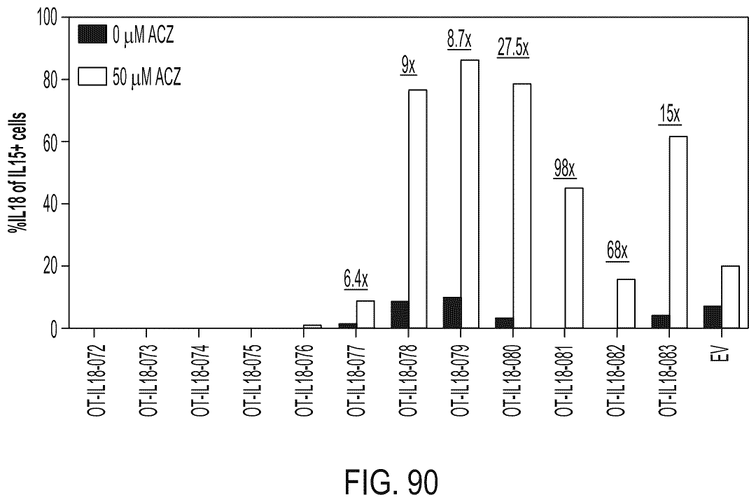

The testing protocol is shown in FIG. 86. As shown in FIGS. 87-90, mbIL18 was regulated 10× in IL18-080 construct. Released IL18 was regulated about 2× in constructs IL18-076 and IL18-082. All constructs with collectin7 showed very low IL18 basal with no/low regulation that might be due to low mbIL18 expression that was below our detection range. VEFGR containing constructs showed nice regulation specially in 100 μl virus titration where it had lowest basal the regulation was as high as 10× for IL18-080.

TABLE 10
IL18 fold change comparison table.

| Name | % IL15 | FC mbIL18 | FC Shed IL18 |
|---|---|---|---|
| IL18-072 | 42 | 0.7 | 1.65 |
| IL18-073 | 40 | 0.5 | 1.42 |
| IL18-074 | 49 | 0.9 | 1.17 |
| IL18-075 | 55 | 0.6 | 1.08 |
| IL18-076 | 56 | 1.3 | 2.12 |
| IL18-077 | 63 | 1.4 | 1.67 |
| IL18-078 | 37 | 5.3 | 1.37 |
| IL18-079 | 50 | 8.8 | 1.27 |
| IL18-080 | 40 | 10.0 | 1.51 |
| IL18-081 | 41 | 7.6 | 1.96 |
| IL18-082 | 43 | 6.2 | 1.61 |
| IL18-083 | 38 | 5.3 | 1.44 |

### Example 23. Oligomerization in Cytoplasmic Settings: Trimerization and Tetramerization of SOX2

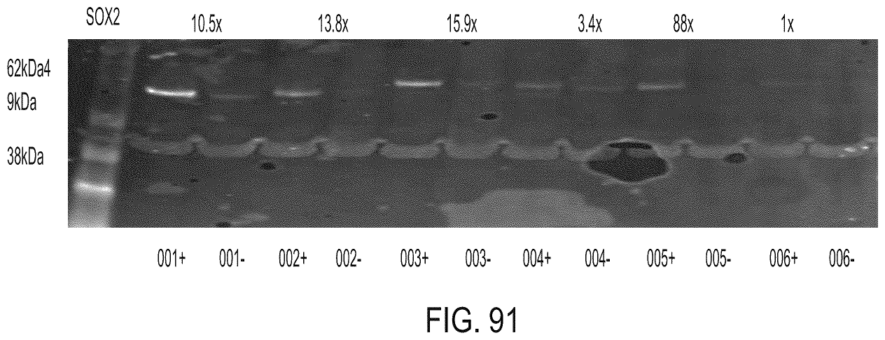

HEK-293T cells were cultured in standard media (DMEM (Fisher Scientific Cat #11-960-044) with 10% FBS (Fisher Scientific Cat #10-082-147) and 1% penicillin/streptomycin (P/S, ThermoFisher Cat #15140122). HEK293T cells were transiently transfected using manufacturer's protocol with Lipofectamine 2000 and 1 μg plasmid DNA each of SOX2-001 through SOX2-006X constructs (wherein X is varied) as shown in Table 12 below. Constructs SOX2-001 and SOX2-002 are not oligomerized. Constructs SOX2-003 and SOX2-005 are trimerized with collectin 7 placed either at the N-terminus (SOX2-003) or C-terminus (SOX2-005). Similarly, SOX2-004 and SOX2-006 were tetramerized with a helix from Kv7.4. 100 μM acetazolamide was added one day after transfection, and the cells were further cultured for 1 more day. mCherry was used as a transfection marker for SOX2-001 and SOX2-002 and GFP as a transfection marker for SOX-003 through SOX2-006 and, after checking expression level by FACs, equal number of transfected cells were pelleted and lysed using T-PER Tissue protein extraction reagent (ThermoFisher cat #78510). Lysate was run on NuPAGE 4-12% Bis-Tris gel (ThermoFisher cat #NP0321BOX) and was transferred onto a nitrocellulose membrane (iBLOT2 NC regular stacks (ThermoFisher IB23001)). SOX2 was detected using anti-FOXP3 SOX2 monoclonal antibody (RnD Cat #AF2018-SP) and IRDye 800CW Goat anti-Mouse IgG (LI-COR P/N 926-32210). The results are shown in FIG. 91 and Table 11. Jurkat cells were successfully transduced with SOX2 constructs.

TABLE 11
Transduction Effeciency of SOX2 Constructs.

| | Transduction Efficiency |
|---|---|
| SOX2-001 | 26.5% |
| SOX2-002 | 20.2% |
| SOX2-003 | 26.0% |
| SOX2-004 | 32.4% |
| SOX2-005 | 34.7% |
| SOX2-006 | 38.7% |

TABLE 12
Constructs Used in Example 23

| | |
|---|---|
| SOX2-001 | pELNS-CA2(L156H)-SOX2v1-p2a-mCherry |
| SOX2-002 | pELNS-CA2(L156H)-SOX2v2-p2a-mCherry |
| SOX2-003 | pELNS-Collectin7trimer-ERlinker2-CA2(L156H)- |
| | ERlinker1-SOX2—p2a-EGFP |
| SOX2-004 | pELNS-Kv7.4 tetramer-ERlinker2-CA2(L156H)- |
| | ERlinker1-SOX2—p2a-EGFP |
| SOX2-005 | pELNS-CA2(L156H)-ERlinker1-SOX2-linker- |
| | collectin7 trimer-p2a-EGFP |
| SOX2-006 | pELNS-CA2(L156H)-ERlinker1-SOX2- |
| | linker-Kv7.4 tetramer-p2a-EGFP |

1. An engineered, regulatable polypeptide monomer comprising
(a) at least one payload having a biological activity;
(b) at least one drug responsive domain (DRD), wherein the DRD is operably linked to the payload and wherein the at least one DRD is responsive to a ligand; and
(c) an oligomerization domain configured to promote oligomerization of the polypeptide monomer.

2. The engineered, regulatable polypeptide monomer of claim 1, wherein the biological activity of the at least one payload is modulated by interaction of the at least one DRD and an effective amount of the ligand.

3. The engineered, regulatable polypeptide monomer of claim 1, wherein the oligomerization domain is chosen from a dimerization domain, a trimerization domain, a tetramerization domain, a pentamerization domain, and a hexamerization domain.

4. An oligomer comprising at least two engineered, regulatable polypeptide monomers of claim 1, wherein the monomers are linked through the oligomerization domain of the monomers.

5. The oligomer of claim 4, comprising at least three engineered, regulatable polypeptide monomers of claim 1, wherein the monomers are linked through the oligomerization domain of the monomers.

6. The oligomer of claim 4, wherein the monomers are non-covalently linked through the oligomerization domain of each of the monomers.

7. The oligomer of claim 4, wherein the at least one payload of each of the monomers is one payload, wherein the one payload of each of the monomers is the same, wherein the DRD of each of the monomers is one DRD, and wherein the one DRD of each of the monomers is responsive to the same ligand.

8. The oligomer of claim 4, wherein the at least one payload is a first payload, wherein the first payload in each of the monomers is the same, wherein the biological activity of the first payload in the oligomer has a range spanning from a basal level of activity in the absence of ligand to a maximum activity in the presence of a saturating amount of ligand, and wherein the basal activity level of the first payload of the oligomer is less than that of the same payload in a control construct.

9. The oligomer of claim 4, wherein the at least one payload is a first payload, wherein the first payload in each of the monomers is the same, wherein the biological activity of the first payload in the oligomer has a range spanning from a basal level of activity in the absence of ligand to a maximum activity in the presence of a saturating amount of ligand, and wherein the range of activity for the first payload in the oligomer is greater than that of the same payload in a control construct.

10. An engineered, regulatable polypeptide comprising
(a) at least one payload having a biological activity; and
(b) a modulation hub comprising at least two drug responsive domains (DRD), wherein the modulation hub is operably linked to the payload, wherein the at least two DRDs are responsive to a ligand.

11. The engineered, regulatable polypeptide of claim 10, wherein the biological activity of the payload is modulated by interaction of the at least two DRDs and an effective amount of the ligand.

12. The engineered, regulatable polypeptide of claim 10, wherein the at least two DRDs are responsive to the same ligand.

13. The engineered, regulatable polypeptide of claim 12, wherein the modulation hub comprises three DRDs and wherein the three DRDs are responsive to the same ligand.

14. The engineered, regulatable polypeptide of claim 10, wherein the biological activity of the at least one payload ranges from a basal activity level in the absence of the ligand to a maximum activity level of the payload in the presence of a saturating amount of the ligand and wherein the range of activity level of the payload in the engineered, regulatable polypeptide is greater than that of the same payload in a control construct.

15. The engineered, regulatable polypeptide of claim 10, wherein the payload has a basal level of biological activity in the absence of ligand that is lower than that of the same payload in a control construct.

16. An engineered, regulatable oligomer comprising at least two polypeptide monomers, wherein each monomer comprises at least one payload or one DRD, wherein the oligomer comprises at least one payload operably linked to at least two DRDs, wherein the at least one payload has a biological activity, wherein the at least two DRDs are responsive to a ligand.

17. The engineered, regulatable oligomer of claim 16, wherein the at least two polypeptide monomers each comprise an oligomerization domain and wherein the oligomerization domain of one of the polypeptide monomers comprises a hetero-oligomerization domain.

18. The engineered, regulatable oligomer of claim 16, wherein the at least two DRDs are responsive to the same ligand.

19. The engineered, regulatable oligomer of claim 16, wherein the biological activity of the payload in the oligomer has a range spanning from a basal level of activity in the absence of ligand to a maximum activity in the presence of a saturating amount of ligand and wherein the basal activity level of the oligomer is less than that of the same payload in a control construct.

20. The engineered, regulatable oligomer of claim 17, wherein the at least two polypeptide monomers comprises a first, a second, and a third polypeptide monomer, wherein the first and second polypeptide monomers are the same, wherein each of the first and second polypeptide monomers comprises a DRD, and wherein the third polypeptide monomer comprises a hetero-oligomerization domain and a payload.

21. The engineered, regulatable oligomer of claim 16, wherein the payload to DRD ratio in the oligomer is 1:1.

22. An expressible nucleic acid construct encoding one or more engineered, regulatable polypeptide monomers of claim 1; one or more engineered, regulatable polypeptides of claim 10; or one or more polypeptide monomers of the oligomer of claim 16.

23. A plurality of expressible nucleic acid constructs, wherein each construct encodes an engineered, regulatable polypeptide monomer of claim 1; one or more engineered, regulatable polypeptides of claim 10; or one or more polypeptide monomers of the oligomer of claim 16.

24. A vector comprising one or more expressible nucleic acid constructs of claim 16.

25. A cell comprising one or more expressible nucleic acid constructs of claim 16.

26. The cell of claim 25, wherein the cell expresses at least two polypeptide monomers and wherein the at least two polypeptide monomers form an oligomer.

27. The cell of claim 26, wherein the oligomer comprises at least three polypeptide monomers.

28. A cell comprising the vector of claim 24.

29. The cell of claim 25, wherein the cell is a human cell.

30. The cell of claim 29, wherein the human cell is an immune cell.

31. The engineered, regulatable polypeptide monomer of claims claim 1; the engineered, regulatable polypeptide of claim 10; the engineered, expressible oligomer of claim 16; the expressible nucleic acid construct of claim 16; the vector of claim 24; or the cells of claim 25, wherein the DRDs are chosen from CA2 (SEQ ID NO: 1), ecDHFR (SEQ ID NO:18), hDHFR (SEQ ID NO:37), FKBP (SEQ ID NO: 93), PDE5 ligand binding domain (SEQ ID NO:94), PDE5 (SEQ ID NO:95), and ER (SEQ ID NO:96), and or an amino acid sequence having at least 85% identity to SEQ ID NOs: 1, 18, 37, 93, 94, 95, or 96.

32. The engineered regulatable polypeptide monomer of any one of claims 1-3, the engineered, regulatable polypeptide of claim 10, the expressible nucleic acid construct of claim 16, the vector of claim 24, or the cells of claim 25, wherein the payload is selected from the group consisting of a cytokine, a cytokine receptor, a TCR, a CAR, an immunomodulatory proteins, and combinations thereof.

33. A method of regulating an amount or activity of a payload in the oligomer of claim 4 or in the engineered, regulatable polypeptide of claim 10, comprising contacting the DRDs with a selected dose of the ligand, wherein the selected dose of the ligand results in a selected amount or activity level of the payload.

34. A method of regulating an amount or activity of the at least two payloads in the engineered, regulatable oligomer of any one of claim 16, comprising contacting the at least two DRDs with the ligand, wherein the selected dose of the ligand results in a selected amount or activity level of the at least two payloads.

35. The method of claim 33, wherein the contacting is performed in vivo.

36. A method of delivering a payload to a subject comprising administering to the subject the nucleic acid construct of claim 16 or the vector of claim 24.

37. A method of delivering a payload to a subject comprising administering to the subject the plurality of expressible nucleic acid constructs of claim 23.

38. The method of claim 36, further comprising administering to the subject a selected amount of the ligand to deliver a selected amount or activity of the payload to the subject.

39. The method of claim 36, wherein expression of two or more nucleic acid constructs in a cell of the subject results in expression of two or more polypeptide monomers, wherein the two or more polypeptide monomers form an oligomer.
